(19) United States
(12) Patent Application Publication
(10) Pub. No.: US 2022/0290200 A1
(43) Pub. Date: Sep. 15, 2022

(54) GENETICALLY MODIFIED HOST CELLS PRODUCING GLYCOSYLATED CANNABINOIDS

(71) Applicant: OCTARINE BIO IVS, Copenhagen (DK)

(72) Inventors: Nicholas Stuart William MILNE, Copenhagen (DK); Camilla Knudsen BADEN, Copenhagen (DK); Nethaji Janeshwari GALLAGE, Copenhagen (DK)

(73) Assignee: OCTARINE BIO IVS, Copenhagen (DK)

(21) Appl. No.: 17/614,483

(22) PCT Filed: May 26, 2020
(86) PCT No.: PCT/EP2020/064605

§ 371 Date: Nov. 26, 2021

(30) Foreign Application Priority Data
May 27, 2019 (EP) 19176773.0

(51) Int. Cl. C12P 19/44; C12N 9/10; C12N 15/52; A61K 31/7048; A61K 31/7034; C12P 19/60

(52) U.S. Cl. CPC C12P 19/44; C12N 9/1048; C12N 15/52; A61K 31/7048; A61K 31/7034; C12P 19/60

(57) ABSTRACT

The present invention relates to a microbial host cell genetically modified to intracellularly produce a cannabinoid glycoside, said cell expressing a heterologous gene encoding a glycosyl transferase which has a at least 70% identity to the glycosyl transferase comprised in SEQ ID NO: 157 or 207, capable of intracellularly glycosylating a cannabinoid acceptor with a glycosyl donor thereby producing the cannabinoid glycoside.

### FIELD OF THE INVENTION

The present invention relates to genetically modified host cells intracellularly producing cannabinoid glycosides; to recombinant polynucleotide constructs and vectors useful for such host cell, to cell cultures of such host cells; to methods of producing cannabinoid glycosides, to fermentation liquids resulting from such methods; to compositions and preparations comprising such fermentation liquid; and to the use of such compositions and preparations.

### BACKGROUND OF THE INVENTION

Cannabinoids derived from plants such as Cannabis sativa have been consumed for their medicinal properties for thousands of years. Over 100 cannabinoid molecules have been isolated from plants, many with therapeutic relevance for a variety of human disease conditions. In recent times cannabinoids, and in particular cannabidiol (CBD) and Δ-9-tetrahydrocannabinol (THC) have been approved and used as therapeutic drugs for a variety of conditions. CBD and THC are the most well studied cannabinoids likely due to the fact that they are the most abundant cannabinoids found in plants.

While cannabinoids are seen as promising for therapeutic treatments, there are several properties that make most cannabinoids less useful as therapeutic molecules. Cannabinoids are highly lipophilic, have low bioavailability and are quickly eliminated from the body. Moreover, some cannabinoids, in particular THC, is psychoactive, meaning that they may have to be administered at sub-optimal dosage to avoid triggering serious side effects. Further, cannabinoids are also chemically unstable and rapidly degrade even under ambient conditions. Accordingly, such undesirable properties are limiting the therapeutic potential of cannabinoids and prevent development of effective treatments. Hence, improvements of the pharmacokinetic and/or therapeutic properties of cannabinoids are needed. WO2017053574 propose making a cannabinoid glycoside prodrug by incubating a cannabinoid aglycone with sugar donors in the presence of a glycosyl transferase. WO2019014395 suggest expressing a glycosyl transferase in a yeast cell culture suspension and then introduce a cannabinoid to the suspension to generate water soluble cannabinoids.

Production of cannabinoids, in planta, requires plant cells to perform a plethora of different enzyme mediated chemical reactions in concert (pathways) and while it is in principle understood that plant enzyme polypeptides and polynucleotides encoding them, are instrumental for in planta synthesis of cannabinoids, many aspects of cannabinoid pathways are yet to be explored, not only which polypeptides are relevant for producing a particular cannabinoid in nature, but also which polypeptides/enzymes can be implemented to produce cannabinoids ex planta, for example in heterologous host cells, and in particular which polypeptides/enzymes are capable of producing better yields of a desired cannabinoid when produced by ex planta biosynthetic manufacturing methods. Accordingly, there remain a need for cannabinoids with improved pharmacokinetic and/or therapeutic properties as well as methods for the efficient production of such improved cannabinoids.

### SUMMARY OF THE INVENTION

The inventors of the present invention have found glycosyl transferases, which not only surprisingly integrate and work to produce cannabinoid glycosides intracellularly in genetically modified host cells, but also exhibit significant improvements in producing cannabinoid glycosides over hitherto known methodology. Accordingly, in a first aspect this invention provides a microbial host cell genetically modified to intracellularly produce a cannabinoid glycoside, said cell expressing a heterologous gene encoding at least one glycosyl transferase capable of intracellularly glycosylating a cannabinoid acceptor with a glycosyl or sugar donor thereby producing the cannabinoid glycoside.

In a further aspect the invention provides a polynucleotide construct comprising a polynucleotide sequence encoding the glycosyl transferase of the invention, operably linked to one or more control sequences heterologous to the glycosyl encoding polynucleotide.

In a further aspect the invention provides an expression vector comprising the polynucleotide construct of the invention.

In a further aspect the invention provides a genetically modified host cell comprising the polynucleotide construct or the vector of the invention.

In a further aspect the invention provides a cell culture, comprising the genetically modified host cell of the invention and a growth medium.

In a further aspect the invention provides a method for producing a cannabinoid glycoside comprising:
a) culturing the cell culture of the invention at conditions allowing the genetically modified host cell to produce the cannabinoid glycoside; and
b) optionally recovering and/or isolating the cannabinoid glycoside.

In a further aspect the invention provides a fermentation liquid comprising the cannabinoid glycosides comprised in the cell culture of of the invention.

In a further aspect the invention provides a composition comprising the fermentation liquids or cannabinoid glycosides of the invention and one or more agents, additives and/or excipients.

In a further aspect the invention provides a cannabinoid glycoside comprising a cannabinoid aglycone or cannabinoid glycoside covalently linked to a sugar selected from xylose; rhamnose; galactose; N-acetylglucosamine; N-acetylgalactosamine; and arabinose or comprising a cannabinoid aglycone or cannabinoid glycoside covalently linked to glycosidic moiety by a 1,4- or 1,6-glycosidic bond.

In a further aspect the invention provides a method for preparing a pharmaceutical preparation comprising mixing the composition of the invention with one or more pharmaceutical grade excipient, additives and/or adjuvants.

In a further aspect the invention provides a pharmaceutical preparation obtainable from the method of the invention for preparing the pharmaceutical preparation.

In a further aspect the invention provides a pharmaceutical preparation obtainable from the method of the invention for preparing the pharmaceutical preparation for use as a medicament.

In a further aspect the invention provides a method for treating a disease in a mammal, comprising administering a therapeutically effective amount of the pharmaceutical preparation of the invention to the mammal.

### DESCRIPTION OF DRAWINGS AND FIGURES

FIG. 1 shows the pathway for microbial production of cannabinoids from glucose.

FIG. 2 shows a schematic demonstrating in vivo homologous recombination of multiple integration fragments in S. cerevisiae.

FIG. 3 shows the biosynthetic pathway for the production of cannabinoids and cannabinoid glycosides resulting from the introduction of plasmids described in Example-17 in S. cerevisiae.

FIG. 4 shows the structures of cannabinoid glycosides validated by LC-MS-QTOF.

FIG. 5 shows an example of LC-MS-QTOF chromatogram from in vitro conversion of CBG to CBG-glycosides by Cs73 Y.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

The term “ACT” as used herein refers to an acetoacetyl-CoA thiolase enzyme (EC 2.3.1.9) capable of converting two acetyl-CoA molecules into acetoacetyl-CoA. ACT is also known as ERG10.

The term “HCS” as used herein refers to hydroxymethylglutaryl-CoA (HMG-CoA) synthase enzyme (EC 4.1.3.5) capable of converting acetoacetyl-CoA and Acetyl-CoA into HMG-CoA. HCS is also known as ERG13.

The term “HCR” as used herein refers to a HMG-CoA reductase (EC1.1.1.34) capable of converting HMG-CoA into Mevalonate.

The term “MVK” as used herein refers to a mevalonate kinase (EC2.7.1.36) capable of converting mevalonate into mevalonate-5-phosphate. MVK is also known as ERG12.

The term “PMK” as used herein refers to a phosphomevalonate kinase (EC2.7.4.2) capable of converting Mevalonate-5-phosphate into Mevalonate diphosphate. PMK is also known as ERGS.

The term “MPC” as used herein refers to a mevalonate pyrophosphate decarboxylase (EC4.1.1.33) capable of converting mevalonate diphosphate into isopentenyl diphosphate (IPP). MPC is also known as MVD1.

The term “IPI” as used herein refers to an isopentenyl diphosphate isomerase (EC5.3.3.2) capable of converting IPP into dimethylallyl diphosphate (DMAPP). IPI is also known as ID11.

The term “GPPS” as used herein refers to a Geranyl diphosphate synthase (EC2.5.1.1) capable of convertion DMAPP and IPP into geranyl diphosphate (GPP).

The term “AAE” as used herein refers to an Acyl activating Enzyme (EC6.2.1.2) capable of converting Acetyl-CoA and hexanoic acid or Acetyl-CoA and butanoic acid into Hexanoyl-CoA or butanoyl-CoA respectively.

The term “TKS” as used herein refers to a 3,5,7-Trioxododecanoyl-CoA synthase (EC2.3.1.206) capable of converting hexanoyl-CoA and malonyl-CoA or butanoyl-CoA and malonoyl-CoA into 3,5,7-trioxododecanoyl-CoA or 3,5,7-trioxoundecanoyl-CoA respectively. TKS is also known as olivetol synthase.

The term “OAC” as used herein refers to a 3,5,7-trioxododecanoyl-CoA cyclase or a 3,5,7-trioxoundecanoyl-CoA cyclase (EC4.4.1.26) capable of converting 3,5,7-trioxododecanoyl-CoA into Olivetolic acid or 3,5,7-trioxoundecanoyl-CoA into divarinolic acid respectively. OAC is also known as Olivetolic Acid Cyclase.

The term “CBGAS” as used herein refers to a cannabigerolic acid synthase (2.5.1.102) capable of converting GPP and Olivetolic acid (OA) or GPP and divarinolic acid (DVA) into to cannabigerolic acid (CBGA) or cannabigerovarinic acid (CBGVA) respectively.

The term “CBDAS” as used herein refers to a cannabidiolic acid synthase (EC1.21.3.8) capable of converting CBGA or CBGVA into cannabidiolic acid (CBDA) or cannabidivarinic acid (CBDVA) respectively.

The term “THCAS” as used herein refers to a tetrahydrocannabinolic acid synthase (EC1.21.3.7) capable of converting CBGA or CBGVA into tetrahydrocannabinolic acid (THCA) or tetrahydrocannabivarinic acid (THCVA) respectively.

The term “CBCAS” as used herein refers to a cannabichromenic acid synthase (EC1.21.99.- or EC1.3.3.-) capable of converting CBGA or CBGVA into cannabichromenic acid (CBCA) or annabichromevarinic acid respectively.

The term “glycosyl transferase” or “GT” as used herein refers to enzymes (EC2.4) that catalyze formation of glycosides by transfer of a glycosyl group (sugar) from an activated glycosyl donor to a nucleophilic glycosyl acceptor molecule, the nucleophile of which can be oxygen- carbon-, nitrogen-, or sulfur-based and in particular. The product of glycosyl transfer may be an O-, N-, S-, or C-glycoside. In the context of the present invention the nucleophilic glycosyl acceptor is a cannabinoid or a cannabinoid glycoside and the product of glycosyl transfer is an O- or C-glycoside.

The term “nucleotide glycoside” as used herein about glycosyl donors refers to compounds comprising a nucleotide moiety covalently linked to a glycosyl group, where the nucleotide comprise a nucleoside covalently linked to one or more phosphate groups. Such compounds are also referred to as “activated glycosides” and where the glycosyl group is a sugar as “nucleotide sugars” or “activated sugars”.

The term “heterologous” or “recombinant” and its grammatical equivalents as used herein refers to entities “derived from a different species or cell”. For example, a heterologous or recombinant polynucleotide gene is a gene in a host cell not naturally containing that gene, i.e. the gene is from a different species or cell type than the host cell.

The term “genetically modified host cell” as used herein refers to host cell comprising and expressing heterologous or recombinant polynucleotide genes.

The term “substrate” or “precursor”, as used herein refers to any compound that can be converted into a different compound. For example, IPP can be a substrate for IPI converting IPP into DMAPP. For clarity, substrates and/or precursors include both compounds generated in situ by an enzymatic reaction in a cell or exogenously provided compounds, such as exogenously provided organic carbon molecules which the host cell can metabolize into a desired compound.

The term “metabolic pathway” as used herein is intended to mean two or more enzymes acting in a chain of reaction (sequentially or interrupted by intermediate steps) in a live cell to convert chemical substrate(s) into chemical product(s). Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds such as proteins for example enzymes (co-enzymes). NADPH and NAD+ are examples of co-factors

The term “operative biosynthetic metabolic pathway” refers to a metabolic pathway that occurs in a live recombinant host, as described herein.

The term “in vivo”, as used herein refers to within a living cell, including, for example, a microorganism or a plant cell (in planta).

The term “in vitro”, as used herein refers to outside a living cell, including, without limitation, for example, in a microwell plate, a tube, a flask, a beaker, a tank, a reactor and the like.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, using these deviating terms can also include a range deviation plus or minus such as plus or minus 9%, 8%, 7%, 6%, 5%, 4%, 3%, 2%, or 1% from a specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The terms “isolated” or “purified” or “extracted” or “recovered” as used herein interchangably about a compound, refers to any compound, which by means of human intervention, has been put in a form or environment that differs from the form or environment in which it is found in nature. Isolated compounds include, but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other exogenous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other exogenous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” is used herein about the relatedness between two amino acid sequences or between two nucleotide sequences. “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

$$\frac{iden tical amino acid residues}{L ength of alignment - total number of gaps in alignment} \times 100$$

“% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.
   Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a linear or circular DNA molecule that comprises a polynucleotide encoding a polypeptide and is operably linked to control sequences that provide for its expression.

The term “host cell” refers to any cell type that is susceptible to transformation, transfection, transduction, or the like with a polynucleotide construct or expression vector comprising a polynucleotide of the present invention. The term “host cell” encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises one or more control sequences.

The term “operably linked” refers to a configuration in which a control sequence is placed at an appropriate position relative to the coding polynucleotide such that the control sequence directs expression of the coding polynucleotide.

The terms “nucleotide sequence” and “polynucleotide” are used herein interchangeably.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The term “cell culture” as used herein refers to a culture medium comprising a plurality of genetically modified host cells of the invention. A cell culture may comprise a single strain of genetically modified host cells or may comprise two or more distinct strains of genetically modified host cells. The culture medium may be any medium suitable for the genetically modified host cells, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

The terms “1′-O” and “3′-O” refers to the OH group at the 1′ and 3′ position on cannabinoids. Due to the symmetrical nature of cannabinoids that contain two OH groups (e.g. CBD, CBDV, CBG) and the free rotation that occurs in these molecules, the terms “1′-O” and “3′-O” can be used interchangeably. E.g. it is understood that CBD-1′-O-β-D-xyloside and CBD-3′-O-β-D-xyloside can be used interchangeably to describe the same molecule.

The terms “di-glycoside”, “tri-glycoside” and “tetra-glycoside” refer to molecules with 2, 3, and 4 glycoside moieties attached together at any O-linkage. E.g. CBD-1′-O-β-D-di-xyloside refers to a CBD molecule with 1 xylose sugar attached at the 1′ position of CBD, and a second xylose sugar attached at any position on the first xylose sugar.

The terms “gentiobioside”, “cellobioside” and “laminaribioside” refer to molecules that are di-glucosides in which two glucose moieties are linked by an O-β-glycosidic bond at the 1,6-, 1,4- or 1,3-position, respectively.

Glycosyltransferases may further be divided into different GT families depending on the 3D structure and reaction mechanism. More specifically the GT1 superfamily refers to UDP glycosyltransferases (UGTs) containing the PSPG box binding UDP-sugars. UGT-superfamily members may further be divided into families and subfamilies as defined by the UGT Nomenclature Committee (Mackenzie et al., 1997) depending on the amino acid identity. Identities >40% belong to the same UGT-family e.g. UGT73 and amino acid identities >60% defines the subfamily e.g. UGT73Y.

### Genetically Modified Host Cells

In one aspect the invention provides a microbial host cell genetically modified to intracellularly produce a cannabinoid glycoside, said cell expressing a heterologous gene encoding at least one glycosyl transferase capable of intracellularly glycosylating a cannabinoid acceptor with a glycosyl donor thereby producing the cannabinoid glycoside.

### Cannabinoid Acceptors

The cannabinoid acceptor may be a condensation product or a derivative thereof a prenyl donor and a prenyl acceptor. The cannabinoid acceptor can be a cannabinoid aglycone or a cannabinoid glycoside.

The prenyl donor can be selected from the group of Gernyl diphosphate, Neryl diphosphate, Farnesyl diphosphate, Dimethylallyl diphosphate and Geranylgeranyl pyrophosphate. In particular the prenyl donor is geranyl diphosphate (GPP). The prenyl acceptor may be a derivative of a fatty acid selected from the group of hexanoic acid, butanoic acid, pentanoic acid, heptanoic acid, octanoic acid, nonanoic acid, decanoic acid; 4-methyl hexanoic acid, 5-hexanoic acid and 6-heptanoic acid. In particular the prenyl acceptor is selected among the group of olivetolic acid, divarinolic acid, olivetol, phlorisovalerophenone, resveratrol, naringenin, phloroglucinol and homogentisic acid and in one embodiment the prenyl acceptor is olivetolic acid and/or divarinolic acid.

Suitable cannabinoid acceptors are those where the cannabinoid acceptor and/or the cannabinoid glycoside have affinity to act as an agonist or an antagonist to a human or animal cannabinoid receptor. Different cannabinoid receptors are known for humans including but not limited to CB1, CB2, GPR55, 5-HT1A, TRPV1 and TRPA1. Some cannabinoid acceptors are known to be psychoactive, such as THC, which is thought to bind to the CB1 Receptor in the brain and through intracellular activation, induce anandamide and 2-arachidonoylglycerol synthesis produced naturally in the body and brain. In one embodiment cannabinoid acceptor is non-psychotropic or at least 25% less psychotropic than THC when assayed for example by using HTS019RTA—READY-TO-ASSAY™ CB1 CANNABINOID RECEPTOR FROZEN CELLS available from Eurofins (https://www.eurofinsdiscovery.com/HTS019RTA-Ready-to-Assay-CB1-Cannabinoid-Receptor-Frozen-Cells/). Preferably the cannabinoid acceptor and/or the cannabinoid glycoside is at least 50% less non-psychotropic than THC, such as at least 75% less psychotropic, or at least 80%, or at least 90% or at least 95% less psychotropic than THC.

The cannabinoid acceptor is typically neutral or acidic and may in an embodiment be selected from the group of cannabichromene-type (CBC), cannabigerol-type (CBG), cannabidiol-type (CBD), Tetrahydrocannabinol-type (THC), cannabicyclol-type (CBL), cannabielsoin-type (CBE), cannabinol-type (CBN), cannabinodiol-type (CBND) and cannabitriol-type (CBT). More specifically, the cannabinoid acceptor may be selected from the group of cannabigerolic acid (CBGA), cannabigerolic acid monomethylether (CBGAM), cannabigerol monomethylether (CBGM), cannabigerovarinic acid (CBGVA), cannabigerovarin (CBGV), cannabichromenic acid (CBCA), cannabichromevarinic acid (CBCVA), cannabichromevarin (CBCV), cannabidiolic acid (CBDA), cannabidiol, monomethylether (CBDM), cannabidiol-C₄ (CBD-C₄), cannabidivarinic acid (CBDVA) cannabidivarin (CBDV), cannabidiorcol (CBD-C₁), Δ⁹-trans-tetrahydrocannabinol (Δ⁹-THC), Δ⁹-tetrahydrocannabinol (Δ⁹-THC), Δ⁹-cis-tetrahydrocannabinol (Δ⁹-THC), tetrahydrocannabinolic acid (THCA), Δ⁹-tetrahydrocannabinolic acid A (THCA-A), Δ⁹-tetrahydrocannabinolic acid B (THCA-B), Δ⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-trans-tetrahydrocannabinol (Δ⁸-THC), Δ⁸-tetrahydrocannabinol (Δ⁸-THC), Δ⁸-cis-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitran, cannabicitranic acid, cannabinolic acid, (CBNA), cannabinol methylether (CBNM), cannabinol-C₄, (CBN-C₄), cannabivarin (CBV), cannabinol-C₂ (CNB-C₂), cannabiorcol (CBN-C₁), cannabinodiol, (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin, (CBTVE), dehydrocannabifuran (DCBF), cannabifuran (CBF), cannabichromanon (CBCN), cannabicivan (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-I-benzoxocin-5-methanol (OH-iso-HHCV), cannabiripsol (CBR), trihydroxy-delta-9-tetrahydrocannabinol (triOH-THC), perrottetinene, perrottetinenic acid, 11-Nor-9-carboxy-THC, 11-hydroxy-Δ⁹-THC, Nor-9-carboxy-Δ⁹-tetrahydrocannabinol, tetrahydrocannabiphorol (THCP), cannabidiphorol (CBDP), Cannabimovone (CBM), and derivatives thereof. In another embodiment the cannabinoid acceptor is an endocannabinoid selected from the group of arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),1 I (Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, and docosahexaenoic acid (DHA). Others are listed in Elsohly M. A. and Slade D.; Life Sci. 2005; 78; pp 539-548.

Acidic cannabinoic acceptors can be decarboxylated to their neutral counterparts by heat, light, or alkaline conditions.

### Glycosyl Donors

Suitable glycosyl donors are nucleotide glycosides. Nucleotide glycosides useful for the present invention includes nucleoside triphosphate glycosides (NTP-glycosides), nucleoside diphosphate glycosides (NDP-glycosides) and nucleoside monophosphate glycosides (NMP-glycosides). Sugar mono- or diphosphonucleotides (sometimes termed Leloir donors); and the corresponding GT's are termed Leloir glycosyltransferases. Particularly preferred nucleosides are Uridine, Adenosin, Guanosin, Cytidin and/or deoxythymidine. Useful nucleotide glycosides include uridine diphosphate glycosides (UDP-glycosides), adenosin diphosphate glycosides (ADP-glycosides), cytidin diphosphate glycosides (CDP-glycosides), cytidin monophosphate glycosides (CMP-glycosides), deoxythymidine diphosphate glycosides (dTDP-glycosides) and guanosin diphosphosphate glycosides (GDP-glycosides).

Particularly useful UDP-glycosyl donors are UDP-D-glucose (UDP-Glc); UDP-galactose (UDP-Gal); UDP-D-xylose (UDP-Xyl); UDP-N-acetyl-D-glucosamine (UDP-GlcNAc); UDP-N-acetyl-D-galactosamine (UDP-GaINAc); UDP-D-glucuronic acid (UDP-GlcA); UDP-L-rhamnose (UDP-Rham); UDP-D-galactofuranose (UDP-Galf); UDP-arabinose; UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate and/or UDP-3-amino-3-deoxy-α-D-glucose. Other useful nucleotide glycoside glycosyl donors are guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo). Also adenosin diphospho sugars (ADP-sugars), such as ADP-Glc, are useful as glycosyl donor. In particular the donor is UDP and the GT is an UDP dependent glycosyl transferase (an UGT).

### Glycosyl Transferases

The glycosyl transferase of the invention may be derived from an eukaryotic, prokaryotic or archaic source. In one embodiment the source is eukaryote such as a mammal (eg. human), plant or a fungus. Useful plants include but are not limited to Oryza sativa, Crocus sativus, Nicotiana tabacum, Stevia rebaudiana, Nicotiana benthamiana and Arabidopsis thaliana. Further, the glycosyl transferase may capable of glycosylating cannabinoids using a nucleotide glycoside such as NTP-glycoside, NDP-glycoside and/or NMP-glycoside as glycosyl donor. In particular glycosyl transferases capable of using nucleotide glycosides where the nucleoside is selected from Uridine, Adenosin, Guanosin, Cytidin and deoxythymidine as glycosyl donors are useful. In a further embodiment, the glycosyl transferease can glycosylate cannabinoids using a glycosyl donor is selected from UDP-glycosides, ADP-glycosides, CDP-glycosides, CMP-glycosides, dTDP-glycosides and GDP-glycosides. Particularly, UDP- and/or an ADP-glycosyl transferases are useful.

Further useful glycosyl transferases are those which can glycosylate cannabinoids using a glycosyl donor selected from one or more of UDP-D-glucose (UDP-Glc); UDP-D-galactose (UDP-Gal); UDP-D-xylose (UDP-Xyl); UDP-L-rhamnose (UDP-Rham); UDP-N-acetyl-D-glucosamine (UDP-GlcNAc); UDP-N-acetyl-D-galactosamine (UDP-GaINAc); UDP-D-glucuronic acid (UDP-GlcA); UDP-D-galactofuranose (UDP-Galf); UDP-L-arabinose; UDP-D-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate and UDP-3-amino-3-deoxy-α-D-glucose. Other useful glycosyl donors are guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo).

Further useful glycosyl transferases are cannabinoid aglycone O-glycosyltransferases; cannabinoid glycoside O-glycosyltransferase; cannabinoid aglycone O-glucosyltransferase; cannabinoid aglycone O-rhamnosyltransferases; cannabinoid aglycone O-xylosyltransferases; cannabinoid aglycone O-arabinosyltransferases; cannabinoid aglycone O—N-acetylgalactosaminyl transferases; cannabinoid aglycone O—N-acetylglucosaminyl transferases; cannabinoid aglycone/glycoside mono-O-glycosyltransferases; cannabinoid aglycone/glycoside di-O-glycosyltransferases; cannabinoid aglycone/glycoside tri-O-glycosyltransferases; cannabinoid aglycone/glycoside tetra-O-glycosyltransferases; cannabinoid O-galactosyltransferases and/or cannabinoid O-glucuronosyltransferases.

Still further use glycosyl transferases are O-glycoside transferases and/or C-glycoside transferases. Useful glycosyl transferases can belong to enzymes classes EC2.4.1.- or EC2.4.2.-. Glycosyl transferases from EC2.4.1.-, such as those from EC2.4.1.17 (using UDP-glucuronic acid donors); EC2.4.1.35 (using UDP-glucose donors); EC2.4.1.159 (using UDP-rhamnose donors); EC2.4.1.203 (using UDP-glucose and/or UDP-xylose donors); EC2.4.1.234 (using UDP-galactose donors); EC2.4.1.236 (using UDP-rhamnose donors) and/or EC2.4.1.294 (using UDP-galactose donors) are particularly useful.

A still further useful glycosyl transferase is a cannabinoid aglycone O-glycosyltransferase and/or cannabinoid glycoside O-glycosyltransferase, optionally a cannabinoid aglycone O-glycosyltransferase and/or cannabinoid glycoside O-glycosyltransferase which is a at least 70%, such at least 75%, such as at least 80%, such as at least 90%, such as at least 95%, such as at least 99%, such as 100% identity to the glycosyl transferase comprised in anyone of SEQ ID NO: 1, 3, 5, 7, 9, 11, 13, 15, 17, 19, 21, 23, 25, 27, 29, 31, 33, 35, 37, 39, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205 or 207.

A still further useful glycosyl transferase has at least 70%, such at least 75%, such as at least 80%, such as at least 90%, such as at least 95%, such as at least 99%, such as 100% identity to the cannabinoid aglycone O-glycosyltransferase comprised in anyone of SEQ ID NO: 107, 109, 111, 113, 117, 119, 121, 125, 127, 129, 131, 133, 135, 137, 139, 141, 143, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207.

A still further useful glycosyl transferase is a cannabinoid glycoside O-glycosyltransferase, optionally a cannabinoid glycoside O-glycosyltransferase which has at least 70%, such at least 75%, such as at least 80%, such as at least 90%, such as at least 95%, such as at least 99%, such as 100% identity to the cannabinoid glycoside O-glycosyltransferase comprised in anyone of SEQ ID NO: 115, 123 or 145.

A still further useful glycosyl transferase is a cannabinoid aglycone O-glucosyltransferase, optionally a cannabinoid aglycone O-glucosyltransferase which has at least 70%, such at least 75%, such as at least 80%, such as at least 90%, such as at least 95%, such as at least 99%, such as 100% identity to the cannabinoid aglycone O-glucosyltransferase comprised in anyone of SEQ ID NO: 107, 109, 111, 117, 119, 121, 125, 127, 129, 131, 133, 135, 137, 139, 141, 143, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205 or 207.

A still further useful glycosyl transferase is a cannabinoid aglycone O-rhamnosyltransferase, optionally a cannabinoid aglycone O-rhamnosyltransferase which has at least 70%, such at least 75%, such as at least 80%, such as at least 90%, such as at least 95%, such as at least 99%, such as 100% identity to the cannabinoid aglycone O-rhamnosyltransferase comprised in anyone of SEQ ID NO: 107, 125, 127, 147, 149, 151, 157, 159, 161, 177, 183, 191, 197 or 207.

A still further useful glycosyl transferase is a cannabinoid aglycone O-xylosyltransferase, optionally a cannabinoid aglycone O-xylosyltransferase which has at least 70%, such at least 75%, such as at least 80%, such as at least 90%, such as at least 95%, such as at least 99%, such as 100% identity to the cannabinoid aglycone O-xylosyltransferase comprised in anyone of SEQ ID NO: 107, 113, 125, 127, 147, 149, 151, 157, 159, 161, 177, 183, 191, 197 or 207.

A still further useful glycosyl transferase is a cannabinoid aglycone O-arabinosyltransferase, optionally a cannabinoid aglycone O-arabinosyltransferase which has at least 70%, such at least 75%, such as at least 80%, such as at least 90%, such as at least 95%, such as at least 99%, such as 100% identity to the cannabinoid aglycone O-arabinosyltransferase comprised in anyone of SEQ ID NO: 107, 125, 127, 147, 149, 151, 157, 159, 161, 177, 183, 191, 197 or 207.

A still further useful glycosyl transferase is a cannabinoid aglycone O—N-acetylgalactosaminyl transferase optionally a cannabinoid aglycone O—N-acetylgalactosaminyl transferase which is at least 70%, such at least 75%, such as at least 80%, such as at least 90%, such as at least 95%, such as at least 99%, such as 100% identity to the cannabinoid aglycone O—N-acetylgalactosaminyl transferase comprised in anyone of SEQ ID NO: 107, 125, 127, 147, 149, 151, 157, 159, 161, 177, 183, 191, 197 or 207.

A still further useful glycosyl transferase is a cannabinoid aglycone O—N-acetylglucosaminyl transferase, optionally a cannabinoid aglycone O—N-acetylglucosaminyl transferase which has at least 70%, such at least 75%, such as at least 80%, such as at least 90%, such as at least 95%, such as at least 99%, such as 100% identity to the cannabinoid aglycone O—N-acetylglucosaminyl transferase comprised in anyone of SEQ ID NO: 107, 125, 127, 147, 149, 151, 157, 159, 161, 177, 183, 191, 197 or 207.

A still further useful glycosyl transferase is a cannabinoid aglycone/glycoside di-O-glycosyltransferase, optionally a cannabinoid aglycone/glycoside di-O-glycosyltransferase which has at least 70%, such at least 75%, such as at least 80%, such as at least 90%, such as at least 95%, such as at least 99%, such as 100% identity to the cannabinoid aglycone/glycoside di-O-glycosyltransferase comprised in anyone of SEQ ID NO: 107, 115, 123, 125, 127, 133, 135, 145, 149, 151, 157, 159, 161, 165, 167, 173, 175, 177, 185, 191, 195 or 207.

A still further useful glycosyl transferase is a cannabinoid aglycone/glycoside tri-O-glycosyltransferase, optionally a cannabinoid aglycone/glycoside tri-O-glycosyltransferase which has at least 70%, such at least 75%, such as at least 80%, such as at least 90%, such as at least 95%, such as at least 99%, such as 100% identity to the cannabinoid aglycone/glycoside tri-O-glycosyltransferase comprised in anyone of SEQ ID NO: 107, 115, 123, 145, 157, 159, 191 or 207.

A still further useful glycosyl transferase is a tetra-O-glycosyltransferase, optionally a tetra-O-glycosyltransferase which has at least 70%, such at least 75%, such as at least 80%, such as at least 90%, such as at least 95%, such as at least 99%, such as 100% identity to the cannabinoid aglycone/glycoside tetra-O-glycosyltransferase comprised in anyone of SEQ ID NO: 207.

Grouping of glycosyl transferases into distinct families under the CAZY system is well known to the skilled person. Among glycosyl transferases capable of glycosylating cannabinoids, glycosyl transferases belonging to enzyme family 73 of the CAZY system performs particularly well, so in one embodiment the glycosyl transferase of the invention is a family 73 glycosyl transferase. In particular among family 73 glycosyl transferases, glycosyl transferases which has at least 70%, such at least 75%, such as at least 80%, such as at least 90%, such as at least 95%, such as at least 99%, such as 100% identity to the glycosyl transferase comprised in anyone of SEQ ID NO: 107, 157, 159, 191 and/or 207 are among top performers.

A further top performing glycosyl transferase has at least 70%, such at least 75%, such as at least 80%, such as at least 90%, such as at least 95%, such as at least 99%, such as 100% identity to the glycosyl transferase comprised in anyone of SEQ ID NO: 135, 143, 147 and/or 171.

A still further useful glycosyl transferase has at least 70%, such at least 75%, such as at least 80%, such as at least 90%, such as at least 95%, such as at least 99%, such as 100% identity to the glycosyl transferase glycosylating CBD, CBDV and/or CBDA comprised in anyone of SEQ ID NO: 107, 109, 111, 113, 117, 125, 127, 129, 135, 137, 139, 141, 147, 149, 151, 153, 157, 159, 161, 177, 179, 183, 191, 193, 197, 201, 205 or 207.

A still further useful glycosyl transferase has at least 70%, such at least 75%, such as at least 80%, such as at least 90%, such as at least 95%, such as at least 99%, such as 100% identity to the glycosyl transferase glycosylating CBG, CBGV and/or CBGA comprised in anyone of SEQ ID NO: 107, 109, 119, 125, 127, 135, 137, 147, 149, 151, 157, 159, 161, 165, 167, 173, 175, 177, 179, 183, 185, 187, 189, 191, 195, 201, 205 or 207,

A still further useful glycosyl transferase has at least 70%, such at least 75%, such as at least 80%, such as at least 90%, such as at least 95%, such as at least 99%, such as 100% identity to the THC glycosylating glycosyl transferase comprised in anyone of SEQ ID NO: 107, 111, 117, 121, 125, 127, 131, 143, 149, 155, 157, 159, 163, 169, 171, 191, 199, 201, 203 or, 207.

A still further useful glycosyl transferase has at least 70%, such at least 75%, such as at least 80%, such as at least 90%, such as at least 95%, such as at least 99%, such as 100% identity to the CBN glycosylating glycosyl transferase comprised in anyone of SEQ ID NO: 125, 127, 133, 135, 149, 151, 157, 159, 175, 177, 181, 191, 195 or 207.

A still further useful glycosyl transferase has at least 70%, such at least 75%, such as at least 80%, such as at least 90%, such as at least 95%, such as at least 99%, such as 100% identity to the CBC glycosylating glycosyl transferase comprised in anyone of SEQ ID NO: 107, 125, 127, 135, 149, 151, 157, 159, 175, 177, 191, 201 or 207.

A still further useful glycosyl transferase has at least 70%, such at least 75%, such as at least 80%, such as is least 90%, such as at least 95%, such as at least 99%, such as 100% identity to the glycosyl transferase comprised in SEQ ID NO: SEQ ID NO: 147, 157, 107, 159, 191, 171, 135, 143.

The sequence identities of the glycosyl transferases of the invention to sequences recited herein is in a further embodiment least 90%, such as at least 95%, such as at least 99%, such as 100%.

In another embodiment the glycosyl transferase is selected from one or more of:
a) a glycosyl transferase having at least 70%, such at least 75%, such as at least 80%, such as at least 90%, such as at least 95%, such as at least 99%, such as 100% identity to the UGT708G3 glycosyl transferase of SEQ ID NO: 1;
b) a glycosyl transferase having at least 70%, such at least 75%, such as at least 80%, such as at least 90%, such as at least 95%, such as at least 99%, such as 100% identity to the UGT708G2 glycosyl transferase of SEQ ID NO: 3;
c) a glycosyl transferase having at least 70%, such at least 75%, such as at least 80%, such as at least 90%, such as at least 95%, such as at least 99%, such as 100% identity to the UGT708G1 glycosyl transferase of SEQ ID NO: 5;
d) a glycosyl transferase having at least 70%, such at least 75%, such as at least 80%, such as at least 90%, such as at least 95%, such as at least 99%, such as 100% identity to the OsCGT glycosyl transferase of SEQ ID NO: 7;
e) a glycosyl transferase having at least 70%, such at least 75%, such as at least 80%, such as at least 90%, such as at least 95%, such as at least 99%, such as 100% identity to the FeUGT708C1 glycosyl transferase of SEQ ID NO: 9;
f) a glycosyl transferase having at least 70%, such at least 75%, such as at least 80%, such as at least 90%, such as at least 95%, such as at least 99%, such as 100% identity to the GmUGT708D1 glycosyl transferase of SEQ ID NO: 11;
g) a glycosyl transferase having at least 70%, such at least 75%, such as at least 80%, such as at least 90%, such as at least 95%, such as at least 99%, such as 100% identity to the ZmUGT708A6 glycosyl transferase of SEQ ID NO: 13;
h) a glycosyl transferase having at least 70%, such at least 75%, such as at least 80%, such as at least 90%, such as at least 95%, such as at least 99%, such as 100% identity to the MiCGT glycosyl transferase of SEQ ID NO: 15;
i) a glycosyl transferase having at least 70%, such at least 75%, such as at least 80%, such as at least 90%, such as at least 95%, such as at least 99%, such as 100% identity to the GtUF6CGT1 glycosyl transferase of SEQ ID NO: 17;
j) a glycosyl transferase having at least 70%, such at least 75%, such as at least 80%, such as at least 90%, such as at least 95%, such as at least 99%, such as 100% identity to the DcUGT2 glycosyl transferase of SEQ ID NO: 19;
k) a glycosyl transferase having at least 70%, such at least 75%, such as at least 80%, such as at least 90%, such as at least 95%, such as at least 99%, such as 100% identity to the DcUGT4 glycosyl transferase of SEQ ID NO: 21;
l) a glycosyl transferase having at least 70%, such at least 75%, such as at least 80%, such as at least 90%, such as at least 95%, such as at least 99%, such as 100% identity to the DcUGT5 glycosyl transferase of SEQ ID NO: 23.
m) a glycosyl transferase having at least 70%, such at least 75%, such as at least 80%, such as at least 90%, such as at least 95%, such as at least 99%, such as 100% identity to the UGT7365 glycosyl transferase of SEQ ID NO: 25;
n) a glycosyl transferase having at least 70%, such at least 75%, such as at least 80%, such as at least 90%, such as at least 95%, such as at least 99%, such as 100% identity to the UGT76C5 glycosyl transferase of SEQ ID NO: 27;
o) a glycosyl transferase having at least 70%, such at least 75%, such as at least 80%, such as at least 90%, such as at least 95%, such as at least 99%, such as 100% identity to the UGT73B3 glycosyl transferase of SEQ ID NO: 29;
p) a glycosyl transferase having at least 70%, such at least 75%, such as at least 80%, such as at least 90%, such as at least 95%, such as at least 99%, such as 100% identity to the UGT71E1 glycosyl transferase of SEQ ID NO: 31;
q) a glycosyl transferase having at least 70%, such at least 75%, such as at least 80%, such as at least 90%, such as at least 95%, such as at least 99%, such as 100% identity to the UGT5 glycosyl transferase of SEQ ID NO: 33;
r) a glycosyl transferase having at least 70%, such at least 75%, such as at least 80%, such as at least 90%, such as at least 95%, such as at least 99%, such as 100% identity to the UGT1A10 glycosyl transferase of SEQ ID NO: 35;
s) a glycosyl transferase having at least 70%, such at least 75%, such as at least 80%, such as at least 90%, such as at least 95%, such as at least 99%, such as 100% identity to the UGT1A9 glycosyl transferase of SEQ ID NO: 37; and
t) a glycosyl transferase having at least 70%, such at least 75%, such as at least 80%, such as at least 90%, such as at least 95%, such as at least 99%, such as 100% identity to the UGT2B7 glycosyl transferase of SEQ ID NO: 39.

More specifically in some embodiments the glycosyl transferase is selected from the group consisting of one or more of:
a) a glycosyl transferase having at least 90%, such as at least 95%, such as at least 99%, such as 100% identity to the UGT71E1 glycosyl transferase of SEQ ID NO: 31;
b) a glycosyl transferase having at least at least 90%, such as at least 95%, such as at least 99%, such as 100% identity to the UGT7365 glycosyl transferase of SEQ ID NO: 25;
c) a glycosyl transferase having at least 90%, such as at least 95%, such as at least 99%, such as 100% identity to the UGT76C5 glycosyl transferase of SEQ ID NO: 27;
d) a glycosyl transferase having at least 90%, such as at least 95%, such as at least 99%, such as 100% identity to the UGT73B3 glycosyl transferase of SEQ ID NO: 29;
e) a glycosyl transferase having at least 90%, such as at least 95%, such as at least 99%, such as 100% identity to the UGT5 glycosyl transferase of SEQ ID NO: 33;
f) a glycosyl transferase having at least 90%, such as at least 95%, such as at least 99%, such as 100% identity to the UGT1A10 glycosyl transferase of SEQ ID NO: 35;
g) a glycosyl transferase having at least 90%, such as at least 95%, such as at least 99%, such as 100% identity to the UGT1A9 glycosyl transferase of SEQ ID NO: 37; and
h) a glycosyl transferase having at least 90%, such as at least 95%, such as at least 99%, such as 100% identity to the UGT2B7 glycosyl transferase of SEQ ID NO: 39.

In further embodiments the glycosyl transferase is selected from the group consisting of:
a) a glycosyl transferase having at least 95%, such as at least 99%, such as 100% identity to the UGT71E1 glycosyl transferase of SEQ ID NO: 31;
b) a glycosyl transferase having at least at least 95%, such as at least 99%, such as 100% identity to the UGT7365 glycosyl transferase of SEQ ID NO: 25;
c) a glycosyl transferase having at least 95%, such as at least 99%, such as 100% identity to the UGT76C5 glycosyl transferase of SEQ ID NO: 27;
d) a glycosyl transferase having at least 95%, such as at least 99%, such as 100% identity to the UGT73B3 glycosyl transferase of SEQ ID NO: 29;
e) a glycosyl transferase having at least 95%, such as at least 99%, such as 100% identity to the UGT5 glycosyl transferase of SEQ ID NO: 33;
f) a glycosyl transferase having at least 95%, such as at least 99%, such as 100% identity to the UGT1A10 glycosyl transferase of SEQ ID NO: 35;
g) a glycosyl transferase having at least 95%, such as at least 99%, such as 100% identity to the UGT1A9 glycosyl transferase of SEQ ID NO: 37; and
h) a glycosyl transferase having at least 95%, such as at least 99%, such as 100% identity to the UGT2B7 glycosyl transferase of SEQ ID NO: 39.

In a non-limiting example, the glycosyl transferase is:
a) the UGT71E1 glycosyl transferase of SEQ ID NO: 31;
b) the UGT7365 glycosyl transferase of SEQ ID NO: 25;
c) the UGT76C5 glycosyl transferase of SEQ ID NO: 27;
d) the UGT73B3 glycosyl transferase of SEQ ID NO: 29;
e) the UGT5 glycosyl transferase of SEQ ID NO: 33;
f) the UGT1A10 glycosyl transferase of SEQ ID NO: 35;
g) the UGT1A9 glycosyl transferase of SEQ ID NO: 37; or
h) the UGT2B7 glycosyl transferase of SEQ ID NO: 39.
   The glycosyl transferase of this invention may advantageously be expressed without a signal peptide to avoid targeting the glycosyl transferase for secretion, and to keep it confined for intracellular glycosylation of the cannabinoid acceptor.

A further useful glycosyl transferase catalyzes formation of a 1,2-; 1,3-; 1,4- and/or 1,6-glycosidic bond between the glycosyl group and the cannabinoid aglycone or cannabinoid glycoside. Particularly useful glycosyl transferases catalyzes formation of a 1,4- and/or 1,6-glycosidic bond between the glycosyl group and the cannabinoid aglycone or cannabinoid glycoside. More particularly useful glycosyl transferase catalyzes formation of a 1,4-glycosidic bond between the glycosyl group and the cannabinoid aglycone or cannabinoid glycoside and is the glycosyl transferase comprised in SEQ ID NO: 115. Alternatively, a useful glycosyl transferase catalyzes formation of a 1,6-glycosidic bond between the glycosyl group and the cannabinoid aglycone or cannabinoid glycoside and is the glycosyl transferase comprised in SEQ ID NO: 145.

The genetically modified cell comprises one or more heterologous genes encoding the glycosyl transferase of the invention. These genes may have at least 70%, such at least 75%, such as at least 80%, such as at least 90%, such as at least 95%, such as at least 99%, such as 100% identity to the glycosyl transferase encoding gene comprised in anyone of SEQ ID NO: 2, 4, 6, 8, 10, 12, 14, 16, 18, 20, 22, 24, 26, 28, 30, 32, 34, 36, 38, 40, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 or 208. Particularly useful genes have at least 70%, such at least 75%, such as at least 80%, such as at least 90%, such as at least 95%, such as at least 99%, such as 100% identity to the glycosyl transferase comprised in SEQ ID NO: 148, 158, 108, 160, 192, 172, 137, 144. Preferably, the sequence identity of the genes encoding the glycosyl transferase of the invention to these selected sequences is least 90%, such as at least 95%, such as at least 99%, such as 100%. More preferably, the sequence identity of the genes encoding the glycosyl transferase of the invention to these selected sequences is at least 99%, such as 100%.

In some embodiments the heterologous gene encoding the glycosyl transferase of this invention is selected from one or more of:
a) a polynucleotide having at least 70%, such at least 75%, such as at least 80%, such as at least 90%, such as at least 95%, such as at least 99%, such as 100% identity to SEQ ID NO: 2;
b) a polynucleotide having at least 70%, such at least 75%, such as at least 80%, such as at least 90%, such as at least 95%, such as at least 99%, such as 100% identity to SEQ ID NO: 4;
c) a polynucleotide having at least 70%, such at least 75%, such as at least 80%, such as at least 90%, such as at least 95%, such as at least 99%, such as 100% identity to SEQ ID NO: 6;
d) a polynucleotide having at least 70%, such at least 75%, such as at least 80%, such as at least 90%, such as at least 95%, such as at least 99%, such as 100% identity to SEQ ID NO: 8;
e) a polynucleotide having at least 70%, such at least 75%, such as at least 80%, such as at least 90%, such as at least 95%, such as at least 99%, such as 100% identity to SEQ ID NO: 10;
f) a polynucleotide having at least 70%, such at least 75%, such as at least 80%, such as at least 90%, such as at least 95%, such as at least 99%, such as 100% identity to SEQ ID NO: 12;
g) a polynucleotide having at least 70%, such at least 75%, such as at least 80%, such as at least 90%, such as at least 95%, such as at least 99%, such as 100% identity to SEQ ID NO: 14;
h) a polynucleotide having at least 70%, such at least 75%, such as at least 80%, such as at least 90%, such as at least 95%, such as at least 99%, such as 100% identity to SEQ ID NO: 16; and
i) a polynucleotide having at least 70%, such at least 75%, such as at least 80%, such as at least 90%, such as at least 95%, such as at least 99%, such as 100% identity to SEQ ID NO: 18
j) a polynucleotide having at least 70%, such at least 75%, such as at least 80%, such as at least 90%, such as at least 95%, such as at least 99%, such as 100% identity to SEQ ID NO: 20;
k) a polynucleotide having at least 70%, such at least 75%, such as at least 80%, such as at least 90%, such as at least 95%, such as at least 99%, such as 100% identity to SEQ ID NO: 22;
l) a polynucleotide having at least 70%, such at least 75%, such as at least 80%, such as at least 90%, such as at least 95%, such as at least 99%, such as 100% identity to SEQ ID NO: 24;
m) a polynucleotide having at least 70%, such at least 75%, such as at least 80%, such as at least 90%, such as at least 95%, such as at least 99%, such as 100% identity to SEQ ID NO: 26;
n) a polynucleotide having at least 70%, such at least 75%, such as at least 80%, such as at least 90%, such as at least 95%, such as at least 99%, such as 100% identity to SEQ ID NO: 28;
o) a polynucleotide having at least 70%, such at least 75%, such as at least 80%, such as at least 90%, such as at least 95%, such as at least 99%, such as 100% identity to SEQ ID NO: 30;
p) a polynucleotide having at least 70%, such at least 75%, such as at least 80%, such as at least 90%, such as at least 95%, such as at least 99%, such as 100% identity to SEQ ID NO: 32;
q) a polynucleotide having at least 70%, such at least 75%, such as at least 80%, such as at least 90%, such as at least 95%, such as at least 99%, such as 100% identity to SEQ ID NO: 34;
r) a polynucleotide having at least 70%, such at least 75%, such as at least 80%, such as at least 90%, such as at least 95%, such as at least 99%, such as 100% identity to SEQ ID NO: 36;
s) a polynucleotide having at least 70%, such at least 75%, such as at least 80%, such as at least 90%, such as at least 95%, such as at least 99%, such as 100% identity to SEQ ID NO: 38; and
t) a polynucleotide having at least 70%, such at least 75%, such as at least 80%, such as at least 90%, such as at least 95%, such as at least 99%, such as 100% identity to SEQ ID NO: 40.

More specifically in some embodiments the heterologous gene encoding the glycosyl transferase is selected from the group consisting of one or more of:
a) a polynucleotide having at least 90%, such as at least 95%, such as at least 99%, such as 100% identity to SEQ ID NO: 32;
b) a polynucleotide having at least 90%, such as at least 95%, such as at least 99%, such as 100% identity to SEQ ID NO: 26;
c) a polynucleotide having at least 90%, such as at least 95%, such as at least 99%, such as 100% identity to SEQ ID NO: 28;
d) a polynucleotide having at least 90%, such as at least 95%, such as at least 99%, such as 100% identity to SEQ ID NO: 30;
e) a polynucleotide having at least 90%, such as at least 95%, such as at least 99%, such as 100% identity to SEQ ID NO: 34;
f) a polynucleotide having at least 90%, such as at least 95%, such as at least 99%, such as 100% identity to SEQ ID NO: 36;
g) a polynucleotide having at least 90%, such as at least 95%, such as at least 99%, such as 100% identity to SEQ ID NO: 38; and
h) a polynucleotide having at least 90%, such as at least 95%, such as at least 99%, such as 100% identity to SEQ ID NO: 40.

In further embodiments the heterologous gene encoding the glycosyl transferase is selected from the group consisting of:
a) a polynucleotide having at least 95%, such as at least 99%, such as 100% identity to SEQ ID NO: 32;
b) a polynucleotide having at least 95%, such as at least 99%, such as 100% identity to SEQ ID NO: 26;
c) a polynucleotide having at least 95%, such as at least 99%, such as 100% identity to SEQ ID NO: 28;
d) a polynucleotide having at least 95%, such as at least 99%, such as 100% identity to SEQ ID NO: 30;
e) a polynucleotide having at least 95%, such as at least 99%, such as 100% identity to SEQ ID NO: 34;
f) a polynucleotide having at least 95%, such as at least 99%, such as 100% identity to SEQ ID NO: 36;
g) a polynucleotide having at least 95%, such as at least 99%, such as 100% identity to SEQ ID NO: 38; and
h) a polynucleotide having at least 95%, such as at least 99%, such as 100% identity to SEQ ID NO: 40.
   In a non-limiting example, the heterologous gene encoding the glycosyl transferase is:
i) SEQ ID NO: 32;
j) SEQ ID NO: 26;
k) SEQ ID NO: 28;
l) SEQ ID NO: 30;
m) SEQ ID NO: 34;
n) SEQ ID NO: 36;
o) SEQ ID NO: 38; or
p) SEQ ID NO: 40.

### Cannabinoid Glycosides

The present invention include all cannabinoid glycosides which are combinations of the aforementioned cannabinoid acceptors with the aforementioned glycosyl groups. Using the glycosyl transferases of the invention it is possible to produce glycosylated cannabinoids not previously known, which possesses a range of desirable properties, and/or producing known glycosylated cannabinoids in a more effective way.

Attractive cannabinoid glycosides those which have at least 10% higher water solubility than the corresponding un-glycosylated cannabinoid. Such cannabinoid glycosides include cannabinoid glycosides which have at least 10%, at least 20% at least 40%, at least 60%, at least 80%, at least 100%, at least 200%, and at least 500% higher water solubility than the corresponding un-glycosylated cannabinoid. Some of the cannabinoid glycosides which can be prepared by using the cannabinoid glycosyl transferases of the invention display increased water solubility as high as up to 25 times, such as up to 50 times, such as up to 100 times, such as up to 250 times, such as up to 500 times, such as up to 1000 times the water solubility of the corresponding un-glycosylated cannabinoid. For some cannabinoid glycosides the increased water solubility may above 1000 times the water solubility of the corresponding un-glycosylated cannabinoid. Increased water solubility has a tremendous beneficial effect on not only production by fermentation, but also on administration of the product to patients.

Other attractive cannabinoid glycosides include those which have at least 10% more resistance to UV or heat degradation than the corresponding un-glycosylated cannabinoid. Such cannabinoid glycosides include cannabinoid glycosides which have at least 10%, at least 20% at least 40%, at least 60%, at least 80%, at least 100%, at least 200%, and at least 500% more resistance to UV or heat degradation than the corresponding un-glycosylated cannabinoid. Still other attractive cannabinoid glycosides include those which have at least 10% higher oral uptake in a mammal than the corresponding un-glycosylated cannabinoid, eg. when equally administered to a mammal. Such cannabinoid glycosides include cannabinoid glycosides which have at least 20% at least 40%, at least 60%, at least 80%, at least 100%, at least 200%, and at least 500% higher oral uptake than the corresponding un-glycosylated cannabinoid. In that context oral uptake is to be understood the percentage of an orally ingested dose of the cannabinoid glycoside which is absorbed in the gastrointestinal tract into the body plasma. Still other attractive cannabinoid glycosides include those which have at least 10% higher biological half-life in a mammal than the corresponding un-glycosylated cannabinoid, eg. when equally administered to a mammal. Such cannabinoid glycosides include cannabinoid glycosides which have at least 20% at least 40%, at least 60%, at least 80%, at least 100%, at least 200%, and at least 500% higher biological half-life than the corresponding un-glycosylated cannabinoid. Still other attractive cannabinoid glycosides include those which have at least 10% higher concentration in the cerebrospinal fluid in a mammal at peak concentration than the corresponding un-glycosylated cannabinoid, eg. when equally administered to a mammal. Such cannabinoid glycosides include cannabinoid glycosides which at least 20% at least 40%, at least 60%, at least 80%, at least 100%, at least 200%, and at least 500% higher concentration in the cerebrospinal fluid at peak concentration than the corresponding un-glycosylated cannabinoid. Still other attractive cannabinoid glycosides include those which have at least 10% improved pharmacokinetics compared to the corresponding un-glycosylated cannabinoid, eg. when equally administered to a mammal. Such cannabinoid glycosides include cannabinoid glycosides which have at at least 20% at least 40%, at least 60%, at least 80%, at least 100%, at least 200%, and at least 500% improved pharmacokinetics compared to the corresponding un-glycosylated cannabinoid, as measured by a solubility assay, chemical stability assay, Caco-2 bi-directional permeability assay, hepatic microsomal clearance assay and/or plasma stability assay. Still other attractive cannabinoid glycosides include those which have at least 10% improved stability in acidic aqueous solution compared to the corresponding un-glycosylated cannabinoid, optionally in solution having a pH of 0 to 7, such as a pH of 0.5 to 4, such as a pH of 0.5 to 2, such as a pH of around 1. Still other attractive cannabinoid glycosides include those which have at least 10% improved stability in alkaline aqueous solution compared to the corresponding un-glycosylated cannabinoid, optionally in solution having a pH of 7 to 14, such as a pH of 9 to 14, such as a pH of 10 to 13, such as a pH of around 12.5. Still other attractive cannabinoid glycosides include those which have at least 10% improved resistance to oxidation in aqueous solution compared to the corresponding un-glycosylated cannabinoid, optionally in a solution having at least 8 mg/L O₂, such as at least 20 mg/L O₂, such as at least 40 mg/L O₂, such as at least 80 mg/L O₂, such as such as a solution saturated with O₂. Still other attractive cannabinoid glycosides include those which are at least 10% less toxic to the genetically modified host cell compared to the corresponding un-glycosylated cannabinoid, optionally having a LC50 which is at least 10% less, such as at least 25% less, such as at least 75% less, such as at least 100% less than the corresponding un-glycosylated cannabinoid.

In some embodiments the cannabinoid glycoside is a C-glycoside or an O-glycoside or a combination thereof, particularly such cannabinoid glycoside selected from glycosides of cannabichromene-type (CBC), cannabigerol-type (CBG), cannabidiol-type (CBD), Tetrahydrocannabinol-type (THC), cannabicyclol-type (CBL), cannabielsoin-type (CBE), cannabinol-type (CBN), cannabinodiol-type (CBND) and cannabitriol-type cannabinoid acceptors. A particularly useful cannabinoid glycoside is selected from glycosides of cannabidiol (CBD), cannabidiolic acid (CBDA), cannabidivarin (CBDV), tetrahydrocannabinol (THC), tetrahydrocannabinolic acid (THCA), tetrahydrocannabivarin (THCV), cannabichromevarin (CBCV), cannabigerol (CBG), cannabinol (CBN), 11-nor-9-carboxy-THC and A8-tetrahydrocannabinol. A still further particularly useful cannabinoid glycoside is selected from cannabinoid-1′-O-β-D-glycoside, cannabinoid-1′-O-β-D-glycosyl-3′-O-β-D-glycoside, and cannabinoid-3′-O-β-D-glycoside. A still further particularly useful cannabinoid glycoside is selected from CBD-1′-O-β-D-glycoside, CBD-1′-O-β-D-glycosyl-3′-O-β-D-glycoside, CBDV-r-O-β-D-glycoside, CBDV-1′-O-β-D-glycosyl-3′-O-β-D-glycoside, CBG-1′-O-β-D-glycoside, CBG-1′-O-β-D-glycosyl-3′-O-β-D-glycoside, THC-1′-O-β-D-glycoside, CBN-1′-O-β-D-glycoside, 11-nor-9-carboxy-THC-1′-O-β-D-glycoside, CBDA-1-O-β-D-glycoside and CBC-r-O-β-D-glycoside. A still further particularly useful cannabinoid glycoside is selected from cannabinoid glucosides; cannabinoid glucuronosides; cannabinoid xylosides; cannabinoid rhamnosides; cannabinoid galactosides; cannabinoid N-acetylglucosaminosides; cannabinoid N-acetylgalactosaminosides and cannabinoid arabinosides. A still further particularly useful cannabinoid glycoside is selected from cannabinoid-1′-O-β-D-glucoside; cannabinoid-1′-O-β-D-glucuroside; cannabinoid-1′-O-β-D-xyloside; cannabinoid-1′-O-α-L-rhamnoside; cannabinoid-1′-O-β-D-galactoside; cannabinoid-1′-O-β-D-N-acetylglucosaminoside; cannabinoid-1′-O-β-D-arabinoside; cannabinoid-1′-O-β-D-N-acetylgalactosamine; cannabinoid-1′-O-β-D-glucosyl-3′-O-β-D-glucoside; cannabinoid-1′-O-β-D-cellobioside; cannabinoid-1′-O-β-D-gentiobioside; cannabinoid-1′-O-β-D-glucurosyl-3′-O-β-D-glucuronoside; cannabinoid-1′-O-β-D-xylosyl-3′-O-β-D-xyloside; cannabinoid-1′-O-α-L-rhamnosyl-3′-O-(3-D-rhamnoside; cannabinoid-1′-O-β-D-galactosyl-3′-O-β-D-galactoside; cannabinoid-1′-O-β-D-N-acetylglucosamine-3′-O-β-D-N-acetylglucosaminoside; cannabinoid-1′-O-β-D-arabinosyl-3′-O-β-D-arabinoside; and cannabinoid-1′-O-β-D-N-acetylgalactosamine-3′-O-β-D-N-acetylgalactosamine.

### Operative Biosynthetic Metabolic Pathway Producing Cannabinoid Acceptors

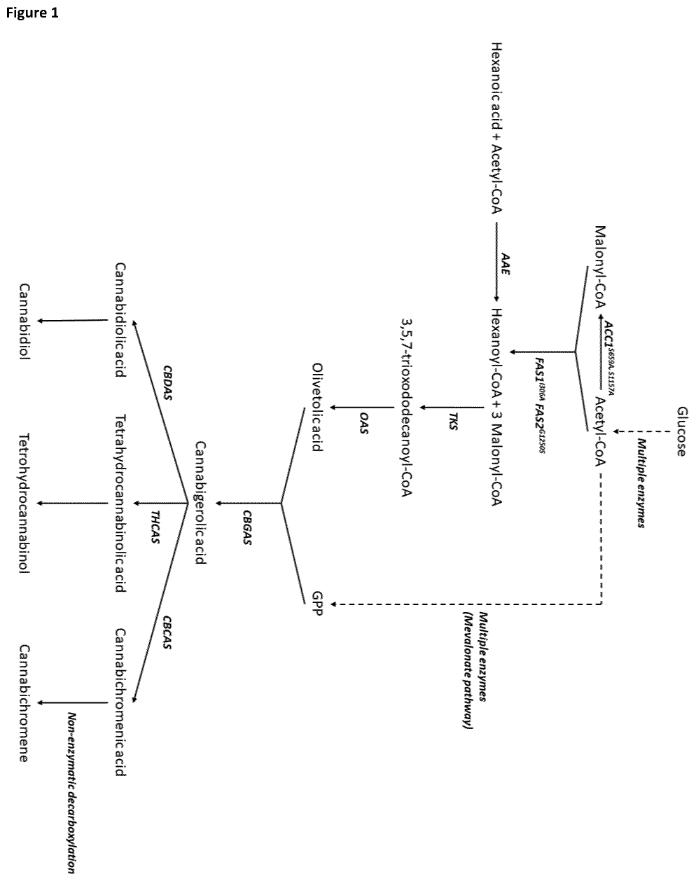

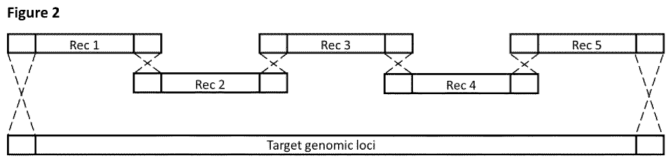

The host cell can advantageously further be modified to include genes producing one or more enzymes in a pathway producing the cannabinoid acceptor from precursors. A flow diagram of the pathway is depicted in FIG. 1. The host cell may comprise all polypeptides required to produce the cannabinoid acceptor from simple nutrient substrates such as glucose, fed from a fermentation medium. However, since substrates and precursors may also be provided to the host cell exogenously, and the host cell pathway may comprise any combination of selected pathway polypeptides, depending on the exogenously provided precursor and the compound desired to be produced by the host cell. The upstream part of the pathway from simple sugars to the basic precursors acetyl-CoA and malonyl-CoA is well known in the art e.g. from van Rossum et al., 2016 and Shi et al., 2014. Further the upstream part of the pathway from simple sugars to fatty acids, such a hexanoic acid is also well known in the art e.g. from Gajewski et al., 2017 or WO2016156548. Downstream from these basic precursors the genetically modified host cell comprises in one embodiment an operative biosynthetic metabolic pathway which comprise one or more polypeptides selected from
a) an acetoacetyl-CoA thiolase (ACT) converting an acetyl-CoA precursor into acetoacetyl-CoA;
b) a HMG-CoA synthase (HCS) converting acetoacetyl-CoA precursor into HMG-CoA;
c) a HMG-CoA reductase (HCR) converting a HMG-CoA precursor into mevalonate;
d) a mevalonate kinase (MVK) converting a mevalonate precursor into Mevalonate-5-phosphate;
e) a phosphomevalonate kinase (PMK) converting a Mevalonate-5-phosphate precursor into Mevalonate diphosphate;
f) a mevalonate pyrophosphate decarboxylase (MPC) converting a Mevalonate diphosphate precursor into isopentenyl diphosphate (IPP);
g) an isopentenyl diphosphate/dimethylallyl diphosphate isomerase (IPI) converting an IPP precursor into dimethylallyl diphosphate (DMAPP);
h) Geranyl diphosphate synthase (GPPS) condensing IPP and DMAPP into into Geranyl diphosphate (GPP);
i) an acyl activating enzyme (AAE) converting a fatty acid precursor into fatty acyl-COA;
j) a 3,5,7-Trioxododecanoyl-CoA synthase (TKS) converting a fatty acid-CoA precursor into 3,5,7-trioxoundecanoyl-CoA;
k) an Olivetolic Acid Cyclase (OAC) converting a 3,5,7-trioxoundecanoyl-CoA precursor into divarinolic acid;
l) an Olivetolic Acid Cyclase (OAC) converting a 3,5,7-trioxododecanoyl-CoA precursor into olivetolic acid;
m) a TKS-OAC fused enzymes converting fatty acid-CoA precursor into 3,5,7-trioxoundecanoyl-CoA, 3,5,7-trioxoundecanoyl-CoA precursor into divarinolic acid and 3,5,7-trioxododecanoyl-CoA precursor into olivetolic acid;
n) a Cannabigerolic acid synthase (CBGAS) condensing GPP and olivetolic acid into Cannabigerolic acid (CBGA);
o) a Cannabigerolic acid synthase (CBGAS) condensing GPP and divarinolic acid into cannabigerovarinic acid (CBGVA);
p) a cannabidiolic acid synthase (CBDAS) converting CBGA acid and/or CBGVA into cannabidiolic acid (CBDA) and/or cannabidivarinic acid (CBDVA), respectively;
q) a tetrahydrocannabinolic acid synthase (THCAS) converting CBGA and/or CBGVA into tetrahydrocannabinolic acid (THCA) and/or tetrahydrocannabivarinic acid (THCVA), respectively;
r) a cannabichromenic acid synthase (CBCAS) converting CBGA and/or CBGVA into cannabichromenic acid (CBCA) and/or cannabichromevarinic acid (CBCVA), respectively;
s) a nucleotide-glucose synthase converting sucrose and nucleotide into fructose and nucleotide-glucose;
t) a nucleotide-galactose 4-epimerase converting nucleotide-glucose into nucleotide-galactose;
u) a nucleotide-(glucuronic acid)-decarboxylase converting nucleotide-glucuronic acid into nucleotide-xylose;
v) a nucleotide-4-keto-6-deoxy-glucose 3,5-epimerase and a nucleotide-4-keto-rhamnose 4-keto-reductase together converting nucleotide-4-keto-6-deoxy-glucose and NADPH into nucleotide-rhamnose and NADP⁺;
w) a nucleotide-glucose 4,6-dehydratase converting nucleotide-glucose and NAD⁺ into nucleotide-4-keto-6-deoxy-glucose and NADH;
x) a nucleotide-glucose 4,6-dehydratase and a nucleotide-4-keto-6-deoxy-glucose 3,5-epimerase and a nucleotide-4-keto-rhamnose-4-keto-reductase together converting nucleotide-glucose and NAD⁺ and NADPH into nucleotide-rhamnose+NADH+NADP⁺;
y) a nucleotide-glucose 6-dehydrogenase converting nucleotide-glucose and 2 NAD⁺ into nucleotide-glucuronic acid and 2 NADH;
z) a nucleotide-arabinose 4-epimerase converting nucleotide-xylose into nucleotide-arabinose; and
aa) a nucleotide-N-acetylglucosamine 4-epimerase converting nucleotide-N-acetylglucosamine into nucleotide-N-acetylgalactosamine.

The nucleotide-glucose synthase of step is also known as a sucrose synthase, due to its ability to also catalyse the reversible reaction.

As examples of specific enzymes which may be comprised in the pathway the
a) ACT has at least 70%, such at least 75%, such as at least 80%, such as at least 90%, such as at least 95%, such as at least 99%, such as 100% identity to the native Erg10 in S. cerevisiae;
b) HCS has at least 70%, such at least 75%, such as at least 80%, such as at least 90%, such as at least 95%, such as at least 99%, such as 100% identity to the native Erg13 in S. cerevisiae;
c) HCR has at least 70%, such at least 75%, such as at least 80%, such as at least 90%, such as at least 95%, such as at least 99%, such as 100% identity to the native HMG1 or HMG2 in S. cerevisiae;
d) MVK has at least 70%, such at least 75%, such as at least 80%, such as at least 90%, such as at least 95%, such as at least 99%, such as 100% identity to the native Erg12 in S. cerevisiae;
e) PMK has at least 70%, such at least 75%, such as at least 80%, such as at least 90%, such as at least 95%, such as at least 99%, such as 100% identity to the native Erg8 in S. cerevisiae;
f) MPC has at least 70%, such at least 75%, such as at least 80%, such as at least 90%, such as at least 95%, such as at least 99%, such as 100% identity to the native MVD1 in S. cerevisiae;
g) IPI has at least 70%, such at least 75%, such as at least 80%, such as at least 90%, such as at least 95%, such as at least 99%, such as 100% identity to the native ID11 in S. cerevisiae;
h) GPPS has at least 70%, such at least 75%, such as at least 80%, such as at least 90%, such as at least 95%, such as at least 99%, such as 100% identity to the GPPS comprised in SEQ ID NO: 45 or 229;
i) AAE has at least 70%, such at least 75%, such as at least 80%, such as at least 90%, such as at least 95%, such as at least 99%, such as 100% identity to the AAE comprised in SEQ ID NO: 47 or 239;
j) TKS has at least 70%, such at least 75%, such as at least 80%, such as at least 90%, such as at least 95%, such as at least 99%, such as 100% identity to the TKS comprised in SEQ ID NO: 49;
k) OAC has at least 70%, such at least 75%, such as at least 80%, such as at least 90%, such as at least 95%, such as at least 99%, such as 100% identity to the OAC comprised in SEQ ID NO: 51;
l) TKS-OAC fused enzyme at least 70%, such at least 75%, such as at least 80%, such as at least 90%, such as at least 95%, such as at least 99%, such as 100% identity to the TKS-OAC fused enzyme comprised in SEQ ID NO 227;
m) CBGAS has at least 70%, such at least 75%, such as at least 80%, such as at least 90%, such as at least 95%, such as at least 99%, such as 100% identity to the CBGAS comprised in SEQ ID NO: 53, 235 or 237;
n) CBDAS has at least 70%, such at least 75%, such as at least 80%, such as at least 90%, such as at least 95%, such as at least 99%, such as 100% identity to the CBDAS comprised in SEQ ID NO: 57 or 233;
o) THCAS has at least 70%, such at least 75%, such as at least 80%, such as at least 90%, such as at least 95%, such as at least 99%, such as 100% identity to the THCAS comprised in SEQ ID NO: 55 or 231;
p) CBCAS has at least 70%, such at least 75%, such as at least 80%, such as at least 90%, such as at least 95%, such as at least 99%, such as 100% identity to the CBCAS comprised in SEQ ID NO: 59;
q) nucleotide-glucose synthase is an UDP-glucose synthase and has at least 70%, such at least 75%, such as at least 80%, such as at least 90%, such as at least 95%, such as at least 99%, such as 100% identity to the UDP-glucose synthase comprised in SEQ ID NO: 209;
r) nucleotide-galactose 4-epimerase is an UDP-galactose 4-epimerase and has at least 70%, such at least 75%, such as at least 80%, such as at least 90%, such as at least 95%, such as at least 99%, such as 100% identity to the UDP-galactose 4-epimerase comprised in SEQ ID NO: 211;
s) nucleotide-(glucuronic acid)-decarboxylase is an UDP-glucuronic acid decarboxylase and has at least 70%, such at least 75%, such as at least 80%, such as at least 90%, such as at least 95%, such as at least 99%, such as 100% identity to the UDP-glucuronic acid decarboxylase comprised in SEQ ID NO: 213;
t) nucleotide-4-keto-6-deoxy-glucose 3,5-epimerase is an UDP-4-keto-6-deoxy-glucose 3,5-epimerase and has at least 70%, such at least 75%, such as at least 80%, such as at least 90%, such as at least 95%, such as at least 99%, such as 100% identity to the UDP-4-keto-6-deoxy-glucose 3,5-epimerase comprised in SEQ ID NO: 215 or 219;
u) nucleotide-4-keto-rhamnose-4-keto reductase is an UDP-4-keto-rhamnose-4-keto reductase and has at least 70%, such at least 75%, such as at least 80%, such as at least 90%, such as at least 95%, such as at least 99%, such as 100% identity to the UDP-4-keto-rhamnose-4-keto reductase comprised in SEQ ID NO: 215 or 219;
v) nucleotide-glucose 4,6 dehydratase is an UDP-glucose 4,6-dehydratase and has at least 70%, such at least 75%, such as at least 80%, such as at least 90%, such as at least 95%, such as at least 99%, such as 100% identity to the UDP-glucose 4,6-dehydratase comprised in SEQ ID NO: 217 or 219;
w) nucleotide-glucose 6-dehydrogenase is an UDP-glucose 6-dehydrogenase and has at least 70%, such at least 75%, such as at least 80%, such as at least 90%, such as at least 95%, such as at least 99%, such as 100% identity to the UDP-glucose 6-dehydrogenase comprised in SEQ ID NO: 221;
x) nucleotide-arabinose 4-epimerase is an UDP-arabinose 4-epimerase and has at least 70%, such at least 75%, such as at least 80%, such as at least 90%, such as at least 95%, such as at least 99%, such as 100% identity to the UDP-arabinose 4-epimerase comprised in SEQ ID NO: 223; and
y) nucleotide-N-acetylglucosamine 4-epimerase is an UDP-N-acetylglucosamine 4-epimerase and has at least 70%, such at least 75%, such as at least 80%, such as at least 90%, such as at least 95%, such as at least 99%, such as 100% identity to the UDP-N-acetylglucosamine 4-epimerase comprised in SEQ ID NO: 225.

SEQ ID NO: 232 and SEQ ID NO: 230 are both N-terminal truncated polypeptides containing a vacuolar localization tag (amino acids 1-24). SEQ ID NO: 215 comprises both epimerase and reductase enzymes, while SEQ ID NO: 219 comprises epimerase and reductase enzymes (amino acids 1-370) and a dehydratase enzyme (amino acids 371-667).

More specifically in a further embodiment the
a) ACT is the native Erg10 in S. cerevisiae;
b) HCS is the native Erg13 in S. cerevisiae;
c) HCR is the native HMG1 in S. cerevisiae;
d) HCR is the native HMG2 in S. cerevisiae;
e) MVK is the native Erg12 in S. cerevisiae;
f) PMK is the native Erg8 in S. cerevisiae;
g) MPC is the native MVD1 in S. cerevisiae;
h) IPI is the native ID11 in S. cerevisiae;
i) GPPS is the GPPS of SEQ ID NO: 45 or 229;
j) AAE is the AAE of SEQ ID NO: 47 or 239;
k) TKS is the TKS of SEQ ID NO: 49;
l) OAC is the OAC of SEQ ID NO: 51;
m) TKS-OAC fused enzyme is the TKS-OAC fused enzyme comprised in SEQ ID NO 227
n) CBGAS is the CBGAS of SEQ ID NO: 53, 235 or 237;
o) CBDAS is the CBDAS of SEQ ID NO: 57 or 233;
p) THCAS is the THCAS of SEQ ID NO: 55 or 231;
q) CBCAS is the CBCAS of SEQ ID NO: 59;
r) UDP-glucose synthase is the UDP-glucose synthase comprised in SEQ ID NO: 209;
s) UDP-galactose 4-epimerase is the UDP-galactose 4-epimerase comprised in SEQ ID NO: 211;
t) UDP-glucuronic acid decarboxylase is the UDP-glucuronic acid decarboxylase comprised in SEQ ID NO: 213;
u) UDP-4-keto-6-deoxy-glucose 3,5-epimerase is the UDP-4-keto-6-deoxy-glucose 3,5-epimerase comprised in SEQ ID NO: 215 or 219;
v) UDP-4-keto-rhamnose-4-keto reductase is the UDP-4-keto-rhamnose-4-keto reductase comprised in SEQ ID NO: 215 or 219;
w) UDP-glucose 4,6-dehydratase is the UDP-glucose 4,6-dehydratase comprised in SEQ ID NO: 217 or 219;
x) UDP-glucose 6-dehydrogenase is the UDP-glucose 6-dehydrogenase comprised in SEQ ID NO: 221;
y) UDP-arabinose 4-epimerase is the UDP-arabinose 4-epimerase comprised in SEQ ID NO: 223; and
z) UDP-N-acetylglucosamine 4-epimerase is the UDP-N-acetylglucosamine 4-epimerase comprised in SEQ ID NO: 225.

The sequence for Erg10 can be found the publically available Saccharomyces Genome Database (www.yeastgenome.org) under SGD ID: SGD:S000005949; the sequence for Erg13 under SGD ID: SGD:S000004595; the sequence for HMG1 under SGD ID: SGD:S000004540; the sequence for HMG2 under SGD ID: SGD:S000004442; the sequence for Erg12 under SGD ID: SGD:S000004821; the sequence for Erg8 under SGD ID: SGD:S000004833; the sequence for MVD1 under SGD ID: SGD:S000005326 and the sequence for ID11 under SGD ID: SGD:S000006038.

Further, a plurality of the polypeptides comprised in the operative biosynthetic metabolic pathway for making the cannabinoid acceptor may be heterologous to the genetically modified host cell. In more specific embodiments 2, 3, 4, 5, 6, 7, 8, 9, 10, 11, 12, 13, 14, 15, 16, 17, 18, 19 or 20 of the pathway polypeptides may are heterologous to the host cell.

The genetically modified host cell may also be further modified to optimize its production of the cannabinoid acceptor. For example, the cell may be genetically modified to increase the amount of one or more substrate or precursors or product for one or more one polypeptide of the operative biosynthetic metabolic pathway. Such modifications include, but is not limited to, incorporating and expressing two or more copies, such as 3, 4, 5 or 6 copies, of the polynucleotide encoding a polypeptide of the cannabinoid acceptor pathway and/or encoding the glycosyl transferase. The cell may also be genetically modified host cell is further genetically modified to exhibit increased tolerance towards one or more substrates, precursors, intermediates, or product molecules from the operative biosynthetic metabolic pathway. In a still further embodiment, the genetically modified host cell is modified to include a heterologous transporter polypeptide facilitating secretion of the intracellularly formed cannabinoid glycoside. In some embodiments one or more native genes are attenuated, disrupted and/or deleted in the genetically modified host cell. For example, where the genetically modified host cell is a S. cerevisiae strain, the PDR12 gene of SGD ID SGD:S000005979 may be attenuated, disrupted and/or deleted.

The genetically modified host cell comprises in some embodiments the polynucleotide construct or the expression vector disclosed, vide infra.

### Host Cells

The genetically modified host cell can be any microbial cell, such as eukaryotic, prokaryotic or archaic cell. However particularly useful host cells are eukaryotes selected from the group consisting of mammalian, insect, plant, or fungal cells. For example, the genetically modified host cell is a plant cell of the genus cannabis and Humulus. In another embodiment, the genetically modified host cell is a fungal host cell selected from the phylas of Ascomycota, Basidiomycota, Neocallimastigomycota, Glomeromycota, Blastocladiomycota, Chytridiomycota, Zygomycota, Oomycota and Microsporidia. More specifically the fungal genetically modified host cell may be a yeast cell selected from ascosporogenous yeast (Endomycetales), basidiosporogenous yeast, and Fungi Imperfecti yeast (Blastomycetes). The yeast may be picked from Saccharomyces, Kluveromyces, Candida, Pichia, Debaromyces, Hansenula, Yarrowia, Zygosaccharomyces, and Schizosaccharomyces, in particular selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii and Yarrowia lipolytica. In another embodiment the genetically modified host cell is a filamentous fungus, in particular a host cell selected from the phylas of Ascomycota, Eumycota and Oomycota. Such filamentous fungal host cell include, but are not limited to, those selected from the genera of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. In more specific embodiments the filamentous fungal host cell is selected from the species of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinurn, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianurn, Trichoderma koningii, Trichoderma longibrachiaturn, Trichoderma reesei, and Trichoderma viride. Further the host cell may also be Blakeslea trispora.

Genetically modified host cell of the invention may also be prokaryote cells, such as bacteria. Accordingly, the host cell may be a bacterium of a genera selected from Escherichia, Lactobacillus, Lactococcus, Cornebacterium, Acetobacter, Acinetobacter, Pseudomonas or Rhodobacter. In particular the host cell may be selected from the species of Escherichia coli, Rhodobacter sphaeroides, Rhodobacter capsulatus, or Rhodotorula toruloides. In one embodiment the bacterium is Escherichia coli. In a further alternative embodiment, the host cell of the invention is a cyanobacterium.

Genetically modified host cell of the invention may also be archaic cells, such as algae. Accordingly, the host cell may be selected from Dunaliella salina, Haematococcus pluvialis, Chlorella sp., Undaria pinnatifida, Sargassum, Laminaria japonica, Scenedesmus almeriensis.

In the alternative the host cell may be a plant cell for example of the genus Cannabis, Humulus or Physcomitrella. In addition to plant cells the invention also provides an isolated plant, e.g., a transgenic plant, plant part comprising the cannabinoid acceptor pathway polypeptides and glycosyl transferase of the invention and producing the cannabinoid glycosides of the invention in useful quantities. The compound may be recovered from the plant or plant part. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention is any the progeny of such plants, plant parts, and plant cells. The transgenic plant or plant cells comprising the operative pathway of the invention and produce the compound of the invention may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell. The expression vector conveniently comprises the polynucleotide construct of the invention. The choice of regulatory sequences, such as promoter and terminator sequences and optionally signal or transit sequences, is determined, for example, on the basis of when, where, and how the pathway polypeptides is desired to be expressed. For instance, the expression of a gene encoding a pathway enzyme polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506. For constitutive expression, the 358-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pint promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals. A promoter enhancer element may also be used to achieve higher expression in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression. The selectable marker gene and any other parts of the expression construct may be chosen from those available in the art. The polynucleotide construct or expression vector is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274). Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both incorporated herein by reference in their entirety).

Following transformation, the transformants having incorporated the expression vector or polynucleotide construct of the invention are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase. In addition to direct transformation of a particular plant genotype with a polynucleotide construct of the invention, transgenic plants may be made by crossing a plant comprising the construct to a second plant lacking the construct. For example, a polynucleotide construct encoding a glycosyl transferease of the invention can be introduced into a particular plant variety by crossing, without the need for ever directly transforming a plant of that given variety. Therefore, the invention encompasses not only a plant directly regenerated from cells which have been transformed in accordance with the invention, but also the progeny of such plants. As used herein, progeny may refer to the offspring of any generation of a parent plant prepared in accordance with the present invention. Such progeny may include a polynucleotide construct of the invention. Crossing results in the introduction of a transgene into a plant line by cross pollinating a starting line with a donor plant line. Non-limiting examples of such steps are described in U.S. Pat. No. 7,151,204. Plants may be generated through a process of backcross conversion. For example, plants include plants referred to as a backcross converted genotype, line, inbred, or hybrid. Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

### Nucleotide Constructs

In a further aspect the invention provides a polynucleotide construct comprising a polynucleotide sequence encoding the glycosyl transferase of the invention, operably linked to one or more control sequences heterologous to the glycosyl encoding polynucleotide.

Polynucleotides may be manipulated in a variety of ways to allow expression of a polypeptide. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may be an inducible promoter.

Examples of suitable promoters for directing transcription of the polynucleotide construct of the invention in a filamentous fungal host cell are promoters obtained from the genes for Aspergillus nidulans acetamidase, Aspergillus niger neutral α-amylase, Aspergillus niger acid stable α-amylase, Aspergillus niger or Aspergillus awamori glucoamylase (glaA), Aspergillus gpdA promoter, Aspergillus oryzae TAKA amylase, Aspergillus oryzae alkaline protease, Aspergillus oryzae triose phosphate isomerase, Aspergillus niger or Aspergillus awamori endoxylanase (xlnA) or β-xylosidase (xlnD), Fusarium oxysporum trypsin-like protease (WO 96/00787), Fusarium venenatum amyloglucosidase (WO2000/56900), Fusarium venenatum Dania (WO 00/56900), Fusarium venenatum Quinn (WO 00/56900), Rhizomucor miehei lipase, Rhizomucor miehei aspartic proteinase, Trichoderma reesei β-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei β-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the polynucleotide construct of the invention in a yeast host include the glyceraldehyde-3-phosphate dehydrogenase promoter, PgpdA or promoters obtained from the genes for Saccharomyces cerevisiae enolase (EN0-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488. Selecting a suitable promoter for expression in yeast is well know and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

Useful terminators for filamentous fungal host cells are obtained from the genes for Aspergillus nidulans anthranilate synthase, Aspergillus niger glucoamylase, Aspergillus niger α-glucosidase, Aspergillus oryzae TAKA amylase, and Fusarium oxysporum trypsin-like protease.

Useful terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (EN0-1), Saccharomyces cerevisiae 3-phosphoglycerate kinase, Saccharomyces cerevisiae α-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Useful polyadenylation sequences for filamentous fungal host cells are obtained from the genes for Aspergillus nidulans anthranilate synthase, Aspergillus niger glucoamylase, Aspergillus niger α-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound.

In filamentous fungi, the Aspergillus niger glucoamylase promoter, Aspergillus oryzae TAKA α-amylase promoter, and Aspergillus oryzae glucoamylase promoter may be used.

In yeast, the ADH2 system or GAL1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

The glycosyl transferase encoding polynucleotide is in one embodiment selected from:
a) a polynucleotide having at least 70%, such at least 75%, such as at least 80%, such as at least 90%, such as at least 95%, such as at least 99%, such as 100% identity to SEQ ID NO: 2;
b) a polynucleotide having at least 70%, such at least 75%, such as at least 80%, such as at least 90%, such as at least 95%, such as at least 99%, such as 100% identity to SEQ ID NO: 4;
c) a polynucleotide having at least 70%, such at least 75%, such as at least 80%, such as at least 90%, such as at least 95%, such as at least 99%, such as 100% identity to SEQ ID NO: 6;
d) a polynucleotide having at least 70%, such at least 75%, such as at least 80%, such as at least 90%, such as at least 95%, such as at least 99%, such as 100% identity to SEQ ID NO: 8;
e) a polynucleotide having at least 70%, such at least 75%, such as at least 80%, such as at least 90%, such as at least 95%, such as at least 99%, such as 100% identity to SEQ ID NO: 10;
f) a polynucleotide having at least 70%, such at least 75%, such as at least 80%, such as at least 90%, such as at least 95%, such as at least 99%, such as 100% identity to SEQ ID NO: 12;
g) a polynucleotide having at least 70%, such at least 75%, such as at least 80%, such as at least 90%, such as at least 95%, such as at least 99%, such as 100% identity to SEQ ID NO: 14;
h) a polynucleotide having at least 70%, such at least 75%, such as at least 80%, such as at least 90%, such as at least 95%, such as at least 99%, such as 100% identity to SEQ ID NO: 16; and
i) a polynucleotide having at least 70%, such at least 75%, such as at least 80%, such as at least 90%, such as at least 95%, such as at least 99%, such as 100% identity to SEQ ID NO: 18
j) a polynucleotide having at least 70%, such at least 75%, such as at least 80%, such as at least 90%, such as at least 95%, such as at least 99%, such as 100% identity to SEQ ID NO: 20;
k) a polynucleotide having at least 70%, such at least 75%, such as at least 80%, such as at least 90%, such as at least 95%, such as at least 99%, such as 100% identity to SEQ ID NO: 22;
l) a polynucleotide having at least 70%, such at least 75%, such as at least 80%, such as at least 90%, such as at least 95%, such as at least 99%, such as 100% identity to SEQ ID NO: 24;
m) a polynucleotide having at least 70%, such at least 75%, such as at least 80%, such as at least 90%, such as at least 95%, such as at least 99%, such as 100% identity to SEQ ID NO: 26;
n) a polynucleotide having at least 70%, such at least 75%, such as at least 80%, such as at least 90%, such as at least 95%, such as at least 99%, such as 100% identity to SEQ ID NO: 28;
o) a polynucleotide having at least 70%, such at least 75%, such as at least 80%, such as at least 90%, such as at least 95%, such as at least 99%, such as 100% identity to SEQ ID NO: 30;
p) a polynucleotide having at least 70%, such at least 75%, such as at least 80%, such as at least 90%, such as at least 95%, such as at least 99%, such as 100% identity to SEQ ID NO: 32; and
q) a polynucleotide having at least 70%, such at least 75%, such as at least 80%, such as at least 90%, such as at least 95%, such as at least 99%, such as 100% identity to SEQ ID NO: 34.

In another embodiment, the glycosyl transferase encoding polynucleotide in the polynucleotide construct of the invention has at least 70%, such at least 75%, such as at least 80%, such as at least 90%, such as at least 95%, such as at least 99%, such as 100% identity to the glycosyl transferase encoding gene comprised in anyone of SEQ ID NO: 2, 4, 6, 8, 10, 12, 14, 16, 18, 20, 22, 24, 26, 28, 30, 32, 34, 36, 38, 40, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 or 208.

### Expression Vectors

In a further aspect the invention provides an expression vector comprising the polynucleotide construct of the invention. Various nucleotide sequences in addition to the polynucleotide construct of the invention may be joined together to produce a recombinant expression vector, which may include one or more convenient restriction sites to allow for insertion or substitution of the polynucleotide sequence encoding the relevant polypeptide at such sites. The recombinant expression vector may be any vector (e.g., a plasmid or virus) that can be conveniently subjected to recombinant DNA procedures and can bring about expression of the relevant polypeptide encoding polynucleotide. The choice of the vector will typically depend on the compatibility of the vector with the host cell into which the vector is to be introduced. The vector may be a linear or closed circular plasmid. The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may, when introduced into the host cell, integrate into the genome and replicate together with the chromosome(s) into which it has been integrated. Furthermore, a single vector or plasmid or two or more vectors or plasmids that together contain the total DNA to be introduced into the genome of the host cell, or a transposon, may be used. The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like.

Useful selectable markers for filamentous fungal host cell include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Aspergillus nidulans or Aspergillus oryzae amdS and pyrG genes and a Streptomyces hygroscopicus bar gene are particularly useful in Aspergillus cells.

Useful selectable markers for yeast host cells include, but are not limited to, ADE2, HIS3, LEU2, LYS2, MET3, TRP1, and URA3.

The vector preferably contains element(s) that permits integration of the vector into the host cell's genome or permits autonomous replication of the vector in the cell independent of the genome. For integration into the host cell genome, the vector may rely on the polynucleotide encoding the polypeptide or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 35 to 10,000 base pairs, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

The origin of replication may be any plasmid replicator mediating autonomous replication that functions in a cell. The term “origin of replication” or “plasmid replicator” refers to a polynucleotide that enables a plasmid or vector to replicate in vivo.

Useful origins of replication for filamentous fungal cell include AMA 1 and ANSI. (Gems et al., 1991, Gene 98: 61-67; Cullen et al., 1987, Nucleic Acids Res. 15: 9163-9175; WO 00/24883). Isolation of the AMA 1 gene and construction of plasmids or vectors comprising the gene can be accomplished using the methods disclosed in WO 00/24883.

Useful origins of replication for yeast host cell are the 2 micron origin of replication, ARS1, ARS4, the combination of ARS1 and CEN3, and the combination of ARS4 and CEN6.

More than one copy of a polynucleotide encoding the glycosyl transferase or other pathway polypeptides of the invention may be inserted into a host cell to increase production of a polypeptide. An increase in the copy number can be obtained by integrating one or more additional copies of the enzyme coding sequence into the host cell genome or by including an amplifiable selectable marker gene with the polynucleotide, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present invention are well known to one skilled in the art (see, e.g., Sambrook et al., 1989, supra).

### Cell Cultures

In a further aspect the invention provides a cell culture, comprising the genetically modified host cell of the invention and a growth medium. Suitable growth mediums for host cells such as plant cell lines, filamentous fungi and/or yeast are known in the art.

Methods of producing compounds of the invention.

In a further aspect the invention provides a method for producing a cannabinoid glycoside comprising:
a) culturing the cell culture of claim of the invention at conditions allowing the genetically modified host cell to produce the cannabinoid glycoside; and
b) optionally recovering and/or isolating the cannabinoid glycoside.

The cell culture can be cultivated in a nutrient medium suitable for production of the compound of the invention and/or propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the pathway to operate to produce the compound of the invention and optionally to be recovered and/or isolated.

The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The selection of the appropriate medium may be based on the choice of host cell and/or based on the regulatory requirements for the host cell. Such media are in the art. The medium may, if desired, contain additional components favoring the transformed expression hosts over other potentially contaminating microorganisms. Accordingly, in an embodiment a suitable nutrient medium comprise a carbon source (e.g. glucose, maltose, molasses, starch, cellulose, xylan, pectin, lignocellolytic biomass hydrolysate, etc.), a nitrogen source (e.g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivating of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeats and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
c) culturing the cell culture for between 10 hours to 30 days; and
d) culturing the cell culture under fed-batch, repeated fed-batch or semi-continuous conditions
e) culturing the cell culture in the presence of an organic solvent to improve the solubility of the cannabinoid aglycone.

Further, in one embodiment the method for producing the cannabinoid glycoside comprises a step of non-enzymatic decarboxylation of the cannabinoid acceptor and/or the cannabinoid glycoside. The decarboxylation may be achieved by heat-, UV- or alkalinity treatment or a combination thereof.

The method may further comprise feeding one or more exogenous cannabinoid acceptors and/or nucleotide-glycosides to the cell culture.

The cannabinoid glycoside of the invention may be recovered and or isolated using methods known in the art. For example, the cannabinoid glycoside may be recovered from the nutrient medium by conventional procedures including, but not limited to, collection, centrifugation, filtration, extraction, spray-drying, evaporation, or precipitation. The cannabinoid glycoside may be isolated by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989).

In a particular embodiment, the recovering and/or isolation step of the method of the invention comprises separating a liquid phase of the host cell or cell culture from a solid phase of the host cell or cell culture to obtain a supernatant comprising the cannabinoid glycoside of the invention by one or more steps selected from:
a) disintegrating the genetically modified host cell to release intracellular cannabinoid glycosides into the supernatant;
b) contacting the supernatant with one or more adsorbent resins in order to obtain at least a portion of the produced cannabinoid glycosides;
c) contacting the supernatant with one or more ion exchange or reversed-phase chromatography columns in order to obtain at least a portion of the cannabinoid glycosides; and
d) crystallizing or extracting the cannabinoid glycosides; and
e) evaporating the solvent of the liquid phase to concentrate or precipitate the cannabinoid glycosides;

thereby recovering and/or isolating the cannabinoid glycoside.

The cannabinoid glycoside yield of the method of the invention is preferably at least 10% higher such as at least 50%, such as at least 100%, such as least 150%, such as at least 200% higher than production by using the glycosyl transferese UGT76G1 from Stevia rebaudiana in the host cell.

Not all conversion steps of pathway to produce the cannabinoid acceptor of the invention need to occur in vivo in the host cell, so in a particular embodiment one or more of these steps are carried out in vitro. Accordingly, in an embodiment the method of the invention comprises at least one cannabinoid acceptor pathway step which is performed in vitro.

In one embodiment the method of producing the cannabinoid glycoside includes steps of working the cannabinoid glycoside into a pharmaceutical cannabinoid formulation comprising feeding a cell culture of the invention comprising non-plant cells with a starting material in a growth medium; producing the pharmaceutical cannabinoid compound from the cell culture to create a mixture comprising the cell culture, the growth medium, and the pharmaceutical cannabinoid compound; processing the pharmaceutical cannabinoid compound, wherein the processing comprises: separating out genetical modified cells using at least one process selected from the group consisting of sedimentation, filtration, and centrifugation; and producing the pharmaceutical cannabinoid formulation that comprises the pharmaceutical cannabinoid, wherein the mixture does not contain a detectable amount of plant impurities selected from the group consisting of polysaccharides, lignin, pigments, flavonoids, phenanthreoids, latex, gum, resin, wax, pesticides, fungicides, herbicides, and pollen.

In a separate aspect the invention also provides a method for producing a cannabinoid glycoside comprising contacting a cannabinoid acceptor with one or more cannabinoid glycosyl transferases of the invention and one or more nucleotide glycosides of the invention at conditions allowing the glycosyl transferase to transfer the glycosyl moiety of the nucleotide glycoside to the cannabinoid. In particular the method of this aspect may be performed in vitro as well as in vivo in a genetically modified cell of the invention.

2. The methods of producing cannabinoid glycosides can further comprise subjecting the cannabinoid glycoside to one or more deglycosylation steps. The deglycosylation can be achieved by incubating the cannabinoid glycoside with one or more enzymes selected from glucosidases, pectinase, arabinase, cellulase, glucanase, hemicellulase, and xylanase. Particularly useful deglycosylating enzymes include β-glucosidase, β-betagluconase, pectolyase, pectozyme and polygalacturonase. The deglycosylating step can in particular be performed in vitro.

### Fermentation Liquids

In a further aspect the invention provides a fermentation liquid comprising the cannabinoid glycosides comprised in the cell culture of the invention. Preferably, at least 50%, such as at least 75%, such as at least 95%, such as at least 99% of the genetically modified host cells are disintegrated and preferably at least 50%, such as at least 75%, such as at least 95%, such as at least 99% of solid cellular material has been separated from the liquid. In an embodiment the fermentation liquid further comprises one or more compounds selected from:
a) Precursor or products of the operative biosynthetic metabolic pathway producing the cannabinoid glycoside;
b) supplemental nutrients comprising trace metals, vitamins, salts, yeast nitrogen base, YNB, and/or amino acids; and
   wherein the concentration of the cannabinoid glycoside is at least 1 mg/I fermentation liquid. Preferably, the cannabinoid concentration in the fermentation liquid is at least 5 mg/L, such as at least 10 mg/L, such as at least 20 mg/I, such as at least 50 mg/L, such as at least 100 mg/L, such as at least 500 mg/L, such as at least 1000 mg/L, such as at least 5000 mg/L, such as at least 10000 mg/L, such as at least 50000 mg/L.

### Compound and Compositions

It has been found that glycosyl transferases of the invention can produce new useful cannabinoid glycosides. Accordingly, in an aspect the invention provides a cannabinoid glycoside comprising a cannabinoid aglycone or cannabinoid glycoside covalently linked to a sugar selected from xylose; rhamnose; galactose; N-acetylglucosamine; N-acetylgalactosamine; and arabinose.

Further these cannabinoid glycosides can be selected from CBD-1′-O-β-D-xylosyl-3′-O-β-D-xyloside; CBD-1′-O-α-L-rhamnosyl-3′-O-α-L-rhamnoside; CBD-1′-O-β-D-galactosyl-3′-O-β-D-galactoside; CBD-1′-O-β-D-N-acetylglucosamine-3′-O-β-D-N-acetylglucosaminoside; CBD-1′-O-β-D-arabinosyl-3′-O-β-D-arabinoside; CBD-1′-O-β-D-N-acetylgalactosamine-3′-O-β-D-N-acetylgalactosamine; CBDV-1′-O-β-D-xylosyl-3′-O-β-D-xyloside; CBDV-1′-O-α-L-rhamnosyl-3′-O-α-L-rhamnoside; CBDV-1′-O-β-D-galactosyl-3′-O-β-D-galactoside; CBDV-1′-O-β-D-N-acetylglucosamine-3′-O-β-D-N-acetylglucosaminoside; CBDV-1′-O-3-D-arabinosyl-3′-O-β-D-arabinoside; CBDV-1′-O-β-D-N-acetylgalactosamine-3′-O-β-D-N-acetylgalactosamine; CBG-1′-O-β-D-xylosyl-3′-O-β-D-xyloside CBG-1′-O-α-L-rhamnosyl-3′-O-α-L-rhamnoside; CBG-1′-O-β-D-galactosyl-3′-O-β-D-galactoside; CBG-1′-O-β-D-N-acetylglucosamine-3′-O-β-D-N-acetylglucosaminoside; CBG-1′-O-β-D-arabinosyl-3′-O-β-D-arabinoside; CBG-1′-O-β-D-N-acetylgalactosamine-3′-O-β-D-N-acetylgalactosamine; THC-1′-O-β-D-xyloside; THC-1′-O-α-L-rhamnoside; THC-1′-O-β-D-galactoside; THC-1′-O-β-D-N-acetylglucosaminoside; THC-1′-O-β-D-arabinoside; THC-1′-O-β-D-N-acetylgalactosaminoside; CBN-1′-O-β-D-xyloside; CBN-1′-O-α-L-rhamnoside; CBN-1′-O-β-D-galactoside; CBN-1′-O-β-D-N-acetylglucosaminoside; CBN-1′-O-β-D-arabinoside; CBN-1′-O-β-D-N-acetylgalactosaminoside; CBDA-1′-O-β-D-xyloside; CBDA-1′-O-α-L-rhamnoside; CBDA-1′-O-β-D-galactoside; CBDA-1′-O-β-D-N-acetylglucosaminoside; CBDA-1′-O-β-D-arabinoside; CBDA-1′-O-β-D-N-acetylgalactosaminoside; CBC-1′-O-β-D-xyloside; CBC-1′-O-α-L-rhamnoside; CBC-1′-O-β-D-galactoside; CBC-1′-O-β-D-N-acetylglucosaminoside; CBC-1′-O-β-D-arabinoside; and CBC-1′-O-β-D-N-acetylgalactosaminoside. Particularly interesting cannabinoid glycoside which have not previously been disclosed are cannabinoid aglycones or cannabinoid glycosides covalently linked to a glycosyl moiety by a 1,4- or a 1,6-glycosidic bond. Still further, the cannabinoid glycoside can be CBD-1′-O-β-D-gentiobioside or CBD-1′-O-β-D-cellobioside.

The new cannabinoid glycoside molecules can be group into the following groups, together with an example of the glycosyltransferease(s) of the invention which catalyzes glycosylation.

| | | | SEQ ID |
|---|---|---|---|
| Group | Exemplary molecule | Enzyme | NO |
| Cannabinoid cellobioside | CBD-1′-O-β-D-cellobioside | Pt88G + | 147, 115 |
| | | OsEUGT11 | |
| Cannabinoid gentiobioside | CBD-1′-O-β-D-gentiobioside | Pt88G + | 147, 145 |
| | | Si94D | |
| Cannabinoid xyloside | THC-1′-O-β-D-xyloside | Cs73Y | 157 |
| Cannabinoid rhamnoside | CBD-1′-O-α-L-rhamnoside | Cp73B | 191 |
| Cannabinoid galactoside | CBD-1′-O-β-D-galactosyl-3′-O-β-D-galactoside | Cs73Y | 157 |
| Cannabinoid N- | CBD-1-O-β-D-N-acetylglucosamine-3′-O-β-D- | Cs73Y | 157 |
| acetylglucosaminoside | N-acetylglucosaminoside | | |
| Cannabinoid arabinoside | CBD-1′-O-β-D-arabinosyl-3′-O-β-D-arabinoside | Cs73Y | 157 |
| Cannabinoid N- | CBD-1′-O-β-D-N-acetylgalactosamine-3′-O-β- | Cs73Y | 157 |
| acetylgalactosaminoside | D-N-acetylgalactosamine | | |

More specifically, new cannabinoid glycoside molecules and examples of glycosyltransferease of the invention which catalyzes glycosylation include:

| Glycoside name | Enzyme(s) | SEQ ID NO |
|---|---|---|
| CBD-1′-O-β-D-cellobioside | Pt88G + OsEUGT11 | 147, 115 |
| CBD-1′-O-β-D-gentiobioside | Pt88G + Si94D | 147, 145 |
| CBD-1′-O-β-D-xyloside | Pt88G | 147 |
| CBD-1′-O-α-L-rhamnoside | Cp73B | 191 |
| CBD-1′-O-β-D-galactoside | Cs73Y | 157 |
| CBD-1′-O-β-D-N-acetylglucosaminoside | Cs73Y | 157 |
| CBD-1′-O-β-D-arabinoside | Cs73Y | 157 |
| CBD-1′-O-β-D-N-acetylgalactosaminoside | Cs73Y | 157 |
| CBDV-1′-O-β-D-cellobioside | Ha72B + OsEUGT11 | 179, 115 |
| CBDV-1′-O-β-D-gentiobioside | Ha72B + Si94D | 179, 145 |
| CBDV-1′-O-β-D-xyloside | Cs73Y | 157 |
| CBDV-1′-O-α-L-rhamnoside | Cs73Y | 157 |
| CBDV-1′-O-β-D-galactoside | Cs73Y | 157 |
| CBDV-1′-O-β-D-N-acetylglucosaminoside | Cs73Y | 157 |
| CBDV-1′-O-β-D-arabinoside | Cs73Y | 157 |
| CBDV-1′-O-β-D-N-acetylgalactosaminoside | Cs73Y | 157 |
| CBDA-1′-O-β-D-cellobioside | Cs73Y + OsEUGT11 | 157, 115 |
| CBDA-1′-O-β-D-gentiobioside | Cs73Y + Si94D | 157, 145 |
| CBDA-1′-O-β-D-xyloside | Cs73Y | 157 |
| CBDA-1′-O-α-L-rhamnoside | Cs73Y | 157 |
| CBDA-1′-O-β-D-galactoside | Cs73Y | 157 |
| CBDA-1′-O-β-D-N-acetylglucosaminoside | Cs73Y | 157 |
| CBDA-1′-O-β-D-arabinoside | Cs73Y | 157 |
| CBDA-1′-O-β-D-N-acetylgalactosaminoside | Cs73Y | 157 |
| CBG-1′-O-β-D-cellobioside | Qs72S + OsEUGT11 | 187, 115 |
| CBG-1′-O-β-D-gentiobioside | Qs72S + Si94D | 187, 145 |
| CBG-1′-O-β-D-xyloside | Cs73Y | 157 |
| CBG-1′-O-α-L-rhamnoside | Cs73Y | 157 |
| CBG-1′-O-β-D-galactoside | Cs73Y | 157 |
| CBG-1′-O-β-D-N-acetylglucosaminoside | Cs73Y | 157 |
| CBG-1′-O-β-D-arabinoside | Cs73Y | 157 |
| CBG-1′-O-β-D-N-acetylgalactosaminoside | Cs73Y | 157 |
| THC-1′-O-β-D-cellobioside | Ha88B_2 + | 149, 115 |
| | OsEUGT11 | |
| THC-1′-O-β-D-gentiobioside | Ha88B_2 + Si94D | 149, 145 |
| THC-1′-O-β-D-xyloside | Cs73Y | 157 |
| THC-1′-O-α-L-rhamnoside | Cs73Y | 157 |
| THC-1′-O-β-D-galactoside | Cs73Y | 157 |
| THC-1′-O-β-D-N-acetylglucosaminoside | Cs73Y | 157 |
| THC-1′-O-β-D-arabinoside | Cs73Y | 157 |
| THC-1′-O-β-D-N-acetylgalactosaminoside | Cs73Y | 157 |
| THCV-1′-O-β-D-cellobioside | Ha88B_2 + | 149, 115 |
| | OsEUGT11 | |
| THCV-1′-O-β-D-gentiobioside | Ha88B_2 + Si94D | 149, 145 |
| THCV-1′-O-β-D-xyloside | Cs73Y | 157 |
| THCV-1′-O-α-L-rhamnoside | Cs73Y | 157 |
| THCV-1′-O-β-D-galactoside | Cs73Y | 157 |
| THCV-1′-O-β-D-N-acetylglucosaminoside | Cs73Y | 157 |
| THCV-1′-O-β-D-arabinoside | Cs73Y | 157 |
| THCV-1′-O-β-D-N-acetylgalactosaminoside | Cs73Y | 157 |
| CBC-1′-O-β-D-cellobioside | Cs73Y + OsEUGT11 | 157, 115 |
| CBC-1′-O-β-D-gentiobioside | Cs73Y + Si94D | 157, 145 |
| CBC-1′-O-β-D-xyloside | Cs73Y | 157 |
| CBC-1′-O-α-L-rhamnoside | Cs73Y | 157 |
| CBC-1′-O-β-D-galactoside | Cs73Y | 157 |
| CBC-1′-O-β-D-N-acetylglucosaminoside | Cs73Y | 157 |
| CBC-1′-O-β-D-arabinoside | Cs73Y | 157 |
| CBC-1′-O-β-D-N-acetylgalactosaminoside | Cs73Y | 157 |
| CBN-1′-O-β-D-cellobioside | Cp73B + OsEUGT11 | 191, 115 |
| CBN-1′-O-β-D-gentiobioside | Cp73B + Si94D | 191, 145 |
| CBN-1′-O-β-D-xyloside | Cs73Y | 157 |
| CBN-1′-O-α-L-rhamnoside | Cs73Y | 157 |
| CBN-1′-O-β-D-galactoside | Cs73Y | 157 |
| CBN-1′-O-β-D-N-acetylglucosaminoside | Cs73Y | 157 |
| CBN-1′-O-β-D-arabinoside | Cs73Y | 157 |
| CBN-1′-O-β-D-N-acetylgalactosaminoside | Cs73Y | 157 |
| 11-nor-9-carboxy-THC-1′-O-β-D-cellobioside | Tc90A + OsEUGT11 | 143, 115 |
| 11-nor-9-carboxy-THC-1′-O-β-D-gentiobioside | Tc90A + Si94D | 143, 145 |
| 11-nor-9-carboxy-THC-1′-O-β-D-xyloside | Cs73Y | 157 |
| 11-nor-9-carboxy-THC-1′-O-α-L-rhamnoside | Cs73Y | 157 |
| 11-nor-9-carboxy-THC-1′-O-β-D-galactoside | Cs73Y | 157 |
| 11-nor-9-carboxy-THC-1′-O-β-D-N-acetylglucosaminoside | Cs73Y | 157 |
| 11-nor-9-carboxy-THC-1′-O-β-D-arabinoside | Cs73Y | 157 |
| 11-nor-9-carboxy-THC-1′-O-β-D-N-acetylgalactosaminoside | Cs73Y | 157 |
| CBD-3′-O-β-D-cellobioside | Pt88G + OsEUGT11 | 147, 115 |
| CBD-3′-O-β-D-gentiobioside | Pt88G + Si94D | 147, 145 |
| CBD-3′-O-β-D-xyloside | Pt88G | 147 |
| CBD-3′-O-α-L-rhamnoside | Cp73B | 191 |
| CBD-3′-O-β-D-galactoside | Cs73Y | 157 |
| CBD-3′-O-β-D-N-acetylglucosaminoside | Cs73Y | 157 |
| CBD-3′-O-β-D-arabinoside | Cs73Y | 157 |
| CBD-3′-O-β-D-N-acetylgalactosaminoside | Cs73Y | 157 |
| CBDV-3′-O-β-D-cellobioside | Ha72B + OsEUGT11 | 179, 115 |
| CBDV-3′-O-β-D-gentiobioside | Ha72B + Si94D | 179, 145 |
| CBDV-3′-O-β-D-xyloside | Cs73Y | 157 |
| CBDV-3′-O-α-L-rhamnoside | Cs73Y | 157 |
| CBDV-3′-O-β-D-galactoside | Cs73Y | 157 |
| CBDV-3′-O-β-D-N-acetylglucosaminoside | Cs73Y | 157 |
| CBDV-3′-O-β-D-arabinoside | Cs73Y | 157 |
| CBDV-3′-O-β-D-N-acetylgalactosaminoside | Cs73Y | 157 |
| CBG-3′-O-β-D-cellobioside | Qs72S + OsEUGT11 | 187, 115 |
| CBG-3′-O-β-D-gentiobioside | Qs72S + Si94D | 187, 145 |
| CBG-3′-O-β-D-xyloside | Cs73Y | 157 |
| CBG-3′-O-α-L-rhamnoside | Cs73Y | 157 |
| CBG-3′-O-β-D-galactoside | Cs73Y | 157 |
| CBG-3′-O-β-D-N-acetylglucosaminoside | Cs73Y | 157 |
| CBG-3′-O-β-D-arabinoside | Cs73Y | 157 |
| CBG-3′-O-β-D-N-acetylgalactosaminoside | Cs73Y | 157 |
| CBD-1′-O-β-D-di-xyloside | Cs73Y | 157 |
| CBD-1′-O-α-L-di-rhamnoside | Cs73Y | 157 |
| CBD-1′-O-β-D-di-galactoside | Cs73Y | 157 |
| CBD-1′-O-β-D-di-N-acetylglucosaminoside | Cs73Y | 157 |
| CBD-1′-O-β-D-di-arabinoside | Cs73Y | 157 |
| CBD-1′-O-β-D-di-N-acetylgalactosaminoside | Cs73Y | 157 |
| CBDV-1′-O-β-D-di-xyloside | Cs73Y | 157 |
| CBDV-1′-O-α-L-di-rhamnoside | Cs73Y | 157 |
| CBDV-1′-O-β-D-di-galactoside | Cs73Y | 157 |
| CBDV-1′-O-β-D-di-N-acetylglucosaminoside | Cs73Y | 157 |
| CBDV-1′-O-β-D-di-arabinoside | Cs73Y | 157 |
| CBDV-1′-O-β-D-di-N-acetylgalactosaminoside | Cs73Y | 157 |
| CBDA-1′-O-β-D-di-xyloside | Cs73Y | 157 |
| CBDA-1′-O-α-L-di-rhamnoside | Cs73Y | 157 |
| CBDA-1′-O-β-D-di-galactoside | Cs73Y | 157 |
| CBDA-1′-O-β-D-di-N-acetylglucosaminoside | Cs73Y | 157 |
| CBDA-1′-O-β-D-di-arabinoside | Cs73Y | 157 |
| CBDA-1′-O-β-D-di-N-acetylgalactosaminoside | Cs73Y | 157 |
| CBG-1′-O-β-D-di-xyloside | Cs73Y | 157 |
| CBG-1′-O-α-L-di-rhamnoside | Cs73Y | 157 |
| CBG-1′-O-β-D-di-galactoside | Cs73Y | 157 |
| CBG-1′-O-β-D-di-N-acetylglucosaminoside | Cs73Y | 157 |
| CBG-1′-O-β-D-di-arabinoside | Cs73Y | 157 |
| CBG-1′-O-β-D-di-N-acetylgalactosaminoside | Cs73Y | 157 |
| THC-1′-O-β-D-di-xyloside | Cs73Y | 157 |
| THC-1′-O-α-L-di-rhamnoside | Cs73Y | 157 |
| THC-1′-O-β-D-di-galactoside | Cs73Y | 157 |
| THC-1′-O-β-D-di-N-acetylglucosaminoside | Cs73Y | 157 |
| THC-1′-O-β-D-di-arabinoside | Cs73Y | 157 |
| THC-1′-O-β-D-di-N-acetylgalactosaminoside | Cs73Y | 157 |
| THCV-1′-O-β-D-di-xyloside | Cs73Y | 157 |
| THCV-1′-O-α-L-di-rhamnoside | Cs73Y | 157 |
| THCV-1′-O-β-D-di-galactoside | Cs73Y | 157 |
| THCV-1′-O-β-D-di-N-acetylglucosaminoside | Cs73Y | 157 |
| THCV-1′-O-β-D-di-arabinoside | Cs73Y | 157 |
| THCV-1′-O-β-D-di-N-acetylgalactosaminoside | Cs73Y | 157 |
| CBC-1′-O-β-D-di-xyloside | Cs73Y | 157 |
| CBC-1′-O-α-L-di-rhamnoside | Cs73Y | 157 |
| CBC-1′-O-β-D-di-galactoside | Cs73Y | 157 |
| CBC-1′-O-β-D-di-N-acetylglucosaminoside | Cs73Y | 157 |
| CBC-1′-O-β-D-di-arabinoside | Cs73Y | 157 |
| CBC-1′-O-β-D-di-N-acetylgalactosaminoside | Cs73Y | 157 |
| CBN-1′-O-β-D-di-xyloside | Cs73Y | 157 |
| CBN-1′-O-α-L-di-rhamnoside | Cs73Y | 157 |
| CBN-1′-O-β-D-di-galactoside | Cs73Y | 157 |
| CBN-1′-O-β-D-di-N-acetylglucosaminoside | Cs73Y | 157 |
| CBN-1′-O-β-D-di-arabinoside | Cs73Y | 157 |
| CBN-1′-O-β-D-di-N-acetylgalactosaminoside | Cs73Y | 157 |
| 11-nor-9-carboxy-THC-1′-O-β-D-di-xyloside | Cs73Y | 157 |
| 11-nor-9-carboxy-THC-1′-O-α-L-di-rhamnoside | Cs73Y | 157 |
| 11-nor-9-carboxy-THC-1′-O-β-D-di-galactoside | Cs73Y | 157 |
| 11-nor-9-carboxy-THC-1′-O-β-D-di-N-acetylglucosaminoside | Cs73Y | 157 |
| 11-nor-9-carboxy-THC-1′-O-β-D-di-arabinoside | Cs73Y | 157 |
| 11-nor-9-carboxy-THC-1′-O-β-D-di-N-acetylgalactosaminoside | Cs73Y | 157 |
| CBD-3′-O-β-D-di-xyloside | Cs73Y | 157 |
| CBD-3′-O-α-L-di-rhamnoside | Cs73Y | 157 |
| CBD-3′-O-β-D-di-galactoside | Cs73Y | 157 |
| CBD-3′-O-β-D-di-N-acetylglucosaminoside | Cs73Y | 157 |
| CBD-3′-O-β-D-di-arabinoside | Cs73Y | 157 |
| CBD-3′-O-β-D-di-N-acetylgalactosaminoside | Cs73Y | 157 |
| CBDV-3′-O-β-D-di-xyloside | Cs73Y | 157 |
| CBDV-3′-O-α-L-di-rhamnoside | Cs73Y | 157 |
| CBDV-3′-O-β-D-di-galactoside | Cs73Y | 157 |
| CBDV-3′-O-β-D-di-N-acetylglucosaminoside | Cs73Y | 157 |
| CBDV-3′-O-β-D-di-arabinoside | Cs73Y | 157 |
| CBDV-3′-O-β-D-di-N-acetylgalactosaminoside | Cs73Y | 157 |
| CBG-3′-O-β-D-di-xyloside | Cs73Y | 157 |
| CBG-3′-O-α-L-di-rhamnoside | Cs73Y | 157 |
| CBG-3′-O-β-D-di-galactoside | Cs73Y | 157 |
| CBG-3′-O-β-D-di-N-acetylglucosaminoside | Cs73Y | 157 |
| CBG-3′-O-β-D-di-arabinoside | Cs73Y | 157 |
| CBG-3′-O-β-D-di-N-acetylgalactosaminoside | Cs73Y | 157 |
| CBD-1′-O-β-D-tri-xyloside | Cs73Y | 157 |
| CBD-1′-O-α-D-tri-rhamnoside | Cs73Y | 157 |
| CBD-1′-O-β-D-tri-galactoside | Cs73Y | 157 |
| CBD-1′-O-β-D-tri-N-acetylglucosaminoside | Cs73Y | 157 |
| CBD-1′-O-β-D-tri-arabinoside | Cs73Y | 157 |
| CBD-1′-O-β-D-tri-N-acetylgalactosaminoside | Cs73Y | 157 |
| CBDV-1′-O-β-D-tri-xyloside | Cs73Y | 157 |
| CBDV-1′-O-α-D-tri-rhamnoside | Cs73Y | 157 |
| CBDV-1′-O-β-D-tri-galactoside | Cs73Y | 157 |
| CBDV-1′-O-β-D-tri-N-acetylglucosaminoside | Cs73Y | 157 |
| CBDV-1′-O-β-D-tri-arabinoside | Cs73Y | 157 |
| CBDV-1′-O-β-D-tri-N-acetylgalactosaminoside | Cs73Y | 157 |
| CBG-1′-O-β-D-tri-xyloside | Cs73Y | 157 |
| CBG-1′-O-α-D-tri-rhamnoside | Cs73Y | 157 |
| CBG-1′-O-β-D-tri-galactoside | Cs73Y | 157 |
| CBG-1′-O-β-D-tri-N-acetylglucosaminoside | Cs73Y | 157 |
| CBG-1′-O-β-D-tri-arabinoside | Cs73Y | 157 |
| CBN-1′-O-β-D-tri-xyloside | Cs73Y | 157 |
| CBN-1′-O-α-D-tri-rhamnoside | Cs73Y | 157 |
| CBN-1′-O-β-D-tri-galactoside | Cs73Y | 157 |
| CBN-1′-O-β-D-tri-N-acetylglucosaminoside | Cs73Y | 157 |
| CBN-1′-O-β-D-tri-arabinoside | Cs73Y | 157 |
| CBN-1′-O-β-D-tri-N-acetylgalactosaminoside | Cs73Y | 157 |
| CBD-3′-O-β-D-tri-xyloside | Cs73Y | 157 |
| CBD-3′-O-α-D-tri-rhamnoside | Cs73Y | 157 |
| CBD-3′-O-β-D-tri-galactoside | Cs73Y | 157 |
| CBD-3′-O-β-D-tri-N-acetylglucosaminoside | Cs73Y | 157 |
| CBD-3′-O-β-D-tri-arabinoside | Cs73Y | 157 |
| CBD-3′-O-β-D-tri-N-acetylgalactosaminoside | Cs73Y | 157 |
| CBDV-3′-O-β-D-tri-xyloside | Cs73Y | 157 |
| CBDV-3′-O-α-D-tri-rhamnoside | Cs73Y | 157 |
| CBDV-3′-O-β-D-tri-galactoside | Cs73Y | 157 |
| CBDV-3′-O-β-D-tri-N-acetylglucosaminoside | Cs73Y | 157 |
| CBDV-3′-O-β-D-tri-arabinoside | Cs73Y | 157 |
| CBDV-3′-O-β-D-tri-N-acetylgalactosaminoside | Cs73Y | 157 |
| CBG-3′-O-β-D-tri-xyloside | Cs73Y | 157 |
| CBG-3′-O-α-D-tri-rhamnoside | Cs73Y | 157 |
| CBG-3′-O-β-D-tri-galactoside | Cs73Y | 157 |
| CBG-3′-O-β-D-tri-N-acetylglucosaminoside | Cs73Y | 157 |
| CBG-3′-O-β-D-tri-arabinoside | Cs73Y | 157 |
| CBG-3′-O-β-D-tri-N-acetylgalactosaminoside | Cs73Y | 157 |
| CBD-1′-O-β-D-tetra-xyloside | Cs73Y | 157 |
| CBD-1′-O-α-D-tetra-rhamnoside | Cs73Y | 157 |
| CBD-1′-O-β-D-tetra-galactoside | Cs73Y | 157 |
| CBD-1′-O-β-D-tetra-N-acetylglucosaminoside | Cs73Y | 157 |
| CBD-1′-O-β-D-tetra-arabinoside | Cs73Y | 157 |
| CBD-1′-O-β-D-tetra-N-acetylgalactosaminoside | Cs73Y | 157 |
| CBDV-1′-O-β-D-tetra-xyloside | Cs73Y | 157 |
| CBDV-1′-O-α-D-tetra-rhamnoside | Cs73Y | 157 |
| CBDV-1′-O-β-D-tetra-galactoside | Cs73Y | 157 |
| CBDV-1′-O-β-D-tetra-N-acetylglucosaminoside | Cs73Y | 157 |
| CBDV-1′-O-β-D-tetra-arabinoside | Cs73Y | 157 |
| CBDV-1′-O-β-D-tetra-N-acetylgalactosaminoside | Cs73Y | 157 |
| CBG-1′-O-β-D-tetra-xyloside | Cs73Y | 157 |
| CBG-1′-O-α-D-tetra-rhamnoside | Cs73Y | 157 |
| CBG-1′-O-β-D-tetra-galactoside | Cs73Y | 157 |
| CBG-1′-O-β-D-tetra-N-acetylglucosaminoside | Cs73Y | 157 |
| CBG-1′-O-β-D-tetra-arabinoside | Cs73Y | 157 |
| CBG-1′-O-β-D-tetra-N-acetylgalactosaminoside | Cs73Y | 157 |
| CBD-3′-O-β-D-tetra-xyloside | Cs73Y | 157 |
| CBD-3′-O-α-D-tetra-rhamnoside | Cs73Y | 157 |
| CBD-3′-O-β-D-tetra-galactoside | Cs73Y | 157 |
| CBD-3′-O-β-D-tetra-N-acetylglucosaminoside | Cs73Y | 157 |
| CBD-3′-O-β-D-tetra-arabinoside | Cs73Y | 157 |
| CBD-3′-O-β-D-tetra-N-acetylgalactosaminoside | Cs73Y | 157 |
| CBDV-3′-O-β-D-tetra-xyloside | Cs73Y | 157 |
| CBDV-3′-O-α-D-tetra-rhamnoside | Cs73Y | 157 |
| CBDV-3′-O-β-D-tetra-galactoside | Cs73Y | 157 |
| CBDV-3′-O-β-D-tetra-N-acetylglucosaminoside | Cs73Y | 157 |
| CBDV-3′-O-β-D-tetra-arabinoside | Cs73Y | 157 |
| CBDV-3′-O-β-D-tetra-N-acetylgalactosaminoside | Cs73Y | 157 |
| CBG-3′-O-β-D-tetra-xyloside | Cs73Y | 157 |
| CBG-3′-O-α-D-tetra-rhamnoside | Cs73Y | 157 |
| CBG-3′-O-β-D-tetra-galactoside | Cs73Y | 157 |
| CBG-3′-O-β-D-tetra-N-acetylglucosaminoside | Cs73Y | 157 |
| CBG-3′-O-β-D-tetra-arabinoside | Cs73Y | 157 |
| CBG-3′-O-β-D-tetra-N-acetylgalactosaminoside | Cs73Y | 157 |
| CBD-1′-O-β-D-glucosyl-3′-O-β-D-cellobiose | Cs73Y + OsEUGT11 | 157, 115 |
| CBD-1′-O-β-D-glucosyl-3′-O-β-D-gentiobiose | Cs73Y + Si94D | 157, 145 |
| CBD-1′-O-β-D-xylosyl-3′-O-β-D-xyloside | Cs73Y | 157 |
| CBD-1′-O-α-L-rhamnosyl-3′-O-α-L-rhamnoside | Cs73Y | 157 |
| CBD-1′-O-β-D-galactosyl-3′-O-β-D-galactoside | Cs73Y | 157 |
| CBD-1′-O-β-D-N-acetylglucosaminyl-3′-O-β-D-N- | Cs73Y | 157 |
| acetylglucosaminoside | | |
| CBD-1′-O-β-D-arabinosyl-3′-O-β-D-arabinoside | Cs73Y | 157 |
| CBD-1′-O-β-D-N-acetylgalactosaminyl-3′-O-β-D-N- | Cs73Y | 157 |
| acetylgalactosaminoside | | |
| CBDV-1′-O-β-D-glucosyl-3′-O-β-D-cellobiose | Cs73Y + OsEUGT11 | 157, 115 |
| CBDV-1′-O-β-D-glucosyl-3′-O-β-D-gentiobiose | Cs73Y + Si94D | 157, 145 |
| CBDV-1′-O-β-D-glucosyl-3′-O-β-D-xyloside | Cs73Y | 157 |
| CBDV-1′-O-α-D-glucosyl-3′-O-α-L-rhamnoside | Cs73Y | 157 |
| CBDV-1′-O-β-D-glucosyl-3′-O-β-D-galactoside | Cs73Y | 157 |
| CBDV-1′-O-β-D-glucosyl-3′-O-β-D-N-acetylglucosaminoside | Cs73Y | 157 |
| CBDV-1′-O-β-D-glucosyl-3′-O-β-D-arabinoside | Cs73Y | 157 |
| CBDV-1′-O-β-D-glucosyl-3′-O-β-D-N-acetylgalactosaminoside | Cs73Y | 157 |
| CBG-1′-O-β-D-glucosyl-3′-O-β-D-cellobiose | Cs73Y + OsEUGT11 | 157, 115 |
| CBG-1′-O-β-D-glucosyl-3′-O-β-D-gentiobiose | Cs73Y + Si94D | 157, 145 |
| CBG-1′-O-β-D-glucosyl-3′-O-β-D-xyloside | Cs73Y | 157 |
| CBG-1′-O-α-D-glucosyl-3′-O-α-L-rhamnoside | Cs73Y | 157 |
| CBG-1′-O-β-D-glucosyl-3′-O-β-D-galactoside | Cs73Y | 157 |
| CBG-1′-O-β-D-glucosyl-3′-O-β-D-N-acetylglucosaminoside | Cs73Y | 157 |
| CBG-1′-O-β-D-glucosyl-3′-O-β-D-arabinoside | Cs73Y | 157 |
| CBG-1′-O-β-D-glucosyl-3′-O-β-D-N-acetylgalactosaminoside | Cs73Y | 157 |
| CBD-1′-O-β-D-cellobiosyl-3′-O-β-D-glucoside | Cs73Y + OsEUGT11 | 157, 115 |
| CBD-1′-O-β-D-gentiobiosyl-3′-O-β-D-glucoside | Cs73Y + Si94D | 157, 145 |
| CBDV-1′-O-β-D-cellobiosyl-3′-O-β-D-glucoside | Cs73Y + OsEUGT11 | 157, 115 |
| CBDV-1′-O-β-D-gentiobiosyl-3′-O-β-D-glucoside | Cs73Y + Si94D | 157, 145 |
| CBDA-1′-O-β-D-cellobiosyl-3′-O-β-D-glucoside | Cs73Y + OsEUGT11 | 157, 115 |
| CBDA-1′-O-β-D-gentiobiosyl-3′-O-β-D-glucoside | Cs73Y + Si94D | 157, 145 |
| CBG-1′-O-β-D-cellobiosyl-3′-O-β-D-glucoside | Cs73Y + OsEUGT11 | 157, 115 |
| CBG-1′-O-β-D-gentiobiosyl-3′-O-β-D-glucoside | Cs73Y + Si94D | 157, 145 |
| CBD-1′-O-β-D-xylosyl-3′-O-β-D-di-xyloside | Cs73Y | 157 |
| CBD-1′-O-α-L-rhamnosyl-3′-O-α-L-di-rhamnoside | Cs73Y | 157 |
| CBD-1′-O-β-D-galactosyl-3′-O-β-D-di-galactoside | Cs73Y | 157 |
| CBD-1′-O-β-D-N-acetylglucosaminyl-3′-O-β-D-di-N- | Cs73Y | 157 |
| acetylglucosaminoside | | |
| CBD-1′-O-β-D-arabinosyl-3′-O-β-D-di-arabinoside | Cs73Y | 157 |
| CBD-1′-O-β-D-N-acetylgalactosaminyl-3′-O-β-D-di-N- | Cs73Y | 157 |
| acetylgalactosaminoside | | |
| CBDV-1′-O-β-D-xylosyl-3′-O-β-D-di-xyloside | Cs73Y | 157 |
| CBDV-1′-O-α-L-rhamnosyl-3′-O-α-L-di-rhamnoside | Cs73Y | 157 |
| CBDV-1′-O-β-D-galactosyl-3′-O-β-D-di-galactoside | Cs73Y | 157 |
| CBDV-1′-O-β-D-N-acetylglucosaminyl-3′-O-β-D-di-N- | Cs73Y | 157 |
| acetylglucosaminoside | | |
| CBDV-1′-O-β-D-arabinosyl-3′-O-β-D-di-arabinoside | Cs73Y | 157 |
| CBDV-1′-O-β-D-N-acetylgalactosaminyl-3′-O-β-D-di-N- | Cs73Y | 157 |
| acetylgalactosaminoside | | |
| CBG-1′-O-β-D-xylosyl-3′-O-β-D-di-xyloside | Cs73Y | 157 |
| CBG-1′-O-α-L-rhamnosyl-3′-O-α-L-di-rhamnoside | Cs73Y | 157 |
| CBG-1′-O-β-D-galactosyl-3′-O-β-D-di-galactoside | Cs73Y | 157 |
| CBG-1′-O-β-D-N-acetylglucosaminyl-3′-O-β-D-di-N- | Cs73Y | 157 |
| acetylglucosaminoside | | |
| CBG-1′-O-β-D-arabinosyl-3′-O-β-D-di-arabinoside | Cs73Y | 157 |
| CBG-1′-O-β-D-N-acetylgalactosaminyl-3′-O-β-D-di-N- | Cs73Y | 157 |
| acetylgalactosaminoside | | |
| CBD-1′-O-β-D-di-xylosyl-3′-O-β-D-xyloside | Cs73Y | 157 |
| CBD-1′-O-α-L-di-rhamnosyl-3′-O-α-L-rhamnoside | Cs73Y | 157 |
| CBD-1′-O-β-D-di-galactosyl-3′-O-β-D-galactoside | Cs73Y | 157 |
| CBD-1′-O-β-D-di-N-acetylglucosaminyl-3′-O-β-D-N- | Cs73Y | 157 |
| acetylglucosaminoside | | |
| CBD-1′-O-β-D-di-arabinosyl-3′-O-β-D-arabinoside | Cs73Y | 157 |
| CBD-1′-O-β-D-di-N-acetylgalactosaminyl-3′-O-β-D-N- | Cs73Y | 157 |
| acetylgalactosaminoside | | |
| CBDV-1′-O-β-D-di-xylosyl-3′-O-β-D-xyloside | Cs73Y | 157 |
| CBDV-1′-O-α-L-di-rhamnosyl-3′-O-α-L-rhamnoside | Cs73Y | 157 |
| CBDV-1′-O-β-D-di-galactosyl-3′-O-β-D-galactoside | Cs73Y | 157 |
| CBDV-1′-O-β-D-di-N-acetylglucosaminyl-3′-O-β-D-N- | Cs73Y | 157 |
| acetylglucosaminoside | | |
| CBDV-1′-O-β-D-di-arabinosyl-3′-O-β-D-arabinoside | Cs73Y | 157 |
| CBDV-1′-O-β-D-di-N-acetylgalactosaminyl-3′-O-β-D-N- | Cs73Y | 157 |
| acetylgalactosaminoside | | |
| CBG-1′-O-β-D-di-xylosyl-3′-O-β-D-xyloside | Cs73Y | 157 |
| CBG-1′-O-α-L-di-rhamnosyl-3′-O-α-L-rhamnoside | Cs73Y | 157 |
| CBG-1′-O-β-D-di-galactosyl-3′-O-β-D-galactoside | Cs73Y | 157 |
| CBG-1′-O-β-D-di-N-acetylglucosaminyl-3′-O-β-D-N- | Cs73Y | 157 |
| acetylglucosaminoside | | |
| CBG-1′-O-β-D-di-arabinosyl-3′-O-β-D-arabinoside | Cs73Y | 157 |
| CBG-1′-O-β-D-di-N-acetylgalactosaminyl-3′-O-β-D-N- | Cs73Y | 157 |
| acetylgalactosaminoside | | |
| CBD-1′-O-β-D-di-xylosyl-3′-O-β-D-di-xyloside | Cs73Y | 157 |
| CBD-1′-O-α-D-di-rhamnosyl-3′-O-α-D-di-rhamnoside | Cs73Y | 157 |
| CBD-1′-O-β-D-di-galactosyl-3′-O-β-D-di-galactoside | Cs73Y | 157 |
| CBD-1′-O-β-D-di-N-acetylglucosaminyl-3′-O-β-D-di-N- | Cs73Y | 157 |
| acetylglucosaminoside | | |
| CBD-1′-O-β-D-di-arabinosyl-3′-O-β-D-di-arabinoside | Cs73Y | 157 |
| CBD-1′-O-β-D-di-N-acetylgalactosaminyl-3′-O-β-D-di-N- | Cs73Y | 157 |
| acetylgalactosaminoside | | |
| CBDV-1′-O-β-D-di-xylosyl-3′-O-β-D-di-xyloside | Cs73Y | 157 |
| CBDV-1′-O-α-D-di-rhamnosyl-3′-O-α-D-di-rhamnoside | Cs73Y | 157 |
| CBDV-1′-O-β-D-di-galactosyl-3′-O-β-D-di-galactoside | Cs73Y | 157 |
| CBDV-1′-O-β-D-di-N-acetylglucosaminyl-3′-O-β-D-di-N- | Cs73Y | 157 |
| acetylglucosaminoside | | |
| CBDV-1′-O-β-D-di-arabinosyl-3′-O-β-D-di-arabinoside | Cs73Y | 157 |
| CBDV-1′-O-β-D-di-N-acetylgalactosaminyl-3′-O-β-D-di-N- | Cs73Y | 157 |
| acetylgalactosaminoside | | |
| CBG-1′-O-β-D-di-xylosyl-3′-O-β-D-di-xyloside | Cs73Y | 157 |
| CBG-1′-O-α-D-di-rhamnosyl-3′-O-α-D-di-rhamnoside | Cs73Y | 157 |
| CBG-1′-O-β-D-di-galactosyl-3′-O-β-D-di-galactoside | Cs73Y | 157 |
| CBG-1′-O-β-D-di-N-acetylglucosaminyl-3′-O-β-D-di-N- | Cs73Y | 157 |
| acetylglucosaminoside | | |
| CBG-1′-O-β-D-di-arabinosyl-3′-O-β-D-di-arabinoside | Cs73Y | 157 |
| CBG-1′-O-β-D-di-N-acetylgalactosaminyl-3′-O-β-D-di-N- | Cs73Y | 157 |
| acetylgalactosaminoside | | |

In a further aspect the invention provides a composition comprising the fermentation liquid of the invention and one or more agents, additives and/or excipients. Agents, additives and/or excipients includes formulation additives, stabilising agent and fillers.

The composition of the invention may be formulated into a dry solid form by using methods known in the art. Further, the composition may be in dry form such as a spray dried, spray cooled, lyophilized, flash frozen, granular, microgranular, capsule or microcapsule form made using methods known in the art.

The composition of the invention may also be formulated into liquid stabilized form using methods known in the art. Further, the composition may be in liquid form such as a stabilized liquid comprising one or more stabilizers such as sugars and/or polyols (e.g. sugar alcohols) and/or organic acids (e.g. lactic acid).

In one particular embodiment, the composition is refined into a beverage suitable for human or animal ingestion and the cannabinoid glycoside has increased water solubility compared to the un-glycosylted cannabinioid. In another particular embodiment, the composition is refined into a solid food item suitable for human or animal ingestion and wherein the cannabinoid glycoside has increased water solubility compared to the unglycosylated cannabinioid.

### Pharmaceutical Preparations

In a further aspect the invention provides a method for preparing a pharmaceutical preparation comprising mixing the composition of the invention with one or more pharmaceutical grade excipient, additives and/or adjuvants. In another aspect the invention provides a method for preparing a pharmaceutical preparation comprising mixing a novel cannabinoid glycoside of the invention or a composition of the invention with one or more pharmaceutical grade excipient, additives and/or adjuvants. Cannabinoid glycosides often acts as prodrugs, where the glycosyl group are cleaved off in the body leaving the cannabinoid as the active pharmaceutical compound.

The pharmaceutical preparation may be in the form of a powder, tablet, capsule, hard chewable and or soft lozenge or a gum. The pharmaceutical preparation may alternatively be in the form of a liquid pharmaceutical solution.

The present invention also provides a pharmaceutical preparation obtainable from the method of the invention for preparing the pharmaceutical preparation. The pharmaceutical preparation can in an embodiment be used as a medicament or a prodrug for preventing, treating, alleviating and/or relieving a disease in a mammal. Such diseases include, but are not limited to NASH, Epilepsy, Vomiting, Nausea, Cancer, Multiple sclerosis, Spasticity, Chronic pain, Anorexia, Loss of appetite, Parkinson's, Dravet Syndrome (Severe Myoclonic Epilepsy of Infancy), Lennox-Gastaut Syndrome, Substance (Drug) Abuse, Diabetes, Seizures, Panic Disorders, Social Anxiety Disorders (SAD), Generalized Anxiety Disorder (GAD), Anxiety Disorders, Agoraphobia, Infantile Spasm (West Syndrome), Psoriasis, Postherpetic Neuralgia, Motor Neuron Diseases, Amyotrophic Lateral Sclerosis, Tourette Syndrome, Tic Disorder, Cerebral Palsy, Graft Versus Host Disease (GVHD), Crohn's Disease (Regional Enteritis), Inflammatory Bowel Disease, Fragile X Syndrome, Bipolar Disorder (Manic Depression), Osteoarthritis, Huntington Disease, Schizophrenia, Autism, Restless Legs Syndrome, Human Immunodeficiency Virus (HIV) Infections (AIDS), Hypertension, Liver Fibrosis, Hepatic Injury, Prader-Willi Syndrome (PWS), Post-Traumatic Stress Disorder (PTSD), Fatty Liver Disease, Glaucoma, Inflammatory disease, Clostridium difficile infection, Colorectal tumor, Inflammatory bowel disease, Intestine disease, Irritable bowel syndrome, Ulcerative colitis, Cognitive disorder, Brain hypoxia, Fibrosis, Sleep apnea and motor neuron disease. Other medical conditions include relief of side effects from other medication including nausea due to chemotherapy, spasticity, neuropathic pain, dizziness, sedation, confusion, dissociation and “feeling high”. The mammal is preferably a human, a livestock and/or pet animal.

Glycosylated cannabinoids can act as prodrugs, since upon administration sugar molecules may be cleaved off the cannabinoid acceptor at various locations in the body by cytosolic glucosidase enzymes found e.g. in the liver, small intestine, spleen and/or kidney. Microbial glucosidase enzymes can also cleave the sugar molecule off from the cannabinoid acceptor and such microbes can be found e.g. in the gastrointestinal tract (gut microbiome) and in human saliva (salivary microbiome). When glycosides or sugars are attached to the cannabinoid acceptor this glycoside may be biologically inert, while it may regain its biological activity and therapeutic effect upon removal of the sugars from cannabinoid acceptor.

### Method of Use

In a final aspect the invention provides a method for using the pharmaceutical preparation of the disclosure for treating a disease in a mammal, comprising administering a therapeutically effective amount of the pharmaceutical preparation to the mammal. Such diseases include, but are not limited to NASH, Epilepsy, Vomiting, Nausea, Cancer, Multiple sclerosis, Spasticity, Chronic pain, Anorexia, Loss of appetite, Parkinson's, Dravet Syndrome (Severe Myoclonic Epilepsy of Infancy), Lennox-Gastaut Syndrome, Substance (Drug) Abuse, Diabetes, Seizures, Panic Disorders, Social Anxiety Disorders (SAD), Generalized Anxiety Disorder (GAD), Anxiety Disorders, Agoraphobia, Infantile Spasm (West Syndrome), Psoriasis, Postherpetic Neuralgia, Motor Neuron Diseases, Amyotrophic Lateral Sclerosis, Tourette Syndrome, Tic Disorder, Cerebral Palsy, Graft Versus Host Disease (GVHD), Crohn's Disease (Regional Enteritis), Inflammatory Bowel Disease, Fragile X Syndrome, Bipolar Disorder (Manic Depression), Osteoarthritis, Huntington Disease, Schizophrenia, Autism, Restless Legs Syndrome, Human Immunodeficiency Virus (HIV) Infections (AIDS), Hypertension, Liver Fibrosis, Hepatic Injury, Prader-Willi Syndrome (PWS), Post-Traumatic Stress Disorder (PTSD), Fatty Liver Disease, Glaucoma, Inflammatory disease, Clostridium difficile infection, Colorectal tumor, Inflammatory bowel disease, Intestine disease, Irritable bowel syndrome, Ulcerative colitis, Cognitive disorder, Brain hypoxia, Fibrosis, Sleep apnea and motor neuron disease. Other medical conditions include relief of side effects from other medication including nausea due to chemotherapy, spasticity, neuropathic pain, dizziness, sedation, confusion, dissociation and “feeling high”.

### Sequences

The present application contains a Sequence Listing prepared in PatentIn version 3.5.1, which is also submitted electronically in ST25 format which is hereby incorporated by reference in its entirety.

Throughout this disclosure short names or abbreviations for genes, primers and/or enzymes may be employed, such short names being linked to sequence identifiers as follows:

| Gene or primer short name | Sequence identifier |
|---|---|
| UGT708G3 | SEQ ID NO: 2 |
| UGT708G2 | SEQ ID NO: 4 |
| UGT708G1 | SEQ ID NO: 6 |
| OsCGT | SEQ ID NO: 8 |
| FeUGT708C1 | SEQ ID NO: 10 |
| GmUGT708D1 | SEQ ID NO: 12 |
| ZmUGT708A6 | SEQ ID NO: 14 |
| MiCGT | SEQ ID NO: 16 |
| GtUF6CGT1 | SEQ ID NO: 18 |
| DcUGT2 | SEQ ID NO: 20 |
| DcUGT4 | SEQ ID NO: 22 |
| DcUGT5 | SEQ ID NO: 24 |
| UGT73B5 | SEQ ID NO: 26 |
| UGT76C5 | SEQ ID NO: 28 |
| UGT73B3 | SEQ ID NO: 30 |
| UGT71E1 | SEQ ID NO: 32 |
| UGT5 | SEQ ID NO: 34 |
| UGT1A10 | SEQ ID NO: 36 |
| UGT1A9 | SEQ ID NO: 38 |
| UGT2B7 | SEQ ID NO: 40 |
| Geranyl diphosphate synthase | SEQ ID NO: 46 |
| Acyl-activating enzyme 1 | SEQ ID NO: 48 |
| olivetol synthase | SEQ ID NO: 50 |
| olivetolic acid cyclase | SEQ ID NO: 52 |
| Aromatic prenyltransferase 3 | SEQ ID NO: 54 |
| Δ9-tetrahydrocannabinolic acid synthase | SEQ ID NO: 56 |
| cannabidiolic acid synthase | SEQ ID NO: 58 |
| cannabichromenic acid synthase | SEQ ID NO: 60 |
| Primer PR0001 | SEQ ID NO: 61 |
| Primer PR0002 | SEQ ID NO: 62 |
| Primer PR0003 | SEQ ID NO: 63 |
| Primer PR0004 | SEQ ID NO: 64 |
| Primer PR0005 | SEQ ID NO: 65 |
| Primer PR0006 | SEQ ID NO: 66 |
| Primer PR0007 | SEQ ID NO: 67 |
| Primer PR0008 | SEQ ID NO: 68 |
| Primer PR0009 | SEQ ID NO: 69 |
| Primer PR0010 | SEQ ID NO: 70 |
| Primer PR0011 | SEQ ID NO: 71 |
| Primer PR0012 | SEQ ID NO: 72 |
| Primer PR0013 | SEQ ID NO: 73 |
| Primer PR0014 | SEQ ID NO: 74 |
| Primer PR0015 | SEQ ID NO: 75 |
| Primer PR0016 | SEQ ID NO: 76 |
| Primer PR0017 | SEQ ID NO: 77 |
| Primer PR0018 | SEQ ID NO: 78 |
| Primer PR0019 | SEQ ID NO: 79 |
| Primer PR0020 | SEQ ID NO: 80 |
| Primer PR0021 | SEQ ID NO: 81 |
| Primer PR0022 | SEQ ID NO: 82 |
| Primer PR0023 | SEQ ID NO: 83 |
| Primer PR0024 | SEQ ID NO: 84 |
| Primer PR0025 | SEQ ID NO: 85 |
| Primer PR0026 | SEQ ID NO: 86 |
| Primer PR0027 | SEQ ID NO: 87 |
| Primer PR0028 | SEQ ID NO: 88 |
| Primer PR0029 | SEQ ID NO: 89 |
| Primer PR0030 | SEQ ID NO: 90 |
| Primer PR0031 | SEQ ID NO: 91 |
| Primer PR0032 | SEQ ID NO: 92 |
| Primer PR0033 | SEQ ID NO: 93 |
| Primer PR0034 | SEQ ID NO: 94 |
| Primer PR0035 | SEQ ID NO: 95 |
| Primer PR0036 | SEQ ID NO: 96 |
| Primer PR0037 | SEQ ID NO: 97 |
| Primer PR0038 | SEQ ID NO: 98 |
| Primer PR0039 | SEQ ID NO: 99 |
| Primer PR0040 | SEQ ID NO: 100 |
| UGT 88G | SEQ ID NO: 102 |
| UGT 88B_2 | SEQ ID NO: 104 |
| UGT 76G1 | SEQ ID NO: 106 |
| At73C5 | SEQ ID NO: 108 |
| At71D1 | SEQ ID NO: 110 |
| At72B1 | SEQ ID NO: 112 |
| Sr71E1 | SEQ ID NO: 114 |
| OsEUGT11 | SEQ ID NO: 116 |
| Sp73E | SEQ ID NO: 118 |
| OsO-1 | SEQ ID NO: 120 |
| At84B1 | SEQ ID NO: 122 |
| Sr76G1 | SEQ ID NO: 124 |
| Pa85 | SEQ ID NO: 126 |
| CrUGT-2 | SEQ ID NO: 128 |
| At73B3 | SEQ ID NO: 130 |
| At71C1-Sr71E1 354 | SEQ ID NO: 132 |
| Pa72 | SEQ ID NO: 134 |
| At73B5 | SEQ ID NO: 136 |
| At71C1_At71C2 353 | SEQ ID NO: 138 |
| Cp89B | SEQ ID NO: 140 |
| Sp89B | SEQ ID NO: 142 |
| Tc90A | SEQ ID NO: 144 |
| Si94D | SEQ ID NO: 146 |
| Pt88G | SEQ ID NO: 148 |
| Ha88B_2 | SEQ ID NO: 150 |
| Ac73T | SEQ ID NO: 152 |
| Si73X | SEQ ID NO: 154 |
| Tc74Z | SEQ ID NO: 156 |
| Cs73Y | SEQ ID NO: 158 |
| Pt73Y | SEQ ID NO: 160 |
| Ac73Z | SEQ ID NO: 162 |
| Bv75C | SEQ ID NO: 164 |
| Pt78G | SEQ ID NO: 166 |
| Si82A | SEQ ID NO: 168 |
| Ad74X | SEQ ID NO: 170 |
| Cs74S | SEQ ID NO: 172 |
| Ad72AA | SEQ ID NO: 174 |
| Si71E_2 | SEQ ID NO: 176 |
| Vv71R | SEQ ID NO: 178 |
| Ha72B | SEQ ID NO: 180 |
| Sp73A | SEQ ID NO: 182 |
| Bv73P | SEQ ID NO: 184 |
| Pt72B | SEQ ID NO: 186 |
| Qs72S_1 | SEQ ID NO: 188 |
| Ad72X | SEQ ID NO: 190 |
| Cp73B | SEQ ID NO: 192 |
| Zj71A | SEQ ID NO: 194 |
| Ha71S | SEQ ID NO: 196 |
| Ac73H | SEQ ID NO: 198 |
| Cp71B | SEQ ID NO: 200 |
| Ha72T | SEQ ID NO: 202 |
| Sp73Q | SEQ ID NO: 204 |
| Sp72T | SEQ ID NO: 206 |
| Cs73Y | SEQ ID NO: 208 |
| GmSuSy | SEQ ID NO: 210 |
| BsGalE | SEQ ID NO: 212 |
| AtUXS3 | SEQ ID NO: 214 |
| AtRHM2-C | SEQ ID NO: 216 |
| AtRHM2-N | SEQ ID NO: 218 |
| AtRHM2 | SEQ ID NO: 220 |
| AtUGDH1 | SEQ ID NO: 222 |
| AtMUR4 | SEQ ID NO: 224 |
| PsWbgU | SEQ ID NO: 226 |
| CsTKS-CsOAC | SEQ ID NO: 228 |
| AgGPPS2 | SEQ ID NO: 230 |
| CsTHCAS (ProA) | SEQ ID NO: 232 |
| CsCBDAS (ProA) | SEQ ID NO: 234 |
| CsPT4ΔN-terminal | SEQ ID NO: 236 |
| SsNphB(Q295F) | SEQ ID NO: 238 |
| CsAAE1 | SEQ ID NO: 240 |
| Primer PR0041 | SEQ ID NO: 241 |
| Primer PR0042 | SEQ ID NO: 242 |
| Primer PR0043 | SEQ ID NO: 243 |
| Primer PR0044 | SEQ ID NO: 244 |
| Primer PR0045 | SEQ ID NO: 245 |
| Primer PR0046 | SEQ ID NO: 246 |
| Primer PR0047 | SEQ ID NO: 247 |
| Primer PR0048 | SEQ ID NO: 248 |
| Primer PR0049 | SEQ ID NO: 249 |
| Primer PR0050 | SEQ ID NO: 250 |
| Primer PR0051 | SEQ ID NO: 251 |
| Primer PR0052 | SEQ ID NO: 252 |
| Primer PR0053 | SEQ ID NO: 253 |
| Primer PR0054 | SEQ ID NO: 254 |
| Primer PR0055 | SEQ ID NO: 255 |
| Primer PR0056 | SEQ ID NO: 256 |
| Primer PR0057 | SEQ ID NO: 257 |
| Primer PR0058 | SEQ ID NO: 258 |
| Primer PR0059 | SEQ ID NO: 259 |
| Primer PR0060 | SEQ ID NO: 260 |
| Primer PR0061 | SEQ ID NO: 261 |
| Primer PR0062 | SEQ ID NO: 262 |
| Primer PR0063 | SEQ ID NO: 263 |
| Primer PR0064 | SEQ ID NO: 264 |
| Primer PR0065 | SEQ ID NO: 265 |
| Primer PR0066 | SEQ ID NO: 266 |
| Primer PR0067 | SEQ ID NO: 267 |
| Primer PR0068 | SEQ ID NO: 268 |
| Primer PR0069 | SEQ ID NO: 269 |
| Primer PR0070 | SEQ ID NO: 270 |
| Primer PR0071 | SEQ ID NO: 271 |
| Primer PR0072 | SEQ ID NO: 272 |
| Primer PR0073 | SEQ ID NO: 273 |
| Primer PR0074 | SEQ ID NO: 274 |
| Primer PR0075 | SEQ ID NO: 275 |
| Primer PR0076 | SEQ ID NO: 276 |
| Primer PR0077 | SEQ ID NO: 277 |
| Primer PR0078 | SEQ ID NO: 278 |
| Primer PR0079 | SEQ ID NO: 279 |
| Primer PR0080 | SEQ ID NO: 280 |
| Primer PR0081 | SEQ ID NO: 281 |
| Primer PR0082 | SEQ ID NO: 282 |
| Primer PR0083 | SEQ ID NO: 283 |
| Primer PR0084 | SEQ ID NO: 284 |
| Primer PR0085 | SEQ ID NO: 285 |
| Primer PR0086 | SEQ ID NO: 286 |
| Primer PR0087 | SEQ ID NO: 287 |
| Primer PR0088 | SEQ ID NO: 288 |
| Primer PR0089 | SEQ ID NO: 289 |
| Primer PR0090 | SEQ ID NO: 290 |
| Primer PR0091 | SEQ ID NO: 291 |
| Primer PR0092 | SEQ ID NO: 292 |
| Primer PR0093 | SEQ ID NO: 293 |
| Primer PR0094 | SEQ ID NO: 294 |
| Primer PR0095 | SEQ ID NO: 295 |
| Primer PR0096 | SEQ ID NO: 296 |
| Primer PR0097 | SEQ ID NO: 297 |
| Primer PR0098 | SEQ ID NO: 298 |
| Primer PR0099 | SEQ ID NO: 299 |
| Primer PR0100 | SEQ ID NO: 300 |
| Primer PR0101 | SEQ ID NO: 301 |
| Primer PR0102 | SEQ ID NO: 302 |
| Primer PR0103 | SEQ ID NO: 303 |
| Primer PR0104 | SEQ ID NO: 304 |
| Primer PR0105 | SEQ ID NO: 305 |
| Primer PR0106 | SEQ ID NO: 306 |
| Primer PR0107 | SEQ ID NO: 307 |
| Primer PR0108 | SEQ ID NO: 308 |
| Primer PR0109 | SEQ ID NO: 309 |
| Primer PR0110 | SEQ ID NO: 310 |
| Primer PR0111 | SEQ ID NO: 311 |
| Primer PR0112 | SEQ ID NO: 312 |
| Primer PR0113 | SEQ ID NO: 313 |
| Primer PR0114 | SEQ ID NO: 314 |
| Primer PR0115 | SEQ ID NO: 315 |
| Primer PR0116 | SEQ ID NO: 316 |
| Primer PR0117 | SEQ ID NO: 317 |
| Primer PR0118 | SEQ ID NO: 318 |
| Primer PR0119 | SEQ ID NO: 319 |
| Primer PR0120 | SEQ ID NO: 320 |

| Enzyme short name | Sequence identifier |
|---|---|
| UGT708G3 | SEQ ID NO: 1 |
| UGT708G2 | SEQ ID NO: 3 |
| UGT708G1 | SEQ ID NO: 5 |
| OsCGT | SEQ ID NO: 7 |
| FeUGT708C1 | SEQ ID NO: 9 |
| GmUGT708D1 | SEQ ID NO: 11 |
| ZmUGT708A6 | SEQ ID NO: 13 |
| MiCGT | SEQ ID NO: 15 |
| GtUF6CGT1 | SEQ ID NO: 17 |
| DcUGT2 | SEQ ID NO: 19 |
| DcUGT4 | SEQ ID NO: 21 |
| DcUGT5 | SEQ ID NO: 23 |
| UGT73B5 | SEQ ID NO: 25 |
| UGT76C5 | SEQ ID NO: 27 |
| UGT73B3 | SEQ ID NO: 29 |
| UGT71E1 | SEQ ID NO: 31 |
| UGT5 | SEQ ID NO: 33 |
| UGT1A10 | SEQ ID NO: 35 |
| UGT1A9 | SEQ ID NO: 37 |
| UGT2B7 | SEQ ID NO: 39 |
| Geranyl diphosphate synthase | SEQ ID NO: 41 |
| Acyl-activating enzyme 1 | SEQ ID NO: 43 |
| olivetol synthase | SEQ ID NO: 45 |
| olivetolic acid cyclase | SEQ ID NO: 47 |
| Aromatic prenyltransferase 3 | SEQ ID NO: 49 |
| Δ9-tetrahydrocannabinolic acid synthase | SEQ ID NO: 51 |
| cannabidiolic acid synthase | SEQ ID NO: 53 |
| cannabichromenic acid synthase | SEQ ID NO: 55 |
| UGT 88G | SEQ ID NO: 101 |
| UGT 88B_2 | SEQ ID NO: 103 |
| UGT 76G1 | SEQ ID NO: 105 |
| At73C5 | SEQ ID NO: 107 |
| At71D1 | SEQ ID NO: 109 |
| At72B1 | SEQ ID NO: 111 |
| Sr71E1 | SEQ ID NO: 113 |
| OsEUGT11 | SEQ ID NO: 115 |
| Sp73E | SEQ ID NO: 117 |
| OsO-1 | SEQ ID NO: 119 |
| At84B1 | SEQ ID NO: 121 |
| Sr76G1 | SEQ ID NO: 123 |
| Pa85 | SEQ ID NO: 125 |
| CrUGT-2 | SEQ ID NO: 127 |
| At73B3 | SEQ ID NO: 129 |
| At71C1-Sr71E1 354 | SEQ ID NO: 131 |
| Pa72 | SEQ ID NO: 133 |
| At73B5 | SEQ ID NO: 135 |
| At71C1_At71C2 353 | SEQ ID NO: 137 |
| Cp89B | SEQ ID NO: 139 |
| Sp89B | SEQ ID NO: 141 |
| Tc90A | SEQ ID NO: 143 |
| Si94D | SEQ ID NO: 145 |
| Pt88G | SEQ ID NO: 147 |
| Ha88B_2 | SEQ ID NO: 149 |
| Ac73T | SEQ ID NO: 151 |
| Si73X | SEQ ID NO: 153 |
| Tc74Z | SEQ ID NO: 155 |
| Cs73Y | SEQ ID NO: 157 |
| Pt73Y | SEQ ID NO: 159 |
| Ac73Z | SEQ ID NO: 161 |
| Bv75C | SEQ ID NO: 163 |
| Pt78G | SEQ ID NO: 165 |
| Si82A | SEQ ID NO: 167 |
| Ad74X | SEQ ID NO: 169 |
| Cs74S | SEQ ID NO: 171 |
| Ad72AA | SEQ ID NO: 173 |
| Si71E_2 | SEQ ID NO: 175 |
| Vv71R | SEQ ID NO: 177 |
| Ha72B | SEQ ID NO: 179 |
| Sp73A | SEQ ID NO: 181 |
| Bv73P | SEQ ID NO: 183 |
| Pt72B | SEQ ID NO: 185 |
| Qs72S_1 | SEQ ID NO: 187 |
| Ad72X | SEQ ID NO: 189 |
| Cp73B | SEQ ID NO: 191 |
| Zj71A | SEQ ID NO: 193 |
| Ha71S | SEQ ID NO: 195 |
| Ac73H | SEQ ID NO: 197 |
| Cp71B | SEQ ID NO: 199 |
| Ha72T | SEQ ID NO: 201 |
| Sp73Q | SEQ ID NO: 203 |
| Sp72T | SEQ ID NO: 205 |
| Cs73Y | SEQ ID NO: 207 |
| GmSuSy | SEQ ID NO: 209 |
| BsGalE | SEQ ID NO: 211 |
| AtUXS3 | SEQ ID NO: 213 |
| AtRHM2-C | SEQ ID NO: 215 |
| AtRHM2-N | SEQ ID NO: 217 |
| AtRHM2 | SEQ ID NO: 219 |
| AtUGDH1 | SEQ ID NO: 221 |
| AtMUR4 | SEQ ID NO: 223 |
| PsWbgU | SEQ ID NO: 225 |
| CsTKS-CsOAC | SEQ ID NO: 227 |
| AgGPPS2 | SEQ ID NO: 229 |
| CsTHCAS (ProA) | SEQ ID NO: 231 |
| CsCBDAS (ProA) | SEQ ID NO: 233 |
| CsPT4ΔN-terminal | SEQ ID NO: 235 |
| SsNphB(Q295F) | SEQ ID NO: 237 |
| CsAAE1 | SEQ ID NO: 239 |

### Itemized Aspects and Embodiments of the Invention

The present invention further provides the following embodiments and items:

1. A microbial host cell genetically modified to intracellularly produce a cannabinoid glycoside, said cell expressing a heterologous gene encoding at least one glycosyl transferase capable of intracellularly glycosylating a cannabinoid acceptor with a glycosyl donor thereby producing the cannabinoid glycoside.

2. The genetically modified host cell of item 1, wherein the cannabinoid acceptor is the condensation product or a derivative thereof a prenyl donor and a prenyl acceptor.

3. The genetically modified host cell of item 1 or 2, wherein the cannabinoid acceptor is a cannabinoid aglycone or a cannabinoid glycoside.

4. The genetically modified host cell of any preceding item, wherein the prenyl donor is selected from the group of gernyl diphosphate, neryl diphosphate, farnesyl diphosphate, dimethylallyl diphosphate and geranylgeranyl pyrophosphate.

5. The genetically modified host cell of item 4, wherein the prenyl donor is geranyl diphosphate.

6. The genetically modified host cell of any preceding item, wherein the prenyl acceptor is a derivative of a fatty acid selected from the group of hexanoic acid, butanoic acid, pentanoic acid, heptanoic acid, octanoic acid, nonanoic acid, decanoic acid; 4-methyl hexanoic acid, 5-hexanoic acid and 6-heptonic acid.

7. The genetically modified host cell of item 6, wherein the prenyl acceptor is selected from the group of olivetolic acid, divarinolic acid, olivetol, phlorisovalerophenone, resveratrol, naringenin, phloroglucinol and homogentisic acid.

8. The genetically modified host cell of item 7, wherein the prenyl acceptor is olivetolic acid and/or divarinolic acid.

9. The genetically modified host cell of any preceding item, wherein the cannabionoid acceptor and/or the cannabinoid glycoside is an agonist or an antagonist to a human or animal cannabinoid receptor.

10. The genetically modified host cell of item 9, wherein the cannabionoid acceptor and/or the cannabinoid glycoside is non-psychotropic or at least 10% less phsychotropic than THC.

11. The genetically modified host cell of any preceding item, wherein the cannabinoid acceptor is neutral or acidic.

12. The genetically modified host cell of any preceding item, wherein the cannabinoid acceptor is selected from the group of cannabichromene-type (CBC), cannabigerol-type (CBG), cannabidiol-type (CBD), Tetrahydrocannabinol-type (THC), cannabicyclol-type (CBL), cannabielsoin-type (CBE), cannabinol-type (CBN), cannabinodiol-type (CBND) and cannabitriol-type (CBT).

13. The genetically modified host cell of item 12, wherein the cannabinoid acceptor is selected from the group of cannabigerolic acid (CBGA), cannabigerolic acid monomethylether (CBGAM), cannabigerol monomethylether (CBGM), cannabigerovarinic acid (CBGVA), cannabigerovarin (CBGV), cannabichromenic acid (CBCA), cannabichromevarinic acid (CBCVA), cannabichromevarin (CBCV), cannabidiolic acid (CBDA), cannabidiol, monomethylether (CBDM), cannabidiol-C4 (CBD-C4), cannabidivarinic acid (CBDVA) cannabidivarin (CBDV), cannabidiorcol (CBD-C1), Δ9-trans-tetrahydrocannabinol (Δ9-THC), Δ9-tetrahydrocannabinol (Δ9-THC), Δ9-cis-tetrahydrocannabinol (A9-THC), tetrahydrocannabinolic acid (THCA), Δ9-tetrahydrocannabinolic acid A (THCA-A), Δ9-tetrahydrocannabinolic acid B (THCA-B), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-trans-tetrahydrocannabinol (Δ8-THC), Δ8-tetrahydrocannabinol (Δ8-THC), A8-cis-tetrahydrocannabinol (Δ8-THC), cannabicyclolic acid (CBLA), cannabicyclol (CBL) cannabicyclovarin (CBLV), cannabielsoic acid A (CBEA-A), cannabielsoic acid B (CBEA-B), cannabielsoin (CBE), cannabielsoinic acid, cannabicitran, cannabicitranic acid, cannabinolic acid, (CBNA), cannabinol methylether (CBNM), cannabinol-C4, (CBN-C4), cannabivarin (CBV), cannabinol-C2 (CNB-C2), cannabiorcol (CBN-C1), cannabinodiol, (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin, (CBTVE), dehydrocannabifuran (DCBF), cannabifuran (CBF), cannabichromanon (CBCN), cannabicivan (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-I-benzoxocin-5-methanol (OH-iso-HHCV), cannabiripsol (CBR), trihydroxy-delta-9-tetrahydrocannabinol (triOH-THC), perrottetinene, perrottetinenic acid, 11-Nor-9-carboxy-THC, 11-hydroxy-Δ9-THC, Nor-9-carboxy-Δ9-tetrahydrocannabinol, tetrahydrocannabiphorol (THCP), cannabidiphorol (CBDP), Cannabimovone (CBM) and derivatives thereof.

14. The genetically modified host cell of items 1 to 11, wherein the cannabinoid acceptor is an endocannabinoid selected from the group of arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),1 I (Z)-eicosatrienoic acid ethanolamide (mead acid ethanolamide), heptadecanoyl ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA).

15. The genetically modified host cell of any preceding item, wherein the glycosyl donor is selected from one or more of NTP-glycoside, NDP-glycoside and NMP-glycoside.

16. The genetically modified host cell of item 15, wherein the nucleoside of the nucleotide glycoside is selected from Uridine, Adenosin, Guanosin, Cytidin and deoxythymidine.

17. The genetically modified host cell of item 16, wherein the glycosyl donor is selected from UDP-glycosides, ADP-glycosides, CDP-glycosides, CMP-glycosides, dTDP-glycosides and GDP-glycosides.

18. The genetically modified host cell of item 17, wherein the glycosyl donor is selected from UDP-D-glucose (UDP-Glc); UDP-galactose (UDP-Gal); UDP-D-xylose (UDP-Xyl); UDP-N-acetyl-D-glucosamine (UDP-GlcNAc); UDP-N-acetyl-D-galactosamine (UDP-GaINAc); UDP-D-glucuronic acid (UDP-GlcA); UDP-D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

19. The genetically modified host cell of any preceding item, wherein the glycosyl transferase is derived from a plant or a fungus.

20. The genetically modified host cell of item 19, wherein the plant is selected from Oryza sativa, Crocus sativus, Nicotiana tabacum, Stevia rebaudiana, Nicotiana benthatamiana and Arabidopsis thaliana.

21. The genetically modified host cell of item 1 to 20, wherein the glycosyl transferase is capable of using nucleotide glycoside selected from NTP-glycoside, NDP-glycoside and/or NMP-glycoside as glycosyl donor for glycosylating the cannabinoid.

22. The genetically modified host cell of item 21, wherein the nucleoside of the nucleotide glycoside is selected from Uridine, Adenosin, Guanosin, Cytidin and deoxythymidine.

23. The genetically modified host cell of item 22, wherein the glycosyl donor is selected from UDP-glycosides, ADP-glycosides, CDP-glycosides, CMP-glycosides, dTDP-glycosides and GDP-glycosides.

24. The genetically modified host cell of any preceding item, wherein the glycosyl transferase is an O-glycoside transferase and/or a C-glycoside transferase.

25. The genetically modified host cell of item 24, wherein the glycosyl transferase is a cannabinoid aglycone O-glycosyltransferase.

26. The genetically modified host cell of item 24, wherein the glycosyl transferase is a cannabinoid glycoside O-glycosyltransferase.

27. The genetically modified host cell of item 24, wherein the glycosyl transferase is a cannabinoid aglycone O-glucosyltransferase.

28. The genetically modified host cell of item 24, wherein the glycosyl transferase is a cannabinoid aglycone O-rhamnosyltransferase.

29. The genetically modified host cell of item 24, wherein the glycosyl transferase is a cannabinoid aglycone O-xylosyltransferase.

30. The genetically modified host cell of item 24, wherein the glycosyl transferase is a cannabinoid aglycone O-arabinosyltransferase.

31. The genetically modified host cell of item 24, wherein the glycosyl transferase is a cannabinoid aglycone O—N-acetylgalactosaminyltransferase.

32. The genetically modified host cell of item 24, wherein the glycosyl transferase is a cannabinoid aglycone O—N-acetylglucosaminyltransferase.

33. The genetically modified host cell of item 24, wherein the glycosyl transferase is a cannabinoid aglycone/glycoside mono-O-glycosyltransferase.

34. The genetically modified host cell of item 24, wherein the glycosyl transferase is a cannabinoid aglycone/glycoside di-O-glycosyltransferase.

35. The genetically modified host cell of item 24, wherein the glycosyl transferase is a cannabinoid aglycone/glycoside tri-O-glycosyltransferase.

36. The genetically modified host cell of item 24, wherein the glycosyl transferase is a cannabinoid aglycone/glycoside tetra-O-glycosyltransferase.

37. The genetically modified host cell of item 24, wherein the glycosyl transferase is a cannabinoid O-galactosyltransferase.

38. The genetically modified host cell of item 24, wherein the glycosyl transferase is a cannabinoid O-glucuronosyltransferase.

39. The genetically modified host cell of any preceding item, wherein the glycosyl transferase is selected from EC2.4.1.-, and EC2.4.2.-

40. The genetically modified host cell of item 39, wherein the glycosyl transferase is selected from EC2.4.1.17, EC2.4.1.35, EC2.4.1.159, EC2.4.1.203. EC2.4.1.234, EC2.4.1.236 and EC2.4.1.294.

41. The genetically modified host cell of item 39, wherein the glycosyl transferase is selected from EC2.4.2.40.

42. The genetically modified host cell of any preceding item, wherein the glycosyl transferase is a cannabinoid aglycone O-glycosyltransferase and/or cannabinoid glycoside O-glycosyltransferase, optionally a cannabinoid aglycone O-glycosyltransferase and/or cannabinoid glycoside O-glycosyltransferase which is a at least 70%, such at least 75%, such as at least 80%, such as at least 90%, such as at least 95%, such as at least 99%, such as 100% identity to the glycosyl transferase comprised in anyone of SEQ ID NO: 1, 3, 5, 7, 9, 11, 13, 15, 17, 19, 21, 23, 25, 27, 29, 31, 33, 35, 37, 39, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205 or 207.

43. The genetically modified host cell of item 42, wherein the glycosyl transferase has at least 70%, such at least 75%, such as at least 80%, such as at least 90%, such as at least 95%, such as at least 99%, such as 100% identity to the cannabinoid aglycone O-glycosyltransferase comprised in anyone of SEQ ID NO: 107, 109, 111, 113, 117, 119, 121, 125, 127, 129, 131, 133, 135, 137, 139, 141, 143, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207.

44. The genetically modified host cell of item 42, wherein the glycosyl transferase is a cannabinoid glycoside O-glycosyltransferase, optionally a cannabinoid glycoside O-glycosyltransferase which has at least 70%, such at least 75%, such as at least 80%, such as at least 90%, such as at least 95%, such as at least 99%, such as 100% identity to the cannabinoid glycoside O-glycosyltransferase comprised in anyone of SEQ ID NO: 115, 123 or 145.

45. The genetically modified host cell of item 42, wherein the glycosyl transferase is a cannabinoid aglycone O-glucosyltransferase, optionally a cannabinoid aglycone O-glucosyltransferase which has at least 70%, such at least 75%, such as at least 80%, such as at least 90%, such as at least 95%, such as at least 99%, such as 100% identity to the cannabinoid aglycone O-glucosyltransferase comprised in anyone of SEQ ID NO: 107, 109, 111, 117, 119, 121, 125, 127, 129, 131, 133, 135, 137, 139, 141, 143, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205 or 207.

46. The genetically modified host cell of item 42, wherein the glycosyl transferase is a cannabinoid aglycone O-rhamnosyltransferase, optionally a cannabinoid aglycone O-rhamnosyltransferase which has at least 70%, such at least 75%, such as at least 80%, such as at least 90%, such as at least 95%, such as at least 99%, such as 100% identity to the cannabinoid aglycone O-rhamnosyltransferase comprised in anyone of SEQ ID NO: 107, 125, 127, 147, 149, 151, 157, 159, 161, 177, 183, 191, 197 or 207.

47. The genetically modified host cell of item 42, wherein the glycosyl transferase is a cannabinoid aglycone O-xylosyltransferase, optionally a cannabinoid aglycone O-xylosyltransferase which has at least 70%, such at least 75%, such as at least 80%, such as at least 90%, such as at least 95%, such as at least 99%, such as 100% identity to the cannabinoid aglycone O-xylosyltransferase comprised in anyone of SEQ ID NO: 107, 113, 125, 127, 147, 149, 151, 157, 159, 161, 177, 183, 191, 197 or 207.

48. The genetically modified host cell of item 42, wherein the glycosyl transferase is a cannabinoid aglycone O-arabinosyltransferase, optionally a cannabinoid aglycone O-arabinosyltransferase which has at least 70%, such at least 75%, such as at least 80%, such as at least 90%, such as at least 95%, such as at least 99%, such as 100% identity to the cannabinoid aglycone O-arabinosyltransferase comprised in anyone of SEQ ID NO: 107, 125, 127, 147, 149, 151, 157, 159, 161, 177, 183, 191, 197 or 207.

49. The genetically modified host cell of item 42, wherein the glycosyl transferase is a cannabinoid aglycone O—N-acetylgalactosaminyl transferase, optionally a cannabinoid aglycone O—N-acetylgalactosaminyl transferase which is at least 70%, such at least 75%, such as at least 80%, such as at least 90%, such as at least 95%, such as at least 99%, such as 100% identity to the cannabinoid aglycone O—N-acetylgalactosaminyl transferase comprised in anyone of SEQ ID NO: 107, 125, 127, 147, 149, 151, 157, 159, 161, 177, 183, 191, 197 or 207.

50. The genetically modified host cell of item 42, wherein the glycosyl transferase is a cannabinoid aglycone O—N-acetylglucosaminyl transferase, optionally a cannabinoid aglycone O—N-acetylglucosaminyl transferase which has at least 70%, such at least 75%, such as at least 80%, such as at least 90%, such as at least 95%, such as at least 99%, such as 100% identity to the cannabinoid aglycone O—N-acetylglucosaminyl transferase comprised in anyone of SEQ ID NO: 107, 125, 127, 147, 149, 151, 157, 159, 161, 177, 183, 191, 197 or 207.

51. The genetically modified host cell of item 42, wherein the glycosyl transferase is a cannabinoid aglycone/glycoside di-O-glycosyltransferase, optionally a cannabinoid aglycone/glycoside di-O-glycosyltransferase which has at least 70%, such at least 75%, such as at least 80%, such as at least 90%, such as at least 95%, such as at least 99%, such as 100% identity to the cannabinoid aglycone/glycoside di-O-glycosyltransferase comprised in anyone of SEQ ID NO: 107, 115, 123, 125, 127, 133, 135, 145, 149, 151, 157, 159, 161, 165, 167, 173, 175, 177, 185, 191, 195 or 207.

52. The genetically modified host cell of item 42, wherein the glycosyl transferase is a cannabinoid aglycone/glycoside tri-O-glycosyltransferase, optionally a cannabinoid aglycone/glycoside tri-O-glycosyltransferase which has at least 70%, such at least 75%, such as at least 80%, such as at least 90%, such as at least 95%, such as at least 99%, such as 100% identity to the cannabinoid aglycone/glycoside tri-O-glycosyltransferase comprised in anyone of SEQ ID NO: 107, 115, 123, 145, 157, 159, 191 or 207.

53. The genetically modified host cell of item 42, wherein the glycosyl transferase is a tetra-O-glycosyltransferase, optionally a tetra-O-glycosyltransferase which has at least 70%, such at least 75%, such as at least 80%, such as at least 90%, such as at least 95%, such as at least 99%, such as 100% identity to the cannabinoid aglycone/glycoside tetra-O-glycosyltransferase comprised in anyone of SEQ ID NO: 207.

54. The genetically modified host cell of item 42, wherein the glycosyl transferase is a family 73 glycosyl transferase.

55. The genetically modified host cell of item 54, wherein the glycosyl transferase has at least 70%, such at least 75%, such as at least 80%, such as at least 90%, such as at least 95%, such as at least 99%, such as 100% identity to the glycosyl transferase comprised in anyone of SEQ ID NO: 107, 157, 159, 191 and/or 207.

56. The genetically modified host cell of item 42, wherein the glycosyl transferase has at least 70%, such at least 75%, such as at least 80%, such as at least 90%, such as at least 95%, such as at least 99%, such as 100% identity to the glycosyl transferase comprised in anyone of SEQ ID NO: 135, 143, 147 and/or 171.

57. The genetically modified host cell of item 42, wherein the glycosyl transferase has at least 70%, such at least 75%, such as at least 80%, such as at least 90%, such as at least 95%, such as at least 99%, such as 100% identity to the glycosyl transferase glycosylating CBD, CBDV and/or CBDA comprised in anyone of SEQ ID NO: 107, 109, 111, 113, 117, 125, 127, 129, 135, 137, 139, 141, 147, 149, 151, 153, 157, 159, 161, 177, 179, 183, 191, 193, 197, 201, 205 or 207.

58. The genetically modified host cell of item 42, wherein the glycosyl transferase has at least 70%, such at least 75%, such as at least 80%, such as at least 90%, such as at least 95%, such as at least 99%, such as 100% identity to the glycosyl transferase glycosylating CBG, CBGV and/or CBGA comprised in anyone of SEQ ID NO: 107, 109, 119, 125, 127, 135, 137, 147, 149, 151, 157, 159, 161, 165, 167, 173, 175, 177, 179, 183, 185, 187, 189, 191, 195, 201, 205 or 207,

59. The genetically modified host cell of item 42, wherein the glycosyl transferase has at least 70%, such at least 75%, such as at least 80%, such as at least 90%, such as at least 95%, such as at least 99%, such as 100% identity to the THC glycosylating glycosyl transferase comprised in anyone of SEQ ID NO: 107, 111, 117, 121, 125, 127, 131, 143, 149, 155, 157, 159, 163, 169, 171, 191, 199, 201, 203 or, 207.

60. The genetically modified host cell of item 42, wherein the glycosyl transferase has at least 70%, such at least 75%, such as at least 80%, such as at least 90%, such as at least 95%, such as at least 99%, such as 100% identity to the CBN glycosylating glycosyl transferase comprised in anyone of SEQ ID NO: 125, 127, 133, 135, 149, 151, 157, 159, 175, 177, 181, 191, 195 or 207.

61. The genetically modified host cell of item 42, wherein the glycosyl transferase has at least 70%, such at least 75%, such as at least 80%, such as at least 90%, such as at least 95%, such as at least 99%, such as 100% identity to the CBC glycosylating glycosyl transferase comprised in anyone of SEQ ID NO: 107, 125, 127, 135, 149, 151, 157, 159, 175, 177, 191, 201 or 207.

62. The genetically modified host cell of item 42, wherein the glycosyl transferase has at least 70%, such at least 75%, such as at least 80%, such as is least 90%, such as at least 95%, such as at least 99%, such as 100% identity to the glycosyl transferase comprised in SEQ ID NO: SEQ ID NO: 147, 157, 107, 159, 191, 171, 135, 143.

63. The genetically modified host cell of items 42 to 62, wherein the sequence identity is least 90%, such as at least 95%, such as at least 99%, such as 100%.

64. The genetically modified host cell of item 63, wherein the sequence identity is at least 99%, such as 100%.

65. The genetically modified host cell of item 42, wherein the glycosyl transferase is least 90%, such as at least 95%, such as at least 99%, such as 100% identity to the glycosyl transferase comprised in SEQ ID NO: 25, 27, 29, 31, 33, 35, 37, 39, 101 or 103.

66. The genetically modified host cell of item 65, wherein the glycosyl transferase has at least 95%, such as at least 99%, such as 100% identity to the glycosyl transferase comprised in anyone of SEQ ID NO: 25, 27, 29, 31, 33, 35, 37, 39, 101 or 103.

67. The genetically modified host cell of item 66, wherein the glycosyl transferase is the glycosyl transferase comprised in anyone of SEQ ID NO: 25, 27, 29, 31, 33, 35, 37, 39, 101 or 103.

68. The genetically modified host cell of any preceding items, wherein the expressed glycosyl transferase is absent a signal peptide targeting the glycosyl transferase for secretion.

69. The genetically modified host cell of any preceding items, wherein the glycosyl transferase catalyzes formation of a 1,2-; 1,3-; 1,4- and/or 1,6-glycosidic bond between the glycosyl group and the cannabinoid aglycone or cannabinoid glycoside.

70. The genetically modified host cell of item 69, wherein the glycosyl transferase catalyzes formation of a 1,4- and/or 1,6-glycosidic bond between the glycosyl group and the cannabinoid aglycone or cannabinoid glycoside.

71. The genetically modified host cell of item 70, wherein the glycosyl transferase is the glycosyl transferase comprised in SEQ ID NO: 115 and catalyzes formation of a 1,4-glycosidic bond between the glycosyl group and the cannabinoid aglycone or cannabinoid glycoside.

72. The genetically modified host cell of item 70, wherein the glycosyl transferase is the glycosyl transferase comprised in SEQ ID NO: 145 and catalyzes formation of a 1,6-glycosidic bond between the glycosyl group and the cannabinoid aglycone or cannabinoid glycoside.

73. The genetically modified host cell of any preceding items, wherein the heterologous gene encoding the glycosyl transferase has at least 70%, such at least 75%, such as at least 80%, such as at least 90%, such as at least 95%, such as at least 99%, such as 100% identity to the glycosyl transferase encoding gene comprised in anyone of SEQ ID NO: 2, 4, 6, 8, 10, 12, 14, 16, 18, 20, 22, 24, 26, 28, 30, 32, 34, 36, 38, 40, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 or 208.

74. The genetically modified host cell of item 73, wherein the heterologous gene encoding the glycosyl transferase has at least 70%, such at least 75%, such as at least 80%, such as at least 90%, such as at least 95%, such as at least 99%, such as 100% identity to the glycosyl transferase comprised in SEQ ID NO: 148, 158, 108, 160, 192, 172, 137, 144.

75. The genetically modified host cell of items 73 to 74, wherein the sequence identity is least 90%, such as at least 95%, such as at least 99%, such as 100%.

76. The genetically modified host cell of item 75, wherein the sequence identity is at least 99%, such as 100%.

77. The genetically modified host cell of item 73, wherein the heterologous gene encoding the glycosyl transferase has at least 90%, such as at least 95%, such as at least 99%, such as 100% identity to the glycosyl transferase encoding gene comprised in anyone of SEQ ID NO: 26, 28, 30, 32, 34, 36, 38, 40, 102 or 104.

78. The genetically modified host cell of item 77, wherein the heterologous gene encoding the glycosyl transferase is at least 95%, such as at least 99%, such as 100% identity to the glycosyl transferase encoding gene comprised in anyone of SEQ ID NO: 26, 28, 30, 32, 34, 36, 38, 40, 102 or 104.

79. The genetically modified host cell of item 78, wherein the heterologous gene encoding the glycosyl transferase is the glycosyl transferase encoding gene comprised in anyone of SEQ ID NO: 26, 28, 30, 32, 34, 36, 38, 40, 102 or 104.

80. The genetically modified host cell of any preceding item, wherein the cannabionoid glycoside has at least 10% higher water solubility than the corresponding un-glycosylated cannabinoid.

81. The genetically modified host cell of any preceding item, wherein the cannabinoid glycoside has at least 10% more resistance to UV or heat degradation than the corresponding un-glycosylated cannabinoid.

82. The genetically modified host cell of any preceding item, wherein the cannabinoid glycoside has at least 10% higher oral uptake than the corresponding un-glycosylated cannabinoid, when equally administered to a mammal.

83. The genetically modified host cell of any preceding item, wherein the cannabinoid glycoside has at least 10% higher biological half-life than the corresponding un-glycosylated cannabinoid, when equally administered to a mammal.

84. The genetically modified host cell of any preceding item, wherein the cannabinoid glycoside has at least 10% higher CNS concentration at peak concentration than the corresponding un-glycosylated cannabinoid, when equally administered to a mammal.

85. The genetically modified host cell of any preceding item, wherein the cannabinoid glycoside has at least 10% improved pharmacokinetics compared to the corresponding un-glycosylated cannabinoid as measured by a solubility assay, chemical stability assay, Caco-2 bi-directional permeability assay, hepatic microsomal clearance assay and/or plasma stability assay.

86. The genetically modified host cell of any preceding item, wherein the cannabinoid glycoside has at least 10% improved stability in acidic aqueous solution compared to the corresponding un-glycosylated cannabinoid, optionally in solution having a pH of 0 to 7, such as a pH of 0.5 to 4, such as a pH of 0.5 to 2, such as a pH of around 1.

87. The genetically modified host cell of any preceding item, wherein the cannabinoid glycoside has at least 10% improved stability in alkaline aqueous solution compared to the corresponding un-glycosylated cannabinoid, optionally in solution having a pH of 7 to 14, such as a pH of 9 to 14, such as a pH of 10 to 13, such as a pH of around 12.5.

88. The genetically modified host cell of any preceding item, wherein the cannabinoid glycoside has at least 10% improved resistance to oxidation in aqueous solution compared to the corresponding un-glycosylated cannabinoid, optionally in a solution having at least 8 mg/L 02, such as at least 20 mg/L 02, such as at least 40 mg/L 02, such as at least 80 mg/L 02, such as such as a solution saturated with 02.

89. The genetically modified host cell of any preceding item, wherein the cannabinoid glycoside is at least 10% less toxic to the genetically modified host cell compared to the corresponding un-glycosylated cannabinoid, optionally having a LC50 which is at least 10% less, such as at least 25% less, such as at least 75% less, such as at least 100% less than the corresponding un-glycosylated cannabinoid.

90. The genetically modified host cell of any preceding item, wherein the cannabinoid glycoside is a C-glycoside or an O-glycoside or a derivative or combination thereof 91. The genetically modified host cell of any preceding item, wherein the cannabinoid glycoside is selected from glycosides of cannabichromene-type (CBC), cannabigerol-type (CBG), cannabidiol-type (CBD), Tetrahydrocannabinol-type (THC), cannabicyclol-type (CBL), cannabielsoin-type (CBE), cannabinol-type (CBN), cannabinodiol-type (CBND) and cannabitriol-type.

92. The genetically modified host cell of item 91, wherein the cannabinoid glycoside is selected from glycosides of cannabidiol (CBD), cannabidiolic acid (CBDA), cannabidivarin (CBDV), tetrahydrocannabinol (THC), tetrahydrocannabinolic acid (THCA), tetrahydrocannabivarin (THCV), cannabichromevarin (CBCV), cannabigerol (CBG), cannabinol (CBN), 11-nor-9-carboxy-THC and Δ8-tetrahydrocannabinol.

93. The genetically modified host cell of any preceding item, wherein the cannabinoid glycoside comprises a cannabinoid aglycone or cannabinoid glycoside covalently linked to a sugar selected from xylose; rhamnose; galactose; N-acetylglucosamine; N-acetylgalactosamine; and arabinose.

94. The genetically modified host cell of any preceding item, wherein the cannabinoid glycoside is selected from cannabinoid-1′-O-β-D-glycoside, cannabinoid-1′-O-β-glycosyl-3′-O-β-glucoside, and cannabinoid-3′-O-β-D-glycoside.

95. The genetically modified host cell of item 93, wherein the cannabinoid glycoside is selected from CBD-1′-O-β-D-glycoside, CBD-1′-O-β-glycosyl-3′-O-β-glycoside, CBDV-r-O-β-D-glycoside, CBDV-1′-O-β-glycosyl-3′-O-β-glycoside, CBG-1′-O-β-D-glycoside, CBG-1′-O-β-glycosyl-3′-O-β-glycoside, THC-1′-O-β-D-glycoside, CBN-1′-O-β-D-glycoside, 11-nor-9-carboxy-THC-1′-O-β-D-glycoside, CBDA-3′-O-β-D-glycoside and CBC-3′-O-β-D-glycoside.

96. The genetically modified host cell of any preceding item, wherein the cannabinoid glycoside is selected from cannabinoid glucosides; cannabinoid glucuronosides; cannabinoid xylosides; cannabinoid rhamnosides; cannabinoid galactosides; cannabinoid N-acetylglucosaminosides; cannabinoid N-acetylgalactosaminosides and cannabinoid arabinosides.

97. The genetically modified host cell of item 96, wherein the cannabinoid glycoside is selected from cannabinoid-1′-O-β-D-glucoside; cannabinoid-1′-O-β-D-glucuroside; cannabinoid-1′-O-β-D-xyloside; cannabinoid-1′-O-α-L-rhamnoside; cannabinoid-1′-O-β-D-galactoside; cannabinoid-1′-O-β-D-N-acetylglucosaminoside; cannabinoid-1′-O-β-D-arabinoside; cannabinoid-1′-O-β-D-N-acetylgalactosamine; cannabinoid-1′-O-β-D-cellobioside; cannabinoid-1′-O-β-D-gentiobioside; cannabinoid-1′-O-β-D-glucosyl-3′-O-β-D-glucoside; cannabinoid-1′-O-β-D-glucurosyl-3′-O-β-D-glucuronoside; cannabinoid-1′-O-β-D-xylosyl-3′-O-β-D-xyloside; cannabinoid-1′-O-α-L-rhamnosyl-3′-O-β-D-rhamnoside; cannabinoid-1′-O-β-D-galactosyl-3′-O-β-D-galactoside; cannabinoid-1′-O-β-D-N-acetylglucosamine-3′-O-β-D-N-acetylglucosaminoside; cannabinoid-1′-O-β-D-arabinosyl-3′-O-β-D-arabinoside; and cannabinoid-1′-O-β-D-N-acetylgalactosamine-3′-O-β-D-N-acetylgalactosamine.

98. The genetically modified host cell of item 97, wherein the cannabinoid glycoside is selected from CBD-1′-O-β-D-cellobioside; CBD-1′-O-β-D-gentiobioside; CBD-1′-O-β-D-glucosyl-3′-O-β-D-glucoside; CBD-1′-O-β-D-glucurosyl-3′-O-β-D-glucuronoside; CBD-1′-O-β-D-xylosyl-3′-O-β-D-xyloside CBD-1′-O-α-L-rhamnosyl-3′-O-α-L-rhamnoside; CBD-1′-O-β-D-galactosyl-3′-O-β-D-galactoside; CBD-1′-O-β-D-N-acetylglucosamine-3′-O-β-D-N-acetylglucosaminoside; CBD-1′-O-β-D-arabinosyl-3′-O-β-D-arabinoside; CBD-1′-O-β-D-N-acetylgalactosamine-3′-O-β-D-N-acetylgalactosamine; CBDV-1′-O-β-D-cellobioside; CBDV-1′-O-β-D-gentiobioside; CBDV-1′-O-β-D-glucosyl-3′-O-β-D-glucoside; CBDV-1′-O-β-D-glucurosyl-3′-O-β-D-glucuronoside; CBDV-1′-O-β-D-xylosyl-3′-O-β-D-xyloside; CBDV-1′-O-α-L-rhamnosyl-3′-O-α-L-rhamnoside; CBDV-1′-O-β-D-galactosyl-3′-O-β-D-galactoside; CBDV-1′-O-β-D-N-acetylglucosamine-3′-O-β-D-N-acetylglucosaminoside; CBDV-1′-O-β-D-arabinosyl-3′-O-β-D-arabinoside; CBDV-1′-O-β-D-N-acetylgalactosamine-3′-O-β-D-N-acetylgalactosamine; CBG-1′-O-β-D-cellobioside; CBG-1′-O-β-D-gentiobioside; CBG-1′-O-β-D-glucosyl-3′-O-β-D-glucoside; CBG-1′-O-β-D-glucurosyl-3′-O-β-D-glucuronoside; CBG-1′-O-β-D-xylosyl-3′-O-β-D-xyloside CBG-1′-O-α-L-rhamnosyl-3′-O-α-L-rhamnoside; CBG-1′-O-β-D-galactosyl-3′-O-β-D-galactoside; CBG-1′-O-β-D-N-acetylglucosamine-3′-O-β-D-N-acetylglucosaminoside; CBG-1′-O-β-D-arabinosyl-3′-O-β-D-arabinoside; CBG-1′-O-β-D-N-acetylgalactosamine-3′-O-β-D-N-acetylgalactosamine; THC-1′-O-β-D-glucoside; THC-1′-O-β-D-cellobioside; THC-1′-O-β-D-gentiobioside; THC-1′-O-β-D-glucuronoside; THC-1′-O-β-D-xyloside; THC-1′-O-α-L-rhamnoside; THC-1′-O-β-D-galactoside; THC-1′-O-β-D-N-acetylglucosaminoside; THC-1′-O-β-D-arabinoside; THC-1′-O-β-D-N-acetylgalactosaminoside; CBN-1′-O-β-D-glucoside; CBN-1′-O-β-D-cellobioside; CBN-1′-O-β-D-gentiobioside; CBN-1′-O-β-D-glucuronoside; CBN-1′-O-β-D-xyloside; CBN-1′-O-α-L-rhamnoside; CBN-1′-O-β-D-galactoside; CBN-1′-O-β-D-N-acetylglucosaminoside; CBN-1′-O-β-D-arabinoside; CBN-1′-O-β-D-N-acetylgalactosaminoside; CBDA-1′-O-β-D-glucoside; CBDA-1′-O-β-D-cellobioside; CBDA-1′-O-β-D-gentiobioside; CBDA-1′-O-β-D-glucuronoside; CBDA-1′-O-β-D-xyloside; CBDA-1′-O-α-L-rhamnoside; CBDA-1′-O-β-D-galactoside; CBDA-1′-O-β-D-N-acetylglucosaminoside; CBDA-1′-O-β-D-arabinoside; CBDA-1′-O-β-D-N-acetylgalactosaminoside; CBC-1′-O-β-D-glucoside; CBC-1′-O-β-D-cellobioside; CBC-1′-O-β-D-gentiobioside; CBC-1′-O-β-D-glucuronoside; CBC-1′-O-β-D-xyloside; CBC-1′-O-α-L-rhamnoside; CBC-1′-O-β-D-galactoside; CBC-1′-O-β-D-N-acetylglucosaminoside; CBC-1′-O-(3-D-arabinoside; and CBC-1′-O-β-D-N-acetylgalactosaminoside.

99. The genetically modified host cell of any preceding item, further comprising an operative biosynthetic metabolic pathway capable of producing the cannabinoid acceptor, wherein the pathway comprises one or more polypeptides selected from:

a) an acetoacetyl-CoA thiolase (ACT) converting an acetyl-CoA precursor into acetoacetyl-CoA;
b) a HMG-CoA synthase (HCS) converting acetoacetyl-CoA precursor into HMG-CoA;
c) a HMG-CoA reductase (HCR) converting a HMG-CoA precursor into mevalonate;
d) a mevalonate kinase (MVK) converting a mevalonate precursor into Mevalonate-5-phosphate;
e) a phosphomevalonate kinase (PMK) converting a Mevalonate-5-phosphate precursor into Mevalonate diphosphate;
f) a mevalonate pyrophosphate decarboxylase (MPC) converting a Mevalonate diphosphate precursor into isopentenyl diphosphate (IPP);
g) an isopentenyl diphosphate/dimethylallyl diphosphate isomerase (IPI) converting an IPP precursor into dimethylallyl diphosphate (DMAPP);
h) Geranyl diphosphate synthase (GPPS) condensing IPP and DMAPP into Geranyl diphosphate (GPP);
i) an acyl activating enzyme (AAE) converting a fatty acid precursor into fatty acyl-COA;
j) a 3,5,7-Trioxododecanoyl-CoA synthase (TKS) converting a fatty acid-CoA precursor into 3,5,7-trioxoundecanoyl-CoA;
k) an Olivetolic Acid Cyclase (OAC) converting a 3,5,7-trioxoundecanoyl-CoA precursor into divarinolic acid;
l) an Olivetolic Acid Cyclase (OAC) converting a 3,5,7-trioxododecanoyl-CoA precursor into olivetolic acid;
m) a TKS-OAC fused enzymes converting fatty acid-CoA precursor into 3,5,7-trioxoundecanoyl-CoA, 3,5,7-trioxoundecanoyl-CoA precursor into divarinolic acid and 3,5,7-trioxododecanoyl-CoA precursor into olivetolic acid;
n) a Cannabigerolic acid synthase (CBGAS) condensing GPP and olivetolic acid into Cannabigerolic acid (CBGA);
o) a Cannabigerolic acid synthase (CBGAS) condensing GPP and divarinolic acid into cannabigerovarinic acid (CBGVA);
p) a cannabidiolic acid synthase (CBDAS) converting CBGA acid and/or CBGVA into cannabidiolic acid (CBDA) and/or cannabidivarinic acid (CBDVA), respectively;
q) a tetrahydrocannabinolic acid synthase (THCAS) converting CBGA and/or CBGVA into tetrahydrocannabinolic acid (THCA) and/or tetrahydrocannabivarinic acid (THCVA), respectively;
r) a cannabichromenic acid synthase (CBCAS) converting CBGA and/or CBGVA into cannabichromenic acid (CBCA) and/or cannabichromevarinic acid (CBCVA), respectively;
s) a nucleotide-glucose synthase converting sucrose and nucleotide into fructose and nucleotide-glucose;
t) a nucleotide-galactose 4-epimerase converting nucleotide-glucose into nucleotide-galactose;
u) a nucleotide-(glucuronic acid) decarboxylase converting nucleotide-glucuronic acid into nucleotide-xylose;
v) a nucleotide-4-keto-6-deoxy-glucose 3,5-epimerase and a nucleotide-4-keto-rhamnose 4-keto-reductase together converting nucleotide-4-keto-6-deoxy-glucose and NADPH into nucleotide-rhamnose and NADP+;
w) a nucleotide-glucose 4,6-dehydratase converting nucleotide-glucose and NAD into nucleotide-4-keto-6-deoxy-glucose and NADH;
x) a nucleotide-glucose 4,6-dehydratase and a nucleotide-4-keto-6-deoxy-glucose 3,5-epimerase and a nucleotide-4-keto-rhamnose-4-keto-reductase together converting nucleotide-glucose and NAD+ and NADPH into nucleotide-rhamnose+NADH+NADP+;
y) a nucleotide-glucose 6-dehydrogenase converting nucleotide-glucose and 2 NAD+ into nucleotide-glucuronic acid and 2 NADH;
z) a nucleotide-arabinose 4-epimerase converting nucleotide-xylose into nucleotide-arabinose; and aa) a nucleotide-N-acetylglucosamine 4-epimerase converting nucleotide-N-acetylglucosamine into nucleotide-N-acetylgalactosamine.

100. The genetically modified host cell of item 99, wherein the:

a) ACT has at least 70%, such at least 75%, such as at least 80%, such as at least 90%, such as at least 95%, such as at least 99%, such as 100% identity to the native Erg10 in S. cerevisiae;
b) HCS has at least 70%, such at least 75%, such as at least 80%, such as at least 90%, such as at least 95%, such as at least 99%, such as 100% identity to the native Erg13 in S. cerevisiae;
c) HCR has at least 70%, such at least 75%, such as at least 80%, such as at least 90%, such as at least 95%, such as at least 99%, such as 100% identity to the native HMG1 or HMG2 in S. cerevisiae;
d) MVK has at least 70%, such at least 75%, such as at least 80%, such as at least 90%, such as at least 95%, such as at least 99%, such as 100% identity to the native Erg12 in S. cerevisiae;
e) PMK has at least 70%, such at least 75%, such as at least 80%, such as at least 90%, such as at least 95%, such as at least 99%, such as 100% identity to the native Erg8 in S. cerevisiae;
f) MPC has at least 70%, such at least 75%, such as at least 80%, such as at least 90%, such as at least 95%, such as at least 99%, such as 100% identity to the native MVD1 in S. cerevisiae;
g) IPI has at least 70%, such at least 75%, such as at least 80%, such as at least 90%, such as at least 95%, such as at least 99%, such as 100% identity to the native ID11 in S. cerevisiae;
h) GPPS has at least 70%, such at least 75%, such as at least 80%, such as at least 90%, such as at least 95%, such as at least 99%, such as 100% identity to the GPPS comprised in SEQ ID NO: 45 or 229;
i) AAE has at least 70%, such at least 75%, such as at least 80%, such as at least 90%, such as at least 95%, such as at least 99%, such as 100% identity to the AAE comprised in SEQ ID NO: 47 or 239;
j) TKS has at least 70%, such at least 75%, such as at least 80%, such as at least 90%, such as at least 95%, such as at least 99%, such as 100% identity to the TKS comprised in SEQ ID NO: 49;
k) OAC has at least 70%, such at least 75%, such as at least 80%, such as at least 90%, such as at least 95%, such as at least 99%, such as 100% identity to the OAC comprised in SEQ ID NO: 51;
l) TKS-OAC fused enzyme at least 70%, such at least 75%, such as at least 80%, such as at least 90%, such as at least 95%, such as at least 99%, such as 100% identity to the TKS-OAC fused enzyme comprised in SEQ ID NO 227;
m) CBGAS has at least 70%, such at least 75%, such as at least 80%, such as at least 90%, such as at least 95%, such as at least 99%, such as 100% identity to the CBGAS comprised in SEQ ID NO: 53, 235 or 237;
n) CBDAS has at least 70%, such at least 75%, such as at least 80%, such as at least 90%, such as at least 95%, such as at least 99%, such as 100% identity to the CBDAS comprised in SEQ ID NO: 57 or 233;
o) THCAS has at least 70%, such at least 75%, such as at least 80%, such as at least 90%, such as at least 95%, such as at least 99%, such as 100% identity to the THCAS comprised in SEQ ID NO: 55 or 231;
p) CBCAS has at least 70%, such at least 75%, such as at least 80%, such as at least 90%, such as at least 95%, such as at least 99%, such as 100% identity to the CBCAS comprised in SEQ ID NO: 59;
q) nucleotide-glucose synthase is an UDP-glucose synthase and has at least 70%, such at least 75%, such as at least 80%, such as at least 90%, such as at least 95%, such as at least 99%, such as 100% identity to the UDP-glucose synthase comprised in SEQ ID NO: 209;
r) nucleotide-galactose 4-epimerase is an UDP-galactose 4-epimerase and has at least 70%, such at least 75%, such as at least 80%, such as at least 90%, such as at least 95%, such as at least 99%, such as 100% identity to the UDP-galactose 4-epimerase comprised in SEQ ID NO: 211;
s) nucleotide-(glucuronic acid)-decarboxylase is an UDP-glucuronic acid decarboxylase and has at least 70%, such at least 75%, such as at least 80%, such as at least 90%, such as at least 95%, such as at least 99%, such as 100% identity to the UDP-glucuronic acid decarboxylase comprised in SEQ ID NO: 213;
t) nucleotide-4-keto-6-deoxy-glucose 3,5-epimerase is an UDP-4-keto-6-deoxy-glucose 3,5-epimerase and has at least 70%, such at least 75%, such as at least 80%, such as at least 90%, such as at least 95%, such as at least 99%, such as 100% identity to the UDP-4-keto-6-deoxy-glucose 3,5-epimerase comprised in SEQ ID NO: 215 or 219;
u) nucleotide-4-keto-rhamnose-4-keto reductase is an UDP-4-keto-rhamnose-4-keto reductase and has at least 70%, such at least 75%, such as at least 80%, such as at least 90%, such as at least 95%, such as at least 99%, such as 100% identity to the UDP-4-keto-rhamnose-4-keto reductase comprised in SEQ ID NO: 215 or 219;
v) nucleotide-glucose 4,6-dehydratase is an UDP-glucose 4,6-dehydratase and has at least 70%, such at least 75%, such as at least 80%, such as at least 90%, such as at least 95%, such as at least 99%, such as 100% identity to the UDP-glucose 4,6-dehydratase comprised in SEQ ID NO: 217 or 219;
w) nucleotide-glucose 6 dehydrogenase is an UDP-glucose 6-dehydrogenase and has at least 70%, such at least 75%, such as at least 80%, such as at least 90%, such as at least 95%, such as at least 99%, such as 100% identity to the UDP-glucose 6 dehydrogenase comprised in SEQ ID NO: 221;
x) nucleotide-arabinose 4-epimerase is an UDP-arabinose 4-epimerase and has at least 70%, such at least 75%, such as at least 80%, such as at least 90%, such as at least 95%, such as at least 99%, such as 100% identity to the UDP-arabinose 4-epimerase comprised in SEQ ID NO: 223; and
y) nucleotide-N-acetylglucosamine 4-epimerase is an UDP-N-acetylglucosamine 4-epimerase and has at least 70%, such at least 75%, such as at least 80%, such as at least 90%, such as at least 95%, such as at least 99%, such as 100% identity to the UDP-N-acetylglucosamine 4-epimerase comprised in SEQ ID NO: 225.

101. The genetically modified host cell of items 100, wherein the:

a) ACT is the native Erg10 in S. cerevisiae;
b) HCS is the native Erg13 in S. cerevisiae;
c) HCR is the native HMG1 in S. cerevisiae;
d) HCR is the native HMG2 in S. cerevisiae;
e) MVK is the native Erg12 in S. cerevisiae;
f) PMK is the native Erg8 in S. cerevisiae;
g) MPC is the native MVD1 in S. cerevisiae;
h) IPI is the native ID11 in S. cerevisiae;

### i) GPPS is the GPPS of SEQ ID NO: 45 or 229;

### j) AAE is the AAE of SEQ ID NO: 47 or 238;

### k) TKS is the TKS of SEQ ID NO: 49;

### l) OAC is the OAC of SEQ ID NO: 51;

m) TKS-OAC fused enzyme is the TKS-OAC fused enzyme comprised in SEQ ID NO 227

### n) CBGAS is the CBGAS of SEQ ID NO: 53, 235 or 237;

### o) CBDAS is the CBDAS of SEQ ID NO: 57 or 233;

### p) THCAS is the THCAS of SEQ ID NO: 55 or 231;

### q) CBCAS is the CBCAS of SEQ ID NO: 59;

r) UDP-glucose synthase is the UDP-glucose synthase comprised in SEQ ID NO: 209;
s) UDP-galactose 4-epimerase is the UDP-galactose 4-epimerase comprised in SEQ ID NO: 211;
t) UDP-glucuronic acid decarboxylase is the UDP-glucuronic acid decarboxylase comprised in SEQ ID NO: 213;
u) UDP-4-keto-6-deoxy-glucose 3,5-epimerase is the UDP-4-keto-6-deoxy-glucose 3,5-epimerase comprised in SEQ ID NO: 215 or 219;
v) UDP-4-keto-rhamnose-4-keto reductase is the UDP-4-keto-rhamnose-4-keto reductase comprised in SEQ ID NO: 215 or 219;
w) UDP-glucose 4,6-dehydratase is the UDP-glucose 4,6-dehydratase comprised in SEQ ID NO: 217 or 219;
x) UDP-glucose 6-dehydrogenase is the UDP-glucose 6-dehydrogenase comprised in SEQ ID NO: 221;
y) UDP-arabinose 4-epimerase is the UDP-arabinose 4-epimerase comprised in SEQ ID NO: 223; and
z) UDP-N-acetylglucosamine 4-epimerase is the UDP-N-acetylglucosamine 4-epimerase comprised in SEQ ID NO: 225.

102. The genetically modified host cell of any preceding item, wherein a plurality of polypeptides comprised in the operative biosynthetic metabolic pathway are heterologous to the genetically modified host cell.

103. The genetically modified host cell of any preceding item, wherein the genetically modified host cell is further genetically modified to provide an increased amount of a substrate for at least one polypeptide of the operative biosynthetic metabolic pathway.

104. The genetically modified host cell of any preceding item, wherein the genetically modified host cell is further genetically modified to exhibit increased tolerance towards one or more substrates, intermediates, or product molecules from the operative biosynthetic metabolic pathway.

105. The genetically modified host cell of any preceding item, wherein the genetically modified host cell is further genetically modified to include a transporter polypeptide facilitating secretion of the intracellularly formed cannabinoid glycoside.

106. The genetically modified host cell of any preceding item, wherein the genetically modified host cell is an eukaryotic, prokaryotic or archaic cell.

107. The genetically modified host cell of item 106, wherein the genetically modified host cell is an eukaryote cell selected from the group consisting of mammalian, insect, plant, or fungal cells.

108. The genetically modified host cell of items 107, wherein the genetically modified host cell is a plant cell of the genus Cannabis, Humulus or Stevia.

109. The genetically modified host cell of items 107, wherein the genetically modified host cell is a fungal host cell selected from phylas consisting of Ascomycota, Basidiomycota, Neocallimastigomycota, Glomeromycota, Blastocladiomycota, Chytridiomycota, Zygomycota, Oomycota and Microsporidia.

110. The genetically modified host cell of items 109, wherein the genetically modified fungal host cell is a yeast selected from the group consisting of ascosporogenous yeast (Endomycetales), basidiosporogenous yeast, and Fungi Imperfecti yeast (Blastomycetes).

111. The genetically modified host cell of items 110, wherein the genetically modified yeast host cell is selected from the genera consisting of Saccharomyces, Kluveromyces, Candida, Pichia, Debaromyces, Hansenula, Yarrowia, Zygosaccharomyces, and Schizosaccharomyces.

112. The genetically modified host cell of items 111, wherein the genetically modified yeast host cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii and Yarrowia lipolytica.

113. The genetically modified host cell of items 109, wherein the genetically modified fungal host cell is filamentous fungus.

114. The genetically modified host cell of item 113, wherein the filamentous fungal genetically modified host cell is selected from the phylas consisting of Ascomycota, Eumycota and Oomycota.

115. The genetically modified host cell of item 114, wherein the filamentous fungal genetically modified host cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

116. The genetically modified host cell of item 115, wherein the filamentous fungal host cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

117. The genetically modified host cell of item 106, wherein the genetically modified host cell is a prokaryotic cell.

118. The genetically modified host cell of item 117, wherein the prokaryotic cell is E. coli.

119. The genetically modified host cell of item 106, wherein the genetically modified host cell is an archaic cell.

120. The genetically modified host cell of item 119, wherein the archaic cell is an algae.

121. A polynucleotide construct comprising a polynucleotide sequence encoding the glycosyl transferase of any preceding item, operably linked to one or more control sequences heterologous to the glycosyl encoding polynucleotide.

122. The polynucleotide construct of item 121, wherein the glycosyl transferase encoding polynucleotide has at least 70%, such at least 75%, such as at least 80%, such as at least 90%, such as at least 95%, such as at least 99%, such as 100% identity to the glycosyl transferase encoding gene comprised in anyone of SEQ ID NO: 2, 4, 6, 8, 10, 12, 14, 16, 18, 20, 22, 24, 26, 28, 30, 32, 34, 36, 38, 40, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 or 208.

123. An expression vector comprising the polynucleotide construct of items 121 or 122.

124. A genetically modified host cell comprising the polynucleotide construct or the vector of item 123.

125. The genetically modified host cell of any preceding item, comprising at least two copies of the genes encoding the glycosyl transferase and/or any pathway enzymes.

126. The genetically modified host cell of any preceding item, wherein one or more native genes are attenuated, disrupted and/or deleted.

127. The genetically modified host cell of any preceding item, wherein the genetically modified host cell is a S. cerevisiae strain modified by attenuating, disrupting and/or deleting PDR12 of SGD ID SGD:5000005979.

128. A cell culture, comprising the genetically modified host cell of any preceding item and a growth medium.

129. A method for producing a cannabinoid glycoside comprising:

a) culturing the cell culture of item 128 at conditions allowing the genetically modified host cell to produce the cannabinoid glycoside; and
b) optionally recovering and/or isolating the cannabinoid glycoside.

130. The method of items 129, further comprising one or more elements selected from:

a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch or semi-continuous conditions
h) culturing the cell culture in the presence of an organic solvent to improve the solubility of the cannabinoid aglycone.

131. The method of item 129 to 130, further comprising a step of non-enzymatic decarboxylation of the cannabinoid acceptor and/or the cannabinoid glycoside.

132. The method of item 131, wherein the decaboxylation is achieved by heat-, UV- or alkalinity treatment or a combination thereof.

133. The method of items 129 to 132, further comprising feeding one or more exogenous cannabinoid acceptors and/or nucleotide-glycosides to the cell culture.

134. The method of items 129 to 133, wherein the recovering and/or isolation step comprises separating a liquid phase of the genetically modified host cell or cell culture from a solid phase of the genetically modified host cell or cell culture to obtain a supernatant comprising the cannabinoid glycoside by one or more steps selected from:

a) disintegrating the genetically modified host cell to release intracellular cannabinoid glycoside into the supernatant;
b) contacting the supernatant with one or more adsorbent resins in order to obtain at least a portion of the produced cannabinoid glycoside;
c) contacting the supernatant with one or more ion exchange or reversed-phase chromatography columns in order to obtain at least a portion of the cannabinoid glycoside; and
d) crystallizing or extracting the cannabinoid glycosides; and
e) evaporating the solvent of the liquid phase to concentrate or precipitate the cannabinoid glycoside; thereby recovering and/or isolating the cannabinoid glycoside.

135. The method of items 129 to 134, wherein the cannabinoid glycoside yield is at least 10% higher such as at least 50%, such as 100%, such as least 150%, such as at least 200% higher than production by UGT76G1 from Stevia rebaudiana.

136. The method of item 138, wherein the glycosylation is performed in vitro.

137. The method of items 129 to 136 comprising steps of working the cannabinoid glycoside into a pharmaceutical cannabinoid formulation comprising feeding a cell culture of item 128 comprising non-plant cells with a starting material in a growth medium; producing the pharmaceutical cannabinoid compound from the cell culture to create a mixture comprising the cell culture, the growth medium, and the pharmaceutical cannabinoid compound; processing the pharmaceutical cannabinoid compound, wherein the processing comprises: separating out genetically modified cells using at least one process selected from the group consisting of sedimentation, filtration, and centrifugation; and producing the pharmaceutical cannabinoid formulation that comprises the pharmaceutical cannabinoid, wherein the mixture does not contain a detectable amount of plant impurities selected from the group consisting of polysaccharides, lignin, pigments, flavonoids, phenanthreoids, latex, gum, resin, wax, pesticides, fungicides, herbicides, and pollen.

138. A method for producing a cannabinoid glycoside comprising contacting a cannabinoid acceptor with one or more cannabinoid glycosyl transferases of items 19 to 72 and one or more nucleotide glycosides of items 15 to 18 at conditions allowing the glycosyl transferase to transfer the glycosyl moiety of the nucleotide glycoside to the cannabinoid.

139. A method of producing a cannabinoid comprising producing a cannabinoid glycoside according to the methods of items 129 to 136 and subjecting the cannabinoid glycoside to one or more deglycosylation steps.

140. The method of item 139, wherein the deglycosylation is achieved by incubating the cannabinoid glycoside with one or more enzymes selected from glucosidases, pectinase, arabinase, cellulase, glucanase, hemicellulase, and xylanase.

141. The method of item 140, wherein the one or more enzymes are selected from β-glucosidase, β-betagluconase, pectolyase, pectozyme and polygalacturonase.

142. The method of items 139 to 141, wherein the deglycosylating step is performed in vitro.

143. A fermentation liquid comprising the cannabinoid glycosides comprised in the cell culture of item 128.

144. The fermentation liquid of item 143, wherein at least 50%, such as at least 75%, such as at least 95%, such as at least 99% of the genetically modified host cells are disintegrated.

145. The fermentation liquid of item 143 to 144, wherein at least 50%, such as at least 75%, such as at least 95%, such as at least 99% of solid cellular material has separated from the liquid.

146. The fermentation liquid of item 144 to 145, further comprising one or more compounds selected from:

a) precursors or products of the operative biosynthetic metabolic pathway producing the cannabinoid glycoside;
b) supplemental nutrients comprising trace metals, vitamins, salts, yeast nitrogen base, YNB, and/or amino acids; and wherein the concentration of the cannabinoid glycoside is at least 1 mg/I liquid.

147. A cannabinoid glycoside comprising a cannabinoid aglycone or cannabinoid glycoside covalently linked to a sugar selected from xylose; rhamnose; galactose; N-acetylglucosamine; N-acetylgalactosamine; and arabinose.

148. The cannabinoid glycoside of item 147, wherein the cannabinoid glycoside is selected from cannabinoid-1′-O-β-D-xyloside; cannabinoid-1′-O-α-L-rhamnoside; cannabinoid-1′-O-β-D-galactoside; cannabinoid-1′-O-β-D-N-acetylglucosaminoside; cannabinoid-1′-O-β-D-arabinoside; cannabinoid-1′-O-β-D-N-acetylgalactosamine; cannabinoid-1′-O-β-D-cellobioside; cannabinoid-1′-O-β-D-gentiobioside; cannabinoid-1′-O-β-D-xylosyl-3′-O-β-D-xyloside; cannabinoid-1′-O-α-L-rhamnosyl-3′-O-β-D-rhamnoside; cannabinoid-1′-O-β-D-galactosyl-3′-O-β-D-galactoside; cannabinoid-1′-O-β-D-N-acetylglucosamine-3′-O-β-D-N-acetylglucosaminoside; cannabinoid-1′-O-β-D-arabinosyl-3′-O-β-D-arabinoside; and cannabinoid-1′-O-β-D-N-acetylgalactosamine-3′-O-β-D-N-acetylgalactosamine.

149. The cannabinoid glycoside of item 148, wherein the cannabinoid glycoside is selected from CBD-1′-O-β-D-cellobioside; CBD-1′-O-β-D-gentiobioside; CBD-1′-O-β-D-xylosyl-3′-O-β-D-xyloside; CBD-1′-O-α-L-rhamnosyl-3′-O-α-L-rhamnoside; CBD-1′-O-β-D-galactosyl-3′-O-β-D-galactoside; CBD-1′-O-β-D-N-acetylglucosamine-3′-O-β-D-N-acetylglucosaminoside; CBD-1′-O-β-D-arabinosyl-3′-O-β-D-arabinoside; CBD-1′-O-β-D-N-acetylgalactosamine-3′-O-β-D-N-acetylgalactosamine; CBDV-1′-O-β-D-cellobioside; CBDV-1′-O-β-D-gentiobioside; CBDV-1′-O-β-D-xylosyl-3′-O-β-D-xyloside; CBDV-1′-O-α-L-rhamnosyl-3′-O-α-L-rhamnoside; CBDV-1′-O-β-D-galactosyl-3′-O-β-D-galactoside; CBDV-1′-O-β-D-N-acetylglucosamine-3′-O-β-D-N-acetylglucosaminoside; CBDV-1′-O-β-D-arabinosyl-3′-O-β-D-arabinoside; CBDV-1′-O-β-D-N-acetylgalactosamine-3′-O-β-D-N-acetylgalactosamine; CBG-1′-O-β-D-cellobioside; CBG-1′-O-β-D-gentiobioside; CBG-1′-O-β-D-xylosyl-3′-O-β-D-xyloside CBG-1′-O-α-L-rhamnosyl-3′-O-α-L-rhamnoside; CBG-1′-O-β-D-galactosyl-3′-O-β-D-galactoside; CBG-1′-O-β-D-N-acetylglucosamine-3′-O-β-D-N-acetylglucosaminoside; CBG-1′-O-β-D-arabinosyl-3′-O-β-D-arabinoside; CBG-1′-O-β-D-N-acetylgalactosamine-3′-O-β-D-N-acetylgalactosamine; THC-1′-O-β-D-cellobioside; THC-1′-O-β-D-gentiobioside; THC-1′-O-β-D-xyloside; THC-1′-O-α-L-rhamnoside; THC-1′-O-β-D-galactoside; THC-1′-O-β-D-N-acetylglucosaminoside; THC-1′-O-β-D-arabinoside; THC-1′-O-β-D-N-acetylgalactosaminoside; CBN-1′-O-β-D-cellobioside; CBN-1′-O-β-D-gentiobioside; CBN-1′-O-β-D-xyloside; CBN-1′-O-α-L-rhamnoside; CBN-1′-O-β-D-galactoside; CBN-1′-O-β-D-N-acetylglucosaminoside; CBN-1′-O-β-D-arabinoside; CBN-1′-O-β-D-N-acetylgalactosaminoside; CBDA-1′-O-β-D-cellobioside; CBDA-1′-O-β-D-gentiobioside; CBDA-1′-O-β-D-xyloside; CBDA-1′-O-α-L-rhamnoside; CBDA-1′-O-β-D-galactoside; CBDA-1′-O-β-D-N-acetylglucosaminoside; CBDA-1′-O-β-D-arabinoside; CBDA-1′-O-β-D-N-acetylgalactosaminoside; CBC-1′-O-β-D-cellobioside; CBC-1′-O-β-D-gentiobioside; CBC-1′-O-β-D-xyloside; CBC-1′-O-α-L-rhamnoside; CBC-1′-O-β-D-galactoside; CBC-1′-O-β-D-N-acetylglucosaminoside; CBC-1′-O-β-D-arabinoside; and CBC-1′-O-3-D-N-acetylgalactosaminoside.

150. A cannabinoid glycoside comprising a cannabinoid aglycone or cannabinoid glycoside covalently linked to glycosyl moiety by a 1,4- or 1,6-glycosidic bond.

151. The cannabinoid glycoside of item 148, wherein the cannabinoid glycoside is selected from CBD-1′-O-β-D-gentiobioside and CBD-1′-O-β-D-cellobioside.

152. A composition comprising the fermentation liquid of item 143 to 146 and/or the cannabinoid glycoside of items 147 to 151 and one or more agents, additives and/or excipients.

153. The composition of item 152, wherein the fermentation liquid and the one or more agents, additives and/or excipients are in a dry solid form.

154. The composition of item 152, wherein the fermentation liquid and the one or more agents, additives and/or excipients are in a liquid stabilized form.

155. The composition of item 154, wherein the composition is refined into a beverage suitable for human or animal ingestion and wherein the cannabinoid glycoside has increased water solubility compared to the un-glycosylated cannabinoid.

156. The composition of item 153, wherein the composition is refined into a food item suitable for human or animal ingestion and wherein the cannabinoid glycoside has increased water solubility compared to the un-glycosylated cannabinoid.

157. A method for preparing a pharmaceutical preparation comprising mixing the cannabinoid glycoside of items 147 to 151 or a prodrug thereof or the composition of items 152 to 156 with one or more pharmaceutical grade excipient, additives and/or adjuvants.

158. The method of item 157, wherein the pharmaceutical preparation is in form of a powder, tablet, capsule, hard chewable and or soft lozenge or a gum.

159. The method of item 157, wherein the pharmaceutical preparation is in form of a liquid pharmaceutical solution.

160. A pharmaceutical preparation obtainable from the method of item 157 to 159.

161. A pharmaceutical preparation obtainable from the method of item 157 to 159 for use as a medicament or a prodrug.

162. The preparation of item 161 for use in the treatment of a disease elected from NASH, Epilepsy, Vomiting, Nausea, Cancer, Multiple sclerosis, Spasticity, Chronic pain, Anorexia, Loss of appetite, Parkinson's, Dravet Syndrome (Severe Myoclonic Epilepsy of Infancy), Lennox-Gastaut Syndrome, Substance (Drug) Abuse, Diabetes, Seizures, Panic Disorders, Social Anxiety Disorders (SAD), Generalized Anxiety Disorder (GAD), Anxiety Disorders, Agoraphobia, Infantile Spasm (West Syndrome), Psoriasis, Postherpetic Neuralgia, Motor Neuron Diseases, Amyotrophic Lateral Sclerosis, Tourette Syndrome, Tic Disorder, Cerebral Palsy, Graft Versus Host Disease (GVHD), Crohn's Disease (Regional Enteritis), Inflammatory Bowel Disease, Fragile X Syndrome, Bipolar Disorder (Manic Depression), Osteoarthritis, Huntington Disease, Schizophrenia, Autism, Restless Legs Syndrome, Human Immunodeficiency Virus (HIV) Infections (AIDS), Hypertension, Liver Fibrosis, Hepatic Injury, Prader-Willi Syndrome (PWS), Post-Traumatic Stress Disorder (PTSD), Fatty Liver Disease, Glaucoma, Inflammatory disease, Clostridium difficile infection, Colorectal tumor, Inflammatory bowel disease, Intestine disease, Irritable bowel syndrome, Ulcerative colitis, Cognitive disorder, Brain hypoxia, Fibrosis, Sleep apnea, motor neuron disease, antibiotic-resistance, bacterial infections and COVID-19 infections in a mammal.

163. A method for treating a disease in a mammal, comprising administering a therapeutically effective amount of the pharmaceutical preparation of item 160 or the cannabinoid glycoside of items 147 to 151 to the mammal.

164. The method of item 163, wherein the disease is selected from NASH, Epilepsy, Vomiting, Nausea, Cancer, Multiple sclerosis, Spasticity, Chronic pain, Anorexia, Loss of appetite, Parkinson's, Dravet Syndrome (Severe Myoclonic Epilepsy of Infancy), Lennox-Gastaut Syndrome, Substance (Drug) Abuse, Diabetes, Seizures, Panic Disorders, Social Anxiety Disorders (SAD), Generalized Anxiety Disorder (GAD), Anxiety Disorders, Agoraphobia, Infantile Spasm (West Syndrome), Psoriasis, Postherpetic Neuralgia, Motor Neuron Diseases, Amyotrophic Lateral Sclerosis, Tourette Syndrome, Tic Disorder, Cerebral Palsy, Graft Versus Host Disease (GVHD), Crohn's Disease (Regional Enteritis), Inflammatory Bowel Disease, Fragile X Syndrome, Bipolar Disorder (Manic Depression), Osteoarthritis, Huntington Disease, Schizophrenia, Autism, Restless Legs Syndrome, Human Immunodeficiency Virus (HIV) Infections (AIDS), Hypertension, Liver Fibrosis, Hepatic Injury, Prader-Willi Syndrome (PWS), Post-Traumatic Stress Disorder (PTSD), Fatty Liver Disease, Glaucoma, Inflammatory disease, Clostridium difficile infection, Colorectal tumor, Inflammatory bowel disease, Intestine disease, Irritable bowel syndrome, Ulcerative colitis, Cognitive disorder, Brain hypoxia, Fibrosis, Sleep apnea, motor neuron disease, antibiotic-resistance, bacterial infections and COVID-19 infections.

### REFERENCES

Gajewski, J., Pavlovic, R., Fischer, M., Boles, E., & Grininger, M. (2017). Engineering fungal de novo fatty acid synthesis for short chain fatty acid production. Nature Communications, 8, 1-8. https://doi.org/10.1038/ncomm514650
Gietz, R. D., & Woods, R. A. (2002). Transformation of yeast by lithium acetate/single-stranded carrier DNA/polyethylene glycol method. Methods in Enzymology, 350(2001), 87-96. https://doi.org/10.1016/S0076-6879(02)50957-5
Grote, A., Hiller, K., Scheer, M., Münch, R., Nörtemann, B., Hempel, D. C., & Jahn, D. (2005). JCat: A novel tool to adapt codon usage of a target gene to its potential expression host. Nucleic Acids Research, 33(SUPPL. 2), 526-531. https://doi.org/10.1093/nar/gki376
Gueldener, U., Heinisch, J., Koehler, G. J., Voss, D., & Hegemann, J. H. (2002). A second set of loxP marker cassettes for Cre-mediated multiple gene knockouts in budding yeast. Nucleic Acids Research, 30(6), e23. Retrieved from http://www.ncbi.nlm.nih.gov/pubmed/11884642%0Ahttp://www.pubmedcentral.nih.gov/articlerender.fcgiΔartid=PMC101367
Jensen, N. B., Strucko, T., Kildegaard, K. R., David, F., Maury, J., Mortensen, U. H., . . . Borodina, I. (2014). EasyClone: Method for iterative chromosomal integration of multiple genes in Saccharomyces cerevisiae. FEMS Yeast Research, 14(2), 238-248. https://doi.org/10.1111/1567-1364.12118
Jessop-Fabre, M. M., Jakočiūnas, T., Stovicek, V., Dai, Z., Jensen, M. K., Keasling, J. D., & Borodina, I. (2016). EasyClone-MarkerFree: A vector tool kit for marker-less integration of genes into Saccharomyces cerevisiae via CRISPR-Cas9. Biotechnology Journal, 11(8), 1110-1117. https://doi.org/10.1002/biot.201600147
van Rossum, H. M., Kozak, B. U., Pronk, J. T., & van Maris, A. J. A. (2016). Engineering cytosolic acetyl-coenzyme A supply in Saccharomyces cerevisiae: Pathway stoichiometry, free-energy conservation and redox-cofactor balancing. Metabolic Engineering, 36, 99-115. https://doi.org/10.1016/j.ymben.2016.03.006
Shi, S., Chen, Y., & Siewers, V. (2014). Improving Production of Malonyl Coenzyme A-Derived Metabolites. MBio, 5(3), e01130-14. https://doi.org/10.1128/mBio.01130-14
Luo, X., Reiter, M. A., d'Espaux, L., Wong, J., Denby, C. M., Lechner, A., . . . Keasling, J. D. (2019). Complete biosynthesis of cannabinoids and their unnatural analogues in yeast. Nature 2019, 1. https://doi.org/10.1038/s41586-019-0978-9
Degenhardt, F., Stehle, F., & Kayser, O. (2017). The Biosynthesis of Cannabinoids. Handbook of Cannabis and Related Pathologies: Biology, Pharmacology, Diagnosis, and Treatment. Elsevier Inc. https://doi.org/10.1016/B978-O-12-800756-3.00002-8
Mackenzie, P. I., Owens, I. S., Burchell, B. et al. (1997) The UDP glycosyltransferase gene superfamily: recommended nomenclature update based on evolutionary divergence. Pharmacogenetics, 7, 255-269.

### EXAMPLES

### Examples

### Materials and Methods

### Materials

Chemicals used in the examples herein e.g. for buffers and substrates are commercial products of at least reagent grade.

### Strains

BY4723 is a common strain of S. cerevisiae derived from S288C and available e.g. from American Type Culture Collection (ATCC #200885).

BY4741 is a common strain of S. cerevisiae derived from S288C and available e.g. from Euroscarf (Y00000).

BL21 (DE3) is a common strain of E. coli available from E.g. New England Biolabs (C2527I).

DH5α is a common strain of E. coli available from E.g. ThermoFisher Scientific (18265017).

XJb (DE3) autolysis strain is a common strain of E. coli available from E.g. Zymo Research (T3051).

Methods for Extraction and Recovery of Cannabinoids from Culture Media for Examples 2, 4, 7, 14-15 and 21:

### Part I.

Following cultivation of S. cerevisiae or E. coli, cannabinoids or cannabinoid glycosides were extracted from the culture media as follows. Samples were initially treated with 2 U/OD zymolyase (Zymo Research) (2 h, 30° C., 800 rpm) (step are skipped for E. coli cultures) followed by ethyl acetate/formic acid (0.05% (v/v)) extraction in a 2:1 ratio and bead-beating (30 s⁻¹, 3 min). Samples were then centrifuged at 12,000 g for 1 min and the inorganic fraction discarded. Extraction with ethyl acetate/formic acid were then repeated. The remaining organic fraction were then evaporated to dryness in a vacuum oven at 50° C., the dried extract were then resuspended in acetonitrile/H2O/formic acid (80%/20%/0.05% (v/v/v). Finally, samples were filtered with Ultrafree-MC columns (0.22 μm pore size, polyvinylidene difluoride (PVDF) membrane.

### Part II.

Alternatively, whole cell broth extraction of cannabinoids or cannabinoid glycosides in E. coli or S. cerevisiae was performed as follows. Cell cultures are mixed 1:1 with 100% methanol, glass beads were added and cells are burst open using a bead-beating machine (e.g. FastPrep). Samples were centrifuged at 12,000 g for 1 min and the supernatant used directly for analysis.

### Analytical Procedures for Examples 2, 4, 7-14, 16-18 and 20-21:

### Part I.

HPLC analysis was performed on an Agilent Technologies 1100 Series equipped with DAD detector. Separation was achieved on a Kinetex 2.6 μm XB-C18 column (100×2.1 mm, 2.6 μm, 100 Å, Phenomenex). Solvents: 0.05% (v/v) trifluoro acetic acid in H₂O and 0.05% (v/v) trifluoro acetic acid in MeCN as mobile phases A and B, respectively. Gradient conditions: 0.0-23 min 1%-99% B; 23.1-25.0 min 99-1% and 25.1-27.0 min 2% B. Mobile phase flow rate was 400 μL/min. The column temperature was maintained at 30° C. UV spectra were acquired at 230 and 254 nm. Autosampler temperature was set at 10° C.±2° C. Cannabinoids were identified using authentic reference standards. Quantification was made using a standard calibration curve plotted with a series of concentrations for the cannabinoid standard solutions.

### Part II.

LC-MS analysis was performed by UPLC coupled to a triple-quadrupole mass spectrometer interfaced with an electrospray ion source (ESI) (Waters, Milford, Mass.). 1 μL of the extracted sample was injected into the LC-MS system and separation was achieved in reversed phase using a C18 BEH (1.7 μm, 2.1×50 mm) column equipped with a C18 BEH (1.7 μm) pre-column (Waters, Milford, Mass.) and mobile phases consisting of 0.1% formic acid (Sigma-Aldrich) in Milli-Q© grade water (A) and 0.1% formic acid in MS grade acetonitrile (B) with a flow rate of 0.6 mL/min. Masslynx software (version 1.6) was used for instrument control, while Markerlynx for data integration. Cannabinoid separation was achieved using a linear gradient from 50% B to 100% B in 1.0 min, and maintained for 0.5 min, then the column was re-equilibrated at 50% B for 0.7 min before the next injection. The total run time for the method was 2.2 min. The mass spectrometer was operated in negative ion mode using Multi Reaction Monitoring (MRM) mode. The two most abundant transitions used were 357.12>178.99 and 357.12>245.06. Cone voltage was set at 54 V for both transitions while the collision energy was set at 22 eV for the first transition and 28 eV for the second one. SIM mode was used for detection. For all the different MS analyses, the capillary voltage was set at 2.2 kV. For quantification, where possible independent stock solutions of cannabinoids were prepared at 1 mg/mL in methanol. Successively, working solutions were prepared in methanol:water (1:1, v/v) to obtain a concentration range of (0.16-20) μM. Cannabinoid glycosides were initially identified in an untargeted approach, and later semi-quantified in SIM mode using predicted m/z values for each glycoside molecule.

### Part III.

Alternatively, for better separation of hydrophilic cannabinoid glycosides with multiple sugars LC-MS/Q-TOF analysis was performed on a Dionex UltiMate 3000 Quaternary Rapid Separation UHPLC⁺ focused system (Thermo Fisher Scientific, Germering, Germany) coupled to a Compact micrOTOF-Q mass spectrometer (Bruker, Bremen, Germany). Separation was achieved on a Kinetex 1.7 μm XB-C18 column (150×2.1 mm, 1.7 μm, 100 Å, Phenomenex). Solvents: 0.05% (v/v) formic acid in H₂O and MeCN as mobile phases A and B, respectively. Gradient conditions: Gradient (A): 0.0-2.0 min 2% B; 2.0-.0-25.0 min 2-100% B, 25.0-27.5 min 100% B, 27.5-28.0 min 100-2% B, and 28.0-30.0 min 2% B. Gradient (B): 0.0-1.0 min 10% B; 1.0-24.0 min 10-85% B; 24.0-25.0 min 85-100% B, 25.0-27.5 min 100% B, 27.5-28.0 min 100-2% B, and 28.0-30.0 min 2% B. Mobile phase flow rate was 300 μL/min. The column temperature was maintained at 30° C. UV spectra were acquired at 220, 230, 240, and 280 nm. The Compact micrOTOF-Q mass spectrometer (Bruker, Bremen, Germany) was equipped with an electrospray ion source operated in positive ion mode. The ion spray voltage was maintained at 4500 V with dry gas temperature at 250° C. Nitrogen was used as dry gas (8 L/min), nebulizing gas (2.5 bar), and collision gas. Collision energy was set to 10 eV. MS and MS/MS spectra were acquired in an m/z range from 50 to 1000 amu at a sampling rate of 2 Hz. Na-formate clusters were used for mass calibration.

### Extraction and Recovery of Cannabinoids and Glycosylated Cannabinoids in In Vitro Enzyme Assays of Example 8, 13, 16, 18 and 20:

### Part I.

Simultaneous hydrophobic cannabinoid and hydrophilic cannabinoid glycoside extraction from in vitro enzyme assays was performed by diluting the entire reaction mixture 4× in 100% methanol. For LC-MS/Q-TOF analysis samples were further diluted 10× in 50% MeOH and analyzed as stated above.

### Part II.

Alternatively, hydrophilic cannabinoid glycosides were extracted from in vitro glycosylation assays and separated from the hydrophobic cannabinoid substrate as follows. Ethyl acetate extraction was performed in a 1:1 ratio with the reaction mixture. The organic and aqueous fraction was separated by gravity and collected separately. The separated aqueous fraction was extracted a further 2 times with ethyl acetate 1:1. A small fraction of both organic and aqueous phases were analyzed by HPLC as described above to confirm presence of cannabinoid glycoside. The phase containing the cannabinoid glycoside was evaporated using a rotary evaporator. The resulting dry fraction was resuspended in 100% methanol and sonicated for 5 minutes. Proteins in the resuspension were precipitated by addition of ice-cold 100% acetone in 1:4 (v/v) ratio and incubation at −20° C. overnight. Protein precipitate was removed by centrifugation for 30 min @ 8000 rpm and supernatant was recovered. Centrifugation was repeated before freeze-drying of the recovered supernatant to evaporate the methanol and acetone. The resulting dry pellet was resuspended in 20% DMSO prior to loading on the Preparative HPLC for purification. Cannabinoid glycosides were purified on an Agilent 1200 preparative HPLC equipped with DAD detector. Separation was achieved on a Luna® 5 μm C18(2) LC column (150×21.2 mm, 5 μm, 100 Å, Phenomenex). Solvents: 0.01% (v/v) trifluoro acetic acid in H₂O and 0.01% (v/v) trifluoro acetic acid in MeCN as mobile phases A and B, respectively. Gradient conditions: 0-1 min 5% B; 1-5 min 5-40% B; 5-20 min 40-80% B; 20-21 min 80-100% B; 21-24 min 100% B; 24-25 min 100-5% B. Mobile phase flow rate was 15 mL/min. Column temperature was at room temperature. UV spectra were acquired at 220, 230 and 280 nm. Fraction collector collected fractions every 0.5 min from 5-20 min depending on cannabinoid glycoside. The fractions containing peaks based on UV spectra at 230 nm were collected and a sub-fraction was analyzed by HPLC (as stated above) to confirm identity and freeze-dried to dryness to recover purified cannabinoid glycoside as powder. Exact mass of purified compound was analyzed by LC-MS/QTOF as stated above.

### Example 1—Construction of Genetically Modified S. cerevisiae Strains for Production of Cannabinoids

### Part I.

Construction of S. cerevisiae strains producing hexanoic acid was performed based on the work described by Gajewski, Pavlovic, Fischer, Boles, & Grininger, Nature Comm; DOI: 10.1038/ncomms14650, 2017. Alternatively, the procedures of WO2016156548 could be used.

Deletion of the PDR12 gene as disclosed in the saccharomyces genome database (SGD) at www.yeastgenome.org was achieved as follows. The LoxP flanked SpHis5 cassette was amplified from pUG27 (Gueldener et al., 2002) with primers with 60 bp added homology to the upstream and downstream regions of PDR12. Transformation and selection on synthetic media with 20 g/L glucose minus histidine supplementation (SC-His) resulted in a strain with PDR12 deleted.

Integration of genes from the cannabinoid biosynthetic pathway(s) were achieved using the EasyClone marker free system described by (Jessop-Fabre et al., 2016) using an endonuclease such as MAD7 (https://www.inscripta.com/). Integration plasmids targeting defined locations in the genome were constructed as described in the tables below (Table 1-3). Plasmid backbones to construct these plasmids were obtained from Addgene (https://www.addgene.org/). Plasmids were linearized by restriction digestion with NotI (New England Bio Labs Inc.) and transformed into S. cerevisiae along with a gRNA plasmid targeting each genomic location according to (Gietz & Woods, 2002). Transformants were plated on selective media.

TABLE 1
Integration plasmids used to construct cannabinoid producing S. cerevisiae strains

| | | Backbone | | Promoter | |
|---|---|---|---|---|---|
| Name | Relevant description | plasmid | Biobrick 1 | biobrick | Biobrick 2 |
| p0001 | CsOAC and CsTKS overexpression and | pCfB2909 | BB0002 | BB0001 | BB0003 |
| | integration at EasyClone site XII-5 | | | | |
| p0002 | CsPT3 and AtGPPS overexpression and | pCfB3035 | BB0005 | BB0004 | BB0006 |
| | integration at EasyClone site X-4 | | | | |
| p0003 | CsAAE1 overexpression and integration at | pCfB3036 | | BB0007 | BB0008 |
| | EasyClone site XI-1 | | | | |
| p0004 | CsTHCAS overexpression and integration at | pCfB3040 | BB0010 | BB0009 | |
| | EasyClone site XII-4 | | | | |
| p0005 | CsCBDAS overexpression and integration at | pCfB3040 | BB0011 | BB0009 | |
| | EasyClone site XII-4 | | | | |
| p0006 | CsCBCAS overexpression and integration at | pCfB3040 | BB0012 | BB0009 | |
| | EasyClone site XII-4 | | | | |

TABLE 2
Biobricks used to construct integration plasmids

| | | Fwd | Rev | |
|---|---|---|---|---|
| Name | Relevant description | primer | primer | Template |
| BB0001 | <−pTEF1-pPGK1−> | PR0001 | PR0002 | pSP-GM1 |
| | double EasyClone | | | |
| | promoter | | | |
| BB0002 | CsOAC_U1 | PR0003 | PR0004 | Synthetic DNA |
| | | | | string |
| BB0003 | CsTKS_U2 | PR0005 | PR0006 | Synthetic DNA |
| | | | | string |
| BB0004 | <−pTDH3-pTEF1−> | PR0007 | PR0008 | p1977 |
| | double EasyClone | | | |
| | promoter | | | |
| BB0005 | CsPT3_U1 | PR0009 | PR0010 | Synthetic DNA |
| | | | | string |
| BB0006 | AtGPPS_U2 | PR0011 | PR0012 | Synthetic DNA |
| | | | | string |
| BB0007 | pPGK1−> EasyClone | PR0013 | PR0014 | pSP-GM1 |
| | promoter | | | |
| BB0008 | CsAAE1_U2 | PR0015 | PR0016 | Synthetic DNA |
| | | | | string |
| BB0009 | <−pTEF1 EasyClone | PR0017 | PR0018 | pSP-GM1 |
| | promoter | | | |
| BB0010 | CsTHCAS_U1 | PR0019 | PR0020 | Synthetic DNA |
| | | | | string |
| BB0011 | CsCBDAS_U1 | PR0021 | PR0022 | Synthetic DNA |
| | | | | string |
| BB0012 | CsCBCAS_U1 | PR0023 | PR0024 | Synthetic DNA |
| | | | | string |

TABLE 3
Primers used to amplify biobricks

| Name | SEQ ID NO | Purpose | Sequence |
|---|---|---|---|
| PR0001 | 61 | Fwd primer to amplify BB0001 (<− | Acctgcacuttgtaattaaaacttag |
| | | pTEF1-pPGK1−> double EasyClone | |
| | | promoter) | |
| PR0002 | 62 | Rev primer to amplify BB0001 (<− | Atgacagauttgttttatatttgttg |
| | | pTEF1-pPGK1−> double EasyClone | |
| | | promoter) | |
| PR0003 | 63 | Fwd primer to amplify BB0002 | AGTGCAGGUAAAACAATGGCTGTTAAGC |
| | | (CsOAC_U1) | ACTTGATCG |
| PR0004 | 64 | Rev primer to amplify BB0002 | CGTGCGAUCTACTTTCTTGGAGTGTAGT |
| | | (CsOAC_U1) | CGAAG |
| PR0005 | 65 | Fwd primer to amplify BB0003 | ATCTGTCAUAAAACAATGAACCACTTGA |
| | | (CsTKS_U2) | GAGCTGAAGG |
| PR0006 | 66 | Rev primer to amplify BB0003 | CACGCGAUCTAGTACTTGATTGGAACAG |
| | | (CsTKS_U2) | ATCTAAC |
| PR0007 | 67 | Fwd primer to amplify BB0004 (<− | ACCTGCACUTTTGTTTGTTTATGTGTGT |
| | | pTDH3-pTEF1−> double EasyClone | TTATTC |
| | | promoter) | |
| PR0008 | 68 | Rev primer to amplify BB0004 (<− | ATGACAGAUTTGTAATTAAAACTTAG |
| | | pTDH3-pTEF1−> double EasyClone | |
| | | promoter) | |
| PR0009 | 69 | Fwd primer to amplify BB0005 | AGTGCAGGUAAAACAATGGGTTTGTCTT |
| | | (CsPT3_U1) | TGGTTTGTACTTTC |
| PR0010 | 70 | Rev primer to amplify BB0005 | CGTGCGAUCTAGATGAAAACGTAAACGA |
| | | (CsPT3_U1) | AGTATTC |
| PR0011 | 71 | Fwd primer to amplify BB0006 | ATCTGTCAUAAAACAATGTTCGACTTCA |
| | | (AtGPPS_U2) | ACAAGTACATGG |
| PR0012 | 72 | Rev primer to amplify BB0006 | CACGCGAUCTACTAGTTTTGTCTGAAAG |
| | | (AtGPPS_U2) | CAACGTAG |
| PR0013 | 73 | Fwd primer to amplify BB0007 | Cgtgcgauggaagtaccttcaaaga |
| | | (pPGK1−> EasyClone promoter) | |
| PR0014 | 74 | Rev primer to amplify BB0007 | Atgacagauttgttttatatttgttg |
| | | (pPGK1−> EasyClone promoter) | |
| PR0015 | 75 | Fwd primer to amplify BB0008 | ATCTGTCAUAAAACAATGGGTAAGAACT |
| | | (C5AAE1_U2) | ACAAGTCTTTGG |
| PR0016 | 76 | Rev primer to amplify BB0008 | CACGCGAUCTATTCGAAGTGAGAGAATT |
| | | (C5AAE1_U2) | GTTGTCTC |
| PR0017 | 77 | Fwd primer to amplify BB0009 (<− | Acctgcacuttgtaattaaaacttag |
| | | pTEF1 EasyClone promoter) | |
| PR0018 | 78 | Rev primer to amplify BB0009 (<− | Cacgcgaugcacacaccatagcttc |
| | | pTEF1 EasyClone promoter) | |
| PR0019 | 79 | Fwd primer to amplify BB0010 | AGTGCAGGUAAAACAATGAACTGTTCTG |
| | | (CsTHCAS_U1) | CTTTCTCTTTCTGG |
| PR0020 | 80 | Rev primer to amplify BB0010 | CGTGCGAUCTAGTGGTGGTGTGGTGGCA |
| | | (CsTHCAS_U1) | ATGG |
| PR0021 | 81 | Fwd primer to amplify BB0011 | AGTGCAGGUAAAACAATGAAGTGTTCTA |
| | | (CsCBDAS_U1) | CTTTCTCTTTCTGG |
| PR0022 | 82 | Rev primer to amplify BB0011 | CGTGCGAUCTAGTGTCTGTGTCTTGGCA |
| | | (CsCBDAS_U1) | ATGG |
| PR0023 | 83 | Fwd primer to amplify BB0012 | AGTGCAGGUAAAACAATGAACTGTTCTA |
| | | (CsCBCAS_U1) | CTTTCTCTTTC |
| PR0024 | 84 | Rev primer to amplify BB0012 | CGTGCGAUCTAGTGGTGTCTTGGTGGCA |
| | | (CsCBCAS_U1) | ATGG |

All heterologous genes are codon-optimized for expression in Saccharomyces cerevisiae using the JCAT algorithm (Grote et al., 2005), synthesized by GeneArt and are placed under the control of strong S. cerevisiae constitutive promoters and terminators. Amplification of biobricks are performed using PhusionU polymerase (ThermoScientific).

### Part II.

Alternatively, cannabinoid producing strains can be constructed as follows. Strains producing hexanoic acid can be constructed as described above or alternatively hexanoic acid can be added exogenously to the cultivation media. Genes for the cannabinoid biosynthetic pathway are integrated into pre-defined genomic “landing pads” using custom-made overexpression plasmids similar to the system described by (Mikkelsen et al., 2012). Linear integration fragments are produced by NotI digestion of custom designed plasmids containing strong constitutive S. cerevisiae promoters and terminators and are flanked by upstream and downstream homology regions to facilitate assembly by homologous recombination. To facilitate assembly of multiple integration plasmids at a single genomic loci, upstream and downstream homology arms are designed so that after NotI digestion (New England Bio Labs Inc.), linear integration fragments can recombine into a single linear integration fragment and integrate in the target genomic loci. To select for transformants that have successfully integrated the fragments of interest, an endonuclease such as MAD7 can be used as described above or alternatively a selection marker such as LEU2 can be incorporated into the linear integration fragments and transformed into S. cerevisiae strains that are auxotrophic for Leucine as is known in the art. To reduce the occurrence of false positives the selection marker can be split across 2 linear integration fragments such as Rec 1 and Rec 2 such that a functional LEU2 selection marker can only be generated upon successful homologous recombination of the Rec 1 and Rec 2 integration fragments as shown in FIG. 1.

Genes are codon-optimized for expression in yeast and synthesized and cloned into custom integration plasmids by Twist Biosciences (Table 4). After linearization by restriction digestion with NotI (New England Bio Labs Inc.) plasmids are transformed into S. cerevisiae according to (Gietz & Woods, 2002). Transformants are plated on selective media.

TABLE 4
Integration plasmids used to construct cannabinoid producing S. cerevisiae strains

| Plasmid name | Gene | Description |
|---|---|---|
| PL-381(Rec1-XI-5-LEU: | CsTKS-CsOAC | Fusion protein with CsTKS and CsOAC |
| CsTKS-CsOAC) | | |
| PL-382(Rec2-LEU: | AgGPPS2 | GPP synthase that is specific for GPP production from |
| AgGPPS2) | | IPP and DMAPP |
| PL-383(Rec3: CsTHCAS) | CsTHCAS (ProA) | Cannabis Sativa THCA synthase with vacuolar |
| | | localization tag added. Converts CBGA to THCA |
| PL-384(Rec3: CsCBDAS) | CsCBDAS (ProA) | Cannabis Sativa CBDA synthase with vacuolar |
| | | localization tag added. Converts CBGA to CBDA |
| PL-385(Rec4: CsPT4) | CsPT4ΔN- | Cannabis Sativa prenyltransferase 4 with predicted N- |
| | terminal | terminal sequence removed. Converts olivetolic acid |
| | | and GPP to CBGA |
| PL-386(Rec4: | SsNphB(Q295F) | Streptomyces sp prenyltransferase with Q295F |
| SsNphB(Q295F)) | | mutation. Soluble prenyltransferase catalyzing |
| | | conversion of olivetolic acid and GPP to CBGA. |
| PL-387(Rec5-XI-5: | CsAAE1 | Cannabis Sativa Acyl activating enzyme. Converts |
| CsAAE1) | | hexanoic acid to hexanoyl-CoA |

### Example 2—Production of Cannabinoids in Genetically Modified S. cerevisiae Strains

### Part I.

The yeast strains were pre-cultured in 500 μL of liquid synthetic complete media (SC) or synthetic complete media with 20 g/L glucose minus uracil supplementation (SC-Ura) for 24 h at 30° C., 300 rpm in 2 mL microtiter plates with air-permeable sealing. Subsequently, 50 μL of yeast preculture was transferred to 450 μL SC, or SC-Ura with 20 g/L feed-in-time (FIT) minimal medium (Enpresso) with 0.3% enzyme, or other suitable carbon source such as 20 g/L glucose and grown for 72 h, 30° C., 300 rpm. Cells were incubated in medium containing hexanoic acid (1 mM), butanoic acid (1 mM), other intermediates of the cannabinoid biosynthetic pathway, or with no supplementation (strains producing fatty acids de novo as described above). After incubation, cannabinoids were extracted and analyzed as described above. HPLC or LC-MS were used for all analyses as described and where possible, authentic analytical standards are used. Since biosynthetic production produced the acid form of cannabinoids whereas the decarboxylated form is typically the bioactive version, in some aspects, decarboxylated cannabinoids were prepared by heating the evaporated cannabinoid extracts at 110° C. for 50 minutes prior to resuspension in acetonitrile/H₂O/formic acid (80%/20%/0.05% (v/v/v)). In some aspects, decarboxylated cannabinoids were prepared by directly heating the cell culture broth at 80° C. for 50 minutes prior to further extraction as described above.

### Part II.

Alternatively, yeast strains were pre-cultured overnight at 30° C. and 300 rpm in synthetic media lacking amino acid supplementation as required to maintain selection on introduced expression plasmids and/or integration cassettes. 10 μL of cell culture was subsequently transferred to 490 μL of synthetic media minus amino acid supplementation supplemented with 20 g/L glucose, 20 g/L ethanol, 1 mM hexanoic acid or 1 mM butanoic acid other intermediates of the cannabinoid biosynthetic pathway as required (or combinations thereof). Cells were incubated for 3 days at 30° C. and 300 rpm, cannabinoids were extracted and analyzed as previously described. Decarboxylated cannabinoids were prepared by heating the evaporated cannabinoid extracts at 110° C. for 50 minutes prior to resuspension in acetonitrile/H₂O/formic acid (80%/20%/0.05% (v/v/v)). In some aspects, decarboxylated cannabinoids were prepared by directly heating the cell culture broth at 80° C. for 50 minutes prior to further extraction as described above.

### Example 3—Construction of Genetically Modified E. coli Strains for Production of Cannabinoids

The cannabinoid biosynthetic pathway was introduced into E. coli as follows. Genes were amplified from synthetic DNA using primers with added restriction digestion sites and cloned into the pETDuet-1, pETACYCDuet-1 and pCDFDuet-1 dual expression vectors (Novagen). Plasmids were transformed into E. coli strain BL21 (DE3) and successful transformants selected on ampicillin, chloramphenicol and streptomycin respectively. Outline of plasmids (Table 5), biobricks (Table 6) and primers (Table 7) used are presented below.

TABLE 5
Plasmids constructed to engineer cannabinoid biosynthesis in E. coli

| | | Backbone | | |
|---|---|---|---|---|
| Name | Relevant description | plasmid | Biobrick 1 | Biobrick 2 |
| p0007 | CsOAC and CsTKS overexpression plasmid for E. coli | pETDuet-1 | CsOAC | CsTKS |
| | expression | | | |
| p0008 | CsPT3 and AtGPPS overexpression plasmid for E. coli | pACYCDuet-1 | CsPT3 | AtGPPS |
| | expression | | | |
| p0009 | CsAAE1 and CsTHCAS overexpression plasmid for E. coli | pCDFDuet-1 | CsAAE1 | CsTHCAS |
| | expression | | | |
| p0010 | CsAAE1 and CsCBDAS overexpression plasmid for E. coli | pCDFDuet-1 | CsAAE1 | CsCBDAS |
| | expression | | | |
| p0011 | CsAAE1 and CsCBCAS overexpression plasmid for E. coli | pCDFDuet-1 | CsAAE1 | CsCBCAS |
| | expression | | | |

TABLE 6
Biobricks used to construct plasmids

| | Relevant | Fwd | Rev | |
|---|---|---|---|---|
| Name | description | primer | primer | Template |
| BB0013 | CsOAC | PR0025 | PR0026 | Synthetic DNA |
| | | | | string |
| BB0014 | CsTKS | PR0027 | PR0028 | Synthetic DNA |
| | | | | string |
| BB0015 | CsPT3 | PR0029 | PR0030 | Synthetic DNA |
| | | | | string |
| BB0016 | AtGPPS | PR0031 | PR0032 | Synthetic DNA |
| | | | | string |
| BB0017 | CsAAE1 | PR0033 | PR0034 | Synthetic DNA |
| | | | | string |
| BB0018 | CsTHCAS | PR0035 | PR0036 | Synthetic DNA |
| | | | | string |
| BB0019 | CsCBDAS | PR0037 | PR0038 | Synthetic DNA |
| | | | | string |
| BB0020 | CsCBCAS | PR0039 | PR0040 | Synthetic DNA |
| | | | | string |

TABLE 7
Primers used to amplify biobricks.

| Name | SEQ ID NO | Purpose | Sequence |
|---|---|---|---|
| PR0025 | 85 | Fwd primer to amplify | GGATCCATGGCTGTTAAGCACTTGATCG |
| | | BB0013 with BamHI | |
| | | site (CsOAC) | |
| PR0026 | 86 | Rev primer to amplify | AAGCTTCTACTTTCTTGGAGTGTAGTCGAAG |
| | | BB0013 with HindIII | |
| | | site (CsOAC) | |
| PR0027 | 87 | Fwd primer to amplify | CGCCGGCGATGAACCACTTGAGAGCTGAAGG |
| | | BB0014 with NotI site | |
| | | (CsTKS) | |
| PR0028 | 88 | Rev primer to amplify | CTTAAGCTAGTACTTGATTGGAACAGATCTAAC |
| | | BB0014 with AflII site | |
| | | (CsTKS) | |
| PR0029 | 89 | Fwd primer to amplify | GGATCCATGGGTTTGTCTTTGGTTTGTACTTTC |
| | | BB0015 with BamHI | |
| | | site (CsPT3) | |
| PR0030 | 90 | Rev primer to amplify | AAGCTTCTAGATGAAAACGTAAACGAAGTATTC |
| | | BB0015 with HindIII | |
| | | site (CsPT3) | |
| PR0031 | 91 | Fwd primer to amplify | CGCCGGCGATGTTCGACTTCAACAAGTACATGG |
| | | BB0016 with NotI site | |
| | | (AtGPPS) | |
| PR0032 | 92 | Rev primer to amplify | CTTAAGCTACTAGTTTTGTCTGAAAGCAACGTAG |
| | | BB0016 with AflII site | |
| | | (AtGPPS) | |
| PR0033 | 93 | Fwd primer to amplify | GGATCCATGGGTAAGAACTACAAGTCTTTGG |
| | | BB0017 with BamHI | |
| | | site (CsAAE1) | |
| PR0034 | 94 | Rev primer to amplify | AAGCTTCTATTCGAAGTGAGAGAATTGTTGTCTC |
| | | BB0017 with HindIII | |
| | | site (CsAAE1) | |
| PR0035 | 95 | Fwd primer to amplify | CGCCGGCGATGAACTGTTCTGCTTTCTCTTTCTGG |
| | | BB0018 with NotI site | |
| | | (CsTHCAS) | |
| PR0036 | 96 | Rev primer to amplify | CTTAAGCTAGTGGTGGTGTGGTGGCAATGG |
| | | BB0018 with AflII site | |
| | | (CsTHCAS) | |
| PR0037 | 97 | Fwd primer to amplify | CGCCGGCGATGAAGTGTTCTACTTTCTCTTTCTGG |
| | | BB0019 with NotI site | |
| | | (CsCBDAS) | |
| PR0038 | 98 | Rev primer to amplify | CTTAAGCTAGTGTCTGTGTCTTGGCAATGG |
| | | BB0019 with AflII site | |
| | | (CsCBDAS) | |
| PR0039 | 99 | Fwd primer to amplify | CGCCGGCGATGAACTGTTCTACTTTCTCTTTC |
| | | BB0020 with NotI site | |
| | | (CsCBCAS) | |
| PR0040 | 100 | Rev primer to amplify | CTTAAGCTAGTGGTGTCTTGGTGGCAATGG |
| | | BB0020 with AflII site | |
| | | (CsCBCAS) | |

### Example 4—Production of Cannabinoids in Genetically Modified E. coli Strains

E. coli strains were pre-cultured in 5004 of liquid LB media supplemented with ampicillin, chloramphenicol and streptomycin (LB+AmpChlorStrep) for 24 h at 37° C., 300 rpm in 2 mL microtiter plates with air-permeable sealing. Subsequently 50 μL of pre-culture was transferred to 450 μl of LB+AmpChlorStrep with 20 g/L glucose supplemented and cultured for 24 h at 37° C., 300 rpm. Cells were further incubated in medium containing hexanoic acid (1 mM), butanoic acid (1 mM), other intermediates of the cannabinoid biosynthetic pathway or with no fatty acid supplementation (strains producing fatty acids de novo as described above) with polypeptide expression inducer added. After incubation, cannabinoids were extracted and analyzed as described above. LC-MS or HPLC were used for all analyses as described and where possible, authentic analytical standards were used. Since biosynthetic production produced the acid form of cannabinoids whereas the decarboxylated form is typically the bioactive version, in some aspects, decarboxylated cannabinoids were prepared by heating the evaporated cannabinoid extracts at 110° C. for 50 minutes prior to resuspension in acetonitrile/H₂O/formic acid (80%/20%/0.05% (v/v/v)). In some aspects, decarboxylated cannabinoids were prepared by directly heating the cell culture broth at 80° C. for 50 minutes prior to further extraction as described above.

### Example 5—Construction of S. cerevisiae Strains for Production of Cannabinoid Glycosides

### Part I.

Genes for expression in S. cerevisiae are codon-optimized and synthesized by GeneArt. Genes are PCR amplified with primers adding the U2 USER cloning site and cloned into the episomal expression vector pCfB132 using the EasyClone system as described by (Jensen et al., 2014) using strong constitutive promoters and terminators. Transformants are selected by plating on media in the absence of uracil. Outline of plasmids (Table 8), biobricks (Table 9) and primers (Table 10) used are outlined below. Plasmid backbone is available from Addgene (https://www.addgene.org/)

TABLE 8
Plasmids constructed to overexpress glycosyl transferases in S. cerevisiae

| | | | | | |
|---|---|---|---|---|---|
| | | Backbone | | Promoter | |
| Name | Relevant description | plasmid | Biobrick 1 | biobrick | Biobrick 2 |

| | | | | |
|---|---|---|---|---|
| p0012 | UGT708G3_U2 overexpression from | pCfB132 | BB0007 | BB0021 |
| | episomal plasmid | | | |
| p0013 | UGT708G2_U2 overexpression from | pCfB132 | BB0007 | BB0022 |
| | episomal plasmid | | | |
| p0014 | UGT708G1_U2 overexpression from | pCfB132 | BB0007 | BB0023 |
| | episomal plasmid | | | |
| p0015 | OsCGT_U2 overexpression from | pCfB132 | BB0007 | BB0024 |
| | episomal plasmid | | | |
| p0016 | FeUGT708C1_U2 overexpression from | pCfB132 | BB0007 | BB0025 |
| | episomal plasmid | | | |
| p0017 | GmUGT708D1_U2 overexpression | pCfB132 | BB0007 | BB0026 |
| | from episomal plasmid | | | |
| p0018 | ZmUGT708A6_U2 overexpression | pCfB132 | BB0007 | BB0027 |
| | from episomal plasmid | | | |
| p0019 | MiCGT_U2 overexpression from | pCfB132 | BB0007 | BB0028 |
| | episomal plasmid | | | |
| p0020 | GtUF6CGT1_U2 overexpression from | pCfB132 | BB0007 | BB0029 |
| | episomal plasmid | | | |
| p0021 | DcUGT2_U2 overexpression from | pCfB132 | BB0007 | BB0030 |
| | episomal plasmid | | | |
| p0022 | DcUGT4_U2 overexpression from | pCfB132 | BB0007 | BB0031 |
| | episomal plasmid | | | |
| p0023 | DcUGT5_U2 overexpression from | pCfB132 | BB0007 | BB0032 |
| | episomal plasmid | | | |
| p0024 | UGT73B5_U2 overexpression from | pCfB132 | BB0007 | BB0033 |
| | episomal plasmid | | | |
| p0025 | UGT76C5_U2 overexpression from | pCfB132 | BB0007 | BB0034 |
| | episomal plasmid | | | |
| p0026 | UGT73B3_U2 overexpression from | pCfB132 | BB0007 | BB0035 |
| | episomal plasmid | | | |
| p0027 | UGT71E1_U2 overexpression from | pCfB132 | BB0007 | BB0036 |
| | episomal plasmid | | | |
| p0028 | UGT5_U2 overexpression from | pCfB132 | BB0007 | BB0037 |
| | episomal plasmid | | | |
| p0029 | UGT1A10_U2 overexpression from | pCfB132 | BB0007 | BB0038 |
| | episomal plasmid | | | |
| p0030 | UGT1A9_U2 overexpression from | pCfB132 | BB0007 | BB0039 |
| | episomal plasmid | | | |
| p0031 | UGT2B7_U2 overexpression from | pCfB132 | BB0007 | BB0040 |
| | episomal plasmid | | | |

TABLE 9
Biobricks to construct glycosyl transferase
plasmids in S. cerevisiae.

| | Relevant | Fwd | Rev | |
|---|---|---|---|---|
| Name | description | primer | primer | Template |
| BB0021 | UGT708G3_U2 | PR0041 | PR0042 | Synthetic DNA string |
| BB0022 | UGT708G2_U2 | PR0043 | PR0044 | Synthetic DNA string |
| BB0023 | UGT708G1_U2 | PR0045 | PR0046 | Synthetic DNA string |
| BB0024 | OsCGT_U2 | PR0047 | PR0048 | Synthetic DNA string |
| BB0025 | FeUGT708C1_U2 | PR0049 | PR0050 | Synthetic DNA string |
| BB0026 | GmUGT708D1_U2 | PR0051 | PR0052 | Synthetic DNA string |
| BB0027 | ZmUGT708A6_U2 | PR0053 | PR0054 | Synthetic DNA string |
| BB0028 | MiCGT_U2 | PR0055 | PR0056 | Synthetic DNA string |
| BB0029 | GtUF6CGT1_U2 | PR0057 | PR0058 | Synthetic DNA string |
| BB0030 | DcUGT2_U2 | PR0059 | PR0060 | Synthetic DNA string |
| BB0031 | DcUGT4_U2 | PR0061 | PR0062 | Synthetic DNA string |
| BB0032 | DcUGT5_U2 | PR0063 | PR0064 | Synthetic DNA string |
| BB0033 | UGT73B5_U2 | PR0065 | PR0066 | Synthetic DNA string |
| BB0034 | UGT76C5_U2 | PR0067 | PR0068 | Synthetic DNA string |
| BB0035 | UGT73B3_U2 | PR0069 | PR0070 | Synthetic DNA string |
| BB0036 | UGT71E1_U2 | PR0071 | PR0072 | Synthetic DNA string |
| BB0037 | UGT5_U2 | PR0073 | PR0074 | Synthetic DNA string |
| BB0038 | UGT1A10_U2 | PR0075 | PR0076 | Synthetic DNA string |
| BB0039 | UGT1A9_U2 | PR0077 | PR0078 | Synthetic DNA string |
| BB0040 | UGT2B7_U2 | PR0079 | PR0080 | Synthetic DNA string |

TABLE 10
Primers used to construct biobricks

| Name | SEQ ID NO | Purpose | Sequence |
|---|---|---|---|
| PR0041 | 241 | Fwd primer to amplify | ATCTGTCAUAAAACAATGTCTGACTCTGGTGGTTTCGAC |
| | | BB0021(UGT708G3_U2) | |
| PR0042 | 242 | Rev primer to | CACGCGAUCTAGTGAGTGTTGTTGTTACACTTCC |
| | | amplifyBB0021(UGT708G3_U2) | |
| PR0043 | 243 | Fwd primer to amplify | ATCTGTCAUAAAACAATGTCTGACTCTGGTGGTTTCGAC |
| | | BB0022(UGT708G2_U2) | |
| PR0044 | 244 | Rev primer to | CACGCGAUCTAGTGAGTGTTGTTGTTACACTTCC |
| | | amplifyBB0022(UGT708G2_U2) | |
| PR0045 | 245 | Fwd primer to amplify | ATCTGTCAUAAAACAATGTCTGACTCTGGTGGTTTCGAC |
| | | BB0023(UGT708G1_U2) | |
| PR0046 | 246 | Rev primer to | CACGCGAUCTAGTGAGTGTTGTTGTTACACTTCC |
| | | amplifyBB0023(UGT708G1_U2) | |
| PR0047 | 247 | Fwd primer to amplify | ATCTGTCAUAAAACAATGCCATCTTCTGGTGACGCTGCTGG |
| | | BB0024(OsCGT_U2) | |
| PR0048 | 248 | Rev primer to | CACGCGAUCTAGTTAGTTCTACAAGTACCACC |
| | | amplifyBB0024(0sCGT_U2) | |
| PR0049 | 249 | Fwd primer to amplify | ATCTGTCAUAAAACAATGATGGGTGACTTGACTACTTC |
| | | BB0025(FeUGT708C1_U2) | |
| PR0050 | 250 | Rev primer to | CACGCGAUCTATCTCTTCAAAGAACCGATG |
| | | amplifyBB0025(FeUGT708C1_U2) | |
| PR0051 | 251 | Fwd primer to amplify | ATCTGTCAUAAAACAATGTCTTCTTCTGAAGGTGTTG |
| | | BB0026(GmUGT7081M_U2) | |
| PR0052 | 252 | Rev primer to | CACGCGAUCTAGTTAGCTTGAGCGTTTCTC |
| | | amplifyBB0026(GmUGT7081M_U2) | |
| PR0053 | 253 | Fwd primer to amplify | ATCTGTCAUAAAACAATGGCTGCTAACGGTGGTGACC |
| | | BB0027(ZmUGT708A6_U2) | |
| PR0054 | 254 | Rev primer to | CACGCGAUCTACTTTCTTTCAGCGTCTCTAC |
| | | amplifyBB0027(ZmUGT708A6_U2) | |
| PR0055 | 255 | Fwd primer to amplify | ATCTGTCAUAAAACAATGTCTGCTTCTGACGCTTTG |
| | | BB0028(MiCGT_U2) | |
| PR0056 | 256 | Rev primer to | CACGCGAUCTAAGTCTTTCTAGAAGTCTTCTTCC |
| | | amplifyBB0028(MiCGT_U2) | |
| PR0057 | 257 | Fwd primer to amplify | ATCTGTCAUAAAACAATGGGTTCTTTGACTAACAACG |
| | | BB0029(GtUF6CGT1_U2) | |
| PR0058 | 258 | Rev primer to | CACGCGAUCTACTTAGTACCAGTCTTTCTAGC |
| | | amplifyBB0029(GtUF6CGT1_U2) | |
| PR0059 | 259 | Fwd primer to amplify | ATCTGTCAUAAAACAATGGAATTCAGATTGTTGATCTTGG |
| | | BB0030(DcUGT2_U2) | |
| PR0060 | 260 | Rev primer to | CACGCGAUCTAGTTCTTCTTCAACTTTTCAG |
| | | amplifyBB0030(DcUGT2_U2) | |
| PR0061 | 261 | Fwd primer to amplify | ATCTGTCAUAAAACAATGACTTTGTTGAGAGACTTGTTG |
| | | BB0031(DcUGT4_U2) | |
| PR0062 | 262 | Rev primer to | CACGCGAUCTACTTAGTCAACATTCTGAAG |
| | | amplifyBB0031(DcUGT4_U2) | |
| PR0063 | 263 | Fwd primer to amplify | ATCTGTCAUAAAACAATGATCTTCTTCTACTTCTTGAC |
| | | BB0032(DcUGT5_U2) | |
| PR0064 | 264 | Rev primer to | CACGCGAUCTAGTTGTCCTTAACCTTCTTAG |
| | | amplifyBB0032(DcUGT5_U2) | |
| PR0065 | 265 | Fwd primer to amplify | ATCTGTCAUAAAACAATGAACAGAGAAGTTTCTGAAAG |
| | | BB0033(UGT73135_U2) | |
| PR0066 | 266 | Rev primer to | CACGCGAUCTACTTTCTACCGTTCAATTCTTCC |
| | | amplifyBB0033(UGT73135_U2) | |
| PR0067 | 267 | Fwd primer to amplify | ATCTGTCAUAAAACAATGGAAAAGTCTAACGGTTTGAG |
| | | BB0034(UGT76C5_U2) | |
| PR0068 | 268 | Rev primer to | CACGCGAUCTAGAAAGAAGAGATGTAGTCG |
| | | amplifyBB0034(UGT76C5_U2) | |
| PR0069 | 269 | Fwd primer to amplify | ATCTGTCAUAAAACAATGTCTTCTGACCCACACAGAAAG |
| | | BB0035(UGT73133_U2) | |
| PR0070 | 270 | Rev primer to | CACGCGAUCTAAGAAGTGAATTCTTCGATG |
| | | amplifyBB0035(UGT73133_U2) | |
| PR0071 | 271 | Fwd primer to amplify | ATCTGTCAUAAAACAATGTCTACTTCTGAATTGGTTTTC |
| | | BB0036(UGT71E1_U2) | |
| PR0072 | 272 | Rev primer to | CACGCGAUCTAGATAGTAACGTTAGAAACG |
| | | amplifyBB0036(UGT71E1_U2) | |
| PR0073 | 273 | Fwd primer to amplify | ATCTGTCAUAAAACAATGAAGCAAACTGTTGTTTTGTAC |
| | | BB0037(UGT5_U2) | |
| PR0074 | 274 | Rev primer to | CACGCGAUCTAGTTTTGAACCAAGTTTTCAAC |
| | | amplifyBB0037(UGT5_U2) | |
| PR0075 | 275 | Fwd primer to amplify | ATCTGTCAUAAAACAATGGCTAGAGCTGGTTGGAC |
| | | BB0038(UGT1A10_U2) | |
| PR0076 | 276 | Rev primer to | CACGCGAUCTAGTGAGTCTTAGACTTGTGAGC |
| | | amplifyBB0038(UGT1A10_U2) | |
| PR0077 | 277 | Fwd primer to amplify | ATCTGTCAUAAAACAATGGCTTGTACTGGTTGGACTTC |
| | | BB0039(UGT1A9_U2) | |
| PR0078 | 278 | Rev primer to | CACGCGAUCTAGTGAGTCTTAGACTTGTGAGC |
| | | amplifyBB0039(UGT1A9_U2) | |
| PR0079 | 279 | Fwd primer to amplify | ATCTGTCAUAAAACAATGTCTGTTAAGTGGACTTC |
| | | BB0040(UGT2B7_U2) | |
| PR0080 | 280 | Rev primer to | CACGCGAUCTAGTCGTTCTTACCCTTCTTAG |
| | | amplifyBB0040(UGT2B7_U2) | |

### Part II.

Alternatively, genes for expression in S. cerevisiae are codon-optimized, synthesized and cloned into plasmids by Twist Biosciences. Genes are cloned into the yeast centromeric expression vector p413TEF which contains the TEF1 strong constitutive promoter, CYC1 terminator and HIS3 auxotrophic market. The p413TEF plasmid backbone is available from ATCC (ATCC #87362). Transformants are selected by plating on media in the absence of histidine. Outline of plasmids are described below, Table 11.

TABLE 11
Plasmids constructed to overexpress glycosyl
transferases in S. cerevisiae

| Plasmid | Backbone | Gene |
|---|---|---|
| pSCUGT-1 | p413TEF | At73C5 |
| pSCUGT-2 | p413TEF | At71D1 |
| pSCUGT-3 | p413TEF | At72B1 |
| pSCUGT-4 | p413TEF | Sr71E1 |
| pSCUGT-5 | p413TEF | OsEUGT11 |
| pSCUGT-6 | p413TEF | Sp73E |
| pSCUGT-7 | p413TEF | OsO-1 |
| pSCUGT-8 | p413TEF | At84B1 |
| pSCUGT-9 | p413TEF | Sr76G1 |
| pSCUGT-10 | p413TEF | Pa85 |
| pSCUGT-11 | p413TEF | CrUGT-2 |
| pSCUGT-12 | p413TEF | At73B3 |
| pSCUGT-13 | p413TEF | At71C1-Sr71E1 354 |
| pSCUGT-14 | p413TEF | Pa72 |
| pSCUGT-15 | p413TEF | At73B5 |
| pSCUGT-16 | p413TEF | At71C1_At71C2 353 |
| pSCUGT-17 | p413TEF | Cp89B |
| pSCUGT-18 | p413TEF | Sp89B |
| pSCUGT-19 | p413TEF | Tc90A |
| pSCUGT-20 | p413TEF | Si94D |
| pSCUGT-21 | p413TEF | Pt88G |
| pSCUGT-22 | p413TEF | Ha88B_2 |
| pSCUGT-23 | p413TEF | Ac73T |
| pSCUGT-24 | p413TEF | Si73X |
| pSCUGT-25 | p413TEF | Tc74Z |
| PL-388(p413TEF: | p413TEF | Cs73Y |
| Cs73Y) | | |
| pSCUGT-26 | p413TEF | Pt73Y |
| pSCUGT-27 | p413TEF | Ac73Z |
| pSCUGT-28 | p413TEF | Bv75C |
| pSCUGT-29 | p413TEF | Pt78G |
| pSCUGT-30 | p413TEF | Si82A |
| pSCUGT-31 | p413TEF | Ad74X |
| pSCUGT-32 | p413TEF | Cs74S |
| pSCUGT-33 | p413TEF | Ad72AA |
| pSCUGT-34 | p413TEF | Si71E_2 |
| pSCUGT-35 | p413TEF | Vv71R |
| pSCUGT-36 | p413TEF | Ha72B |
| pSCUGT-37 | p413TEF | Sp73A |
| pSCUGT-38 | p413TEF | Bv73P |
| pSCUGT-39 | p413TEF | Pt72B |
| pSCUGT-40 | p413TEF | Qs72S_1 |
| pSCUGT-41 | p413TEF | Ad72X |
| pSCUGT-42 | p413TEF | Cp73B |
| pSCUGT-43 | p413TEF | Zj71A |
| pSCUGT-44 | p413TEF | Ha71S |
| pSCUGT-45 | p413TEF | Ac73H |
| pSCUGT-46 | p413TEF | Cp71B |
| pSCUGT-47 | p413TEF | Ha72T |
| pSCUGT-48 | p413TEF | Sp73Q |
| pSCUGT-49 | p413TEF | Sp72T |

### Example 6—Construction of E. coli Strains for Production of Cannabinoid Glycosides

### Part I.

Glycosyl transferase genes for expression in E. coli were synthesized by GeneArt. Genes were PCR amplified with primers adding restriction sites and cloned into the pRSFDuet-1 expression plasmid using standard restriction/ligation cloning. Transformants were selected by plating on media containing kanamycin. Plasmids were transformed into DH5a, “Arctic express” (Agilent technologies), or Xjb-autolysis BL21 (Zymo research) E. coli strains or the constructed E. coli strains of previous examples. Outline of plasmids (Table 12), biobricks (Table 13) and plasmids (Table 14) used are outlined below

TABLE 12
Plasmids constructed to introduce glycosyl transferases into E. coli.

| | | Backbone | Biobrick |
|---|---|---|---|
| Name | Relevent description | plasmid | 1 |
| p0032 | UGT708G3 overexpression | pRSFDuet-1 | BB0041 |
| | plasmid for E. coli expression | | |
| p0033 | UGT708G2 overexpression | pRSFDuet-1 | BB0042 |
| | plasmid for E. coli expression | | |
| p0034 | UGT708G1 overexpression | pRSFDuet-1 | BB0043 |
| | plasmid for E. coli expression | | |
| p0035 | OsCGT overexpression | pRSFDuet-1 | BB0044 |
| | plasmid for E. coli expression | | |
| p0036 | FeUGT708C1 overexpression | pRSFDuet-1 | BB0045 |
| | plasmid for E. coli expression | | |
| p0037 | GmUGT708D1 overexpression | pRSFDuet-1 | BB0046 |
| | plasmid for E. coli expression | | |
| p0038 | ZmUGT708A6 overexpression | pRSFDuet-1 | BB0047 |
| | plasmid for E. coli expression | | |
| p0039 | MiCGT overexpression | pRSFDuet-1 | BB0048 |
| | plasmid for E. coli expression | | |
| p0040 | GtUF6CGT1 overexpression | pRSFDuet-1 | BB0049 |
| | plasmid for E. coli expression | | |
| p0041 | DcUGT2 overexpression | pRSFDuet-1 | BB0050 |
| | plasmid for E. coli expression | | |
| p0042 | DcUGT4 overexpression | pRSFDuet-1 | BB0051 |
| | plasmid for E. coli expression | | |
| p0043 | DcUGT5 overexpression | pRSFDuet-1 | BB0052 |
| | plasmid for E. coli expression | | |
| p0044 | UGT73B5 overexpression | pRSFDuet-1 | BB0053 |
| | plasmid for E. coli expression | | |
| p0045 | UGT76C5 overexpression | pRSFDuet-1 | BB0054 |
| | plasmid for E. coli expression | | |
| p0046 | UGT73B3 overexpression | pRSFDuet-1 | BB0055 |
| | plasmid for E. coli expression | | |
| p0047 | UGT71E1 overexpression | pRSFDuet-1 | BB0056 |
| | plasmid for E. coli expression | | |
| p0048 | UGT5 overexpression | pRSFDuet-1 | BB0057 |
| | plasmid for E. coli expression | | |
| p0049 | UGT1A10 overexpression | pRSFDuet-1 | BB0058 |
| | plasmid for E. coli expression | | |
| p0050 | UGT1A9 overexpression | pRSFDuet-1 | BB0059 |
| | plasmid for E. coli expression | | |
| p0051 | UGT2B7 overexpression | pRSFDuet-1 | BB0060 |
| | plasmid for E. coli expression | | |

TABLE 13
Biobricks used to construct glycosyl transferase plasmids in E. coli

| Name | Relevant description | Fwd primer | Rev primer | Template |
|---|---|---|---|---|
| BB0041 | UGT708G3 | PR0081 | PR0082 | Synthetic DNA string |
| BB0042 | UGT708G2 | PR0083 | PR0084 | Synthetic DNA string |
| BB0043 | UGT708G1 | PR0085 | PR0086 | Synthetic DNA string |
| BB0044 | OsCGT | PR0087 | PR0088 | Synthetic DNA string |
| BB0045 | FeUGT708C1 | PR0089 | PR0090 | Synthetic DNA string |
| BB0046 | GmUGT708D1 | PR0091 | PR0092 | Synthetic DNA string |
| BB0047 | ZmUGT708A6 | PR0093 | PR0094 | Synthetic DNA string |
| BB0048 | MiCGT | PR0095 | PR0096 | Synthetic DNA string |
| BB0049 | GtUF6CGT1 | PR0097 | PR0098 | Synthetic DNA string |
| BB0050 | DcUGT2 | PR0099 | PR0100 | Synthetic DNA string |
| BB0051 | DcUGT4 | PR0101 | PR0102 | Synthetic DNA string |
| BB0052 | DcUGT5 | PR0103 | PR0104 | Synthetic DNA string |
| BB0053 | UGT73B5 | PR0105 | PR0106 | Synthetic DNA string |
| BB0054 | UGT76C5 | PR0107 | PR0108 | Synthetic DNA string |
| BB0055 | UGT73B3 | PR0109 | PR0110 | Synthetic DNA string |
| BB0056 | UGT71E1 | PR0111 | PR0112 | Synthetic DNA string |
| BB0057 | UGT5 | PR0113 | PR0114 | Synthetic DNA string |
| BB0058 | UGT1A10 | PR0115 | PR0116 | Synthetic DNA string |
| BB0059 | UGT1A9 | PR0117 | PR0118 | Synthetic DNA string |
| BB0060 | UGT2B7 | PR0119 | PR0120 | Synthetic DNA string |

TABLE 14
Primers used to construct biobricks.

| Name | SEQ ID NO | Purpose | Sequence |
|---|---|---|---|
| PR0081 | 281 | Fwd primer to amplify | GGATCCATGTCTGACTCTGGTGGTTTCGAC |
| | | BB0041with BamHI | |
| | | site(UGT708G3) | |
| PR0082 | 282 | Rev primer to amplifyBB0041with | AAGCTTCTAGTGAGTGTTGTTGTTACACTTCC |
| | | HindIII site(UGT708G3) | |
| PR0083 | 283 | Fwd primer to amplify | GGATCCATGTCTGACTCTGGTGGTTTCGAC |
| | | BB0042with BamHI | |
| | | site(UGT708G2) | |
| PR0084 | 284 | Rev primer to amplifyBB0042with | AAGCTTCTAGTGAGTGTTGTTGTTACACTTCC |
| | | HindIII site(UGT708G2) | |
| PR0085 | 285 | Fwd primer to amplify | GGATCCATGTCTGACTCTGGTGGTTTCGAC |
| | | BB0043with BamHI | |
| | | site(UGT708G1) | |
| PR0086 | 286 | Rev primer to amplifyBB0043with | AAGCTTCTAGTGAGTGTTGTTGTTACACTTCC |
| | | HindIII site(UGT708G1) | |
| PR0087 | 287 | Fwd primer to amplify | GGATCCATGCCATCTTCTGGTGACGCTGCTGG |
| | | BB0044with BamHI site(OsCGT) | |
| PR0088 | 288 | Rev primer to amplifyBB0044with | AAGCTTCTAGTTAGTTCTACAAGTACCACC |
| | | HindIII site(OsCGT) | |
| PR0089 | 289 | Fwd primer to amplify | GGATCCATGATGGGTGACTTGACTACTTC |
| | | BB0045with BamHI | |
| | | site(FeUGT708C1) | |
| PR0090 | 290 | Rev primer to amplifyBB0045with | AAGCTTCTATCTCTTCAAAGAACCGATG |
| | | HindIII site(FeUGT708C1) | |
| PR0091 | 291 | Fwd primer to amplify | GGATCCATGTCTTCTTCTGAAGGTGTTG |
| | | BB0046with BamHI | |
| | | site(GmUGT708D1) | |
| PR0092 | 292 | Rev primer to amplifyBB0046with | AAGCTTCTAGTTAGCTTGAGCGTTTCTC |
| | | HindIII site(GmUGT708D1) | |
| PR0093 | 293 | Fwd primer to amplify | GGATCCATGGCTGCTAACGGTGGTGACC |
| | | BB0047with BamHI | |
| | | site(ZmUGT708A6) | |
| PR0094 | 294 | Rev primer to amplifyBB0047with | AAGCTTCTACTTTCTTTCAGCGTCTCTAC |
| | | HindIII site(ZmUGT708A6) | |
| PR0095 | 295 | Fwd primer to amplify | GGATCCATGTCTGCTTCTGACGCTTTG |
| | | BB0048with BamHI site(MiCGT) | |
| PR0096 | 296 | Rev primer to amplifyBB0048with | AAGCTTCTAAGTCTTTCTAGAAGTCTTCTTCC |
| | | HindIII site(MiCGT) | |
| PR0097 | 297 | Fwd primer to amplify | GGATCCATGGGTTCTTTGACTAACAACG |
| | | BB0049with BamHI | |
| | | site(GtUF6CGT1) | |
| PR0098 | 298 | Rev primer to amplifyBB0049with | AAGCTTCTACTTAGTACCAGTCTTTCTAGC |
| | | HindIII site(GtUF6CGT1) | |
| PR0099 | 299 | Fwd primer to amplify | GGATCCATGGAATTCAGATTGTTGATCTTGG |
| | | BB0050with BamHI site(DcUGT2) | |
| PR0100 | 300 | Rev primer to amplifyBB0050with | AAGCTTCTAGTTCTTCTTCAACTTTTCAG |
| | | HindIII site(DcUGT2) | |
| PR0101 | 301 | Fwd primer to amplify | GGATCCATGACTTTGTTGAGAGACTTGTTG |
| | | BB0051with BamHI site(DcUGT4) | |
| PR0102 | 302 | Rev primer to amplifyBB0051with | AAGCTTCTACTTAGTCAACATTCTGAAG |
| | | HindIII site(DcUGT4) | |
| PR0103 | 303 | Fwd primer to amplify | GGATCCATGATCTTCTTCTACTTCTTGAC |
| | | BB0052with BamHI site(DcUGT5) | |
| PR0104 | 304 | Rev primer to amplifyBB0052with | AAGCTTCTAGTTGTCCTTAACCTTCTTAG |
| | | HindIII site(DcUGT5) | |
| PR0105 | 305 | Fwd primer to amplify | GGATCCATGAACAGAGAAGTTTCTGAAAG |
| | | BB0053with BamHI site(UGT7365) | |
| PR0106 | 306 | Rev primer to amplifyBB0053with | AAGCTTCTACTTTCTACCGTTCAATTCTTCC |
| | | HindIII site(UGT7365) | |
| PR0107 | 307 | Fwd primer to amplify | GGATCCATGGAAAAGTCTAACGGTTTGAG |
| | | BB0054with BamHI site(UGT76C5) | |
| PR0108 | 308 | Rev primer to amplifyBB0054with | AAGCTTCTAGAAAGAAGAGATGTAGTCG |
| | | HindIII site(UGT76C5) | |
| PR0109 | 309 | Fwd primer to amplify | GGATCCATGTCTTCTGACCCACACAGAAAG |
| | | BB0055with BamHI site(UGT7363) | |
| PR0110 | 310 | Rev primer to amplifyBB0055with | AAGCTTCTAAGAAGTGAATTCTTCGATG |
| | | HindIII site(UGT7363) | |
| PR0111 | 311 | Fwd primer to amplify | GGATCCATGTCTACTTCTGAATTGGTTTTC |
| | | BB0056with BamHI site(UGT71E1) | |
| PR0112 | 312 | Rev primer to amplifyBB0056with | AAGCTTCTAGATAGTAACGTTAGAAACG |
| | | HindIII site(UGT71E1) | |
| PR0113 | 313 | Fwd primer to amplify | GGATCCATGAAGCAAACTGTTGTTTTGTAC |
| | | BB0057with BamHI site(UGT5) | |
| PR0114 | 314 | Rev primer to amplifyBB0057with | AAGCTTCTAGTTTTGAACCAAGTTTTCAAC |
| | | HindIII site(UGT5) | |
| PR0115 | 315 | Fwd primer to amplify | GGATCCATGGCTAGAGCTGGTTGGAC |
| | | BB0058with BamHI site(UGT1A10) | |
| PR0116 | 316 | Rev primer to amplifyBB0058with | AAGCTTCTAGTGAGTCTTAGACTTGTGAGC |
| | | HindIII site(UGT1A10) | |
| PR0117 | 317 | Fwd primer to amplify | GGATCCATGGCTTGTACTGGTTGGACTTC |
| | | BB0059with BamHI site(UGT1A9) | |
| PR0118 | 318 | Rev primer to amplifyBB0059with | AAGCTTCTAGTGAGTCTTAGACTTGTGAGC |
| | | HindIII site(UGT1A9) | |
| PR0119 | 319 | Fwd primer to amplify | GGATCCATGTCTGTTAAGTGGACTTC |
| | | BB0060with BamHI site(UGT2B7) | |
| PR0120 | 320 | Rev primer to amplifyBB0060with | AAGCTTCTAGTCGTTCTTACCCTTCTTAG |
| | | HindIII site(UGT2B7) | |

### Part II.

Alternatively, glycosyl transferase genes for expression in E. coli were codon optimized for E. coli expression and were synthesized and cloned by Twist Bioscience into a custom-made plasmid vector (pRSGLY, synthesized by GeneArt) using standard restriction ligation using SpeI/XhoI restriction sites. This custom-made vector contained a LacI operon, AmpR cassette, replication origin and a multiple cloning site flanked by the T7 promoter and terminator. Additionally, the 5′ end also contained a ribozyme binding site (RBS) and a 6×His tag for subsequent protein purification. Fully assembled plasmids were transformed into E. coli DH5α strains or E. coli XJb (DE3) autolysis strains (Zymo Research). Plasmids used were as shown in Table 15.

TABLE 15
Plasmids constructed for expression of
glycosyl transferases in E. coli

| Plasmid | Backbone | Gene |
|---|---|---|
| PL-5(At73C5_GA) | pRSGLY | At73C5 |
| PL-16(At71D1_GA) | pRSGLY | At71D1 |
| PL-28(At72B1_GA) | pRSGLY | At72B1 |
| PL-31(Sr71E1_GA) | pRSGLY | Sr71E1 |
| PL-32(OsEUGT11_GA) | pRSGLY | OsEUGT11 |
| PL-35(Sp73E_GA) | pRSGLY | Sp73E |
| PL-38(OsO-1_GA) | pRSGLY | OsO-1 |
| PL-42(At84B1_GA) | pRSGLY | At84B1 |
| PL-55(Sr76G1_GA) | pRSGLY | Sr76G1 |
| PL-68(Pa85_GA) | pRSGLY | Pa85 |
| PL-69(CrUGT-2_GA) | pRSGLY | CrUGT-2 |
| PL-74(At73B3_GA) | pRSGLY | At73B3 |
| PL-78(At71C1-Sr71E1_354_GA) | pRSGLY | At71C1-Sr71E1 354 |
| PL-79(Pa72_GA) | pRSGLY | Pa72 |
| PL-85(At73B5_GA) | pRSGLY | At73B5 |
| PL-89(At71C1_At71C2_353_GA) | pRSGLY | At71C1_At71C2 353 |
| PL-100(Cp89B_GA) | pRSGLY | Cp89B |
| PL-112(Sp89B_GA) | pRSGLY | Sp89B |
| PL-113(Tc90A_GA) | pRSGLY | Tc90A |
| PL-152(Si94D_GA) | pRSGLY | Si94D |
| PL-159(Pt88G_GA) | pRSGLY | Pt88G |
| PL-182(Ha88B_2_GA) | pRSGLY | Ha88B_2 |
| PL-189(Ac73T_GA) | pRSGLY | Ac73T |
| PL-202(Si73X_GA) | pRSGLY | Si73X |
| PL-206(Tc74Z_GA) | pRSGLY | Tc74Z |
| PL-214(Cs73Y_GA) | pRSGLY | Cs73Y |
| PL-226(Pt73Y_GA) | pRSGLY | Pt73Y |
| PL-238(Ac73Z_GA) | pRSGLY | Ac73Z |
| PL-254(Bv75C_GA) | pRSGLY | Bv75C |
| PL-258(Pt78G_GA) | pRSGLY | Pt78G |
| PL-259(Si82A_GA) | pRSGLY | Si82A |
| PL-265(Ad74X_GA) | pRSGLY | Ad74X |
| PL-276(Cs74S_GA) | pRSGLY | Cs74S |
| PL-290(Ad72AA_GA) | pRSGLY | Ad72AA |
| PL-300(Si71E_2_GA) | pRSGLY | Si71E_2 |
| PL-325(Vv71R_GA) | pRSGLY | Vv71R |
| PL-326(Ha72B_GA) | pRSGLY | Ha72B |
| PL-330(Sp73A_GA) | pRSGLY | Sp73A |
| PL-332(Bv73P_GA) | pRSGLY | Bv73P |
| PL-338(Pt72B_GA) | pRSGLY | Pt72B |
| PL-340(Qs72S_1_GA) | pRSGLY | Qs72S_1 |
| PL-341(Ad72X_GA) | pRSGLY | Ad72X |
| PL-342(Cp73B_GA) | pRSGLY | Cp73B |
| PL-347(Zj71A_GA) | pRSGLY | Zj71A |
| PL-349(Ha71S_GA) | pRSGLY | Ha71S |
| PL-355(Ac73H_GA) | pRSGLY | Ac73H |
| PL-359(Cp71B_GA) | pRSGLY | Cp71B |
| PL-364(Ha72T_GA) | pRSGLY | Ha72T |
| PL-368(Sp73Q_GA) | pRSGLY | Sp73Q |
| PL-376(Sp72T_GA) | pRSGLY | Sp72T |

### Example 7—Production of Cannabinoids Compounds in Genetically Modified Strains

### Part I.

Cannabinoid glycosides were produced in E. coli or S. cerevisiae strains either by feeding glucose (de novo production), fatty acids (e.g. hexanoic and butanoic acid), other intermediates in the cannabinoid biosynthetic pathway (e.g. olivetolic acid, divarinolic acid, cannabigerolic acid), the final cannabinoid itself (bio-conversion), or combinations thereof. E. coli cells were incubated in Lysogeny broth with appropriate antibiotics with polypeptide expression inducer added for 72 h at 30° C. with constant shaking. S. cerevisiae cells were incubated in synthetic media with required amino acid supplementation to complement auxotrophies for 72 h at 30° C. with constant shaking. Cannabinoids and cannabinoid glycosides were extracted and analyzed as described above. If required, a UDP-sugar substrate was added to the growth media. Alternatively, enzymes which catalyze the conversion of sugars to activated sugars (e.g. conversion of sucrose to UDP-glucose) and/or enzymes which catalyze the interconversion of activated sugars (e.g. conversion of UDP-glucose to UDP-rhamnose) were introduced into the genetically modified strains.

### Part II.

Alternatively, the cells endogenous pool of UDP-sugar (e.g. UDP-glucose natively produced by both S. cerevisiae and E. coli) could be used.

### Example 8—In Vitro Testing of Glycosyl Transferase Performance in Glycosylating Cannabinoid Acceptors

For in vitro studies of glycosyl transferase performance, crude lysates of E. coli strains constructed to express Glycosyl transferases were prepared by placing the strains into sterile 96 deep well plates with 1 mL of NZCYM bacterial culture broth with kanamycin. Samples were incubated overnight at 37° C., shaking at 200 rpm. The following day, 50 μl of each culture was transferred to a new sterile 96 deep well plate with 1 mL of NZCYM bacterial culture broth with kanamycin and polypeptide expression inducers. Samples were incubated at 20° C., shaking at 200 rpm for 20 h. Following this, the plate was centrifuged at 4000 rpm for 10 min at 4° C. After decanting the supernatant, 50 μl of a buffer comprising Tris-HCl, MgCl₂, CaCl₂, and protease inhibitors were added to each well and cells were resuspended by shaking at 200 rpm for 5 min at 4° C. The contents of each well (i.e., cell slurries) were then transferred to a PCR plate and frozen at −80° C. overnight. Frozen cell slurries were thawed at room temperature for up to 30 min. If the thawing mix was not viscous due to cell lysing, samples were frozen and thawed again. When samples were nearly thawed, 25 μl of binding buffer comprising DNase and MgCl₂ are added to each well. The PCR plate was incubated at room temperature for 5 min, shaking at 500 rpm, until samples became less viscous. Finally, samples were centrifuged at 4000 rpm for 5 min, and supernatants were used to convert cannabinoids to their glycosylated derivatives. Conversion was carried out in vitro according to table 16. Alkaline phosphatase was provided by New England Biolabs (M0371S). Cannabinoid acceptors were dissolved in DMSO.

TABLE 16
Reaction setup for measuring glycosyl transferase activity in vitro.

| Component | Volume (μL) |
|---|---|
| H₂0 | 4.2 |
| Alkaline phosphatase (1000 U/mL) | 0.3 |
| 4X Buffer (10 mM Tris-HCl, 5 mM | 7.5 |
| MgCl₂, 1 mM CaCl₂) | |
| UDP-Glucose (1 mM) | 9 |
| Cannabinoid acceptor (10 mM) | 3 |
| Glycosyl transferase containing | 6 |
| supernatant | |

The reaction mixture was incubated overnight at 30° C. The reaction was stopped by adding 30 μl of 100% DMSO. The resultant mixture was diluted further with 90 μl 50% DMSO for LC-MS analysis and ranking of best performing glycosyltransferases.

Alternatively, the protocol of example 13 below was used for this in vitro testing.

### Example 9—Test of Aqueous Solubility of Glycosylated Cannabinoids

### Part I.

Aqueous solubility was determined using a MultiScreen®HTS-PCF Filter Plates for Solubility Assay (Merck) following the manufacturer's instructions. Purified cannabinoid glycosides were dissolved in DMSO to an initial concentration of 20 mM. Quantification of cannabinoid glycoside in solution was determined using LC-MS/QTOF as described above.

### Part II.

Alternatively, a qualitative measurement of aqueous solubility could be performed by measuring the retention time of a compound during LC-MS/QTOF analysis. Since polar compounds would elute at earlier retention times during a run, and since polarity is a direct indicator of aqueous solubility, a comparative assessment could be made. A qualitative measurement of aqueous solubility could also be performed by calculating the partition coefficient (c Log P) of a molecule. c Log P is a measure of how much of a solute dissolves in a water portion vs. an organic portion, molecules with a lower c Log P are better able to dissolve in water than molecules with a higher c Log P. c Log P could be calculated using the molecular structure of a compound and using specialized software. ChemSketch (ACD Labs) was used to calculate the c Log P of cannabinoids and cannabinoid glycosides.

A range of cannabinoid glucosides were analyzed by LC-MS/QTOF as described above and the retention times (RT) measured and compared with their calculated Log P (c Log P) values. As shown in table 17 below cannabinoid glucosides had shorter retention times than cannabinoids indicating they are more water soluble. Furthermore, cannabinoid-di-glucosides had shorter retention times than mono-glucosides, and cannabinoid tri-glucosides had shorter retention times than di-glucosides, overall indicating that addition of sugar groups to cannabinoids results in a successive increase in water solubility. The measured retention times also correlated well with the calculated Log P values.

TABLE 17
Retention time (RT) during QTOF analysis and calculated
LogP of cannabinoids and cannabinoid glycosides

| | Calculated | Measured |
|---|---|---|
| Molecule | LogP | RT |
| CBD | 7.03 | 19.7 |
| CBD-1′-O-β-D-glucoside | 5.04 | 14.3 |
| CBD-1′-O-β-D-glucosyl-3′-O-β-D-glucoside | 3.59 | 9.5 |
| CBD-tri-glucoside | 1.85 | 8.6 |
| CBDV | 5.97 | 17.9 |
| CBDV-1′-O-β-D-glucoside | 3.98 | 12.6 |
| CBDV-1′-O-β-D-glucosyl-3′-O-β-D-glucoside | 2.53 | 8.2 |
| CBDV-1′-O-β-D-glucosyl-3′-O-β-D-di-glucoside | 0.78 | 7.5 |
| CBDA | 7.87 | 19.4 |
| CBDA-1′-O-β-D-glucoside | 5.87 | 10.9 |
| CBG | 7.47 | 19.7 |
| CBG-1′-O-β-D-glucoside | 5.48 | 14.8 |
| CBG-1′-O-β-D-glucosyl-3′-O-β-D-glucoside | 4.03 | 13.8 |
| CBG-1′-O-β-D-glucosyl-3′-O-β-D-di-glucoside | 2.29 | 9.9 |
| THC | 7.68 | 21.8 |
| THC-1′-O-β-D-glucoside | 5.64 | 16.3 |
| CBN | 7.35 | 21 |
| CBN-1′-O-β-D-di-glucoside | 3.86 | 16 |
| 11-nor-9-carboxy-THC | 6.21 | 17.9 |
| 11-nor-9-carboxy-THC-1′-O-β-D-glucoside | 4.17 | 15 |
| 11-nor-9-carboxy-THC-1′-O-β-D-di-glucoside | 4.08 | 14.8 |

### Part III.

Alternatively, aqueous solubility was determined by a thermodynamic solubility assay as follows. 2.5 mg of test compound was weighed in a glass vial, 0.5 mL of phosphate buffered saline (pH=7.4) was added and the sample briefly vortexed. Samples were then incubated overnight at room temperature on a vial roller system to dissolve as much of the compound as possible into solution. Following incubation, the aqueous solutions were filtered in duplicate (0.45 μM pore size) and the filtrate diluted 1:1 with 100% methanol. Samples were further diluted where necessary and analyzed by HPLC. The concentration of compound in solution was determined by comparison to a standard curve made with authentic analytical standards.

The aqueous thermodynamic solubility of CBD and CBD-1′-O-β-D-glucosyl-3′-O-β-D-glucoside (OB6) was measured as described above and quantitative measurements of their solubility determined. As shown in table 18 below, OB6 has a significantly higher aqueous solubility than CBD reaching a solubility of 11.4±0.75 mM at room temperature in PBS (pH=7.4). The solubility of CBD was below the detection limit of the HPLC machine, by diluting an authentic analytical CBD standard it was found that the limit of detection was 0.5 μM indicating that the maximum solubility of CBD was 0.5 μM.

TABLE 18
Thermodynamic solubility of CBD and CBD-1′-O-β-D-glucosyl-
3′-O-β-D-glucoside (OB6) in mM at room temperature in
PBS buffer pH 7.4. BDL: Below detection limit. Data presented
as average and standard deviation of duplicate experiments.

| CBD | Below Detection Limit |
|---|---|
| CBD-1′-O-β-D-glucosyl-3′-O-β-D-glucoside | 11.4 ± 0.75 |

### Example 10—Test of Chemical Stability of Glycosylated Cannabinoids

### Part I.

Chemical stability of cannabinoid glycosides was determined by preparing 10 mM stock solutions in DMSO then diluting to 5 μM in glycine buffer (pH 8-11), PBS (pH 7-8) and acetate buffer (pH 4-6). Solutions were incubated at 37° C. with samples taken at 0, 60, 120, 180, 240 and 300 minute intervals. All samples were analyzed using LC-MS as described above.

### Part II.

Alternatively, chemical stability of cannabinoid glycosides was determined under alkaline, acidic, oxidative and heat stress as follows. 25 mM stock solutions of cannabinoids and cannabinoid glycosides were prepared in 100% methanol. 15 μL is mixed with 5 μL of 400 mM HCl solution (final pH=1.1), 400 mM NaOH solution (Final pH=12.5), 12% H₂O₂ solution (final concentration 3%), or H₂O pH 7.0. Acidic, alkaline and oxidative samples were incubated at 30° C. for 24 h while samples in water were incubated at 80° C. for 24 h. A control under ambient conditions was also prepared where 15 μL of the cannabinoid or cannabinoid glycoside was added to 5 μL H₂O pH 7.0 and incubated at 30° C. After 24 h samples were placed on ice and 60 μL of ice-cold 100% methanol is added to each sample. Samples were centrifuged and transferred to HPLC vials for analysis. The remaining concentration of cannabinoid or cannabinoid glycoside was quantified by comparing to authentic analytical standards. Determining the presence of degradation products were determined by comparing with authentic analytical standards.

CBD, CBD-1′-O-β-D-glucoside (OB1), and CBD-1′-O-β-D-glucosyl-3′-O-β-D-glucoside (OB6) were exposed to oxidative, alkaline, acidic and heat conditions as described above, and their degradation quantified by HPLC analysis by measuring the amount of compound remaining in solution after 24 h exposure to a given condition and expressed as percent (%) remaining after 24 h exposure relative to a control at ambient conditions. Also measured was the accumulation of the known CBD degradation product THC, expressed as percent accumulated after 24 h exposure. As shown in table 19, CBD was unstable under all conditions tested and in particular, degrades to THC under acidic and alkaline conditions. CBD was particularly unstable under alkaline conditions with only 2.26% remaining after 24 h exposure. In contrast, a significantly higher amount of OB1 and OB6 was remaining after 24 h exposure under all conditions tested, particularly under alkaline conditions where 100% remained. While a small amount of THC-1′-O-β-D-glucoside (OB20) was detected for OB1 under acidic conditions, no THC or THC-glucoside was detected for OB6 samples exposed to any of the conditions. Also of relevance, no CBD aglycone was detected for OB1 and OB6 under any condition, thereby indicating the stability of the glucoside bond under extreme conditions.

TABLE 19
Chemical stability of CBD, CBD-1′-O-β-D-glucoside (OB1),
and CBD-1′-O-β-D-glucosyl-3′-O-β-D-glucoside (OB6)
under acidic, alkaline, oxidative and heat stress. Substrates
were incubated in each condition for 24 h then analyzed by HPLC.
Shown is the % of substrate remaining in solution and % accumulation
of the known degradation product THC (and THC-1′-O-β-D-
glucoside (OB20)) relative to a control (substrates incubated
at 30° c. without stress at pH 7.0).

| | Product | | | | |
|---|---|---|---|---|---|
| | CBD | OB1 | OB6 | THC | OB20 |
| CBD | | | | | |
| Acidic (pH 1.1) | 63.90 | NA | NA | 5.88 | NA |
| Alkaline (pH 12.5) | 2.26 | NA | NA | 15.83 | NA |
| Heat (60° c.) | 72.76 | NA | NA | ND | NA |
| Oxidative (H₂O₂ 3%) | 70.56 | NA | NA | ND | NA |
| CBD-1′-O-β-D-glucoside (OB1) | | | | | |
| Acidic (pH 1.1) | ND | 80.02 | NA | ND | 1.61 |
| Alkaline (pH 12.5) | ND | 100.27 | NA | ND | ND |
| Heat (60° c.) | ND | 84.35 | NA | ND | ND |
| Oxidative (H₂O₂ 3%) | ND | 92.98 | NA | ND | ND |
| CBD-1′-O-β-D-glucosyl-3′- | | | | | |
| O-β-D-glucoside (OB6) | | | | | |
| Acidic (pH 1.1) | ND | ND | 91.90 | ND | ND |
| Alkaline (pH 12.5) | ND | ND | 100.62 | ND | ND |
| Heat (60° c.) | ND | ND | 80.79 | ND | ND |
| Oxidative (H₂O₂ 3%) | ND | ND | 74.98 | ND | ND |

Substrate used in each assay is indicated in bold.
Data shown as averages of biological replicates.
ND; Not detected, NA; Not applicable.

### Example 11—Test of Plasma Stability of Glycosylated Cannabinoids

Plasma stability of cannabinoid glycosides are determined by incubating 1 μM in human plasma (Sigma) at 37° C. with samples taken at 0, 60, 120, 180, 240 and 300 minute intervals. All samples are analyzed using LC-MS as described above. Verapamil and Propantheline are used as high stability and low stability references.

### Example 12—Test of Hepatic Microsomal Stability of Glycosylated Cannabinoids

### Part I.

Hepatic microsomal stability of cannabinoid glycosides were determined by incubating 2 μM of molecule with HepaRG™ human liver microsomes (Sigma) supplemented with NADPH at 37° C. Samples were taken at 0, 5, 15, 30, 45, and 60 minute intervals and analyzed as described above. Verapamil (rapid clearance) and Diazepam (low clearance) were used as references.

### Part II.

Alternatively, hepatic microsomal stability of cannabinoid glycosides was determined as follows. HepaRG™ pooled human liver microsomes (Sigma) (final protein concentration=0.5 mg/mL) were mixed with alamethicin (25 μg/mg), 0.1 M phosphate buffer (pH=7.4) and the test compound (1 μM final in DMSO) and incubated at 37° C. prior to addition of NADPH (final concentration 1 mM) and UDP-glucuronic acid (final concentration 1 mM) to initiate the reaction. The compound was incubated for 0, 5, 15, 30, and 45 minutes and the reaction terminated by adding acetonitrile in a 1:3 ratio (v/v). Reactions were centrifuged at 3000 rpm for 20 min at 4° C. to precipitate the protein. Following protein precipitation, internal standards were added to the sample supernatants and analyzed by LC-MS to measure the concentration of compound remaining at each time point, quantification was achieved by comparison to authentic analytical standards.

In vitro hepatic microsomal stability was performed for CBD, CBD-1′-O-β-D-glucoside (OB1), and CBD-1′-O-β-D-glucosyl-3′-O-β-D-glucoside (OB6) as described above and the intrinsic clearance (CLᵢₙₜ) and half-life (t_{1/2}) of each compound was determined. As shown in table 20 below, it was found that while OB1 had a lower hepatic microsomal stability than CBD (indicated by the higher intrinsic clearance and shorter half-life), OB6 had a significantly higher hepatic microsomal stability as shown by the 50 fold increase in half-life and corresponding 50 fold decrease in intrinsic clearance.

TABLE 20
Hepatic microsomal stability of CBD, CBD-1′-O-β-D-glucoside
(OB1), and CBD-1′-O-β-D- glucosyl-3′-O-β-D-glucoside (OB6).
Shown is the intrinsic clearance (CLᵢₙₜ) and half-life (t_{1/2}) of
each compound. Data presented as averages and standard deviations
from 5 biological replicates at different time points (0, 5, 15, 40, 45 mins)

| | | t_{1/2} |
|---|---|---|
| | CLᵢₙₜ (μL/min/mg protein) | (min) |
| CBD | 368 ± 0.684 | 3.77 |
| OB1 | 1110 ± 0.312 | 1.24 |
| OB6 | 7.39 ± 1.32 | 188 |

### Example 13—In Vitro Testing of Glycosyl Transferase Performance in Glycosylating Cannabinoids

For in vitro studies of glycosyl transferase performance in glycosylating cannabinoids, purified Glycosyl transferases were prepared as follows:

5 mL of 2× concentrated LB medium+Ampicillin (50 μg/m L) was inoculated with E. coli XJb (DE3) strains expressing a glycosyl transferase of interest and incubated overnight at 30° C. with shaking. The following day, cell cultures were transferred into 500 mL of 2× concentrated LB medium+Ampicillin (50 μg/mL) and incubated overnight at 30° C. with shaking. The following day, the cell cultures were transferred to 1 L of 2× concentrated LB medium+Ampicillin (50 μg/mL)+3 mM arabinose+0.1 mM IPTG. Cells were incubated for 24 h at 20° C. with shaking. The following day, the cells were collected by centrifugation at 46500×g for 10 mins at 4° C. Cells were resuspended in 20 mL ice-cold GT buffer (50 mM Tris-HCl pH7.4+1 mM phenylmethanesulfonyl fluoride+1 cOmplete™, mini, EDTA-free Protease Inhibitor Cocktail tablet (Roche)). The resuspended material was transferred to a 50 mL falcon tube and kept at −80° C. for at least 15 mins. Falcon tubes were then thawed at room temperature, as the tubes were thawing the following reagents were added; 2.6 mM MgCl₂, 1 mM CaCl₂, 250 μL of a 1.4 mg/ml DNase solution (Sigma) dissolved in MilliQ water. Tubes were gently inverted to mix then were incubated for 5 mins at 37° C. Binding buffer was then added to the tubes (50 mM Tris-HCl pH7.4, 10 mM imidazole, 500 mM NaCl. 11.25 mL MilliQ water) and the pH adjusted to 7.4 with HCl. The mix was centrifuged at 15550×g for 15 mins at 4° C., the supernatant transferred to a fresh 50 mL falcon tubes and centrifuged again to remove any remaining cellular debris at 48400×g for 20 minutes at 4° C. While the enzyme prep was centrifuging, 3 mL of HIS-Select (available from Sigma P6611) column material was added to a fresh 50 mL tube and washed by adding MilliQ water up to 50 mL, centrifuging at 2000×g for 2 mins and discarding the supernatant. This washing step was repeated. Finally, MilliQ water was added to the HIS-Select material to an approximate 50% volume. Collected supernatant from the centrifuged enzyme preparation was transferred to the tube containing the HIS-Select material through a Miracloth (available from Merck Millipore), and then incubated at 4° C. with gently shaking by inversion for 2 h. After 2 h the mix was centrifuged at 2000×g for 4 minutes at 4° C. and the supernatant discarded. The remaining HIS-Select material was washed twice with 1× binding buffer (50 mM Tris-HCl, 0.5M NaCl, 10 mM Imidazole, pH 7.4) with centrifugation at 2000×g for 4 minutes at 4° C. The HIS-Select material was resuspended in 5 mL 1× binding buffer and transferred to a Poly-Prep®Chromatography Column (available from BioRad, 7311550). The HIS-Select material was kept at 4° C. and washed twice with 1× binding buffer by filling up the column and allowing it to drip through. Finally, purified Glycosyl transferases were eluted from the HIS-Select material by adding 7.5 mL of elution buffer (50 mM Tris-HCl, 500 mM Imidazole, pH7.4) and collecting the flow through. Enzymes were used immediately in in vitro enzyme assays or stored at −20° C. in 50% glycerol until needed.

In vitro conversion of various cannabinoids to cannabinoid glycosides was carried out according to table 21. Alkaline phosphatase was provided by New England Biolabs (M0371S). Cannabinoids were dissolved in methanol. The UDP-sugar (e.g. UDP-glucose) was provided by a commercial supplier (e.g. Sigma) or produced by in vitro enzymatic conversion from a commercially available UDP-sugar as shown in Example 21.

TABLE 21
Reaction setup to measure glycosyl transferase
activity with various cannabinoids in vitro.

| | Volume |
|---|---|
| Reagent | (μL) |
| Purified glycosyl transferase enzyme | 5 |
| 25 mM Cannabinoid substrate | 0.4 |
| 1M Tris-HCl pH 7.4 | 2 |
| Milli-Q water | 11.9 |
| FastAP phosphatase (1 U/μL) | 0.2 |
| 50 mM UDP-sugar | 0.5 |
| TOTAL | 20 |

The reaction mixture was scaled up or down as required. The reaction mixture was incubated without shaking at 30° C. for 24 hours. Extraction and analysis were performed as described above for this example. To confirm the identity of the produced cannabinoid glycosides LC-MS/QTOF was used as described above to confirm the expected mass and fragmentation pattern of each detected molecule. Quantification of cannabinoid glycoside production was done by comparing the peak area of the cannabinoid substrate and the cannabinoid glycoside with authentic analytical standards (where available), where a substrate was unavailable, quantification was achieved by comparing with an authentic analytical standard of the cannabinoid aglycone. % conversion of substrates to cannabinoid glycosides by specific Glycosyl transferases was calculated by measuring the decrease in substrate and increase in product after 24 h incubation. In total, cannabinoid glycosylation was tested with the cannabinoids CBD, CBDV, CBDA, THC, CBN, CBG and 11-nor-9-carboxy-THC using UDP-glucose, UDP-rhamnose, UDP-xylose, UDP-galactose, UDP-glucuronic acid and UDP-N-acetylglucosamine.

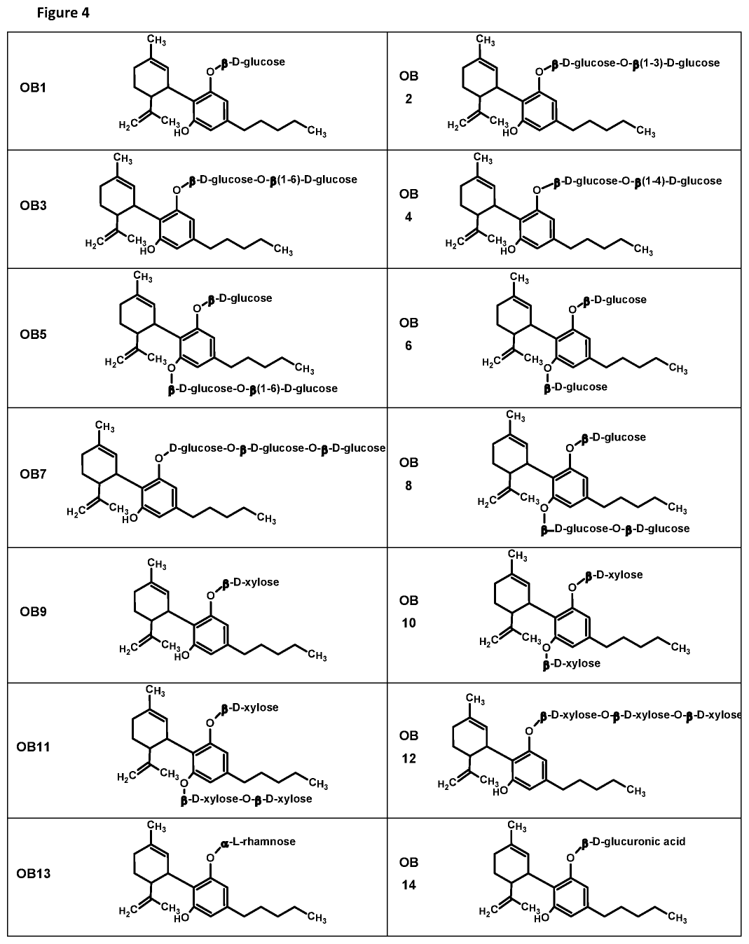

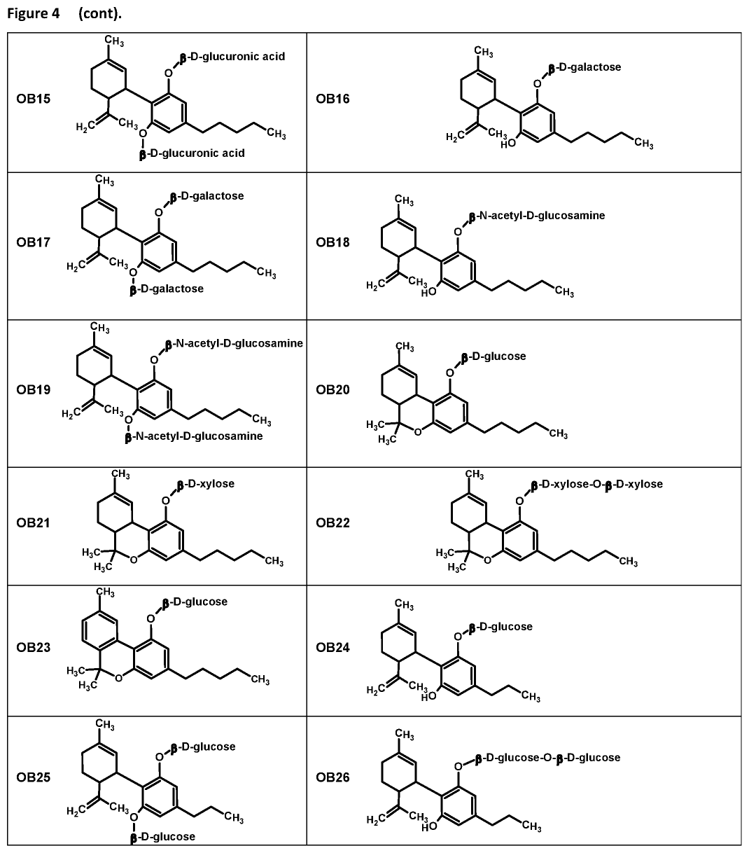

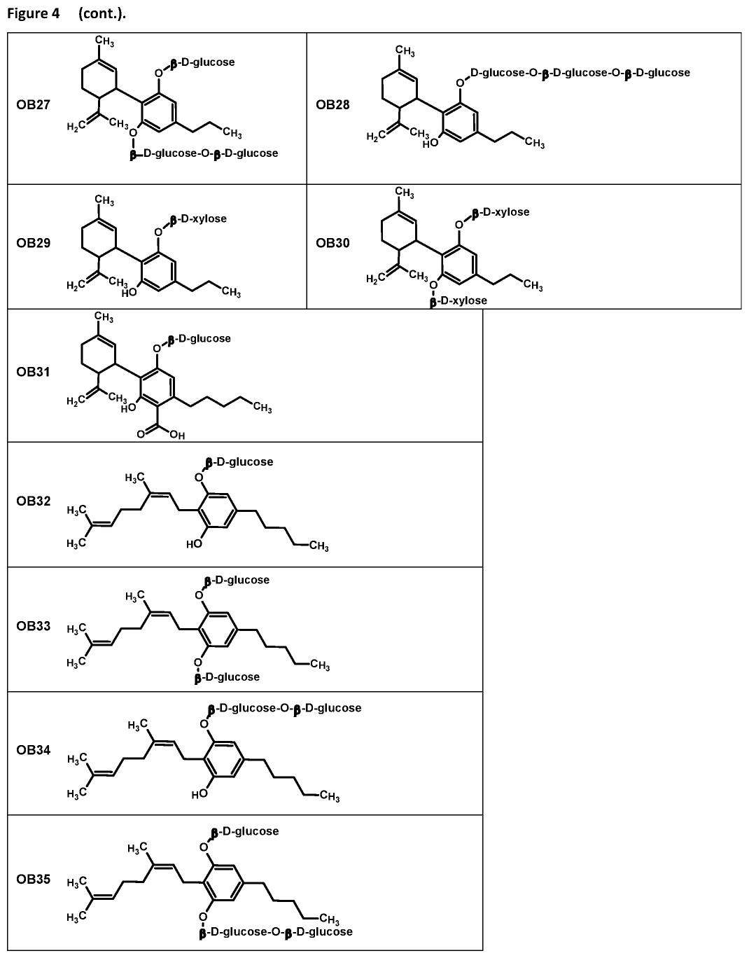

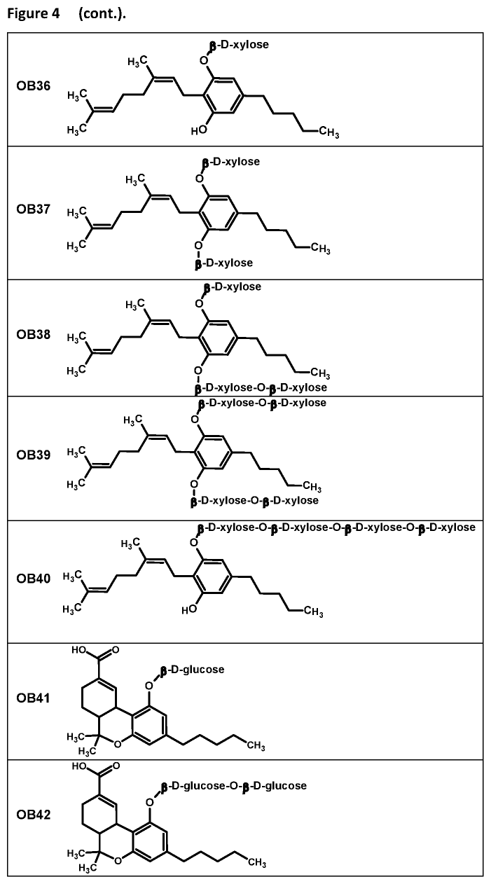

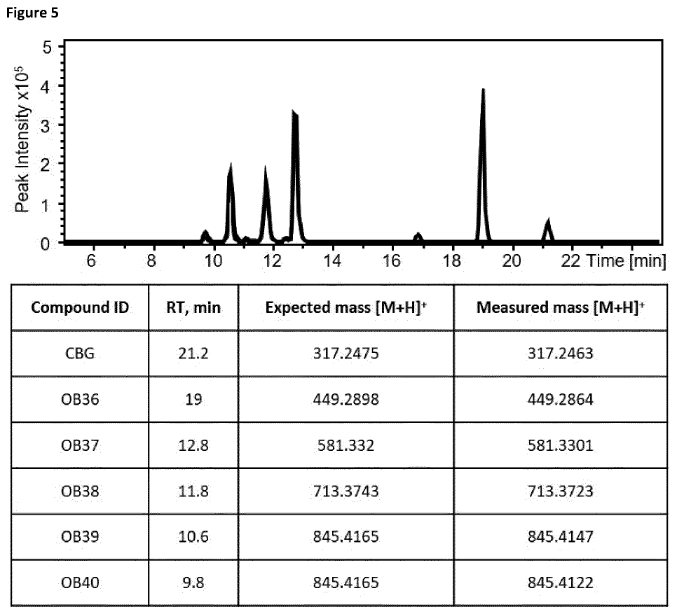

A corresponding structure ID was given for each cannabinoid glycoside produced in this screen, structures of each molecule is shown in FIG. 4. An example of the resulting LC-MS/QTOF chromatogram produced is given in FIG. 5.

### Cannabinoid Glycosides Produced Using CBD as Cannabinoid Acceptor.

A range of glycosyl transferases were found to catalyze the conversion of CBD to a range of different CBD-glycosides. Table 22 shows all the CBD-glycosides produced and exemplary glycosyl transferases which catalyzed each reaction with corresponding conversion %.

TABLE 22
CBD-glycosides produced by glycosyl transferases in vitro

| Structure | | | | Conversion |
|---|---|---|---|---|
| ID | Common name | Sugar donor | Enzyme(s) | % |
| OB1 | CBD-1′-O-β-D-glucoside | UDP-Glucose | PL-159(Pt88G_GA) | 75 |
| OB2 | CBD-1′-O-β-D-laminaribioside | UDP-Glucose | PL-159(Pt88G_GA) + | 80.7 |
| | | | PL-55(Sr76G1_GA) | |
| OB3 | CBD-1′-O-β-D-gentiobioside | UDP-Glucose | PL-159(Pt88G_GA) + | 96.4 |
| | | | PL-152(Si94D_GA) | |
| OB4 | CBD-1′-O-β-D-cellobioside | UDP-Glucose | PL-159(Pt88G_GA) + | 3.1 |
| | | | PL-32(OsEUGT11_GA) | |
| OB5 | CBD-1′-O-β-D-glycosyl-3′-O-β- | UDP-Glucose | PL-159(Pt88G_GA) + | 1.5 |
| | D-gentiobioside | | PL-152(Si94D_GA) | |
| OB6 | CBD-1′-O-β-D-glucosyl-3′-O- | UDP-Glucose | PL-214(Cs73Y_GA) | 57.5 |
| | β-D-glucoside | | | |
| OB7 | CBD-1′-O-β-D-tri-glucoside | UDP-Glucose | PL-214(Cs73Y_GA) | 27.3 |
| OB8 | CBD-1′-O-β-D-glucosyl-3′-O- | UDP-Glucose | PL-214(Cs73Y_GA) | 12.3 |
| | β-D-di-glucoside | | | |
| OB9 | CBD-1′-O-β-D-xyloside | UDP-Xylose | PL-159(Pt88G_GA) | 12.4 |
| OB10 | CBD-1′-O-β-D-xylosyl-3′-O-β- | UDP-Xylose | PL-214(Cs73Y_GA) | 97.4 |
| | D-xyloside | | | |
| OB11 | CBD-1′-O-β-D-xylosyl-3′-O-β- | UDP-Xylose | PL-214(Cs73Y_GA) | 1.6 |
| | D-di-xyloside | | | |
| OB12 | CBD-1′-O-β-D-tri-xyloside | UDP-Xylose | PL-214(Cs73Y_GA) | 1.0 |
| OB13 | CBD-1′-O-α-L-rhamnoside | UDP- | PL-342(Cp73B_GA) | 5.7 |
| | | Rhamnose | | |
| OB14 | CBD-1′-O-β-D-glucuronide | UDP- | PL-214(Cs73Y_GA) | 2.0 |
| | | Glucuronic | | |
| | | Acid | | |
| OB15 | CBD-1′-O-β-D-glucurosyl-3′- | UDP- | PL-214(Cs73Y_GA) | 31.2 |
| | O-β-D-glucuronide | Glucuronic | | |
| | | Acid | | |
| OB16 | CBD-1′-O-β-D-galactoside | UDP- | PL-214(Cs73Y_GA) | 62 |
| | | Galactose | | |
| OB17 | CBD-1′-O-β-D-galactosyl-3′-O- | UDP- | PL-214(Cs73Y_GA) | 33.6 |
| | β-D-galactoside | Galactose | | |
| OB18 | CBD-1′-O-β-D-N- | UDP-N- | PL-214(Cs73Y_GA) | 77.4 |
| | acetylglucosaminoside | acetyl- | | |
| | | glucosamine | | |
| OB19 | CBD-1′-O-β-D-N- | UDP-N- | PL-214(Cs73Y_GA) | 14.3 |
| | acetylglucosamine-3′-O-β-D- | acetyl- | | |
| | N-acetylglucosaminoside | glucosamine | | |

Table 23 further shows the retention time (RT) calculated Log P (c log P), expected and measured mass of each compound and fragmentation pattern as determined by LC-MS/QTOF analysis thereby confirming the structure of each CBD-glycoside.

TABLE 23
Retention time, cLogP, expected and measured mass, and fragmentation pattern
of each CBD-glycoside produced by glycosyl transferases in vitro.

| | | Expected | Measured | | |
|---|---|---|---|---|---|
| Structure | | mass | mass | | |
| ID | RT | [M + H]⁺ | [M + H]⁺ | Fragmentation pattern | clogP |
| OB1 | 14.3 | 477.2847 | 477.2828 | MS2(639.3371): loss of glucose −> m/z | 5.04 +/− 0.39 |
| | | | | 315.2316 | |
| OB2 | 13.5 | 639.3375 | 639.3371 | MS2(639.3371): loss of 2x glucose −> m/z | 3.67 +/− 0.54 |
| | | | | 315.2320 | |
| OB3 | 12.5 | 639.3375 | 639.3368 | MS2(639.3368): loss of 2x glucose −> m/z | 4.00 +/− 0.56 |
| | | | | 315.2317 | |
| OB4 | 12.1 | 639.3375 | 639.3345 | MS2(639.3345): loss of 2x glucose −> m/z | 3.89 +/− 0.55 |
| | | | | 315.2324 | |
| OB5 | 11.4 | 801.3903 | 801.3884 | MS2(801.3884): loss of 2x glucose −> m/z | 3.62 +/− 0.73 |
| | | | | 639.3368 −> loss of glucose −> | |
| | | | | m/z315.2310 | |
| OB6 | 9.1 | 639.3375 | 639.3372 | MS2(639.3372): loss of glucose −> m/z | 3.59 +/− 0.42 |
| | | | | 477.2850 −> loss of glucose −> m/z | |
| | | | | 315.2324 | |
| OB7 | 8.4 | 801.3903 | 801.3909 | MS2(801.3912): loss of 3x glucose −> m/z | 3.87 +/− 0.72 |
| | | | | 315.2323 | |
| OB8 | 8 | 801.3903 | 801.3892 | MS2(801.3899): loss of glucose −> m/z | 1.85 +/− 0.63 |
| | | | | 639.3376 −> loss of 2xglucose −> m/z | |
| | | | | 315.2324 | |
| OB9 | 15.5 | 447.2733 | 447.2741 | MS2(447.2741): loss of xylose −> m/z | 6.44 +/− 0.51 |
| | | | | 315.2317 | |
| OB10 | 11.4 | 579.3164 | 579.3168 | MS2(579.3168): loss of 2x xylose −> m/z | 5.07 +/− 0.65 |
| | | | | 315.2324 | |
| OB11 | 10.4 | 711.3586 | 711.3561 | MS2(711.3558): loss of 2x xylose −> m/z | 5.15 +/− 0.78 |
| | | | | 447.2728 −> loss of xylose −> 315.2305 | |
| OB12 | 9.9 | 711.3586 | 711.3561 | MS2(711.3557): loss of xylose −> m/z | 4.78 +/− 0.92 |
| | | | | 579.3129 −> loss of xylose −> | |
| | | | | m/z 447.2728 −> loss of xylose −> 315.2292 | |
| OB13 | 16.1 | 461.2883 | 461.2898 | MS2(461.2882): loss of rhamnose −> m/z | 6.93 +/− 0.51 |
| | | | | 315.2316 | |
| OB14 | 14.4 | 491.2639 | 491.2635 | MS2(491.2632): loss of GlcA −> m/z | 4.88 +/− 0.51 |
| | | | | 315.2316 | |
| OB15 | 9.7 | 667.296 | 667.2939 | MS2(667.2938): loss of GlcA −> m/z | 2.39 +/− 0.66 |
| | | | | 315.2305 | |
| OB16 | 14.2 | 477.2847 | 477.2851 | MS2(477.2858): loss of galactose −> m/z | 5.04 +/− 0.39 |
| | | | | 315.2312 | |
| OB17 | 9.1 | 639.3375 | 639.3378 | MS2(639.3378): loss of galactose −> m/z | 3.67 +/− 0.54 |
| | | | | 315.2325 | |
| OB18 | 13.8 | 518.3112 | 518.3114 | MS2(518.3114): loss of GlcNAc −> m/z | 5.75 +/− 0.59 |
| | | | | 315.2325 | |
| OB19 | 8.3 | 721.3906 | 721.3907 | MS2(721.3907): loss of GlcNAc −> m/z | 3.83 +/− 0.78 |
| | | | | 518.3108 −> loss of GlcNAc −> m/z | |
| | | | | 315.2315 | |

For several CBD-glycosides, it was found that multiple glycosyl transferases could catalyze the reaction in varying conversion efficiencies. Tables 24-30 shows glycosyl transferases which produced the CBD-glycoside indicated along with the % conversion efficiency.

TABLE 24
Glycosyl transferases catalyzing the conversion of
CBD to OB1 (CBD→CBD-1′-O-β-D-glucoside) with
calculated conversion efficiency.

| Plasmid | % conversion |
|---|---|
| PL-159(Pt88G_GA) | 97.4 |
| PL-347(Zj71A_GA) | 55.3 |
| PL-182(Ha88B_2_GA) | 50.0 |
| PL-5(At73C5_GA) | 48.2 |
| PL-189(Ac73T_GA) | 21.1 |
| PL-226(Pt73Y_GA) | 5.0 |
| PL-55(Sr76G1_GA) | ND |

ND: Not detected.

TABLE 25
Glycosyl transferases catalyzing the conversion of
CBD to OB13 (CBD→CBD-1′-O-α-L- rhamnoside)
with calculated conversion efficiency.

| Plasmid | % conversion |
|---|---|
| PL-342(Cp73B_GA) | 5.7 |
| PL-226(Pt73Y_GA) | 5.1 |
| PL-214(Cs73Y_GA) | 4.4 |
| PL-238(Ac73Z_GA) | 3.5 |
| PL-189(Ac73T_GA) | 2.8 |
| PL-5(At73C5_GA) | 2.4 |
| PL-159(Pt88G_GA) | 2.0 |
| PL-55(Sr76G1_GA) | ND |

ND: Not detected.

TABLE 26
Glycosyl transferases catalyzing the conversion
of CBD to OB9 (CBD→CBD-1′-O-β-D-xyloside)
with calculated conversion efficiency.

| Plasmid | % conversion |
|---|---|
| PL-342(Cp73B_GA) | 25.1 |
| PL-189(Ac73T_GA) | 17.3 |
| PL-238(Ac73Z_GA) | 17.3 |
| PL-5(At73C5_GA) | 14.1 |
| PL-159(Pt88G_GA) | 12.4 |
| PL-182(Ha88B_2_GA) | 9.6 |
| PL-332(Bv73P_GA) | 7.6 |
| PL-214(Cs73Y_GA) | 6.9 |
| PL-69(CrUGT-2_GA) | 3.8 |
| PL-31(Sr71E1_GA) | 3.6 |
| PL-355(Ac73H_GA) | 3.2 |
| PL-68(Pa85_GA) | 2.3 |
| PL-55(Sr76G1_GA) | ND |

ND: Not detected.

TABLE 27
Glycosyl transferases catalyzing the conversion of
CBD to OB6 (CBD→ CBD-1′-O-β-D-glucosyl-
3′-O-β-D-glucoside) with calculated conversion efficiency.

| Plasmid | % conversion |
|---|---|
| PL-214(Cs73Y_GA) | 95.8 |
| PL-342(Cp73B_GA) | 92.3 |
| PL-226(Pt73Y_GA) | 82.0 |
| PL-5(At73C5_GA) | 46.4 |
| PL-55(Sr76G1_GA) | ND |

ND: Not detected.

TABLE 28
Glycosyl transferases catalyzing the conversion of
CBD to OB10 (CBD→ CBD-1′-O-β-D-xylosyl-
3′-O-β-D-xyloside) with calculated conversion efficiency.

| Plasmid | % conversion |
|---|---|
| PL-214(Cs73Y_GA) | 98.5 |
| PL-226(Pt73Y_GA) | 88.1 |
| PL-342(Cp73B_GA) | 30.2 |
| PL-238(Ac73Z_GA) | 19.8 |
| PL-5(At73C5_GA) | 16.6 |
| PL-189(Ac73T_GA) | 11.4 |
| PL-69(CrUGT-2_GA) | 5.6 |
| PL-325(Vv71R_GA) | 2.4 |
| PL-55(Sr76G1_GA) | ND |

ND: Not detected.

TABLE 29
Glycosyl transferases catalyzing the conversion of CBD
to OB7 (CBD→ CBD-1′-O-β-D-tri-glucoside) with
calculated conversion efficiency.

| Plasmid | % conversion |
|---|---|
| PL-214(Cs73Y_GA) | 27.3 |
| PL-226(Pt73Y_GA) | 10.2 |
| PL-342(Cp73B_GA) | 5.2 |
| PL-55(Sr76G1_GA) | ND |

ND: Not detected.

TABLE 30
Glycosyl transferases catalyzing the conversion of
CBD to OB8 (CBD→ CBD-1′-O-β-D-glucosyl-
3′-O-β-D-di-glucoside) with calculated conversion efficiency.

| Plasmid | % conversion |
|---|---|
| PL-214(Cs73Y_GA) | 12.3 |
| PL-226(Pt73Y_GA) | 4.2 |
| PL-55(Sr76G1_GA) | ND |

ND: Not detected.

### Cannabinoid Glycosides Produced Using CBDV as Cannabinoid Acceptor.

A range of glycosyl transferases were found to catalyze the conversion of CBDV to a range of different CBDV-glycosides. Table 31 shows all the CBDV-glycosides produced and exemplary glycosyl transferases which catalyzed each reaction with corresponding conversion %.

TABLE 31
CBDV-glycosides produced by glycosyl transferases in vitro

| Structure | | Sugar | | Conversion |
|---|---|---|---|---|
| ID | Common name | donor | Enzyme(s) | % |
| OB24 | CBDV-1′-O-β-D-glucoside | UDP- | PL- | 92.6 |
| | | Glucose | 326(Ha72B_GA) | |
| OB25 | CBDV-1′-O-β-D-glucosyl-3′-O-β-D- | UDP- | PL- | 4.5 |
| | glucoside | Glucose | 342(Cp73B_GA) | |
| OB26 | CBDV-1′-O-β-D-di-glucoside | UDP- | PL- | 22.5 |
| | | Glucose | 342(Cp73B_GA) | |
| OB27 | CBDV-1′-O-β-D-glucosyl-3′-O-β-D- | UDP- | PL- | 6.4 |
| | di-glucoside | Glucose | 226(Pt73Y_GA) | |
| OB28 | CBDV-1′-O-β-D-tri-glucoside | UDP- | PL- | 6.5 |
| | | Glucose | 226(Pt73Y_GA) | |
| OB29 | CBDV-1′-O-β-D-xyloside | UDP- | PL- | 12.1 |
| | | Xylose | 214(Cs73Y_GA) | |
| OB30 | CBDV-1′-O-β-D-xylosyl-3′-O-β-D- | UDP- | PL- | 87.9 |
| | xyloside | Xylose | 214(Cs73Y_GA) | |

Table 32 further shows the retention time (RT) calculated Log P (c log P), expected and measured mass of each compound and fragmentation pattern as determined by LC-MS/QTOF analysis thereby confirming the structure of each CBDV-glycoside.

TABLE 32
Retention time, cLogP, expected and measured mass, and fragmentation pattern
of each CBDV-glycoside produced by glycosyl transferases in vitro.

| | | Expected | Measured | | |
|---|---|---|---|---|---|
| Structure | | mass | mass | | |
| ID | RT | [M + H]⁺ | [M + H]⁺ | Fragmentation pattern | clogP |
| OB24 | 12.6 | 449.2534 | 449.2534 | MS2(449.2542): loss of glucose −> | 3.98 +/− 0.39 |
| | | | | m/z 287.2010 | |
| OB25 | 11.8 | 611.3062 | 611.3063 | MS2(611.3065): loss of 2x glucose −> | 2.53 +/− 0.42 |
| | | | | m/z 287.2009 | |
| OB26 | 8.2 | 611.3067 | 611.3062 | MS2(611.3068): loss of 2x glucose −> | 2.82 +/− 0.55 |
| | | | | m/z 287.2011 | |
| OB27 | 6.6 | 773.3590 | 773.3579 | MS2(773.3583): loss of 2x glucose −> | 0.78 +/− 0.63 |
| | | | | m/z 449.2522 −> loss of glucose −> | |
| | | | | 287.1996 | |
| OB28 | 7.1 | 773.3590 | 773.3577 | MS2(773.3567): loss of 3x glucose −> | 2.81 +/− 0.72 |
| | | | | m/z 287.2009 | |
| OB29 | 14.1 | 419.2428 | 419.2415 | MS2(419.2424): loss of xylose −> m/z | 5.37 +/− 0.51 |
| | | | | 287.2005 | |
| OB30 | 9.6 | 551.2851 | 551.2852 | MS2(551.2834): loss of xylose −> m/z | 4.01 +/− 0.65 |
| | | | | 419.2406 −> loss of xylose −> m/z | |
| | | | | 287.2000 | |

For several CBDV-glycosides, it was found that multiple glycosyl transferases could catalyze the reaction in varying conversion efficiencies. Tables 33-34 provide a list of glycosyl transferases which were shown to produce the CBDV-glycoside indicated along with the % conversion efficiency.

TABLE 33
Glycosyl transferases catalyzing the conversion of
CBDV to OB24 (CBDV→CBDV-1′-O-β-D-glucoside)
with calculated conversion efficiency. ND: Not detected.

| | % |
|---|---|
| Plasmid | conversion |
| PL-326(Ha72B_GA) | 92.6 |
| PL-159(Pt88G_GA) | 89.2 |
| PL-182(Ha88B_2_GA) | 89.0 |
| PL-364(Ha72T_GA) | 78.4 |
| PL-5(At73C5_GA) | 64.6 |
| PL-342(Cp73B_GA) | 59.6 |
| PL-68(Pa85_GA) | 56.3 |
| PL-332(Bv73P_GA) | 39.5 |
| PL-238(Ac73Z_GA) | 39.1 |
| PL-69(CrUGT-2_GA) | 15.9 |
| PL-189(Ac73T_GA) | 13.6 |
| PL-325(Vv71R_GA) | 10.5 |
| PL-28(At72B1_GA) | 9.9 |
| PL-355(Ac73H_GA) | 5.4 |
| PL-89(At71C1_At71C2_353_GA) | 4.0 |
| PL-376(Sp72T_GA) | 2.5 |
| PL-55(Sr76G1_GA) | ND |

TABLE 34
Glycosyl transferases catalyzing the conversion of
CBDV to OB25 (CBDV→ CBDV-1′-O-β-O-glucosyl-
3′-O-β-D-glucoside) with calculated conversion efficiency.

| | % |
|---|---|
| Plasmid | conversion |
| PL-214(Cs73Y_GA) | 91.0 |
| PL-226(Pt73Y_GA) | 79.1 |
| PL-69(CrUGT-2_GA) | 74.4 |
| PL-238(Ac73Z_GA) | 38.1 |
| PL-342(Cp73B_GA) | 22.5 |
| PL-68(Pa85_GA) | 11.5 |
| PL-5(At73C5_GA) | 9.1 |
| PL-325(Vv71R_GA) | 7.8 |
| PL-55(Sr76G1_GA) | ND |

ND: Not detected.

### Cannabinoid Glycosides Produced Using CBDA as Substrate.

A range of glycosyl transferases were found to catalyze the conversion of CBDA to 01331. Table 35 shows the CBDA-glycoside produced and an exemplary glycosyl transferase which catalyzed each reaction with corresponding conversion %.

TABLE 35
CBDA-glycosides produced by glycosyl transferases in vitro

| Structure | | Sugar | | Conversion |
|---|---|---|---|---|
| ID | Common name | donor | Enzyme(s) | % |
| OB31 | CBDA-1′-O-β-D- | UDP- | PL- | 92 |
| | glucoside | Glucose | 214(Cs73Y_GA) | |

Table 36 further shows the retention time (RT), calculated Log P (c log P), expected and measured mass of the compound and fragmentation pattern as determined by LC-MS/QTOF analysis thereby confirming the structure of the CBDA-glycoside.

TABLE 36
Retention time, cLogP, expected and measured mass, and fragmentation pattern
of the CBDA-glycoside produced by glycosyl transferases in vitro

| | | Expected | Measured | | |
|---|---|---|---|---|---|
| Structure | | mass | mass | | |
| ID | RT | [M + H]⁺ | [M + H]⁺ | Fragmentation pattern | clogP |
| OB31 | 14.2 | 521.2745 | 521.2743 | MS2(521.2744): loss of glucose −> m/z | 5.87 +/− 0.41 |
| | | | | 359.2220 −> loss of water −> m/z | |
| | | | | 341.2112 | |

It was found that multiple glycosyl transferases could catalyze this reaction in varying conversion efficiencies. Tables 37 provides a list of glycosyl transferases which were shown to produce the CBDA-glycoside indicated along with the % conversion efficiency.

TABLE 37
Glycosyl transferases catalyzing the conversion of CBDA
to OB31 (CBDA→ CBDA-1′-O-β-D-glucoside) with
calculated conversion efficiency.

| | % |
|---|---|
| Plasmid | conversion |
| PL-214(Cs73Y_GA) | 98.6 |
| PL-238(Ac73Z_GA) | 86.0 |
| PL-226(Pt73Y GA) | 82.0 |
| PL-112(Sp89B_GA) | 78.8 |
| PL-342(Cp73B_GA) | 76.4 |
| PL-100(Cp89B_GA) | 71.1 |
| PL-69(CrUGT-2_GA) | 64.0 |
| PL-189(Ac73T_GA) | 56.6 |
| PL-332(Bv73P_GA) | 54.9 |
| PL-85(At73B5_GA) | 33.2 |
| PL-74(At73B3_GA) | 17.8 |
| PL-35(Sp73E_GA) | 17.1 |
| PL-202(Si73X_GA) | 15.7 |
| PL-182(Ha88B_2_GA) | 15.5 |
| PL-159(Pt88G_GA) | 12.0 |
| PL-16(At71D1_GA) | 11.4 |
| PL-68(Pa85_GA) | 11.1 |
| PL-55(Sr76Gl GA) | ND |

### Cannabinoid Glycosides Produced Using CBG as Substrate.

A range of glycosyl transferases were found to catalyze the conversion of CBG to a range of different CBG-glycosides. Table 38 shows all the CBG-glycosides produced and exemplary glycosyl transferases which catalyzed each reaction with corresponding conversion %.

Table 38. CBG-glycosides produced by glycosyl transferases in vitro.

| Structure | | Sugar | | Conversion |
|---|---|---|---|---|
| ID | Common name | donor | Enzyme(s) | % |
| OB32 | CBG-1′-O-β-D-glucoside | UDP- | PL-340(Qs72S_1_GA) | 98.9 |
| | | Glucose | | |
| OB33 | CBG-1′-O-β-D-glucosyl-3′-O-β-D- | UDP- | PL-5(At73C5_GA) | 4.5 |
| | glucoside | Glucose | | |
| OB34 | CBG-1′-O-β-D-di-glucoside | UDP- | PL-5(At73C5_GA) | 0.6 |
| | | Glucose | | |
| OB35 | CBG-1′-O-β-D-glucosyl-3′-O-β-D- | UDP- | PL-5(At73C5_GA) | 42.3 |
| | di-glucoside | Glucose | | |
| OB36 | CBG-1′-O-β-D-xyloside | UDP-Xylose | PL-214(Cs73Y_GA) | 1.0 |
| OB37 | CBG-1′-O-β-D-xylosyl-3′-O-β-D- | UDP-Xylose | PL-214(Cs73Y_GA) | 44.9 |
| | xyloside | | | |
| OB38 | CBG-1′-O-β-D-xylosyl-3′-O-β-D- | UDP-Xylose | PL-214(Cs73Y_GA) | 21.6 |
| | di-xyloside | | | |
| OB39 | CBG-1′-O-β-D-di-xylosyl-3′-O-β- | UDP-Xylose | PL-214(Cs73Y_GA) | 24.9 |
| | D-di-xyloside | | | |
| OB40 | CBG-1′-O-β-D-tetra-xyloside | UDP-Xylose | PL-214(Cs73Y_GA) | 1.2 |

ND: Not detected.

Table 39 further shows the retention time (RT), calculated Log P (c log P), expected and measured mass of each compound and fragmentation pattern as determined by LC-MS/QTOF analysis thereby confirming the structure of each CBG-glycoside.

TABLE 39
Retention time, cLogP, expected and measured mass, and fragmentation pattern
of each CBG-glycoside produced by glycosyl transferases in vitro.

| | | Expected | Measured | | |
|---|---|---|---|---|---|
| Structure | | mass | mass | | |
| ID | RT | [M + H]⁺ | [M + H]⁺ | Fragmentation pattern | clogP |
| OB32 | 14.9 | 479.3003 | 479.3011 | MS2(479.3013): loss of glucose −> | 5.48 +/− 0.33 |
| | | | | m/z 317.2483 | |
| OB33 | 14.3 | 641.3532 | 641.3514 | MS2(641.3510): loss of 2x glucose | 4.03 +/− 0.39 |
| | | | | −> m/z 317.2470 | |
| OB34 | 13.3 | 641.3532 | 641.3498 | MS2(641.3459): loss of 2x glucose | 4.33 +/− 0.54 |
| | | | | −> m/z 317.2458 | |
| OB35 | 10.7 | 803.406 | 803.4074 | MS2(803.4075): loss of 2x glucose | 2.29 +/− 0.61 |
| | | | | −> m/z 479.3003 −> loss of glucose | |
| | | | | −> m/z 317.2478 | |
| OB36 | 19 | 449.2898 | 449.2864 | MS2(449.2864): loss of xylose −> | 6.88 +/− 0.49 |
| | | | | m/z 315.1796 | |
| OB37 | 12.8 | 581.332 | 581.3301 | MS2(581.3300): loss of 2x xylose - | 5.51 +/− 0.64 |
| | | | | > m/z 317.2474 | |
| OB38 | 11.8 | 713.3743 | 713.3723 | MS2(713.3742): loss of xylose −> | 5.59 +/− 0.77 |
| | | | | m/z 581.3300 −> loss of xylobiose | |
| | | | | −> m/z 317.2466 | |
| OB39 | 10.6 | 845.4165 | 845.4147 | MS2(845.4136): loss of xylcose −> | 5.58 +/− 0.86 |
| | | | | m/z 713.3722 −> loss of xylose −> | |
| | | | | m/z 581.3298 −> loss of 2x | |
| | | | | xylobiose −> m/z 317.2462 | |
| OB40 | 9.8 | 845.4165 | 845.4122 | MS2(845.4119): loss of xylcose −> | 5.38 +/− 0.95 |
| | | | | m/z 713.3720 −> loss of xylose −> | |
| | | | | m/z 581.3293 −> loss of 2x | |
| | | | | xylobiose −> m/z 317.2458 | |

For several CBG-glycosides, it was found that multiple glycosyl transferases could catalyze the reaction in varying conversion efficiencies. Tables 40-41 provide a list of glycosyl transferases which were shown to produce the CBG-glycoside indicated along with the % conversion efficiency.

TABLE 40
Glycosyl transferases catalyzing the conversion of
CBG to OB32 (CBG→CBG-1′-O-β-D-glucoside)
with calculated conversion efficiency.

| | % |
|---|---|
| Plasmid | conversion |
| PL-340(Qs72S_1_GA) | 98.9 |
| PL-182(Ha88B_2_GA) | 82.9 |
| PL-259(Si82A_GA) | 78.2 |
| PL-38(OsO-1_GA) | 76.9 |
| PL-89(At71C1_At71C2_353_GA) | 60.1 |
| PL-338(Pt72B_GA) | 53.9 |
| PL-159(Pt88G_GA) | 51.9 |
| PL-16(At71D1_GA) | 41.4 |
| PL-376(Sp72T_GA) | 29.1 |
| PL-290(Ad72AA_GA) | 28.8 |
| PL-341(Ad72X_GA) | 26.9 |
| PL-5(At73C5_GA) | 15.4 |
| PL-332(Bv73P_GA) | 9.6 |
| PL-364(Ha72T_GA) | 4.7 |
| PL-326(Ha72B_GA) | 4.4 |
| PL-55(Sr76G1_GA) | ND |

ND: Not detected.

TABLE 41
Glycosyl transferases catalyzing the conversion of
CBG to OB33 (CBG→ CBG-1′-O-β-D-glucosyl-
3′-O-β-D-glucoside) with calculated conversion efficiency.

| | % |
|---|---|
| Plasmid | conversion |
| PL-342(Cp73B_GA) | 100.0 |
| PL-258(Pt78G_GA) | 100.0 |
| PL-189(Ac73T_GA) | 100.0 |
| PL-214(Cs73Y_GA) | 100.0 |
| PL-226(Pt73Y_GA) | 100.0 |
| PL-238(Ac73Z_GA) | 100.0 |
| PL-349(Ha71S_GA) | 99.7 |
| PL-69(CrUGT-2 GA) | 85.2 |
| PL-325(Vv71R_GA) | 82.1 |
| PL-300(Si71E_2_GA) | 78.3 |
| cPL-68(Pa85_GA) | 70.1 |
| PL-85(At73B5_GA) | 57.2 |
| PL-259(Si82A_GA) | 39.0 |
| PL-5(At73C5_GA) | 34.6 |
| PL-290(Ad72AA_GA) | 33.1 |
| PL-182(Ha88B_2_GA) | 26.5 |
| PL-338(Pt72B_GA) | 13.7 |
| PL-55(Sr76G1_GA) | ND |

ND: Not detected.

### Cannabinoid Glycosides Produced Using THC as Substrate.

A range of glycosyl transferases were found to catalyze the conversion of THC to a range of different THC-glycosides. Table 42 shows all the THC-glycosides produced and exemplary glycosyl transferases which catalyzed each reaction with corresponding conversion %.

TABLE 42
THC-glycosides produced by glycosyl transferases in vitro.

| Structure | | | | Conversion |
|---|---|---|---|---|
| ID | Common name | Sugar donor | Enzyme(s) | % |
| OB20 | THC-1′-O-β-D-glucoside | UDP- | PL-182(Ha88B_2_GA) | 74.9 |
| | | Glucose | | |
| OB21 | THC-1′-O-β-D-xyloside | UDP-Xylose | PL-214(Cs73Y_GA) | 19.5 |
| OB22 | THC-1′-O-β-D-di- | UDP-Xylose | PL-214(Cs73Y_GA) | 2.1 |
| | xyloside | | | |

Table 43 further shows the retention time (RT), calculated Log P (c log P), expected and measured mass of each compound and fragmentation pattern as determined by LC-MS/QTOF analysis thereby confirming the structure of each THC-glycoside.

TABLE 43
Retention time, cLogP, expected and measured mass, and fragmentation pattern
of each THC-glycoside produced by glycosyl transferases in vitro.

| | | Expected | Measured | | |
|---|---|---|---|---|---|
| Structure | | mass | mass | | |
| ID | RT | [M + H]⁺ | [M + H]⁺ | Fragmentation pattern | clogP |
| OB20 | 16.3 | 477.2847 | 477.2846 | MS2(477.2846): loss of | 5.64 +/− 0.41 |
| | | | | glucose −> m/z 315.2316 | |
| OB21 | 19.2 | 447.2741 | 447.2713 | MS2(447.2713): loss of | 6.74 +/− 0.49 |
| | | | | xylose −> m/z 315.2320 | |
| OB22 | 18.3 | 579.3164 | 579.3122 | MS2(579.3122): loss of 2x | 6.99 +/− 0.65 |
| | | | | xylose −> m/z 315.2297 | |

For 01320, it was found that multiple glycosyl transferases could catalyze the reaction in varying conversion efficiencies. Tables 44 provide a list of glycosyl transferases which were shown to produce the THC-glycoside indicated along with the % conversion efficiency.

TABLE 44
Glycosyl transferases catalyzing the conversion of THC
to OB20 (THC→ THC-1′-O-β-D-glucoside) with
calculated conversion efficiency.

| | % |
|---|---|
| Plasmid | conversion |
| PL-182(Ha88B_2_GA) | 80.3 |
| PL-226(Pt73Y_GA) | 33.0 |
| PL-214(Cs73Y_GA) | 29.5 |
| PL-78(At71C1-Sr71E1_354_GA) | 26.7 |
| PL-342(Cp73B_GA) | 24.7 |
| PL-55(Sr76G1_GA) | ND |

ND: Not detected.

### Cannabinoid Glycosides Produced Using CBN as Substrate.

A range of glycosyl transferases were found to catalyze the conversion of CBN to at least one CBN-glycosides. Table 45 shows all the CBN-glycosides produced and exemplary enzymes which catalyze each reaction with corresponding conversion %.

TABLE 45
CBN-glycosides produced by glycosyl transferases in vitro.

| Structure | | Sugar | | Conversion |
|---|---|---|---|---|
| ID | Common name | donor | Enzyme(s) | % |
| OB23 | CBN-1′-O-β-D- | UDP- | PL- | 100 |
| | di-glucoside | Glucose | 342(Cp73B_GA) | |

Table 46 further shows the retention time (RT), calculated Log P (c log P), expected and measured mass of each compound and fragmentation pattern as determined by LC-MS/QTOF analysis thereby confirming the structure of each CBN-glycoside.

TABLE 46
Retention time, cLogP, expected and measured mass,
and fragmentation pattern of each CBN-glycoside
produced by glycosyl transferases in vitro.

| | | Expected | Measured | | |
|---|---|---|---|---|---|
| Structure | | mass | mass | Fragmentation | |
| ID | RT | [M + H]⁺ | [M + H]⁺ | pattern | clogP |
| OB23 | 16.7 | 635.3062 | 635.3034 | MS2(635.3039): | 3.86 +/− |
| | | | | loss of 2x | 0.56 |
| | | | | glucose −> m/z | |
| | | | | 311.1990 | |

For OB23, it was found that multiple glycosyl transferases could catalyze the reaction in varying conversion efficiencies. Tables 47 provide a list of glycosyl transferases which were shown to produce the CBN-glycoside indicated along with the % conversion efficiency.

TABLE 47
Glycosyl transferases catalyzing the conversion of CBN
to OB23 (CBN→ CBN-1′-O-β-D-di-glucoside) with
calculated conversion efficiency.

| | % |
|---|---|
| Plasmid | conversion |
| PL-342(Cp73B_GA) | 100.0 |
| PL-214(Cs73Y_GA) | 98.6 |
| PL-226(Pt73Y_GA) | 84.0 |
| PL-85(At73B5_GA) | 80.3 |
| PL-300(Si71E_2_GA) | 78.1 |
| PL-182(Ha88B_2_GA) | 68.0 |
| PL-69(CrUGT-2_GA) | 61.1 |
| PL-349(Ha71S_GA) | 53.9 |
| PL-79(Pa72_GA) | 51.9 |
| PL-330(Sp73A_GA) | 47.5 |
| PL-189(Ac73T_GA) | 32.3 |
| PL-325(Vv71R_GA) | 21.9 |
| PL-68(Pa85_GA) | 18.0 |
| PL-55(Sr76G1_GA) | ND |

ND: Not detected.

### Cannabinoid Glycosides Produced Using 11-Nor-9-Carboxy-THC as Substrate.

A range of glycosyl transferases were found to catalyze the conversion of 11-nor-9-carboxy-THC to a range of 11-nor-9-carboxy-THC-glycosides. Table 48 shows all the 11-nor-9-carboxy-THC-glycosides produced and exemplary glycosyl transferases which catalyzed each reaction with corresponding conversion %.

TABLE 48
11-nor-9-carboxy-THC-glycosides produced
by glycosyl transferases in vitro.

| Structure | | Sugar | | Conversion |
|---|---|---|---|---|
| ID | Common name | donor | Enzyme(s) | % |
| OB41 | 11-nor-9-carboxy- | UDP- | PL- | 70.2 |
| | THC-1′-O-β- | Glucose | 113(Tc90A_GA) | |
| | D-glucoside | | | |
| OB42 | 11-nor-9-carboxy- | UDP- | PL- | 3.4 |
| | THC-1′-O-β- | Glucose | 113(Tc90A_GA) | |
| | D-di-glucoside | | | |

Table 49 further shows the retention time (RT), calculated Log P (c log P), expected and measured mass of each compound and fragmentation pattern as determined by LC-MS/QTOF analysis thereby confirming the structure of each 11-nor-9-carboxy-THC-glycoside (OB41, 42).

TABLE 49
Retention time, cLogP, expected and measured mass, and
fragmentation pattern of each 11-nor-9-carboxy-THC-
glycoside produced by glycosyl transferases in vitro.

| | | Expected | Measured | | |
|---|---|---|---|---|---|
| Structure | | mass | mass | Fragmentation | |
| ID | RT | [M + H]⁺ | [M + H]⁺ | pattern | clogP |
| OB41 | 14.9 | 507.2589 | 507.2581 | MS2(507.2594): | 4.17 +/− |
| | | | | loss of | 0.44 |
| | | | | glucose −> m/z | |
| | | | | 327.1961 | |
| OB42 | 15.2 | 669.3117 | 669.3104 | MS2(669.3128): | 4.08 +/− |
| | | | | loss of 2x | 0.63 |
| | | | | glucose −> m/z | |
| | | | | 27.1931 | |

For OB41, it was found that multiple glycosyl transferases could catalyze the reaction in varying conversion efficiencies. Tables 50 provide a list of glycosyl transferases which were shown to produce the 11-nor-9-carboxy-THC-glycoside indicated along with the % conversion efficiency.

TABLE 50
Glycosyl transferases catalyzing the conversion of 11-nor-9-carboxy-
THC to OB41 (11-nor-9-carboxy-THC→ 11-nor-9-carboxy-THC-
1′-O-β-D-glucoside) with calculated conversion efficiency.

| | % |
|---|---|
| Plasmid | conversion |
| PL-276(Cs74S_GA) | 88.8 |
| PL-113(Tc90A_GA) | 70.2 |
| PL-42(At84B1_GA) | 65.9 |
| PL-359(Cp71B_GA) | 56.4 |
| PL-254(Bv75C_GA) | 44.9 |
| PL-206(Tc74Z_GA) | 29.2 |
| PL-265(Ad74X_GA) | 28.6 |
| PL-368(Sp73Q_GA) | 26.4 |
| PL-342(Cp73B_GA) | 25.8 |
| PL-69(CrUGT-2_GA) | 20.2 |
| PL-78(At71C1-Sr71E1_354_GA) | 11.5 |
| PL-226(Pt73Y_GA) | 9.9 |
| PL-364(Ha72T_GA) | 9.0 |
| PL-5(At73C5_GA) | 5.8 |
| PL-68(Pa85_GA) | 5.3 |
| PL-35(Sp73E_GA) | 2.4 |
| PL-214(Cs73Y_GA) | 2.0 |
| PL-28(At72B1_GA) | 0.9 |
| PL-341(Ad72X_GA) | 0.4 |
| PL-55(Sr76G1_GA) | ND |

ND: Not detected.

It was further discovered that a range of glycosyl transferases could use cannabinoids as sugar acceptors resulting in the production of a considerable range of new cannabinoid glycosides. In the screen, enzymes were found which could catalyze a wide variety of different and highly specific reactions. Glycosyl transferases were found that could specifically produce mono-glycosides (e.g. CBD-1′-O-β-D-glucoside (OB1) produced by Pt88G (SEQ ID NO: 147, 148)), di-glycosides (e.g. CBD-1′-O-β-D-glucosyl-3′-O-β-D-glucoside (OB6) produced by Cp7.38 (SEQ ID NO: 191, 192), tri-glycosides (e.g. CBG-1′-O-β-D-glucosyl-3′-O-β-D-di-glucoside (OB33) produced by At73C5 (SEQ ID NO: 107, 108) and even tetra-glycosides (e.g. CBG-1′-O-β-D-tetra-xyloside (OB40) produced by Cs73Y (SEQ ID NO: 157, 158).

It was also found that a range of glycosyl transferases could utilize a range of different UDP-sugars, Cs73Y (SEQ ID NO: 157, 158) for example was found to utilize UDP-glucose, UDP-xylose, UDP-rhamnose, UDP-glucuronic acid, UDP-galactose and UDP-N-acetylglucosamine and attach these sugars to various cannabinoids.

Based on the calculated conversion %, it was found that many glycosyl transferases were highly active, able to catalyze the production of cannabinoid glycosides with remarkably high efficiency. Several enzymes converted 100% of a cannabinoid aglycone to a corresponding cannabinoid glycoside in 24 h (e.g. CBN-1′-O-β-D-di-glucoside (OB23) produced by Cp7.38 (SEQ ID NO: 191, 192) and CBG-1′-O-β-D-glucosyl-3′-O-β-D-glucoside (OB33) produced by Pt78G (SEQ ID NO: 165, 166)).

It was also found that a large number of enzymes could catalyse the production of cannabinoid glycosides. In total this in vitro screen identified 51 enzymes.

Additionally, the glycosyl transferase Sr76G1 isolated from S. rebaudiana (SEQ ID NO: 123, 124) and codon-optimized for expression in E. coli described in prior art as being able to glycosylate a range of cannabinoids was also tested for glycosyltransferase activity on a range of cannabinoid and cannabinoid glycoside substrates. While it was found that Sr76G1 (SEQ ID NO: 123, 124) could attach glucose to the glucose moiety of cannabinoid glucosides (e.g. converting CBD-1′-O-β-D-glucoside (OB1) to CBD-1′-O-β-D-laminaribioside (OB2). However surprisingly, no glycosyltransferase activity was detected using any cannabinoid aglycones as substrate.

### Example 14—In Vivo Bioconversion of Cannabinoid Substrate to Glycosylated Derivative in E. coli

To demonstrate the conversion of cannabinoids to cannabinoid glycosides in vivo, E. coli strains harboring the glycosyl transferases expression plasmids PL-5(At73C5_GA) (SEQ ID NO: 107,108), PL-182(Ha88B_2_GA) (SEQ ID NO: 149,150) and PL-214(Cs73Y_GA) (SEQ ID NO: 157,158) were constructed according to example 6, part II, resulting in E. coli strains EC-5, EC-182 and EC-214. The Sr76G1 expression plasmid (PL-55(Sr76G1_GA (SEQ ID NO:123,124)) was also included (resulting in E. coli strain EC-55) to test whether the absence of activity observed in vitro was also observed in vivo. Strains were subsequently incubated overnight in 5 mL of LB media supplemented with ampicillin in 10 mL pre-culture tubes at 37° C. Subsequently, cells were inoculated to a starting OD600 of 0.1 in 500 μL of LB media supplemented with ampicillin in a 96 deep-well plate and incubated at 30° C. for 6 hours. A cannabinoid substrate was then dissolved in ethanol and added to the culture media along with a suitable inducing agent (IPTG) in the following final concentrations:

### Ethanol: 20 g/L

Cannabinoid substrate: 250 μM

### IPTG: 0.15 mM

Cells were cultivated with the added ethanol, cannabinoid substrate and IPTG for a further 66 hours. Cannabinoid glycosides were extracted and analyzed by HPLC analysis as described above. The decrease in cannabinoid concentration and accumulation of cannabinoid glycosides were quantified and percent conversion calculated for each glycoside. As shown in table 51 below E. coli strains expressing glycosyl transferases could convert a range of cannabinoids into their corresponding glycosides.

TABLE 51
In vivo bioconversion of cannabinoids to cannabinoid glycosides by E. coli strains expressing
glycosyl transferases. Shown is conversion % of cannabinoid to cannabinoid glycoside.

| | | Cannabinoid substrate | | | | | | | | | | |
|---|---|---|---|---|---|---|---|---|---|---|---|---|
| | | | | | | | | | 11-nor-9- | | | |
| | | CBG | | CBN | CBDV | | CBDA | THC | carboxy-THC | | CBD | |
| | | Glycoside produced | | | | | | | | | | |
| | | OB33 | OB35 | OB23 | OB24 | OB25 | OB31 | OB20 | OB41 | OB42 | OB1 | OB6 |
| E. coli | WT control | ND | ND | ND | ND | ND | ND | ND | ND | ND | ND | ND |
| [symbol] | EC-5 | 18.7 | ND | ND | 52.7 | 8.2 | ND | ND | ND | ND | 24.9 | ND |
| | EC-182 | 22.6 | ND | ND | 18.4 | ND | ND | ND | ND | ND | 31.5 | ND |
| | EC-214 | 21.4 | 42.9 | 100.0 | 74.1 | 19.9 | 47.8 | ND | ND | ND | 43.2 | ND |
| | EC-55 | ND | ND | ND | ND | ND | ND | ND | ND | ND | ND | ND |

ND; Not Detected, WT control; XJb (DE3) parental strain. [symbol]
indicates data missing or illegible when filed

The results showed that the selected glycosyl transferases could produce a range of cannabinoid glycosides in vivo, the results also confirmed the lack of activity of Sr76G1 (SEQ ID NO:123,124) observed in vitro was replicated in vivo. As seen in the in vitro assays, some glycosyl transferases could produce cannabinoid glycosides with remarkably high-efficiency, e.g. Cs73Y(SEQ ID NO: 157,158) converted 100% of the fed CBN to OB23. Furthermore, the results showed that the glycosyl transferases expressed in E. coli could utilize the cells endogenous UDP-glucose pool to carry out the reaction, requiring no additional supplementation of this substrate. No activity was detected using THC and 11-nor-9-carboxy-THC as substrate even though activity was detected in vitro indicating that E. coli may be limited in its ability to convert cannabinoids to cannabinoid glycosides.

### Example 15—In Vivo Bioconversion of Cannabinoid Substrate to Glycosylated Derivative in S. cerevisiae

In previous examples it was shown that purified glycosyl transferases could convert a range of substrates to cannabinoid glycosides in vitro, and also glycosyl transferases expressed in E. coli could also carry out these reactions in vivo by feeding a cannabinoid substrate in the cultivation media and using the cells endogenous supply of UDP-glucose. To demonstrate bioconversion of cannabinoids to cannabinoid glycosides in vivo in S. cerevisiae, the glycosyl transferases Cs73Y (SEQ ID NO: 207, 208), previously shown to catalyze the conversion of a range of cannabinoids to cannabinoids glycosides in vitro and in vivo in E. coli was codon-optimized for expression in S. cerevisiae, cloned into the centromeric expression vector p413TEF (resulting in plasmid PL-388(p413TEF: Cs73Y)) and transformed into S. cerevisiae strain BY4741 (resulting in strain SC-1). SC-1 was pre-cultured overnight at 30° C. in SC-His media with 20 g/L glucose then 10 μl of cell culture was transferred to 490 μl of SC-His media with 20 g/L glucose supplemented with various cannabinoids dissolved in 100% ethanol and incubated for 3 days at 30° C. The final concentration of cannabinoids in media was 250 μM and the final ethanol concentration was 20 g/L. Samples were prepared and analyzed as described above. As shown in table 52, SC-1 expressing the glycosyl transferase Cs73Y could convert a range of cannabinoids into their respective mono-, di-, and tri-glycosides with high efficiency.

TABLE 52
In vivo bioconversion of cannabinoids to cannabinoid glycosides by
S. cerevisiae strain SC-1 expressing the glycosyl transferase Cs73Y.
Shown is conversion % of cannabinoid to cannabinoid glycoside.

| | Cannabinoid substrate | | | | | | | | | | |
|---|---|---|---|---|---|---|---|---|---|---|---|
| | | | | | | | | 11-nor-9- | | | |
| | CBG | | CBN | CBDV | | CBDA | THC | carboxy-THC | | CBD | |
| | Glycoside produced | | | | | | | | | | |
| | OB33 | OB35 | OB23 | OB24 | OB25 | OB31 | OB20 | OB41 | OB42 | OB1 | OB6 |
| WT control | ND | ND | ND | ND | ND | ND | ND | ND | ND | ND | ND |
| SC-1 | 45.7 | 51.0 | 98.3 | 93.0 | 7.0 | 100.0 | 14.4 | 11.2 | 5.4 | 57.3 | 42.7 |

ND; Not detected, WT control; BY4741 parental strain.

It was found that SC-1 could convert all cannabinoids tested into cannabinoid glycosides with remarkably high efficiency. For all cannabinoids tested except THC and 11-nor-9-carboxy-THC it was found that SC-1 converted all of the added cannabinoid to cannabinoid-glycosides. Furthermore, while production of THC and 11-nor-9-carboxy-THC glycosides was not detected in E. coli cultures expressing glycosyl transferases, THC and 11-nor-9-carboxy-THC glycosides were detected in S. cerevisiae cultures. This not only indicated that the cannabinoids successfully were imported into the cell and that the cells endogenous supply of UDP-glucose was sufficient to carry out the reactions, it also demonstrated that S. cerevisiae was a superior host for the production of cannabinoid glycosides compared to E. coli.

### Example 16—Test of Intestinal Permeability of Glycosylated Cannabinoids

Intestinal permeability of cannabinoids and glycosylated cannabinoids was determined by measuring bi-directional transport across Caco-2 cell membranes. Caco-2 cells are used as an in vitro model of the human intestinal epithelium and permit assessment of the intestinal permeability of potential drugs. The test compound is added to either the apical or basolateral side of a confluent monolayer of Caco-2 cells and permeability is measured by monitoring the appearance of the test compound on the opposite side of the monolayer using LC-MS/QTOF. When performing a bi-directional assay, the efflux ratio (ER) is calculated from the ratio of B−A and A−B permeabilities. Caco-2 cells obtained from the ATCC are used between passage numbers 40-60. Cells are seeded onto Millipore Multiscreen Transwell plates at 1×105 cells/cm2. The cells are cultured in DMEM and media is changed every two or three days. On day 20 the permeability study is performed. Cell culture and assay incubations are carried out at 37° C. in an atmosphere of 5% CO2 with a relative humidity of 95%. On the day of the assay, the monolayers are prepared by rinsing both apical and basolateral surfaces twice with Hanks Balanced Salt Solution (HBSS) at the desired pH warmed to 37° C. Cells are then incubated with HBSS at the desired pH in both apical and basolateral compartments for 40 min to stabilize physiological parameters. 10 mM solutions of cannabinoids and cannabinoid glycosides are prepared in DMSO then diluted with assay buffer to give a final test compound concentration of 10 μM (final DMSO concentration of 1% v/v). The fluorescent integrity marker lucifer yellow is also included in the solution.

Analytical standards are prepared from test compound DMSO dilutions and transferred to buffer, maintaining a 1% v/v DMSO concentration. For assessment of A−B permeability, HBSS is removed from the apical compartment and replaced with test compound solution. The apical compartment insert is then placed into a companion plate containing fresh buffer (containing 1% v/v DMSO). For assessment of B−A permeability, HBSS is removed from the companion plate and replaced with test compound solution. Fresh buffer (containing 1% v/v DMSO) is added to the apical compartment insert, which is then placed into the companion plate. At 120 min the apical compartment inserts and the companion plates are separated and apical and basolateral samples diluted for analysis. Test compound permeability is assessed in duplicate. Compounds of known permeability characteristics are run as controls on each assay plate. Test and control compounds are quantified by LC-MS/QTOF as described above. The starting concentration (C0) is determined from the solution and the experimental recovery calculated from C0 and both apical and basolateral compartment concentrations. The integrity of the monolayer throughout the experiment is checked by monitoring lucifer yellow permeation using fluorometric analysis. The permeability coefficient (Pₐₚₚ) for each compound is calculated from the following equation: Pₐₚₚ=(dQ/dt)/(C₀×A) Where dQ/dt is the rate of permeation of the drug across the cells, C₀ is the donor compartment concentration at time zero and A is the area of the cell monolayer. C₀ is obtained from analysis of the dosing solution. The efflux ratio (ER) is calculated from mean Pₐₚₚ values from A−B and B−A data. This is derived from: ER=Pₐₚₚ(B−A)/Pₐₚₚ(A−B). The % recovery is calculated from the following equation; % recovery=(Total compound in donor and receiver compartment at end of experiment)/(initial compound present)×100.

The mean permeability coefficient (Pₐₚₚ) both in the A to B and B to A direction, mean substrate recovery, and corresponding efflux ratio for CBD, CBD-r-O-β-D-glucoside (OB1) and CBD-1′-O-β-D-glucosyl-3′-O-β-D-glucoside (OB6) was measured. CBD glycosides were produced using glycosyl transferases and purified as described above. As shown in table 53 below compared to unmodified CBD, OB1 had significantly higher permeability coefficients in both directions and a higher efflux ratio, overall indicating improved intestinal permeability and efflux. For OB6, while the permeability coefficients were lower, the resulting efflux ratio was higher than both CBD and OB1 indicating improved efflux of the molecule from the intestine. Furthermore, the results clearly showed that glycosylation improves the % recovery with successively higher rates of recovery in both compartments observed for OB1 and OB6. Low recovery of compound in a Caco-2 permeability assay can indicate problems with poor solubility, binding of the compound to the plate, metabolism by the Caco-2 cells or accumulation of the compound in the cell monolayer.

TABLE 53
In vitro measurement of intestinal permeability of CBD, CBD-1′-O-β-D-glucoside (OB1) and CBD-
1′-O-β-D-glucosyl-3′-O-β-D-glucoside (OB6) in a Caco-2 bi-directional permeability assay. Results
calculated as mean and standard deviation from duplicate experiments. Direction A→B; Diffusion from
apical to basolateral compartment, Direction B→A; Diffusion from basolateral to apical compartment.
Pₐₚₚ; permeability coefficient.

| | Direction A→B | | Direction B→A | | Efflux ratio |
|---|---|---|---|---|---|
| Compound | Mean Pₐₚₚ (10⁻⁶ cms⁻¹) | Mean recovery (%) | Mean Pₐₚₚ (10⁻⁶ cms⁻¹) | Mean recovery (%) | $\frac{Mean Papp B \to A}{Mean Papp A \to B}$ |
| CBD | 0.61 ± 0.03 | 16.4 | 1.45 ± 0.64 | 48.8 | 2.37 |
| OB1 | 10.40 ± 0.31 | 69.8 | 35.70 ± 0.34 | 79.7 | 3.43 |
| OB6 | 0.10 ± 0.05 | 91.9 | 0.44 ± 0.17 | 87.8 | 4.31 |

### Example 17—De Novo Production of Glycosylated Cannabinoids in S. cerevisiae

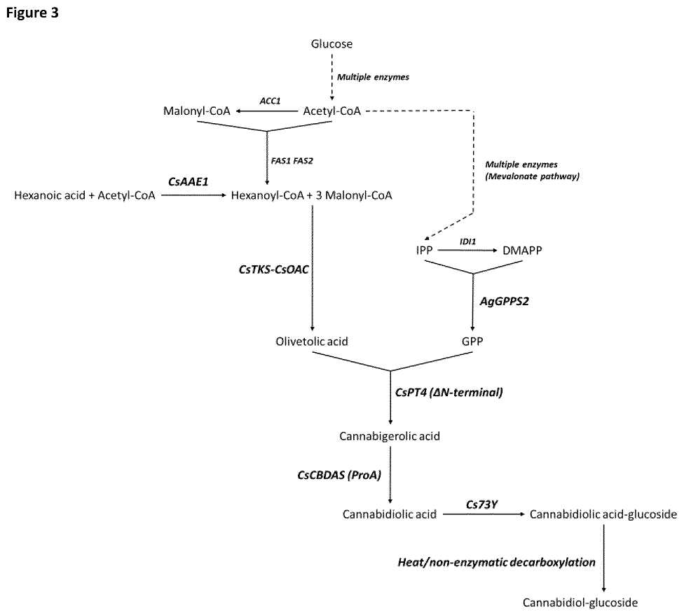

To demonstrate the de novo production of cannabinoid glycosides a heterologous biosynthetic pathway for the production of CBDA was introduced into S. cerevisiae wild-type strain BY4741 as described previously, resulting in strain SC-CBDA. Additionally, the glycosyl transferase Cs73Y (SEQ ID NO: 207, 208), shown to glycosylate a range of cannabinoids expressed on plasmid PL-388(p413TEF: Cs73Y) was transferred into this strain resulting in strain SC-CBDAGLY. The plasmids used to construct these strains is shown in Table 54 and the resulting biosynthetic pathway that was introduced is shown in FIG. 3.

TABLE 54
Plasmids used to construct SC-CBDA and SC-CBDAGLY cannabinoid producing S. cerevisiae strains.

| Plasmid name | Plasmid backbone | Gene(s) overexpressed | Marker |
|---|---|---|---|
| PL-381(Rec1-XI-5-LEU: CsTKS-CsOAC) | Recombinator_1_XI-5_LEU2 | CsTKS-CsOAC | LEU2 |
| PL-382(Rec2-LEU: AgGPPS2) | Recombinator_2_LEU2 | AgGPPS2 | |
| PL-383(Rec3: CsTHCAS) | Recombinator_3 | CsTHCAS | |
| PL-384(Rec3: CsCBDAS) | Recombinator_3 | CsCBDAS | |
| PL-385(Rec4: CsPT4) | Recombinator_4 | CsPT4 (ΔN-terminal) | |
| PL-386(Rec4: SsNphB(Q295F)) | Recombinator_4 | SsNphB(Q295F) | |
| PL-387(Rec5-XI-5: CsAAE1) | Recombinator_5_XI-5 | CsAAE1 | |
| PL-388(p413TEF: Cs73Y) | p413TEF | Cs73Y | HIS3 |

Strains were subsequently cultivated as previously described in synthetic medium minus leucine and histidine supplementation (SC-Ura+His) with 20 g/L glucose and 1 mM hexanoic acid added and samples prepared and analyzed as previously described. As shown in table 55 below, introduction of the cannabinoid biosynthetic pathway (SC-CBDA) resulted in the production of 1.97 μM CBDA, further introduction of the glycosyl transferase Cs73Y resulted in the production of 2.03 μM CBDA-1′-O-β-D-glucoside (OB31). Heating of the cell culture broth as described above resulted in the production of 0.87 μM CBD from SC-CBDA cell cultures and 1.54 μM CBD-1′-O-β-D-glucoside (OB1) from SC-CBDAGLY cell cultures.

TABLE 55
De novo production of cannabinoids and cannabinoid
glycosides in engineered S. cerevisiae strains.

| | CBDA | OB31 | CBD | OB1 |
|---|---|---|---|---|
| SC-CBDA | 1.97 | ND | 0.87 | ND |
| SC-CBDAGLY | ND | 2.03 | ND | 1.54 |

ND; Not Detected.
Data presented in μM and as averages of duplicate experiments. Cells were cultivated for 3 days in SC-Ura + His media supplemented with 20 g/L glucose and 1 mM hexanoic acid.

### Example 18—In Vitro Enzymatic Cascade for Production of Cannabinoid Glycosides from Sucrose and a Cannabinoid Substrate

In the previous examples, in vitro glycosyl transferase assays required the addition of an “activated” sugar (e.g. UDP-glucose), which is typically an extremely expensive reagent, furthermore, other activated sugars e.g. UDP-rhamnose are not available commercially and must be custom synthesized at high-cost and difficulty. In vivo, while S. cerevisiae and E. coli are able to natively produce UDP-glucose, they do so in low amounts, and further, do not produce other activated sugars thereby limiting their applicability for the in vivo production of diverse cannabinoid glycosides. To facilitate the low-cost production of cannabinoid glycosides not only with glucose, but with alternative sugars, an enzymatic cascade was set up to convert cannabinoids and the simple sugar sucrose into various cannabinoid glycosides. The cascade is divided into 3 steps, in step 1 sucrose and uridine diphosphate (UDP) is converted to UDP-glucose by GmSuSy (SEQ ID NO: 209, 210), additionally generating fructose as a bi-product. In step 2, UDP-glucose is interconverted to alternative UDP-sugars using a range of enzymes. For example, conversion of UDP-glucose to UDP-galactose by BsGa/E, multiple enzymes can also be used to produce UDP-sugars via other UDP-sugar intermediates. For example, conversion of UDP-glucose to UDP-glucuronic acid by AtUGDH1 combined with conversion of UDP-glucuronic acid to UDP-xylose by AtUXS3. In step 3, glycosyl transferases convert the activated sugar and a cannabinoid acceptor to the corresponding cannabinoid glycoside. For example, conversion of UDP-rhamnose and CBD to CBD-1′-O-β-D-rhamnoside (OB13) by Cs73Y (SEQ ID NO: 157, 158). Examples of enzymes which can interconvert UDP-sugars is shown in the table below, table 56.

TABLE 56
Enzymes for the interconversion of UDP-sugars.

| Enzyme | Gene | Reaction |
|---|---|---|
| UDP-galactose 4-epimerase | BsGalE | UDP-glucose −> UDP- |
| | | galactose |
| UDP-glucuronic acid | AtUXS3 | UDP-glucuronic acid −> |
| decarboxylase | | UDP-xylose |
| UDP-glucose 4,6-dehydratase/ | AtRHM2 | UDP-glucose + NAD⁺ + |
| UDP-4-keto-6-deoxy-glucose | | NADPH −> UDP-rhamnose + |
| 3,5-epimerase/UDP-4-keto- | | NADH + NADP⁺ |
| rhamnose 4-keto-reductase | | |
| UDP-glucose 6- | AtUGDH1 | UDP-glucose + 2NAD+ −> |
| dehydrogenase | | UDP-glucuronic acid + |
| | | 2NADH |
| UDP-arabinose 4-epimerase | AtMUR4 | UDP-xylose −> UDP- |
| | | arabinose |

Alternatively, for the production of UDP-rhamnose, instead of using a full length AtRHM2 gene (SEQ ID NO: 219, 220), for better expression and higher activity AtRHM2 may be divided into the N- and C-terminal domains AtRHM2-N(SEQ ID NO: 217, 218) and AtRHM2-C(SEQ ID NO: 215, 216) catalyzing the dehydration, and the epimerization and reduction, respectively. Alternatively, all three (full-length AtRHM2 (covering amino acids 1-667), AtRHM2-N (covering amino acids 1-370) and AtRHM2-C (covering amino acids 371-667)) may be mixed to increase the production of UDP-rhamnose.

The cascade reaction can be performed in a single reaction, alternatively, steps 1, 2 and 3 can be split into different reactions and combined as needed.

This enzyme cascade for the production of cannabinoid glycosides was demonstrated in vitro with CBD using purified GmSuSy and Cs73Y enzyme with different combinations of UDP-sugar interconverting enzymes and required co-factors. Enzymes were purified and the in vitro assay performed as described in Example 13 and the reaction mixture set up as shown in table 57. Enzymes and co-factors were added as required for each individual reaction. Samples were extracted and analyzed as stated above.

TABLE 57
Reaction setup to produce cannabinoid glycosides
with alternative sugars in vitro.

| Reagent | Volume (μL) |
|---|---|
| Purified enzyme(s) | 5 per enzyme |
| 25 mM Cannabinoid substrate | 0.4 |
| 1M Tris-HCl pH7.4 | 2 |
| Milli-Q water | Up to 20 |
| 50 mM UDP | 0.5 |
| 50 mM Sucrose | 0.5 |
| 50 mM nicotinamide co-factors | 0.5 |
| TOTAL | 20 |

As shown in table 58 below, various CBD-di-glycosides could be produced from sucrose and CBD by adding different combinations of enzymes in high-efficiency.

TABLE 58
Conversion of CBD and sucrose to various CBD glycosides by
adding different combinations of sugar conversion enzymes.

| | Enzymes added to reaction mix | | | % Conversion | | | |
|---|---|---|---|---|---|---|---|
| CBD glycoside produced | GmSuSy | BsGalE | AtUGDH1 | AtUXS3 | AtRHM2 | Cs73Y | |
| CBD no UDP-sugar | | | | | | + | ND |
| control | | | | | | | |
| CBD no glycosyl | + | | | | | | ND |
| transferases control | | | | | | | |
| CBD-1′-O-β-D-glucosyl-3′- | + | | | | | + | 91.3 |
| O-β-D-glucoside (OB6) | | | | | | | |
| CBD-1′-O-β-D-galactosyl- | + | + | | | | + | 38.2 |
| 3′-O-β-D-galactoside (OB17) | | | | | | | |
| CBD-1′-O-β-D-glucurosyl- | + | | + | | | + | 29.4 |
| 3′-O-β-D-glucuronide (OB15) | | | | | | | |
| CBD-1′-O-β-D-xylosyl-3′- | + | | + | + | | + | 72.3 |
| O-β-D-xyloside (OB10) | | | | | | | |
| CBD-1′-O-β-O- | + | | | | + | + | 15.2 |
| rhamnoside (OB13) | | | | | | | |

ND; Not Detected

### Example 19—Use of Glycosyl Transferases to Produce Novel Molecules

The glycosyl transferases of the invention has revealed and made possible to produce a range of hitherto unknown cannabinoid glycosides that can be broadly grouped into the following categories:

TABLE 59
Categories of novel cannabinoid glycosides produced by enzymes
of the invention. Also displayed is an exemplary molecule
of each category and the corresponding enzyme(s) and SEQ
ID NO’s which can be used to produce the molecule.

| | | | SEQ |
|---|---|---|---|
| Group | Exemplary molecule | Enzyme | ID NO |
| Cannabinoid | CBD-1′-O-β-D-cellobioside | Pt88G + | 147, 115 |
| cellobioside | | OsEUGT11 | |
| Cannabinoid | CBD-1′-O-β-D-gentiobioside | Pt88G + | 147, 145 |
| gentiobioside | | Si94D | |
| Cannabinoid | THC-1′-O-β-D-xyloside | Cs73Y | 157 |
| xyloside | | | |
| Cannabinoid | CBD-1′-O-α-L-rhamnoside | Cp73B | 191 |
| rhamnoside | | | |
| Cannabinoid | CBD-1′-O-β-D-galactosyl-3′- | Cs73Y | 157 |
| galactoside | O-β-D-galactoside | | |
| Cannabinoid | CBD-1′-O-β-D-N-acetyl- | Cs73Y | 157 |
| N-acetylglu- | glucosamine-3′-O-β-D-N- | | |
| cosaminoside | acetylglucosaminoside | | |
| Cannabinoid | CBD-1′-O-β-D-arabinosyl-3′- | Cs73Y | 157 |
| arabinoside | O-β-D-arabinoside | | |
| Cannabinoid | CBD-1′-O-β-D-N-acetyl- | Cs73Y | 157 |
| N-acetylgalac- | galactosamine-3′-O-β- | | |
| tosaminoside | D-N-acetylgalactosamine | | |

Enzymes of the invention can be used to produce the following molecules:

TABLE 60
List of novel cannabinoid glycosides produced by enzymes of the invention. Also shown
are enzyme which can be used to produce each molecule and corresponding SEQ ID NO′s.

| | | SEQ |
|---|---|---|
| Glycoside name | Enzyme(s) | ID NO |
| CBD-1′-O-β-D-cellobioside | Pt88G + OsEUGT11 | 147, 115 |
| CBD-1′-O-β-D-gentiobioside | Pt88G + Si94D | 147, 145 |
| CBD-1′-O-β-D-xyloside | Pt88G | 147 |
| CBD-1′-O-α-L-rhamnoside | Cp73B | 191 |
| CBD-1′-O-β-D-galactoside | Cs73Y | 157 |
| CBD-1′-O-β-D-N-acetylglucosaminoside | Cs73Y | 157 |
| CBD-1′-O-β-D-arabinoside | Cs73Y | 157 |
| CBD-1′-O-β-D-N-acetylgalactosaminoside | Cs73Y | 157 |
| CBDV-1′-O-β-D-cellobioside | Ha72B + OsEUGT11 | 179, 115 |
| CBDV-1′-O-β-D-gentiobioside | Ha72B + Si94D | 179, 145 |
| CBDV-1′-O-β-D-xyloside | Cs73Y | 157 |
| CBDV-1′-O-α-L-rhamnoside | Cs73Y | 157 |
| CBDV-1′-O-β-D-galactoside | Cs73Y | 157 |
| CBDV-1′-O-β-D-N-acetylglucosaminoside | Cs73Y | 157 |
| CBDV-1′-O-β-D-arabinoside | Cs73Y | 157 |
| CBDV-1′-O-β-D-N-acetylgalactosaminoside | Cs73Y | 157 |
| CBDA-1′-O-β-D-cellobioside | Cs73Y + OsEUGT11 | 157, 115 |
| CBDA-1′-O-β-D-gentiobioside | Cs73Y + Si94D | 157, 145 |
| CBDA-1′-O-β-D-xyloside | Cs73Y | 157 |
| CBDA-1′-O-α-L-rhamnoside | Cs73Y | 157 |
| CBDA-1′-O-β-D-galactoside | Cs73Y | 157 |
| CBDA-1′-O-β-D-N-acetylglucosaminoside | Cs73Y | 157 |
| CBDA-1′-O-β-D-arabinoside | Cs73Y | 157 |
| CBDA-1′-O-β-D-N-acetylgalactosaminoside | Cs73Y | 157 |
| CBG-1′-O-β-D-cellobioside | Qs72S + OsEUGT11 | 187, 115 |
| CBG-1′-O-β-D-gentiobioside | Qs72S + Si94D | 187, 145 |
| CBG-1′-O-β-D-xyloside | Cs73Y | 157 |
| CBG-1′-O-α-L-rhamnoside | Cs73Y | 157 |
| CBG-1′-O-β-D-galactoside | Cs73Y | 157 |
| CBG-1′-O-β-D-N-acetylglucosaminoside | Cs73Y | 157 |
| CBG-1′-O-β-D-arabinoside | Cs73Y | 157 |
| CBG-1′-O-β-D-N-acetylgalactosaminoside | Cs73Y | 157 |
| THC-1′-O-β-D-cellobioside | Ha88B_2 + OsEUGT11 | 149, 115 |
| THC-1′-O-β-D-gentiobioside | Ha88B_2 + Si94D | 149, 145 |
| THC-1′-O-β-D-xyloside | Cs73Y | 157 |
| THC-1′-O-α-L-rhamnoside | Cs73Y | 157 |
| THC-1′-O-β-D-galactoside | Cs73Y | 157 |
| THC-1′-O-β-D-N-acetylglucosaminoside | Cs73Y | 157 |
| THC-1′-O-β-D-arabinoside | Cs73Y | 157 |
| THC-1′-O-β-D-N-acetylgalactosaminoside | Cs73Y | 157 |
| THCV-1′-O-β-D-cellobioside | Ha88B_2 + OsEUGT11 | 149, 115 |
| THCV-1′-O-β-D-gentiobioside | Ha88B_2 + Si94D | 149, 145 |
| THCV-1′-O-β-D-xyloside | Cs73Y | 157 |
| THCV-1′-O-α-L-rhamnoside | Cs73Y | 157 |
| THCV-1′-O-β-D-galactoside | Cs73Y | 157 |
| THCV-1′-O-β-D-N-acetylglucosaminoside | Cs73Y | 157 |
| THCV-1′-O-β-D-arabinoside | Cs73Y | 157 |
| THCV-1′-O-β-D-N-acetylgalactosaminoside | Cs73Y | 157 |
| CBC-1′-O-β-D-cellobioside | Cs73Y + OsEUGT11 | 157, 115 |
| CBC-1′-O-β-D-gentiobioside | Cs73Y + Si94D | 157, 145 |
| CBC-1′-O-β-D-xyloside | Cs73Y | 157 |
| CBC-1′-O-α-L-rhamnoside | Cs73Y | 157 |
| CBC-1′-O-β-D-galactoside | Cs73Y | 157 |
| CBC-1′-O-β-D-N-acetylglucosaminoside | Cs73Y | 157 |
| CBC-1′-O-β-D-arabinoside | Cs73Y | 157 |
| CBC-1′-O-β-D-N-acetylgalactosaminoside | Cs73Y | 157 |
| CBN-1′-O-β-D-cellobioside | Cp73B + OsEUGT11 | 191, 115 |
| CBN-1′-O-β-D-gentiobioside | Cp73B + Si94D | 191, 145 |
| CBN-1′-O-β-D-xyloside | Cs73Y | 157 |
| CBN-1′-O-α-L-rhamnoside | Cs73Y | 157 |
| CBN-1′-O-β-D-galactoside | Cs73Y | 157 |
| CBN-1′-O-β-D-N-acetylglucosaminoside | Cs73Y | 157 |
| CBN-1′-O-β-D-arabinoside | Cs73Y | 157 |
| CBN-1′-O-β-D-N-acetylgalactosaminoside | Cs73Y | 157 |
| 11-nor-9-carboxy-THC-1′-O-β-D-cellobioside | Tc90A + OsEUGT11 | 143, 115 |
| 11-nor-9-carboxy-THC-1′-O-β-D-gentiobioside | Tc90A + Si94D | 143, 145 |
| 11-nor-9-carboxy-THC-1′-O-β-D-xyloside | Cs73Y | 157 |
| 11-nor-9-carboxy-THC-1′-O-α-L-rhamnoside | Cs73Y | 157 |
| 11-nor-9-carboxy-THC-1′-O-β-D-galactoside | Cs73Y | 157 |
| 11-nor-9-carboxy-THC-1′-O-β-D-N-acetylglucosaminoside | Cs73Y | 157 |
| 11-nor-9-carboxy-THC-1′-O-β-D-arabinoside | Cs73Y | 157 |
| 11-nor-9-carboxy-THC-1′-O-β-D-N-acetylgalactosaminoside | Cs73Y | 157 |
| CBD-3′-O-β-D-cellobioside | Pt88G + OsEUGT11 | 147, 115 |
| CBD-3′-O-β-D-gentiobioside | Pt88G + Si94D | 147, 145 |
| CBD-3′-O-β-D-xyloside | Pt88G | 147 |
| CBD-3′-O-α-L-rhamnoside | Cp73B | 191 |
| CBD-3′-O-β-D-galactoside | Cs73Y | 157 |
| CBD-3′-O-β-D-N-acetylglucosaminoside | Cs73Y | 157 |
| CBD-3′-O-β-D-arabinoside | Cs73Y | 157 |
| CBD-3′-O-β-D-N-acetylgalactosaminoside | Cs73Y | 157 |
| CBDV-3′-O-β-D-cellobioside | Ha72B + OsEUGT11 | 179, 115 |
| CBDV-3′-O-β-D-gentiobioside | Ha72B + Si94D | 179, 145 |
| CBDV-3′-O-β-D-xyloside | Cs73Y | 157 |
| CBDV-3′-O-α-L-rhamnoside | Cs73Y | 157 |
| CBDV-3′-O-β-D-galactoside | Cs73Y | 157 |
| CBDV-3′-O-β-D-N-acetylglucosaminoside | Cs73Y | 157 |
| CBDV-3′-O-β-D-arabinoside | Cs73Y | 157 |
| CBDV-3′-O-β-D-N-acetylgalactosaminoside | Cs73Y | 157 |
| CBG-3′-O-β-D-cellobioside | Qs72S + OsEUGT11 | 187, 115 |
| CBG-3′-O-β-D-gentiobioside | Qs72S + Si94D | 187, 145 |
| CBG-3′-O-β-D-xyloside | Cs73Y | 157 |
| CBG-3′-O-α-L-rhamnoside | Cs73Y | 157 |
| CBG-3′-O-β-D-galactoside | Cs73Y | 157 |
| CBG-3′-O-β-D-N-acetylglucosaminoside | Cs73Y | 157 |
| CBG-3′-O-β-D-arabinoside | Cs73Y | 157 |
| CBG-3′-O-β-D-N-acetylgalactosaminoside | Cs73Y | 157 |
| CBD-1′-O-β-D-di-xyloside | Cs73Y | 157 |
| CBD-1′-O-α-L-di-rhamnoside | Cs73Y | 157 |
| CBD-1′-O-β-D-di-galactoside | Cs73Y | 157 |
| CBD-1′-O-β-D-di-N-acetylglucosaminoside | Cs73Y | 157 |
| CBD-1′-O-β-D-di-arabinoside | Cs73Y | 157 |
| CBD-1′-O-β-D-di-N-acetylgalactosaminoside | Cs73Y | 157 |
| CBDV-1′-O-β-D-di-xyloside | Cs73Y | 157 |
| CBDV-1′-O-α-L-di-rhamnoside | Cs73Y | 157 |
| CBDV-1′-O-β-D-di-galactoside | Cs73Y | 157 |
| CBDV-1′-O-β-D-di-N-acetylglucosaminoside | Cs73Y | 157 |
| CBDV-1′-O-β-D-di-arabinoside | Cs73Y | 157 |
| CBDV-1′-O-β-D-di-N-acetylgalactosaminoside | Cs73Y | 157 |
| CBDA-1′-O-β-D-di-xyloside | Cs73Y | 157 |
| CBDA-1′-O-α-L-di-rhamnoside | Cs73Y | 157 |
| CBDA-1′-O-β-D-di-galactoside | Cs73Y | 157 |
| CBDA-1′-O-β-D-di-N-acetylglucosaminoside | Cs73Y | 157 |
| CBDA-1′-O-β-D-di-arabinoside | Cs73Y | 157 |
| CBDA-1′-O-β-D-di-N-acetylgalactosaminoside | Cs73Y | 157 |
| CBG-1′-O-β-D-di-xyloside | Cs73Y | 157 |
| CBG-1′-O-α-L-di-rhamnoside | Cs73Y | 157 |
| CBG-1′-O-β-D-di-galactoside | Cs73Y | 157 |
| CBG-1′-O-β-D-di-N-acetylglucosaminoside | Cs73Y | 157 |
| CBG-1′-O-β-D-di-arabinoside | Cs73Y | 157 |
| CBG-1′-O-β-D-di-N-acetylgalactosaminoside | Cs73Y | 157 |
| THC-1′-O-β-D-di-xyloside | Cs73Y | 157 |
| THC-1′-O-α-L-di-rhamnoside | Cs73Y | 157 |
| THC-1′-O-β-D-di-galactoside | Cs73Y | 157 |
| THC-1′-O-β-D-di-N-acetylglucosaminoside | Cs73Y | 157 |
| THC-1′-O-β-D-di-arabinoside | Cs73Y | 157 |
| THC-1′-O-β-D-di-N-acetylgalactosaminoside | Cs73Y | 157 |
| THCV-1′-O-β-D-di-xyloside | Cs73Y | 157 |
| THCV-1′-O-α-L-di-rhamnoside | Cs73Y | 157 |
| THCV-1′-O-β-D-di-galactoside | Cs73Y | 157 |
| THCV-1′-O-β-D-di-N-acetylglucosaminoside | Cs73Y | 157 |
| THCV-1′-O-β-D-di-arabinoside | Cs73Y | 157 |
| THCV-1′-O-β-D-di-N-acetylgalactosaminoside | Cs73Y | 157 |
| CBC-1′-O-β-D-di-xyloside | Cs73Y | 157 |
| CBC-1′-O-α-L-di-rhamnoside | Cs73Y | 157 |
| CBC-1′-O-β-D-di-galactoside | Cs73Y | 157 |
| CBC-1′-O-β-D-di-N-acetylglucosaminoside | Cs73Y | 157 |
| CBC-1′-O-β-D-di-arabinoside | Cs73Y | 157 |
| CBC-1′-O-β-D-di-N-acetylgalactosaminoside | Cs73Y | 157 |
| CBN-1′-O-β-D-di-xyloside | Cs73Y | 157 |
| CBN-1′-O-α-L-di-rhamnoside | Cs73Y | 157 |
| CBN-1′-O-β-D-di-galactoside | Cs73Y | 157 |
| CBN-1′-O-β-D-di-N-acetylglucosaminoside | Cs73Y | 157 |
| CBN-1′-O-β-D-di-arabinoside | Cs73Y | 157 |
| CBN-1′-O-β-D-di-N-acetylgalactosaminoside | Cs73Y | 157 |
| 11-nor-9-carboxy-THC-1′-O-β-D-di-xyloside | Cs73Y | 157 |
| 11-nor-9-carboxy-THC-1′-O-α-L-di-rhamnoside | Cs73Y | 157 |
| 11-nor-9-carboxy-THC-1′-O-β-D-di-galactoside | Cs73Y | 157 |
| 11-nor-9-carboxy-THC-1′-O-β-D-di-N-acetylglucosaminoside | Cs73Y | 157 |
| 11-nor-9-carboxy-THC-1′-O-β-D-di-arabinoside | Cs73Y | 157 |
| 11-nor-9-carboxy-THC-1′-O-β-D-di-N-acetylgalactosaminoside | Cs73Y | 157 |
| CBD-3′-O-β-D-di-xyloside | Cs73Y | 157 |
| CBD-3′-O-α-L-di-rhamnoside | Cs73Y | 157 |
| CBD-3′-O-β-D-di-galactoside | Cs73Y | 157 |
| CBD-3′-O-β-D-di-N-acetylglucosaminoside | Cs73Y | 157 |
| CBD-3′-O-β-D-di-arabinoside | Cs73Y | 157 |
| CBD-3′-O-β-D-di-N-acetylgalactosaminoside | Cs73Y | 157 |
| CBDV-3′-O-β-D-di-xyloside | Cs73Y | 157 |
| CBDV-3′-O-α-L-di-rhamnoside | Cs73Y | 157 |
| CBDV-3′-O-β-D-di-galactoside | Cs73Y | 157 |
| CBDV-3′-O-β-D-di-N-acetylglucosaminoside | Cs73Y | 157 |
| CBDV-3′-O-β-D-di-arabinoside | Cs73Y | 157 |
| CBDV-3′-O-β-D-di-N-acetylgalactosaminoside | Cs73Y | 157 |
| CBG-3′-O-β-D-di-xyloside | Cs73Y | 157 |
| CBG-3′-O-α-L-di-rhamnoside | Cs73Y | 157 |
| CBG-3′-O-β-D-di-galactoside | Cs73Y | 157 |
| CBG-3′-O-β-D-di-N-acetylglucosaminoside | Cs73Y | 157 |
| CBG-3′-O-β-D-di-arabinoside | Cs73Y | 157 |
| CBG-3′-O-β-D-di-N-acetylgalactosaminoside | Cs73Y | 157 |
| CBD-1′-O-β-D-tri-xyloside | Cs73Y | 157 |
| CBD-1′-O-α-D-tri-rhamnoside | Cs73Y | 157 |
| CBD-1′-O-β-D-tri-galactoside | Cs73Y | 157 |
| CBD-1′-O-β-D-tri-N-acetylglucosaminoside | Cs73Y | 157 |
| CBD-1′-O-β-D-tri-arabinoside | Cs73Y | 157 |
| CBD-1′-O-β-D-tri-N-acetylgalactosaminoside | Cs73Y | 157 |
| CBDV-1′-O-β-D-tri-xyloside | Cs73Y | 157 |
| CBDV-1′-O-α-D-tri-rhamnoside | Cs73Y | 157 |
| CBDV-1′-O-β-D-tri-galactoside | Cs73Y | 157 |
| CBDV-1′-O-β-D-tri-N-acetylglucosaminoside | Cs73Y | 157 |
| CBDV-1′-O-β-D-tri-arabinoside | Cs73Y | 157 |
| CBDV-1′-O-β-D-tri-N-acetylgalactosaminoside | Cs73Y | 157 |
| CBG-1′-O-β-D-tri-xyloside | Cs73Y | 157 |
| CBG-1′-O-α-D-tri-rhamnoside | Cs73Y | 157 |
| CBG-1′-O-β-D-tri-galactoside | Cs73Y | 157 |
| CBG-1′-O-β-D-tri-N-acetylglucosaminoside | Cs73Y | 157 |
| CBG-1′-O-β-D-tri-arabinoside | Cs73Y | 157 |
| CBN-1′-O-β-D-tri-xyloside | Cs73Y | 157 |
| CBN-1′-O-α-D-tri-rhamnoside | Cs73Y | 157 |
| CBN-1′-O-β-D-tri-galactoside | Cs73Y | 157 |
| CBN-1′-O-β-D-tri-N-acetylglucosaminoside | Cs73Y | 157 |
| CBN-1′-O-β-D-tri-arabinoside | Cs73Y | 157 |
| CBN-1′-O-β-D-tri-N-acetylgalactosaminoside | Cs73Y | 157 |
| CBD-3′-O-β-D-tri-xyloside | Cs73Y | 157 |
| CBD-3′-O-α-D-tri-rhamnoside | Cs73Y | 157 |
| CBD-3′-O-β-D-tri-galactoside | Cs73Y | 157 |
| CBD-3′-O-β-D-tri-N-acetylglucosaminoside | Cs73Y | 157 |
| CBD-3′-O-β-D-tri-arabinoside | Cs73Y | 157 |
| CBD-3′-O-β-D-tri-N-acetylgalactosaminoside | Cs73Y | 157 |
| CBDV-3′-O-β-D-tri-xyloside | Cs73Y | 157 |
| CBDV-3′-O-α-D-tri-rhamnoside | Cs73Y | 157 |
| CBDV-3′-O-β-D-tri-galactoside | Cs73Y | 157 |
| CBDV-3′-O-β-D-tri-N-acetylglucosaminoside | Cs73Y | 157 |
| CBDV-3′-O-β-D-tri-arabinoside | Cs73Y | 157 |
| CBDV-3′-O-β-D-tri-N-acetylgalactosaminoside | Cs73Y | 157 |
| CBG-3′-O-β-D-tri-xyloside | Cs73Y | 157 |
| CBG-3′-O-α-D-tri-rhamnoside | Cs73Y | 157 |
| CBG-3′-O-β-D-tri-galactoside | Cs73Y | 157 |
| CBG-3′-O-β-D-tri-N-acetylglucosaminoside | Cs73Y | 157 |
| CBG-3′-O-β-D-tri-arabinoside | Cs73Y | 157 |
| CBG-3′-O-β-D-tri-N-acetylgalactosaminoside | Cs73Y | 157 |
| CBD-1′-O-β-D-tetra-xyloside | Cs73Y | 157 |
| CBD-1′-O-α-D-tetra-rhamnoside | Cs73Y | 157 |
| CBD-1′-O-β-D-tetra-galactoside | Cs73Y | 157 |
| CBD-1′-O-β-D-tetra-N-acetylglucosaminoside | Cs73Y | 157 |
| CBD-1′-O-β-D-tetra-arabinoside | Cs73Y | 157 |
| CBD-1′-O-β-D-tetra-N-acetylgalactosaminoside | Cs73Y | 157 |
| CBDV-1′-O-β-D-tetra-xyloside | Cs73Y | 157 |
| CBDV-1′-O-α-D-tetra-rhamnoside | Cs73Y | 157 |
| CBDV-1′-O-β-D-tetra-galactoside | Cs73Y | 157 |
| CBDV-1′-O-β-D-tetra-N-acetylglucosaminoside | Cs73Y | 157 |
| CBDV-1′-O-β-D-tetra-arabinoside | Cs73Y | 157 |
| CBDV-1′-O-β-D-tetra-N-acetylgalactosaminoside | Cs73Y | 157 |
| CBG-1′-O-β-D-tetra-xyloside | Cs73Y | 157 |
| CBG-1′-O-α-D-tetra-rhamnoside | Cs73Y | 157 |
| CBG-1′-O-β-D-tetra-galactoside | Cs73Y | 157 |
| CBG-1′-O-β-D-tetra-N-acetylglucosaminoside | Cs73Y | 157 |
| CBG-1′-O-β-D-tetra-arabinoside | Cs73Y | 157 |
| CBG-1′-O-β-D-tetra-N-acetylgalactosaminoside | Cs73Y | 157 |
| CBD-3′-O-β-D-tetra-xyloside | Cs73Y | 157 |
| CBD-3′-O-α-D-tetra-rhamnoside | Cs73Y | 157 |
| CBD-3′-O-β-D-tetra-galactoside | Cs73Y | 157 |
| CBD-3′-O-β-D-tetra-N-acetylglucosaminoside | Cs73Y | 157 |
| CBD-3′-O-β-D-tetra-arabinoside | Cs73Y | 157 |
| CBD-3′-O-β-D-tetra-N-acetylgalactosaminoside | Cs73Y | 157 |
| CBDV-3′-O-β-D-tetra-xyloside | Cs73Y | 157 |
| CBDV-3′-O-α-D-tetra-rhamnoside | Cs73Y | 157 |
| CBDV-3′-O-β-D-tetra-galactoside | Cs73Y | 157 |
| CBDV-3′-O-β-D-tetra-N-acetylglucosaminoside | Cs73Y | 157 |
| CBDV-3′-O-β-D-tetra-arabinoside | Cs73Y | 157 |
| CBDV-3′-O-β-D-tetra-N-acetylgalactosaminoside | Cs73Y | 157 |
| CBG-3′-O-β-D-tetra-xyloside | Cs73Y | 157 |
| CBG-3′-O-α-D-tetra-rhamnoside | Cs73Y | 157 |
| CBG-3′-O-β-D-tetra-galactoside | Cs73Y | 157 |
| CBG-3′-O-β-D-tetra-N-acetylglucosaminoside | Cs73Y | 157 |
| CBG-3′-O-β-D-tetra-arabinoside | Cs73Y | 157 |
| CBG-3′-O-β-D-tetra-N-acetylgalactosaminoside | Cs73Y | 157 |
| CBD-1′-O-β-D-glucosyl-3′-O-β-D-cellobiose | Cs73Y + OsEUGT11 | 157, 115 |
| CBD-1′-O-β-D-glucosyl-3′-O-β-D-gentiobiose | Cs73Y + Si94D | 157, 145 |
| CBD-1′-O-β-D-xylosyl-3′-O-β-D-xyloside | Cs73Y | 157 |
| CBD-1′-O-a-L-rhamnosyl-3′-O-α-L-rhamnoside | Cs73Y | 157 |
| CBD-1′-O-β-D-galactosyl-3′-O-β-D-galactoside | Cs73Y | 157 |
| CBD-1′-O-β-D-N-acetylglucosaminyl-3′-O-β-D-N-acetylglucosaminoside | Cs73Y | 157 |
| CBD-1′-O-β-D-arabinosyl-3′-O-β-D-arabinoside | Cs73Y | 157 |
| CBD-1′-O-β-D-N-acetylgalactosaminyl-3′-O-β-D-N-acetylgalactosaminoside | Cs73Y | 157 |
| CBDV-1′-O-β-D-glucosyl-3′-O-β-D-cellobiose | Cs73Y + OsEUGT11 | 157, 115 |
| CBDV-1′-O-β-D-glucosyl-3′-O-β-D-gentiobiose | Cs73Y + Si94D | 157, 145 |
| CBDV-1′-O-β-D-glucosyl-3′-O-β-D-xyloside | Cs73Y | 157 |
| CBDV-1′-O-α-D-glucosyl-3′-O-α-L-rhamnoside | Cs73Y | 157 |
| CBDV-1′-O-β-D-glucosyl-3′-O-β-D-galactoside | Cs73Y | 157 |
| CBDV-1′-O-β-D-glucosyl-3′-O-β-D-N-acetylglucosaminoside | Cs73Y | 157 |
| CBDV-1′-O-β-D-glucosyl-3′-O-β-D-arabinoside | Cs73Y | 157 |
| CBDV-1′-O-β-D-glucosyl-3′-O-β-D-N-acetylgalactosaminoside | Cs73Y | 157 |
| CBG-1′-O-β-D-glucosyl-3′-O-β-D-cellobiose | Cs73Y + OsEUGT11 | 157, 115 |
| CBG-1′-O-β-D-glucosyl-3′-O-β-D-gentiobiose | Cs73Y + Si94D | 157, 145 |
| CBG-1′-O-β-D-glucosyl-3′-O-β-D-xyloside | Cs73Y | 157 |
| CBG-1′-O-α-D-glucosyl-3′-O-α-L-rhamnoside | Cs73Y | 157 |
| CBG-1′-O-β-D-glucosyl-3′-O-β-D-galactoside | Cs73Y | 157 |
| CBG-1′-O-β-D-glucosyl-3′-O-β-D-N-acetylglucosaminoside | Cs73Y | 157 |
| CBG-1′-O-β-D-glucosyl-3′-O-β-D-arabinoside | Cs73Y | 157 |
| CBG-1′-O-β-D-glucosyl-3′-O-β-D-N-acetylgalactosaminoside | Cs73Y | 157 |
| CBD-1′-O-β-D-cellobiosyl-3′-O-β-D-glucoside | Cs73Y + OsEUGT11 | 157, 115 |
| CBD-1′-O-β-D-gentiobiosyl-3′-O-β-D-glucoside | Cs73Y + Si94D | 157, 145 |
| CBDV-1′-O-β-D-cellobiosyl-3′-O-β-D-glucoside | Cs73Y + OsEUGT11 | 157, 115 |
| CBDV-1′-O-β-D-gentiobiosyl-3′-O-β-D-glucoside | Cs73Y + Si94D | 157, 145 |
| CBDA-1′-O-β-D-cellobiosyl-3′-O-β-D-glucoside | Cs73Y + OsEUGT11 | 157, 115 |
| CBDA-1′-O-β-D-gentiobiosyl-3′-O-β-D-glucoside | Cs73Y + Si94D | 157, 145 |
| CBG-1′-O-β-D-cellobiosyl-3′-O-β-D-glucoside | Cs73Y + OsEUGT11 | 157, 115 |
| CBG-1′-O-β-D-gentiobiosyl-3′-O-β-D-glucoside | Cs73Y + Si94D | 157, 145 |
| CBD-1′-O-β-D-xylosyl-3′-O-β-D-di-xyloside | Cs73Y | 157 |
| CBD-1′-O-α-L-rhamnosyl-3′-O-α-L-di-rhamnoside | Cs73Y | 157 |
| CBD-1′-O-β-D-galactosyl-3′-O-β-D-di-galactoside | Cs73Y | 157 |
| CBD-1′-O-β-D-N-acetylglucosaminyl-3′-O-β-D-di-N-acetylglucosaminoside | Cs73Y | 157 |
| CBD-1′-O-β-D-arabinosyl-3′-O-β-D-di-arabinoside | Cs73Y | 157 |
| CBD-1′-O-β-D-N-acetylgalactosaminyl-3′-O-β-D-di-N-acetylgalactosaminoside | Cs73Y | 157 |
| CBDV-1′-O-β-D-xylosyl-3′-O-β-D-di-xyloside | Cs73Y | 157 |
| CBDV-1′-O-α-L-rhamnosyl-3′-O-α-L-di-rhamnoside | Cs73Y | 157 |
| CBDV-1′-O-β-D-galactosyl-3′-O-β-D-di-galactoside | Cs73Y | 157 |
| CBDV-1′-O-β-D-N-acetylglucosaminyl-3′-O-β-D-di-N-acetylglucosaminoside | Cs73Y | 157 |
| CBDV-1′-O-β-D-arabinosyl-3′-O-β-D-di-arabinoside | Cs73Y | 157 |
| CBDV-1′-O-β-D-N-acetylgalactosaminyl-3′-O-β-D-di-N-acetylgalactosaminoside | Cs73Y | 157 |
| CBG-1′-O-β-D-xylosyl-3′-O-β-D-di-xyloside | Cs73Y | 157 |
| CBG-1′-O-α-L-rhamnosyl-3′-O-α-L-di-rhamnoside | Cs73Y | 157 |
| CBG-1′-O-β-D-galactosyl-3′-O-β-D-di-galactoside | Cs73Y | 157 |
| CBG-1′-O-β-D-N-acetylglucosaminyl-3′-O-β-D-di-N-acetylglucosaminoside | Cs73Y | 157 |
| CBG-1′-O-β-D-arabinosyl-3′-O-β-D-di-arabinoside | Cs73Y | 157 |
| CBG-1′-O-β-D-N-acetylgalactosaminyl-3′-O-β-D-di-N-acetylgalactosaminoside | Cs73Y | 157 |
| CBD-1′-O-β-D-di-xylosyl-3′-O-β-D-xyloside | Cs73Y | 157 |
| CBD-1′-O-α-L-di-rhamnosyl-3′-O-α-L-rhamnoside | Cs73Y | 157 |
| CBD-1′-O-β-D-di-galactosyl-3′-O-β-D-galactoside | Cs73Y | 157 |
| CBD-1′-O-β-D-di-N-acetylglucosaminyl-3′-O-β-D-N-acetylglucosaminoside | Cs73Y | 157 |
| CBD-1′-O-β-D-di-arabinosyl-3′-O-β-D-arabinoside | Cs73Y | 157 |
| CBD-1′-O-β-D-di-N-acetylgalactosaminyl-3′-O-β-D-N-acetylgalactosaminoside | Cs73Y | 157 |
| CBDV-1′-O-β-D-di-xylosyl-3′-O-β-D-xyloside | Cs73Y | 157 |
| CBDV-1′-O-α-L-di-rhamnosyl-3′-O-α-L-rhamnoside | Cs73Y | 157 |
| CBDV-1′-O-β-D-di-galactosyl-3′-O-β-D-galactoside | Cs73Y | 157 |
| CBDV-1′-O-β-D-di-N-acetylglucosaminyl-3′-O-β-D-N- acetylglucosaminoside | Cs73Y | 157 |
| CBDV-1′-O-β-D-di-arabinosyl-3′-O-β-D-arabinoside | Cs73Y | 157 |
| CBDV-1′-O-β-D-di-N-acetylgalactosaminyl-3′-O-β-D-N-acetylgalactosaminoside | Cs73Y | 157 |
| CBG-1′-O-β-D-di-xylosyl-3′-O-β-D-xyloside | Cs73Y | 157 |
| CBG-1′-O-α-L-di-rhamnosyl-3′-O-α-L-rhamnoside | Cs73Y | 157 |
| CBG-1′-O-β-D-di-galactosyl-3′-O-β-D-galactoside | Cs73Y | 157 |
| CBG-1′-O-β-D-di-N-acetylglucosaminyl-3′-O-β-D-N- acetylglucosaminoside | Cs73Y | 157 |
| CBG-1′-O-β-D-di-arabinosyl-3′-O-β-D-arabinoside | Cs73Y | 157 |
| CBG-1′-O-β-D-di-N-acetylgalactosaminyl-3′-O-β-D-N-acetylgalactosaminoside | Cs73Y | 157 |
| CBD-1′-O-β-D-di-xylosyl-3′-O-β-D-di-xyloside | Cs73Y | 157 |
| CBD-1′-O-α-D-di-rhamnosyl-3′-O-α-D-di-rhamnoside | Cs73Y | 157 |
| CBD-1′-O-β-D-di-galactosyl-3′-O-β-D-di-galactoside | Cs73Y | 157 |
| CBD-1′-O-β-D-di-N-acetylglucosaminyl-3′-O-β-D-di-N-acetylglucosaminoside | Cs73Y | 157 |
| CBD-1′-O-β-D-di-arabinosyl-3′-O-β-D-di-arabinoside | Cs73Y | 157 |
| CBD-1′-O-β-D-di-N-acetylgalactosaminyl-3′-O-β-D-di-N-acetylgalactosaminoside | Cs73Y | 157 |
| CBDV-1′-O-β-D-di-xylosyl-3′-O-β-D-di-xyloside | Cs73Y | 157 |
| CBDV-1′-O-α-D-di-rhamnosyl-3′-O-α-D-di-rhamnoside | Cs73Y | 157 |
| CBDV-1′-O-β-D-di-galactosyl-3′-O-β-D-di-galactoside | Cs73Y | 157 |
| CBDV-1′-O-β-D-di-N-acetylglucosaminyl-3′-O-β-D-di-N-acetylglucosaminoside | Cs73Y | 157 |
| CBDV-1′-O-β-D-di-arabinosyl-3′-O-β-D-di-arabinoside | Cs73Y | 157 |
| CBDV-1′-O-β-D-di-N-acetylgalactosaminyl-3′-O-β-D-di-N-acetylgalactosaminoside | Cs73Y | 157 |
| CBG-1′-O-β-D-di-xylosyl-3′-O-β-D-di-xyloside | Cs73Y | 157 |
| CBG-1′-O-α-D-di-rhamnosyl-3′-O-α-D-di-rhamnoside | Cs73Y | 157 |
| CBG-1′-O-β-D-di-galactosyl-3′-O-β-D-di-galactoside | Cs73Y | 157 |
| CBG-1′-O-β-D-di-N-acetylglucosaminyl-3′-O-β-D-di-N-acetylglucosaminoside | Cs73Y | 157 |
| CBG-1′-O-β-D-di-arabinosyl-3′-O-β-D-di-arabinoside | Cs73Y | 157 |
| CBG-1′-O-β-D-di-N-acetylgalactosaminyl-3′-O-β-D-di-N-acetylgalactosaminoside | Cs73Y | 157 |

### Example 20—Combining Multiple Glycosyl Transferases Catalyzes Conversion of Cannabinoid Substrates to Cannabinoid Glycosides with Alternate Sugar-Sugar Linkages

The glycosyl transferases described herein can broadly be grouped into either glycosyl transferases active on the cannabinoid aglycones or glycosyl transferases active on cannabinoid glycosides. The latter group, instead of attaching a sugar moiety onto a free hydroxy group on the cannabinoid molecule, attaches a sugar moiety onto the sugar group of the cannabinoid glycoside. In Example 13 a range of glycosyl transferases were discovered that were active only on cannabinoid aglycones (e.g. PL-159(Pt88G_GA) (SEQ ID NO: 147, 148)) as well a range of glycosyl transferases which were active on both cannabinoid aglycones and cannabinoid glycosides. For example, PL-214(Cs73Y_GA) (SEQ ID NO: 157, 158) was found to produce a range of multi-sugar cannabinoid glycosides which included sugar on cannabinoid linkages as well as sugar on sugar linkages. In Example 13 it was also found that some glycosyl transferases were only active on cannabinoid glycosides and specifically catalyzed sugar on sugar glycosylation reactions. Two of these enzymes (PL-55(Sr76G1_GA) (SEQ ID NO: 123, 124) and PL-32(OsEUGT11_GA) (SEQ ID NO: 115, 116)) are described in prior art and are well known to catalyze a range of sugar on sugar reactions and were recently described as being able to perform sugar on sugar reactions on cannabinoid glycosides. A third enzyme (PL-152(Si94D_GA) (SEQ ID NO: 145, 146)) however is not described in prior art, but in our screen was found to efficiently perform sugar on sugar reactions. Combining multiple glycosyl transferases in a single reaction enables the generation of more a diverse range of cannabinoid glycosides that are not produced by enzymes expressed individually. To demonstrate this, in vitro enzyme assays were performed using CBD and UDP-glucose as substrates. PL-159(Pt88G_GA), previously demonstrated to produce CBD-1′-O-β-D-glucoside (OB1) was combined with enzymes previously demonstrated to attach a second glucose molecule to the glucose moiety of CBD-1′-O-β-D-glucoside (OB1) (PL-55(Sr76G1_GA) (SEQ ID NO: 123, 124), PL-32(OsEUGT11_GA) (SEQ ID NO: 115, 116), PL-152(Si94D_GA) (SEQ ID NO: 145, 146)). In vitro assays were performed and analyzed as described previously. In the prior art, Sr76G1 was described as being able to convert cannabinoid aglycones into cannabinoid glycosides, while surprisingly we did not detect any activity with this enzyme using cannabinoid aglycones as substrate, we did detect activity using cannabinoid glycosides as substrates. It was found that when combined with Pt88G, all 3 enzymes could convert OB1 to CBD-di-glucoside derivatives (OB2-4). By comparing the LC-MS/QTOF retention time, measured mass and fragmentation pattern as well as the c Log P it could be elucidated that Sr76G1, OsEUGT11 and Si94D were catalysing sugar on sugar reactions with different linkages. Sr76G1 was shown to catalyse 1→3 glucose-glucose linkages (laminaribioside), while OsEUGT11 was shown to catalyse both 1→4 glucose-glucose linkages and 1→6 glucose-glucose linkages (gentiobioside). Interestingly, Si94D was shown to catalyse 1-6 glucose-glucose linkages (gentiobioside) with exceptionally high efficiency (100%) as shown in the table below, Table 59. The results conclusively show that Sr76G1 is not active on cannabinoid aglycones but in fact active on glucose molecules. The discovery of enzymes which catalyse sugar-sugar reactions with different linkages greatly expands the diversity of cannabinoid glycosides that can produced with different combinations of Glycosyl transferases.

TABLE 61
In vitro enzymatic conversion of CBD to multi-sugar CBD-glucosides
with different sugar linkages by combining a glycosyl transferase
active on cannabinoid aglycones with glycosyl transferases
active on cannabinoid glucosides. Shown is the amount of CBD
converted to each respective product expressed as a percentage.
Laminaribioside, di-glucoside with 1→3 linkage (OB2); gentiobioside,
di-glucoside with 1→6 linkage (OB3); cellobioside, di-glucoside
with 1→4 linkage (OB4).

| | Structure ID and common name | | | |
|---|---|---|---|---|
| | OB1 | OB2 | OB3 | OB4 |
| | CBD-1′- | CBD-1′- | CBD-1′- | CBD-1′- |
| | O-β- | O-β- | O-β- | O-β- |
| | D-gluco- | D-laminari- | D-gentio- | D-cello- |
| Enzyme(s) | side | bioside | bioside | bioside |
| PL-159(Pt88G_GA) | 97.5 | ND | ND | ND |
| PL-32(OsEUGT11_GA) | ND | ND | ND | ND |
| PL-55(Sr76G1_GA) | ND | ND | ND | ND |
| PL-152(Si94D_GA) | ND | ND | ND | ND |
| PL-159(Pt88G_GA) + | 11.2 | 85.6 | ND | 3.1 |
| PL-32(OsEUGT11_GA) | | | | |
| PL-159(Pt88G_GA) + | 19.3 | 80.7 | ND | ND |
| PL-55(Sr76G1_GA) | | | | |
| PL-159(Pt88G_GA) + | ND | ND | 100.0 | ND |
| PL-152(Si94D_GA) | | | | |

ND; not detected.

### Example 21—Test of Toxicity of Cannabinoids and Cannabinoid Glycosides in S. cerevisiae

It is well known that cannabinoids are toxic to microbes, and it is thought that these compounds are produced by cannabis plants as a defense mechanism against infection. Further, a growing body of evidence is showing various cannabinoids are potent anti-microbials with demonstrated effectiveness against a range of pathogenic bacteria and fungal species. Product toxicity in microbial strains engineered to produce cannabinoids will hinder high-level production of these molecules, glycosylating these molecules can be used to detoxify them and facilitate higher production titers in engineered microbial strains. To measure the toxicity effects of cannabinoids and cannabinoid glycosides wild-type S. cerevisiae strain BY4741 was cultivated in YP media supplemented with 2% glucose and different concentrations of CBD and CBD-1′-O-β-D-glucosyl-3′-O-β-D-glucoside (OB6) dissolved in ethanol, the concentrations were adjusted so that the final concentration of ethanol in all cell cultures was 3%. Cells were inoculated to a starting OD600 of 0.1 and incubated at 30° C. and 200 RPM and the final OD600 was measured after 72 h. As shown in table 60 below, increasing the concentration of CBD in solution results in a progressive decrease in final OD600, while for OB6 the final OD600 remains relatively constant across all concentrations tested. This demonstrates that while CBD is toxic to yeast, OB6 is non-toxic at the concentration range tested.

TABLE 62
Final OD600 of S. cerevisiae cultivated in the
presence of different concentrations of CBD and
CBD-1′-O-β-D-glucosyl-3′-O-β-D-glucoside (OB6).

| Substrate | Concentration (μM) | | | | |
|---|---|---|---|---|---|
| added | 0 | 100 | 200 | 400 | 800 |
| CBD | 9.8 | 9.3 | 8.2 | 6.7 | 4.7 |
| OB6 | 10.1 | 9.1 | 8.8 | 9.5 | 8.8 |

1. A microbial host cell genetically modified to intracellularly produce a cannabinoid glycoside, said cell expressing a heterologous gene encoding a glycosyl transferase which has at least 70% identity to the glycosyl transferase of SEQ ID No: 157 or 207, wherein the glycosyl transferase is capable of intracellularly glycosylating a cannabinoid acceptor with a glycosyl donor thereby producing the cannabinoid glycoside.

2. The microbial host cell of claim 1, wherein the cannabinoid acceptor is a cannabinoid aglycone or a cannabinoid glycoside selected from the group of cannabichromene-type (CBC), cannabigerol-type (CBG), cannabidiol-type (CBD), Tetrahydrocannabinol-type (THC), cannabicyclol-type (CBL), cannabielsoin-type (CBE), cannabinol-type (CBN), cannabinodiol-type (CBND) and cannabitriol-type (CBT).

3. The microbial host cell of claim 1, wherein the cannabinoid acceptor is selected from the group of cannabigerolic acid (CBGA), cannabigerolic acid monomethylether (CBGAM), cannabigerol monomethylether (CBGM), cannabigerovarinic acid (CBGVA), cannabigerovarin (CBGV), cannabichromenic acid (CBCA), cannabichromevarinic acid (CBCVA), cannabichromevarin (CBCV), cannabidiolic acid (CBDA), cannabidiol, monomethylether (CBDM), cannabidiol-C4 (CBD-C4), cannabidivarinic acid (CBDVA), cannabidivarin (CBDV), cannabidiorcol (CBD-C1), Δ9-trans-tetrahydrocannabinol (Δ9-THC), Δ9-tetrahydrocannabinol (Δ9-THC), Δ9-cis-tetrahydrocannabinol (Δ9-THC), tetrahydrocannabinolic acid (THCA), Δ9-tetrahydrocannabinolic acid A (THCA-A), Δ9-tetrahydrocannabinolic acid B (THCA-B), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-trans-tetrahydrocannabinol (Δ8-THC), Δ8-tetrahydrocannabinol (Δ8-THC), Δ8-cis-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitran, cannabicitranic acid, cannabinolic acid, (CBNA), cannabinol methylether (CBNM), cannabinol-C4, (CBN-C4), cannabivarin (CBV), cannabinol-C2 (CNB-C2), cannabiorcol (CBN-C1), cannabinodiol, (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin, (CBTVE), dehydrocannabifuran (DCBF), cannabifuran (CBF), cannabichromanon (CBCN), cannabiciuan (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), perrottetinene, perrottetinenic acid, 11-Nor-9-carboxy-THC, 11-hydroxy-Δ9-THC, Nor-9-carboxy-Δ9-tetrahydrocannabinol, tetrahydrocannabiphorol (THCP), cannabidiphorol (CBDP), Cannabimovone (CBM) and derivatives thereof or the cannabinoid acceptor is an endocannabinoid selected from the group of arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),11(Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, and docosahexaenoic acid (DHA).

4. The microbial host cell of claim 1, wherein the glycosyl donor is selected from one or more of NTP-glycoside, NDP-glycoside and NMP-glycoside, and optionally wherein the nucleoside of the nucleotide glycoside is selected from Uridine, Adenosin, Guanosin, Cytidin and deoxythymidine, and optionally wherein the glycosyl donor is selected from UDP-glycosides, ADP-glycosides, CDP-glycosides, CMP-glycosides, dTDP-glycosides and GDP-glycosides, and optionally wherein the glycosyl donor is selected from UDP-D-glucose (UDP-Glc); UDP-galactose (UDP-Gal); UDP-rhamnose (UDP-Rhm) UDP-D-xylose (UDP-Xyl); UDP-N-acetyl-D-glucosamine (UDP-GlcNAc); UDP-N-acetyl-D-galactosamine (UDP-GalNAc); UDP-D-glucuronic acid (UDP-GlcA); UDP-D-galactofuranose (UDP-Galf); UDP-arabinose; UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc), guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

5. The microbial host cell of claim 1, wherein the cannabinoid glycoside is selected from a glycoside of cannabichromene-type (CBC); cannabigerol-type (CBG); cannabidiol-type (CBD); Tetrahydrocannabinol-type (THC); cannabicyclol-type (CBL); cannabielsoin-type (CBE); cannabinol-type (CBN); cannabinodiol-type (CBND) and cannabitriol-type (CBT), linked to a glycosyl group selected from glucose; cannabionoid glucuronosides; cannabinoid xylosides; cannabinoid rhamnosides; cannabinoid galactosides; cannabinoid N-acetylglucosaminosides; cannabinoid N-acetylgalactosaminosides and cannabinoid arabinosides.

6. The microbial host cell of claim 1, wherein the cannabinoid glycoside is selected from cannabinoid-1′-O-β-D-glucoside; cannabinoid-1′-O-β-D-glucuroside; cannabinoid-1′-O-β-D-xyloside; cannabinoid-1′-O-α-L-rhamnoside; cannabinoid-1′-O-β-D-galactoside; cannabinoid-1′-O-β-D-N-acetylglucosaminoside; cannabinoid-1′-O-β-D-arabinoside; cannabinoid-1′-O-β-D-N-acetylgalactosamine; cannabinoid-1′-O-β-D-cellobioside; cannabinoid-1′-O-β-D-gentiobioside, cannabinoid-1′-O-β-D-glucosyl-3′-O-β-D-glucoside; cannabinoid-1′-O-β-D-glucurosyl-3′-O-β-D-glucuronoside; cannabinoid-1′-O-β-D-xylosyl-3′-O-β-D-xyloside; cannabinoid-1′-O-α-L-rhamnosyl-3′-O-β-D-rhamnoside; cannabinoid-1′-O-β-D-galactosyl-3′-O-β-D-galactoside; cannabinoid-1′-O-β-D-N-acetylglucosamine-3′-O-β-D-N-acetylglucosaminoside; cannabinoid-1′-O-β-D-arabinosyl-3′-O-β-D-arabinoside; and cannabinoid-1′-O-β-D-N-acetylgalactosamine-3′-O-β-D-N-acetylgalactosamine.

7. The microbial host cell of claim 1, wherein the cannabinoid glycoside comprises a cannabinoid aglycone or cannabinoid glycoside covalently linked to a glycosyl moiety by a 1,4 or a 1,6 glycosidic bond.

8. The microbial host cell of claim 1, further comprising an operative biosynthetic metabolic pathway capable of producing the cannabinoid acceptor, wherein the pathway comprises one or more polypeptides selected from
a) an acetoacetyl-CoA thiolase (ACT) converting an acetyl-CoA precursor into acetoacetyl-CoA, optionally an ACT that has at least 70%, identity to the native Erg10 in S. cerevisiae;
b) a HMG-CoA synthase (HCS) converting acetoacetyl-CoA precursor into HMG-CoA, optionally a HCS that has at least 70% identity to the native Erg13 in S. cerevisiae;
c) a HMG-CoA reductase (HCR) converting a HMG-CoA precursor into mevalonate, optionally a HCR that has at least 70% identity to the native HMG1 or HMG2 in S. cerevisiae;
d) a mevalonate kinase (MVK) converting a mevalonate precursor into Mevalonate-5-phosphate, optionally a MVK that has at least 70% identity to the native Erg12 in S. cerevisiae;
e) a phosphomevalonate kinase (PMK) converting a Mevalonate-5-phosphate precursor into Mevalonate diphosphate, optionally a PMK that has at least 70% identity to the native Erg8 in S. cerevisiae;
f) a mevalonate pyrophosphate decarboxylase (MPC) converting a Mevalonate diphosphate precursor into isopentenyl diphosphate (IPP), optionally a MPC that has at least 70% identity to the native MVD1 in S. cerevisiae;
g) an isopentenyl diphosphate/dimethylallyl diphosphate isomerase (IPI) converting an IPP precursor into dimethylallyl diphosphate (DMAPP), optionally an IPI that has at least 70% identity to the native IDI1 in S. cerevisiae;
h) Geranyl diphosphate synthase (GPPS) condensing IPP and DMAPP into Geranyl diphosphate (GPP), optionally a GPPS that has at least 70% identity to the GPPS comprised in SEQ ID NO: 45 or 229;
i) an acyl activating enzyme (AAE) converting a fatty acid precursor into fatty acyl-COA, optionally an AAE that has at least 70% identity to the AAE comprised in SEQ ID NO: 47 or 239;
j) a 3,5,7-Trioxododecanoyl-CoA synthase (TKS) converting a fatty acid-CoA precursor into 3,5,7-trioxoundecanoyl-CoA, optionally a TKS that has at least 70% identity to the TKS comprised in SEQ ID NO: 49;
k) an Olivetolic Acid Cyclase (OAC) converting a 3,5,7-trioxoundecanoyl-CoA precursor into divarinolic acid, optionally an OAC that has at least 70% identity to the OAC comprised in SEQ ID NO: 51;
l) an Olivetolic Acid Cyclase (OAC) converting a 3,5,7-trioxododecanoyl-CoA precursor into olivetolic acid, optionally an OAC that has at least 70% identity to the OAC comprised in SEQ ID NO: 51;
m) a TKS-OAC fused enzyme converting fatty acid-CoA precursor into 3,5,7-trioxoundecanoyl-CoA, 3,5,7-trioxoundecanoyl-CoA precursor into divarinolic acid and 3,5,7-trioxododecanoyl-CoA precursor into olivetolic acid, optionally a TKS-OAC fused enzyme at least 70% identity to the TKS-OAC fused enzyme comprised in SEQ ID NO 227;
n) a Cannabigerolic acid synthase (CBGAS) condensing GPP and olivetolic acid into Cannabigerolic acid (CBGA), optionally a CBGAS that has at least 70% identity to the CBGAS comprised in SEQ ID NO: 53, 235 or 237;
o) a Cannabigerolic acid synthase (CBGAS) condensing GPP and divarinolic acid into cannabigerovarinic acid (CBGVA), optionally optionally a CBGAS that has at least 70% identity to the CBGAS comprised in SEQ ID NO: 53, 235 or 237;
p) a cannabidiolic acid synthase (CBDAS) converting CBGA acid and/or CBGVA into cannabidiolic acid (CBDA) and/or cannabidivarinic acid (CBDVA) respectively, optionally a CBDAS that has at least 70% identity to the CBDAS comprised in SEQ ID NO: 57 or 233;
q) a tetrahydrocannabinolic acid synthase (THCAS) converting CBGA and/or CBGVA into tetrahydrocannabinolic acid (THCA) and/or tetrahydrocannabivarinic acid (THCVA) respectively, optionally a THCAS that has at least 70% identity to the THCAS comprised in SEQ ID NO: 55 or 231;
r) a cannabichromenic acid synthase (CBCAS) converting CBGA and/or CBGVA into cannabichromenic acid (CBCA) and/or cannabichromevarinic acid (CBCVA) respectively, optionally a CBCAS that has at least 70% identity to the CBCAS comprised in SEQ ID NO: 59;
s) a nucleotide-glucose synthase converting sucrose and nucleotide into fructose and nucleotide-glucose, optionally an UDP-glucose synthase that has at least 70% identity to the UDP-glucose synthase comprised in SEQ ID NO: 209;
t) a nucleotide-galactose 4 epimerase converting nucleotide-glucose into nucleotide-galactose, optionally an UDP-galactose 4-epimerase that has at least 70% identity to the UDP-galactose 4-epimerase comprised in SEQ ID NO: 211;
u) a nucleotide-(glucuronic acid) decarboxylase converting nucleotide-glucuronic acid into nucleotide-xylose, optionally an UDP-glucuronic acid decarboxylase that has at least 70% identity to the UDP-glucuronic acid decarboxylase comprised in SEQ ID NO: 213;
v) a nucleotide-4-keto-6-deoxy-glucose 3,5 epimerase and a nucleotide-4-keto-rhamnose 4-keto-reductase together converting nucleotide-4-keto-6-deoxy-glucose and NADPH into nucleotide-rhamnose and NADP+, optionally an UDP-4-keto-6-deoxy-glucose 3,5 epimerase that has at least 70% identity to the UDP-4-keto-6-deoxy-glucose 3,5 epimerase comprised in SEQ ID NO: 215 or 219 and an UDP-4-keto-rhamnose-4-keto reductase that has at least 70% identity to the UDP-4-keto-rhamnose-4-keto reductase comprised in SEQ ID NO: 215 or 219;
w) a nucleotide-glucose 4,6 dehydratase converting nucleotide-glucose and NAD into nucleotide-4-keto-6-deoxy-glucose and NADH, optionally an UDP-glucose 4,6 dehydratase that has at least 70% identity to the UDP-glucose 4,6 dehydratase comprised in SEQ ID NO: 217 or 219;
x) a nucleotide-glucose 4,6-dehydratase and a nucleotide-4-keto-6-deoxy-glucose 3,5 epimerase and a nucleotide-4-keto-rhamnose-4-keto-reductase together converting nucleotide-glucose and NAD+ and NADPH into nucleotide-rhamnose+NADH+NADP+, optionally an UDP-4-keto-6-deoxy-glucose 3,5 epimerase that has at least 70% identity to the UDP-4-keto-6-deoxy-glucose 3,5 epimerase comprised in SEQ ID NO: 215 or 219 and an UDP-4-keto-rhamnose-4-keto reductase that has at least 70% identity to the UDP-4-keto-rhamnose-4-keto reductase comprised in SEQ ID NO: 215 or 219 and an UDP-glucose 4,6 dehydratase that has at least 70% identity to the UDP-glucose 4,6 dehydratase comprised in SEQ ID NO: 217 or 219;
y) a nucleotide-glucose 6 dehydrogenase converting nucleotide-glucose and 2 NAD+ into nucleotide-glucuromic acid and 2 NADH, optionally an UDP-glucose 6 dehydrogenase that has at least 70% identity to the UDP-glucose 6 dehydrogenase comprised in SEQ ID NO: 221;
z) a nucleotide-arabinose 4 epimerase converting nucleotide-xylose into nucleotide-arabinose, optionally an UDP-arabinose 4 epimerase that has at least 70% identity to the UDP-arabinose 4 epimerase comprised in SEQ ID NO: 223; and
aa) a nucleotide-N-acetylglucosamine 4 epimerase converting nucleotide-N-acetylglucosamine into nucleotide-N-acetylgalactosamine, optionally an UDP-N-acetylglucosamine 4 epimerase that has at least 70% identity to the UDP-N-acetylglucosamine 4 epimerase comprised in SEQ ID NO: 225.

9. A cell culture, comprising the microbial host cell of claim 1 and a growth medium.

10. A method for producing a cannabinoid glycoside comprising contacting a cannabinoid acceptor with a glycosyl transferase which has at least 70% identity to the glycosyl transferase of SEQ ID NO: 157 or 207 and with one or more nucleotide glycosides at conditions allowing the glycosyl transferase to transfer the glycosyl moiety of the nucleotide glycoside to the cannabinoid acceptor.

11. The method of claim 10, wherein the glycosylation is performed in vitro.

12. The method of claim 10 further comprising the steps of:
a) culturing a cell culture comprising a microbial host cell genetically modified to intracellularly produce a cannabinoid glycoside and a growth medium, wherein the microbial host cell expresses a heterologous ene encoding the glycosyl transferase, at conditions allowing the genetically microbial host cell to produce the cannabinoid glycoside; and
b) optionally recovering and/or isolating the cannabinoid glycoside.

13. A fermentation liquid comprising the cannabinoid glycosides comprised in the cell culture of claim 9.

14. The fermentation liquid of claim 13, further comprising one or more compounds selected from:
a) precursors or products of the operative biosynthetic metabolic pathway producing the Cannabinoid glycoside;
b) supplemental nutrients comprising trace metals, vitamins, salts, yeast nitrogen base, YNB, and/or amino acids; and
wherein the concentration of the cannabinoid glycoside is at least 1 mg/l liquid.

15. A cannabinoid glycoside comprising a cannabinoid aglycone or cannabinoid glycoside covalently linked to a sugar selected from xylose; rhamnose; galactose; N-acetylglucosamine; N-acetylgalactosamine; and arabinose.

16. The cannabinoid glycoside of claim 15, wherein the cannabinoid glycoside is selected from cannabinoid-1′-O-β-D-xyloside; cannabinoid-1′-O-α-L-rhamnoside; cannabinoid-1′-O-β-D-galactoside; cannabinoid-1′-O-β-D-N-acetylglucosaminoside; cannabinoid-1′-O-β-D-arabinoside; cannabinoid-1′-O-β-D-N-acetylgalactosamine; cannabinoid-1′-O-β-D-cellobioside, cannabinoid-1′-β-D-gentiobioside; cannabinoid-1′-O-β-D-xylosyl-3′-O-β-D-xyloside; cannabinoid-1′-O-α-L-rhamnosyl-3′-O-β-D-rhamnoside; cannabinoid-1′-O-β-D-galactosyl-3′-O-β-D-galactoside; cannabinoid-1′-O-β-D-N-acetylglucosamine-3′-O-β-D-N-acetylglucosaminoside; cannabinoid-1′-O-β-D-arabinosyl-3′-O-β-D-arabinoside; and cannabinoid-1′-O-β-D-N-acetylgalactosamine-3′-O-β-D-N-acetylgalactosamine.

17. A cannabinoid glycoside comprising a cannabinoid aglycone or cannabinoid glycoside covalently linked to a glycosyl moiety by a 1,4 or a 1,6 glycosidic bond.

18. A composition comprising the fermentation liquid of claim 13 and a cannabinoid glycoside and one or more agents, additives and/or excipients.

19. A method for preparing a pharmaceutical preparation comprising mixing the cannabionoid glycoside of claim 15 with one or more pharmaceutical grade excipients, additives and/or adjuvants.

20. A pharmaceutical preparation obtainable from the method of claim 19.

21. A pharmaceutical preparation obtainable from the method of claim 19 for use as a medicament or a prodrug.

22. A method for treating a disease in a mammal, comprising administering a therapeutically effective amount of the pharmaceutical preparation of claim 20 to the mammal.


SEQUENCE LISTING (320 sequences)

SEQ ID NO: 1 - protein, 472 aa, Citrus hanaju
MSDSGGFDSHPHVALIPSAGMGHLTPFLRLAASLVQHHCRVTLITTYPTVSLAETQHVSHFLSAYPQVTENRFHLLPFDPNSANATDPFLLRWEAIRRSAHLLAPLLSPPLSALITDVTLISAVLPVTINLHLPNYVLFTASAKMFSLTASFPAIVASKSTSSGSVEFDDDFIEIPGLPPIPLSSVPPAVMDSKSLFATSFLENGNSFVKSNGVLINSFDALEADTLVALNGRRVVAGLPPVYAVGPLLPCEFEKRDDPSTSLILKWLDDQPEGSVVYVSFGSRLALSMEQTKELGDGLLSSGCRFLWVVKGKNVDKEDEESLKNVLGHELTEKIKDQGLVVKNWVDQDKVLSHRAVGGFVSHGGWNSLVEAARHGVPVLVWPHFGDQKINAEAVERAGLGMWVRSWGWGTELRAKGDEIGLKIKDLMANDFLREQAKRSEEEARKAIGVGGSSERTFKELIDKWKCNNNTH

SEQ ID NO: 2 - DNA, 1419 bp, Citrus hanaju
atgtctgactctggtggtttcgactctcacccacacgttgctttgatcccatctgctggtatgggtcacttgactccattcttgagattggctgcttctttggttcaacaccactgtagagttactttgatcactacttacccaactgtttctttggctgaaactcaacacgtttctcacttcttgtctgcttacccacaagttactgaaaacagattccacttgttgccattcgacccaaactctgctaacgctactgacccattcttgttgagatgggaagctatcagaagatctgctcacttgttggctccattgttgtctccaccattgtctgctttgatcactgacgttactttgatctctgctgttttgccagttactatcaacttgcacttgccaaactacgttttgttcactgcttctgctaagatgttctctttgactgcttctttcccagctatcgttgcttctaagtctacttcttctggttctgttgaattcgacgacgacttcatcgaaatcccaggtttgccaccaatcccattgtcttctgttccaccagctgttatggactctaagtctttgttcgctacttctttcttggaaaacggtaactctttcgttaagtctaacggtgttttgatcaactctttcgacgctttggaagctgacactttggttgctttgaacggtagaagagttgttgctggtttgccaccagtttacgctgttggtccattgttgccatgtgaattcgaaaagagagacgacccatctacttctttgatcttgaagtggttggacgaccaaccagaaggttctgttgtttacgtttctttcggttctagattggctttgtctatggaacaaactaaggaattgggtgacggtttgttgtcttctggttgtagattcttgtgggttgttaagggtaagaacgttgacaaggaagacgaagaatctttgaagaacgttttgggtcacgaattgactgaaaagatcaaggaccaaggtttggttgttaagaactgggttgaccaagacaaggttttgtctcacagagctgttggtggtttcgtttctcacggtggttggaactctttggttgaagctgctagacacggtgttccagttttggtttggccacacttcggtgaccaaaagatcaacgctgaagctgttgaaagagctggtttgggtatgtgggttagatcttggggttggggtactgaattgagagctaagggtgacgaaatcggtttgaagatcaaggacttgatggctaacgacttcttgagagaacaagctaagagatctgaagaagaagctagaaaggctatcggtgttggtggttcttctgaaagaactttcaaggaattgatcgacaagtggaagtgtaacaacaacactcactag

SEQ ID NO: 3 - protein, 472 aa, Citrus hanaju
MSDSGGFDSHPHVALIPSAGMGHLTPFLRLAASLVQHHCRVTLITTYPTVSLAETQHVSHFLSAYPQVTEKRFHLLPFDPNSANATDPFLLRWEAIRRSAHLLAPLLSPPLSALITDVTLISAVLPVTINLHLPNYVLFTASAKMFSLTASFPAIVASKSTSSGSVEFDDDFIEIPGLPPIPLSSVPPAVMDSKSLFATSFLENGNSFVKSNGVLINSFDALEADTLVALNGRRVVAGLPPVYAVGPLLPCEFEKRDDPSTSLILKWLDDQPEGSVVYVSFGSRLALSMEQTKELGDGLLSSGCRFLWVVKGKIVDKEDEESLKNVLGHELTEKIKDQGLVVKNWVDQDKVLSHRAVGGFVSHGGWNSLVEAARHGVPLLVWPHFGDQKINAEAVERAGLGMWVRSWGWGTELRAKGDEIGLKIKDLMANDFLREQAKRIEEEARKAIGVGGSSERTFKELIDKWKCNNNTH

SEQ ID NO: 4 - DNA, 1419 bp, Citrus hanaju
atgtctgactctggtggtttcgactctcacccacacgttgctttgatcccatctgctggtatgggtcacttgactccattcttgagattggctgcttctttggttcaacaccactgtagagttactttgatcactacttacccaactgtttctttggctgaaactcaacacgtttctcacttcttgtctgcttacccacaagttactgaaaagagattccacttgttgccattcgacccaaactctgctaacgctactgacccattcttgttgagatgggaagctatcagaagatctgctcacttgttggctccattgttgtctccaccattgtctgctttgatcactgacgttactttgatctctgctgttttgccagttactatcaacttgcacttgccaaactacgttttgttcactgcttctgctaagatgttctctttgactgcttctttcccagctatcgttgcttctaagtctacttcttctggttctgttgaattcgacgacgacttcatcgaaatcccaggtttgccaccaatcccattgtcttctgttccaccagctgttatggactctaagtctttgttcgctacttctttcttggaaaacggtaactctttcgttaagtctaacggtgttttgatcaactctttcgacgctttggaagctgacactttggttgctttgaacggtagaagagttgttgctggtttgccaccagtttacgctgttggtccattgttgccatgtgaattcgaaaagagagacgacccatctacttctttgatcttgaagtggttggacgaccaaccagaaggttctgttgtttacgtttctttcggttctagattggctttgtctatggaacaaactaaggaattgggtgacggtttgttgtcttctggttgtagattcttgtgggttgttaagggtaagatcgttgacaaggaagacgaagaatctttgaagaacgttttgggtcacgaattgactgaaaagatcaaggaccaaggtttggttgttaagaactgggttgaccaagacaaggttttgtctcacagagctgttggtggtttcgtttctcacggtggttggaactctttggttgaagctgctagacacggtgttccattgttggtttggccacacttcggtgaccaaaagatcaacgctgaagctgttgaaagagctggtttgggtatgtgggttagatcttggggttggggtactgaattgagagctaagggtgacgaaatcggtttgaagatcaaggacttgatggctaacgacttcttgagagaacaagctaagagaatcgaagaagaagctagaaaggctatcggtgttggtggttcttctgaaagaactttcaaggaattgatcgacaagtggaagtgtaacaacaacactcactag

SEQ ID NO: 5 - protein, 472 aa, Fortunella crassifolia
MSDSGGFDSHPHVALIPSAGMGHLTPFLRLAASLVQHHCRVTLITTYPTVSLAETQHVSHFLSAYPQVTEKRFHLLPFDPNSANATDPFFLRWEAIRRSAHLLAPLLSPPLSALITDVTLISAVLPVTINLHLPNYVLFTASARMFSLTASFPAIVASKSTSSGSVEFDDDFIEIPGLPPIPLSSVPPAVMDSKSLFATSFLENGNSFVKSNGVLINSFDALEADTLVALNGRRVVAGLPPVYAVGPLLPCEFEKRDDPSTSLILKWLDDQPEGSVVYVSFGSRLALSMEQTKELGNGLLSSGCRFLWVVKGKTVDKEDEESLKNVLGHELMEKIKDQGLVVKNWVDQDKVLSHRAVGGFVSHGGWNSLVEAARHGVPVLVWPQFGDQKINAEAVESAGLGMWVRSWGWGTELRAKGDEIGLKIKDLMANDFLREQAKRIEEEARKAIGVGGSSERTFKELIDKWKCNNNTH

SEQ ID NO: 6 - DNA, 1419 bp, Fortunella crassifolia
atgtctgactctggtggtttcgactctcacccacacgttgctttgatcccatctgctggtatgggtcacttgactccattcttgagattggctgcttctttggttcaacaccactgtagagttactttgatcactacttacccaactgtttctttggctgaaactcaacacgtttctcacttcttgtctgcttacccacaagttactgaaaagagattccacttgttgccattcgacccaaactctgctaacgctactgacccattcttcttgagatgggaagctatcagaagatctgctcacttgttggctccattgttgtctccaccattgtctgctttgatcactgacgttactttgatctctgctgttttgccagttactatcaacttgcacttgccaaactacgttttgttcactgcttctgctagaatgttctctttgactgcttctttcccagctatcgttgcttctaagtctacttcttctggttctgttgaattcgacgacgacttcatcgaaatcccaggtttgccaccaatcccattgtcttctgttccaccagctgttatggactctaagtctttgttcgctacttctttcttggaaaacggtaactctttcgttaagtctaacggtgttttgatcaactctttcgacgctttggaagctgacactttggttgctttgaacggtagaagagttgttgctggtttgccaccagtttacgctgttggtccattgttgccatgtgaattcgaaaagagagacgacccatctacttctttgatcttgaagtggttggacgaccaaccagaaggttctgttgtttacgtttctttcggttctagattggctttgtctatggaacaaactaaggaattgggtaacggtttgttgtcttctggttgtagattcttgtgggttgttaagggtaagactgttgacaaggaagacgaagaatctttgaagaacgttttgggtcacgaattgatggaaaagatcaaggaccaaggtttggttgttaagaactgggttgaccaagacaaggttttgtctcacagagctgttggtggtttcgtttctcacggtggttggaactctttggttgaagctgctagacacggtgttccagttttggtttggccacaattcggtgaccaaaagatcaacgctgaagctgttgaatctgctggtttgggtatgtgggttagatcttggggttggggtactgaattgagagctaagggtgacgaaatcggtttgaagatcaaggacttgatggctaacgacttcttgagagaacaagctaagagaatcgaagaagaagctagaaaggctatcggtgttggtggttcttctgaaagaactttcaaggaattgatcgacaagtggaagtgtaacaacaacactcactag

SEQ ID NO: 7 - protein, 471 aa, Oryzae sativa
MPSSGDAAGRRPHVVLIPSAGMGHLVPFGRLAVALSSGHGCDVSLVTVLPTVSTAESKHLDALFDAFPAVRRLDFELAPFDASEFPGADPFFLRFEAMRRSAPLLGPLLTGAGASALATDIALTSVVIPVAKEQGLPCHILFTASAAMLSLCAYFPTYLDANAGGGGGVGDVDIPGVYRIPKASIPQALHDPNHLFTRQFVANGRSLTSAAGILVNTFDALEPEAVAALQQGKVASGFPPVFAVGPLLPASNQAKDPQANYMEWLDAQPARSVVYVSFGSRKAISREQLRELAAGLEGSGHRFLWVVKSTVVDRDDAAELGELLDEGFLERVEKRGLVTKAWVDQEEVLKHESVALFVSHCGWNSVTEAAASGVPVLALPRFGDQRVNSGVVARAGLGVWADTWSWEGEAGVIGAEEISEKVKAAMADEALRMKAASLAEAAAKAVAGGGSSHRCLAEFARLCQGGTCRTN

SEQ ID NO: 8 - DNA, 1416 bp, Oryzae sativa
atgccatcttctggtgacgctgctggtagaagaccacacgttgttttgatcccatctgctggtatgggtcacttggttccattcggtagattggctgttgctttgtcttctggtcacggttgtgacgtttctttggttactgttttgccaactgtttctactgctgaatctaagcacttggacgctttgttcgacgctttcccagctgttagaagattggacttcgaattggctccattcgacgcttctgaattcccaggtgctgacccattcttcttgagattcgaagctatgagaagatctgctccattgttgggtccattgttgactggtgctggtgcttctgctttggctactgacatcgctttgacttctgttgttatcccagttgctaaggaacaaggtttgccatgtcacatcttgttcactgcttctgctgctatgttgtctttgtgtgcttacttcccaacttacttggacgctaacgctggtggtggtggtggtgttggtgacgttgacatcccaggtgtttacagaatcccaaaggcttctatcccacaagctttgcacgacccaaaccacttgttcactagacaattcgttgctaacggtagatctttgacttctgctgctggtatcttggttaacactttcgacgctttggaaccagaagctgttgctgctttgcaacaaggtaaggttgcttctggtttcccaccagttttcgctgttggtccattgttgccagcttctaaccaagctaaggacccacaagctaactacatggaatggttggacgctcaaccagctagatctgttgtttacgtttctttcggttctagaaaggctatctctagagaacaattgagagaattggctgctggtttggaaggttctggtcacagattcttgtgggttgttaagtctactgttgttgacagagacgacgctgctgaattgggtgaattgttggacgaaggtttcttggaaagagttgaaaagagaggtttggttactaaggcttgggttgaccaagaagaagttttgaagcacgaatctgttgctttgttcgtttctcactgtggttggaactctgttactgaagctgctgcttctggtgttccagttttggctttgccaagattcggtgaccaaagagttaactctggtgttgttgctagagctggtttgggtgtttgggctgacacttggtcttgggaaggtgaagctggtgttatcggtgctgaagaaatctctgaaaaggttaaggctgctatggctgacgaagctttgagaatgaaggctgcttctttggctgaagctgctgctaaggctgttgctggtggtggttcttctcacagatgtttggctgaattcgctagattgtgtcaaggtggtacttgtagaactaactag

SEQ ID NO: 9 - protein, 457 aa, Fagopyrum esculentum
MMGDLTTSFPATTLTTNDQPHVVVCSGAGMGHLTPFLNLASALSSAPYNCKVTLLIVIPLITDAESHHISSFFSSHPTIHRLDFHVNLPAPKPNVDPFFLRYKSISDSAHRLPVHLSALSPPISAVFSDFLFTQGLNTTLPHLPNYTFTTTSARFFTLMSYVPHLAKSSSSSPVEIPGLEPFPTDNIPPPFFNPEHIFTSFTISNAKYFSLSKGILVNTFDSFEPETLSALNSGDTLSDLPPVIPIGPLNELEHNKQEELLPWLDQQPEKSVLYVSFGNRTAMSSDQILELGMGLERSDCRFIWVVKTSKIDKDDKSELRKLFGEELYLKLSEKGKLVKWVNQTEILGHTAVGGFLSHCGWNSVMEAARRGVPILAWPQHGDQRENAWVVEKAGLGVWEREWASGIQAAIVEKVKMIMGNNDLRKSAMKVGEEAKRACDVGGSSATALMNIIGSLKR

SEQ ID NO: 10 - DNA, 1374 bp, Fagopyrum esculentum
atgatgggtgacttgactacttctttcccagctactactttgactactaacgaccaaccacacgttgttgtttgttctggtgctggtatgggtcacttgactccattcttgaacttggcttctgctttgtcttctgctccatacaactgtaaggttactttgttgatcgttatcccattgatcactgacgctgaatctcaccacatctcttctttcttctcttctcacccaactatccacagattggacttccacgttaacttgccagctccaaagccaaacgttgacccattcttcttgagatacaagtctatctctgactctgctcacagattgccagttcacttgtctgctttgtctccaccaatctctgctgttttctctgacttcttgttcactcaaggtttgaacactactttgccacacttgccaaactacactttcactactacttctgctagattcttcactttgatgtcttacgttccacacttggctaagtcttcttcttcttctccagttgaaatcccaggtttggaaccattcccaactgacaacatcccaccaccattcttcaacccagaacacatcttcacttctttcactatctctaacgctaagtacttctctttgtctaagggtatcttggttaacactttcgactctttcgaaccagaaactttgtctgctttgaactctggtgacactttgtctgacttgccaccagttatcccaatcggtccattgaacgaattggaacacaacaagcaagaagaattgttgccatggttggaccaacaaccagaaaagtctgttttgtacgtttctttcggtaacagaactgctatgtcttctgaccaaatcttggaattgggtatgggtttggaaagatctgactgtagattcatctgggttgttaagacttctaagatcgacaaggacgacaagtctgaattgagaaagttgttcggtgaagaattgtacttgaagttgtctgaaaagggtaagttggttaagtgggttaaccaaactgaaatcttgggtcacactgctgttggtggtttcttgtctcactgtggttggaactctgttatggaagctgctagaagaggtgttccaatcttggcttggccacaacacggtgaccaaagagaaaacgcttgggttgttgaaaaggctggtttgggtgtttgggaaagagaatgggcttctggtatccaagctgctatcgttgaaaaggttaagatgatcatgggtaacaacgacttgagaaagtctgctatgaaggttggtgaagaagctaagagagcttgtgacgttggtggttcttctgctactgctttgatgaacatcatcggttctttgaagagatag

SEQ ID NO: 11 - protein, 480 aa, Glycine max
MSSSEGVVHVAFLPSAGMGHLNPFLRLAATFIRYGCKVTLITPKPTVSLAESNLISRFCSSFPHQVTQLDLNLVSVDPTTVDTIDPFFLQFETIRRSLHLLPPILSLLSTPLSAFIYDITLITPLLSVIEKLSCPSYLYFTSSARMFSFFARVSVLSASNPGQTPSSFIGDDGVKIPGFTSPIPRSSVPPAILQASSNLFQRIMLEDSANVTKLNNGVFINSFEELEGEALAALNGGKVLEGLPPVYGVGPLMACEYEKGDEEGQKGCMSSIVKWLDEQSKGSVVYVSLGNRTETRREQIKDMALGLIECGYGFLWVVKLKRVDKEDEEGLEEVLGSELSSKVKEKGVVVKEFVDQVEILGHPSVGGFLSHGGWNSVTETVWKGVPCLSWPQHSDQKMSAEVIRMSGMGIWPEEWGWGTQDVVKGDEIAKRIKEMMSNESLRVKAGELKEAALKAAGVGGSCEVTIKRQIEEWKRNAQAN

SEQ ID NO: 12 - DNA, 1443 bp, Glycine max
atgtcttcttctgaaggtgttgttcacgttgctttcttgccatctgctggtatgggtcacttgaacccattcttgagattggctgctactttcatcagatacggttgtaaggttactttgatcactccaaagccaactgtttctttggctgaatctaacttgatctctagattctgttcttctttcccacaccaagttactcaattggacttgaacttggtttctgttgacccaactactgttgacactatcgacccattcttcttgcaattcgaaactatcagaagatctttgcacttgttgccaccaatcttgtctttgttgtctactccattgtctgctttcatctacgacatcactttgatcactccattgttgtctgttatcgaaaagttgtcttgtccatcttacttgtacttcacttcttctgctagaatgttctctttcttcgctagagtttctgttttgtctgcttctaacccaggtcaaactccatcttctttcatcggtgacgacggtgttaagatcccaggtttcacttctccaatcccaagatcttctgttccaccagctatcttgcaagcttcttctaacttgttccaaagaatcatgttggaagactctgctaacgttactaagttgaacaacggtgttttcatcaactctttcgaagaattggaaggtgaagctttggctgctttgaacggtggtaaggttttggaaggtttgccaccagtttacggtgttggtccattgatggcttgtgaatacgaaaagggtgacgaagaaggtcaaaagggttgtatgtcttctatcgttaagtggttggacgaacaatctaagggttctgttgtttacgtttctttgggtaacagaactgaaactagaagagaacaaatcaaggacatggctttgggtttgatcgaatgtggttacggtttcttgtgggttgttaagttgaagagagttgacaaggaagacgaagaaggtttggaagaagttttgggttctgaattgtcttctaaggttaaggaaaagggtgttgttgttaaggaattcgttgaccaagttgaaatcttgggtcacccatctgttggtggtttcttgtctcacggtggttggaactctgttactgaaactgtttggaagggtgttccatgtttgtcttggccacaacactctgaccaaaagatgtctgctgaagttatcagaatgtctggtatgggtatctggccagaagaatggggttggggtactcaagacgttgttaagggtgacgaaatcgctaagagaatcaaggaaatgatgtctaacgaatctttgagagttaaggctggtgaattgaaggaagctgctttgaaggctgctggtgttggtggttcttgtgaagttactatcaagagacaaatcgaagaatggaagagaaacgctcaagctaactag

SEQ ID NO: 13 - protein, 475 aa, Zea mays
MAANGGDHTSARPHVVLLPSAGMGHLVPFARLAVALSEGHGCNVSVAAVQPTVSSAESRLLDALFVAAAPAVRRLDFRLAPFDESEFPGADPFFLRFEATRRSAPLLGPLLDAAEASALVTDIVLASVALPVARERGVPCYVLFTSSAAMLSLCAYFPAYLDAHAAAGSVGVGVGNVDIPGVFRIPKSSVPQALHDPDHLFTQQFVANGRCLVACDGILVNTFDAFEPDAVTALRQGSITVSGGFPPVFTVGPMLPVRFQAEETADYMRWLSAQPPRSVVYVSFGSRKAIPRDQLRELAAGLEASGKRFLWVVKSTIVDRDDTADLGGLLGDGFLERVQGRAFVTMGWVEQEEILQHGSVGLFISHCGWNSLTEAAAFGVPVLAWPRFGDQRVNAALVARSGLGAWEEGWTWDGEEGLTTRKEVAKKIKGMMGYDAVAEKAAKVGDAAAAAIAKCGTSYQSLEEFVQRCRDAERK

SEQ ID NO: 14 - DNA, 1428 bp, Zea mays
atggctgctaacggtggtgaccacacttctgctagaccacacgttgttttgttgccatctgctggtatgggtcacttggttccattcgctagattggctgttgctttgtctgaaggtcacggttgtaacgtttctgttgctgctgttcaaccaactgtttcttctgctgaatctagattgttggacgctttgttcgttgctgctgctccagctgttagaagattggacttcagattggctccattcgacgaatctgaattcccaggtgctgacccattcttcttgagattcgaagctactagaagatctgctccattgttgggtccattgttggacgctgctgaagcttctgctttggttactgacatcgttttggcttctgttgctttgccagttgctagagaaagaggtgttccatgttacgttttgttcacttcttctgctgctatgttgtctttgtgtgcttacttcccagcttacttggacgctcacgctgctgctggttctgttggtgttggtgttggtaacgttgacatcccaggtgttttcagaatcccaaagtcttctgttccacaagctttgcacgacccagaccacttgttcactcaacaattcgttgctaacggtagatgtttggttgcttgtgacggtatcttggttaacactttcgacgctttcgaaccagacgctgttactgctttgagacaaggttctatcactgtttctggtggtttcccaccagttttcactgttggtccaatgttgccagttagattccaagctgaagaaactgctgactacatgagatggttgtctgctcaaccaccaagatctgttgtttacgtttctttcggttctagaaaggctatcccaagagaccaattgagagaattggctgctggtttggaagcttctggtaagagattcttgtgggttgttaagtctactatcgttgacagagacgacactgctgacttgggtggtttgttgggtgacggtttcttggaaagagttcaaggtagagctttcgttactatgggttgggttgaacaagaagaaatcttgcaacacggttctgttggtttgttcatctctcactgtggttggaactctttgactgaagctgctgctttcggtgttccagttttggcttggccaagattcggtgaccaaagagttaacgctgctttggttgctagatctggtttgggtgcttgggaagaaggttggacttgggacggtgaagaaggtttgactactagaaaggaagttgctaagaagatcaagggtatgatgggttacgacgctgttgctgaaaaggctgctaaggttggtgacgctgctgctgctgctatcgctaagtgtggtacttcttaccaatctttggaagaattcgttcaaagatgtagagacgctgaaagaaagtag

SEQ ID NO: 15 - protein, 470 aa, Mangifera indica
MSASDALNSCPHVALLLSSGMGHLTPCLRFAATLVQHHCRVTIITNYPTVSVAESRAISLLLSDFPQITEKQFHLLPFDPSTANTTDPFFLRWEAIRRSAHLLNPLLSSISPPLSALVIDSSLVSSFVPVAANLDLPSYVLFTSSTRMCSLEETFPAFVASKTNFDSIQLDDVIEIPGFSPVPVSSVPPVFLNLNHLFTTMLIQNGQSFRKANGILINTFEALEGGILPGINDKRAADGLPPYCSVGPLLPCKFEKTECSAPVKWLDDQPEGSVVYVSFGSRFALSSEQIKELGDGLIRSGCRFLWVVKCKKVDQEDEESLDELLGRDVLEKIKKYGFVIKNWVNQQEILDHRAVGGFVTHGGWNSSMEAVWHGVPMLVWPQFGDQKINAEVIERSGLGMWVKRWGWGTQQLVKGEEIGERIKDLMGNNPLRVRAKTLREEARKAIEVGGSSEKTLKELIENWKKTSRKT

SEQ ID NO: 16 - DNA, 1413 bp, Mangifera indica
atgtctgcttctgacgctttgaactcttgtccacacgttgctttgttgttgtcttctggtatgggtcacttgactccatgtttgagattcgctgctactttggttcaacaccactgtagagttactatcatcactaactacccaactgtttctgttgctgaatctagagctatctctttgttgttgtctgacttcccacaaatcactgaaaagcaattccacttgttgccattcgacccatctactgctaacactactgacccattcttcttgagatgggaagctatcagaagatctgctcacttgttgaacccattgttgtcttctatctctccaccattgtctgctttggttatcgactcttctttggtttcttctttcgttccagttgctgctaacttggacttgccatcttacgttttgttcacttcttctactagaatgtgttctttggaagaaactttcccagctttcgttgcttctaagactaacttcgactctatccaattggacgacgttatcgaaatcccaggtttctctccagttccagtttcttctgttccaccagttttcttgaacttgaaccacttgttcactactatgttgatccaaaacggtcaatctttcagaaaggctaacggtatcttgatcaacactttcgaagctttggaaggtggtatcttgccaggtatcaacgacaagagagctgctgacggtttgccaccatactgttctgttggtccattgttgccatgtaagttcgaaaagactgaatgttctgctccagttaagtggttggacgaccaaccagaaggttctgttgtttacgtttctttcggttctagattcgctttgtcttctgaacaaatcaaggaattgggtgacggtttgatcagatctggttgtagattcttgtgggttgttaagtgtaagaaggttgaccaagaagacgaagaatctttggacgaattgttgggtagagacgttttggaaaagatcaagaagtacggtttcgttatcaagaactgggttaaccaacaagaaatcttggaccacagagctgttggtggtttcgttactcacggtggttggaactcttctatggaagctgtttggcacggtgttccaatgttggtttggccacaattcggtgaccaaaagatcaacgctgaagttatcgaaagatctggtttgggtatgtgggttaagagatggggttggggtactcaacaattggttaagggtgaagaaatcggtgaaagaatcaaggacttgatgggtaacaacccattgagagttagagctaagactttgagagaagaagctagaaaggctatcgaagttggtggttcttctgaaaagactttgaaggaattgatcgaaaactggaagaagacttctagaaagacttag

SEQ ID NO: 17 - protein, 477 aa, Gentiana triflora
MGSLTNNDNLHIFLVCFIGQGVVNPMLRLGKAFASKGLLVTLSAPEIVGTEIRKANNLNDDQPIKVGSGMIRFEFFDDGWESVNGSKPFDVWVYINHLDQTGRQKLPIMLKKHEETGTPVSCLILNPLVPWVADVADSLQIPCATLWVQSCASFSAYYHYHHGLVPFPTESEPEIDVQLPGMPLLKYDEVPDYLHPRTPYPFFGTNILGQFKNLSKNFCILMDTFYELEHEIIDNMCKLCPIKPIGPLFKIPKDPSSNGITGNFMKVDDCKEWLDSRPTSTVVYVSVGSVVYLKQEQVTEMAYGILNSEVSFLWVLRPPSKRIGTEPHVLPEEFWEKAGDRGKVVQWSPQEQVLAHPATVGFLTHCGWNSTQEAISSGVPVITFPQFGDQVTNAKFLVEEFKVGVRLGRGELENRIITRDEVERALREITSGPKAEEVKENALKWKKKAEETVAKGGYSERNLVGFIEEVARKTGTK

SEQ ID NO: 18 - DNA, 1434 bp, Gentiana triflora
atgggttctttgactaacaacgacaacttgcacatcttcttggtttgtttcatcggtcaaggtgttgttaacccaatgttgagattgggtaaggctttcgcttctaagggtttgttggttactttgtctgctccagaaatcgttggtactgaaatcagaaaggctaacaacttgaacgacgaccaaccaatcaaggttggttctggtatgatcagattcgaattcttcgacgacggttgggaatctgttaacggttctaagccattcgacgtttgggtttacatcaaccacttggaccaaactggtagacaaaagttgccaatcatgttgaagaagcacgaagaaactggtactccagtttcttgtttgatcttgaacccattggttccatgggttgctgacgttgctgactctttgcaaatcccatgtgctactttgtgggttcaatcttgtgcttctttctctgcttactaccactaccaccacggtttggttccattcccaactgaatctgaaccagaaatcgacgttcaattgccaggtatgccattgttgaagtacgacgaagttccagactacttgcacccaagaactccatacccattcttcggtactaacatcttgggtcaattcaagaacttgtctaagaacttctgtatcttgatggacactttctacgaattggaacacgaaatcatcgacaacatgtgtaagttgtgtccaatcaagccaatcggtccattgttcaagatcccaaaggacccatcttctaacggtatcactggtaacttcatgaaggttgacgactgtaaggaatggttggactctagaccaacttctactgttgtttacgtttctgttggttctgttgtttacttgaagcaagaacaagttactgaaatggcttacggtatcttgaactctgaagtttctttcttgtgggttttgagaccaccatctaagagaatcggtactgaaccacacgttttgccagaagaattctgggaaaaggctggtgacagaggtaaggttgttcaatggtctccacaagaacaagttttggctcacccagctactgttggtttcttgactcactgtggttggaactctactcaagaagctatctcttctggtgttccagttatcactttcccacaattcggtgaccaagttactaacgctaagttcttggttgaagaattcaaggttggtgttagattgggtagaggtgaattggaaaacagaatcatcactagagacgaagttgaaagagctttgagagaaatcacttctggtccaaaggctgaagaagttaaggaaaacgctttgaagtggaagaagaaggctgaagaaactgttgctaagggtggttactctgaaagaaacttggttggtttcatcgaagaagttgctagaaagactggtactaagtag

SEQ ID NO: 19 - protein, 515 aa, Dactylopius coccus
MEFRLLILALFSVLMSTSNGAEILALFPIHGISNYNVAEALLKTLANRGHNVTVVTSFPQKKPVPNLYEIDVSGAKGLATNSIHFERLQTIIQDVKSNFKNMVRLSRTYCEIMFSDPRVLNIRDKKFDLVINAVFGSDCDAGFAWKSQAPLISILNARHTPWALHRMGNPSNPAYMPVIHSRFPVKMNFFQRMINTGWHLYFLYMYFYYGNGEDANKMARKFFGNDMPDINEMVFNTSLLFVNTHFSVDMPYPLVPNCIEIGGIHVKEPQPLPLEIQKFMDEAEHGVIFFTLGSMVRTSTFPNQTIQAFKEAFAELPQRVLWKFENENEDMPSNVLIRKWFPQNDIFGHKNIKAFISHGGNSGALEAVHFGVPIIGIPLFYDQYRNILSFVKEGVAVLLDVNDLTKDNILSSVRTVVNDKSYSERMKALSQLFRDRPMSPLDTAVYWTEYVIRHRGAHHLKTAGAFLHWYQYLLLDVITFLLVTFCAFCFIVKYICKALIHHYWSSSKSEKLKKN

SEQ ID NO: 20 - DNA, 1548 bp, Dactylopius coccus
atggaattcagattgttgatcttggctttgttctctgttttgatgtctacttctaacggtgctgaaatcttggctttgttcccaatccacggtatctctaactacaacgttgctgaagctttgttgaagactttggctaacagaggtcacaacgttactgttgttacttctttcccacaaaagaagccagttccaaacttgtacgaaatcgacgtttctggtgctaagggtttggctactaactctatccacttcgaaagattgcaaactatcatccaagacgttaagtctaacttcaagaacatggttagattgtctagaacttactgtgaaatcatgttctctgacccaagagttttgaacatcagagacaagaagttcgacttggttatcaacgctgttttcggttctgactgtgacgctggtttcgcttggaagtctcaagctccattgatctctatcttgaacgctagacacactccatgggctttgcacagaatgggtaacccatctaacccagcttacatgccagttatccactctagattcccagttaagatgaacttcttccaaagaatgatcaacactggttggcacttgtacttcttgtacatgtacttctactacggtaacggtgaagacgctaacaagatggctagaaagttcttcggtaacgacatgccagacatcaacgaaatggttttcaacacttctttgttgttcgttaacactcacttctctgttgacatgccatacccattggttccaaactgtatcgaaatcggtggtatccacgttaaggaaccacaaccattgccattggaaatccaaaagttcatggacgaagctgaacacggtgttatcttcttcactttgggttctatggttagaacttctactttcccaaaccaaactatccaagctttcaaggaagctttcgctgaattgccacaaagagttttgtggaagttcgaaaacgaaaacgaagacatgccatctaacgttttgatcagaaagtggttcccacaaaacgacatcttcggtcacaagaacatcaaggctttcatctctcacggtggtaactctggtgctttggaagctgttcacttcggtgttccaatcatcggtatcccattgttctacgaccaatacagaaacatcttgtctttcgttaaggaaggtgttgctgttttgttggacgttaacgacttgactaaggacaacatcttgtcttctgttagaactgttgttaacgacaagtcttactctgaaagaatgaaggctttgtctcaattgttcagagacagaccaatgtctccattggacactgctgtttactggactgaatacgttatcagacacagaggtgctcaccacttgaagactgctggtgctttcttgcactggtaccaatacttgttgttggacgttatcactttcttgttggttactttctgtgctttctgtttcatcgttaagtacatctgtaaggctttgatccaccactactggtcttcttctaagtctgaaaagttgaagaagaactag

SEQ ID NO: 21 - protein, 504 aa, Dactylopius coccus
MTLLRDLLLLYINSLLFINPSIGENILVFLPTKTYSHFKPLEPLFQELAMRGHNVTVFSGFSLTKNISNYSSIVFSAEIEFVNIGMGNLRKQSRIYNWIYVHNELQNYFTQLISDNQLQELLSNKDTQFDLIFIELYHVDGVFALSHRFNCPIIGLSFQPVLPIYNWLIGNPTTFSYIPHVYLPFTDIMSFWKRIINAVFSIFTAAFYNFVSTKGYQKHVDLLLRQTESPKLNIEELSESLSLILAEFHFSSAYTRPNLPNVIDIAGIHIQSPKPLPQDLLDFLDQSEHGVIYVSLGTLIDPIHTDHLGLNLINVFRKLRQRVIWKWKKEFFHDVPKNVLIGEWFPQIDILNHPRCKLFISHGGYHSMLESIYSSVPILGIPFFTDQHHNTAIIEKLKIGKKASTEASEEDLLTAVKELLSNETFKRNSQHQSSIFRDRPMSPMDTAIYWTEYILRYKGASHMKSAVIDLYWFQYILLDIILFYSLIVLILLCILRIFFRMLTK

SEQ ID NO: 22 - DNA, 1515 bp, Dactylopius coccus
atgactttgttgagagacttgttgttgttgtacatcaactctttgttgttcatcaacccatctatcggtgaaaacatcttggttttcttgccaactaagacttactctcacttcaagccattggaaccattgttccaagaattggctatgagaggtcacaacgttactgttttctctggtttctctttgactaagaacatctctaactactcttctatcgttttctctgctgaaatcgaattcgttaacatcggtatgggtaacttgagaaagcaatctagaatctacaactggatctacgttcacaacgaattgcaaaactacttcactcaattgatctctgacaaccaattgcaagaattgttgtctaacaaggacactcaattcgacttgatcttcatcgaattgtaccacgttgacggtgttttcgctttgtctcacagattcaactgtccaatcatcggtttgtctttccaaccagttttgccaatctacaactggttgatcggtaacccaactactttctcttacatcccacacgtttacttgccattcactgacatcatgtctttctggaagagaatcatcaacgctgttttctctatcttcactgctgctttctacaacttcgtttctactaagggttaccaaaagcacgttgacttgttgttgagacaaactgaatctccaaagttgaacatcgaagaattgtctgaatctttgtctttgatcttggctgaattccacttctcttctgcttacactagaccaaacttgccaaacgttatcgacatcgctggtatccacatccaatctccaaagccattgccacaagacttgttggacttcttggaccaatctgaacacggtgttatctacgtttctttgggtactttgatcgacccaatccacactgaccacttgggtttgaacttgatcaacgttttcagaaagttgagacaaagagttatctggaagtggaagaaggaattcttccacgacgttccaaagaacgttttgatcggtgaatggttcccacaaatcgacatcttgaaccacccaagatgtaagttgttcatctctcacggtggttaccactctatgttggaatctatctactcttctgttccaatcttgggtatcccattcttcactgaccaacaccacaacactgctatcatcgaaaagttgaagatcggtaagaaggcttctactgaagcttctgaagaagacttgttgactgctgttaaggaattgttgtctaacgaaactttcaagagaaactctcaacaccaatcttctatcttcagagacagaccaatgtctccaatggacactgctatctactggactgaatacatcttgagatacaagggtgcttctcacatgaagtctgctgttatcgacttgtactggttccaatacatcttgttggacatcatcttgttctactctttgatcgttttgatcttgttgtgtatcttgagaatcttcttcagaatgttgactaagtag

SEQ ID NO: 23 - protein, 526 aa, Dactylopius coccus
MIFFYFLTLTSFISVAFSYNILGVFPFQAKSHFGFIDPLLVRLAELGHNVTIYDPYPKSEKLPNYNEIDVSECFVFNTLYEEIDTFIKTAASPFSSLWYSFEETLAVFQKENFDKCAPLRELLNSTVKYDLLITETFLTDITLLFVNKFKIPFITSTPNVPFPWLADRMGNPLNPSYIPNLFSDYPFDKMTFFNRLWNTLFYVMALGGHNAIILKNEEKINKYYFGSSVPSLYNIARETSIMLINAHETLNPVIPLVPGMIPVSGIHIKQPAALPQNIEKFINESTHGVVYFCMGSLLRGETFPAEKRDAFLYAFSKIPQRVLWKWEGEVLPGKSENIMTSKWMPQRDILAHPNVKLFISHGGLLGTSEAVYEGVPVIGIPIFGDQRTNIKALEANGAGELLDYNDISGEVVLEKIQRLINDPKYKESARQLSIRYKDRPMSPLDTAVYWTEYVIRHKGAPHLKTAAVDMPWYQYLLLDVIAFLIFILVSVILIIYYGVKISLRYLCALIFGNSSSLKPTKKVKDN

SEQ ID NO: 24 - DNA, 1581 bp, Dactylopius coccus
atgatcttcttctacttcttgactttgacttctttcatctctgttgctttctcttacaacatcttgggtgttttcccattccaagctaagtctcacttcggtttcatcgacccattgttggttagattggctgaattgggtcacaacgttactatctacgacccatacccaaagtctgaaaagttgccaaactacaacgaaatcgacgtttctgaatgtttcgttttcaacactttgtacgaagaaatcgacactttcatcaagactgctgcttctccattctcttctttgtggtactctttcgaagaaactttggctgttttccaaaaggaaaacttcgacaagtgtgctccattgagagaattgttgaactctactgttaagtacgacttgttgatcactgaaactttcttgactgacatcactttgttgttcgttaacaagttcaagatcccattcatcacttctactccaaacgttccattcccatggttggctgacagaatgggtaacccattgaacccatcttacatcccaaacttgttctctgactacccattcgacaagatgactttcttcaacagattgtggaacactttgttctacgttatggctttgggtggtcacaacgctatcatcttgaagaacgaagaaaagatcaacaagtactacttcggttcttctgttccatctttgtacaacatcgctagagaaacttctatcatgttgatcaacgctcacgaaactttgaacccagttatcccattggttccaggtatgatcccagtttctggtatccacatcaagcaaccagctgctttgccacaaaacatcgaaaagttcatcaacgaatctactcacggtgttgtttacttctgtatgggttctttgttgagaggtgaaactttcccagctgaaaagagagacgctttcttgtacgctttctctaagatcccacaaagagttttgtggaagtgggaaggtgaagttttgccaggtaagtctgaaaacatcatgacttctaagtggatgccacaaagagacatcttggctcacccaaacgttaagttgttcatctctcacggtggtttgttgggtacttctgaagctgtttacgaaggtgttccagttatcggtatcccaatcttcggtgaccaaagaactaacatcaaggctttggaagctaacggtgctggtgaattgttggactacaacgacatctctggtgaagttgttttggaaaagatccaaagattgatcaacgacccaaagtacaaggaatctgctagacaattgtctatcagatacaaggacagaccaatgtctccattggacactgctgtttactggactgaatacgttatcagacacaagggtgctccacacttgaagactgctgctgttgacatgccatggtaccaatacttgttgttggacgttatcgctttcttgatcttcatcttggtttctgttatcttgatcatctactacggtgttaagatctctttgagatacttgtgtgctttgatcttcggtaactcttcttctttgaagccaactaagaaggttaaggacaactag

SEQ ID NO: 25 - protein, 484 aa, Arabidopsis thaliana
MNREVSERIHILFFPFMAQGHMIPILDMAKLFSRRGAKSTLLTTPINAKIFEKPIEAFKNQNPDLEIGIKIFNFPCVELGLPEGCENADFINSYQKSDSGDLFLKFLFSTKYMKQQLESFIETTKPSALVADMFFPWATESAEKLGVPRLVFHGTSFFSLCCSYNMRIHKPHKKVATSSTPFVIPGLPGDIVITEDQANVAKEETPMGKFMKEVRESETNSFGVLVNSFYELESAYADFYRSFVAKRAWHIGPLSLSNRELGEKARRGKKANIDEQECLKWLDSKTPGSVVYLSFGSGTNFTNDQLLEIAFGLEGSGQSFIWVVRKNENQGDNEEWLPEGFKERTTGKGLIIPGWAPQVLILDHKAIGGFVTHCGWNSAIEGIAAGLPMVTWPMGAEQFYNEKLLTKVLRIGVNVGATELVKKGKLISRAQVEKAVREVIGGEKAEERRLWAKKLGEMAKAAVEEGGSSYNDVNKFMEELNGRK

SEQ ID NO: 26 - DNA, 1455 bp, Arabidopsis thaliana
atgaacagagaagtttctgaaagaatccacatcttgttcttcccattcatggctcaaggtcacatgatcccaatcttggacatggctaagttgttctctagaagaggtgctaagtctactttgttgactactccaatcaacgctaagatcttcgaaaagccaatcgaagctttcaagaaccaaaacccagacttggaaatcggtatcaagatcttcaacttcccatgtgttgaattgggtttgccagaaggttgtgaaaacgctgacttcatcaactcttaccaaaagtctgactctggtgacttgttcttgaagttcttgttctctactaagtacatgaagcaacaattggaatctttcatcgaaactactaagccatctgctttggttgctgacatgttcttcccatgggctactgaatctgctgaaaagttgggtgttccaagattggttttccacggtacttctttcttctctttgtgttgttcttacaacatgagaatccacaagccacacaagaaggttgctacttcttctactccattcgttatcccaggtttgccaggtgacatcgttatcactgaagaccaagctaacgttgctaaggaagaaactccaatgggtaagttcatgaaggaagttagagaatctgaaactaactctttcggtgttttggttaactctttctacgaattggaatctgcttacgctgacttctacagatctttcgttgctaagagagcttggcacatcggtccattgtctttgtctaacagagaattgggtgaaaaggctagaagaggtaagaaggctaacatcgacgaacaagaatgtttgaagtggttggactctaagactccaggttctgttgtttacttgtctttcggttctggtactaacttcactaacgaccaattgttggaaatcgctttcggtttggaaggttctggtcaatctttcatctgggttgttagaaagaacgaaaaccaaggtgacaacgaagaatggttgccagaaggtttcaaggaaagaactactggtaagggtttgatcatcccaggttgggctccacaagttttgatcttggaccacaaggctatcggtggtttcgttactcactgtggttggaactctgctatcgaaggtatcgctgctggtttgccaatggttacttggccaatgggtgctgaacaattctacaacgaaaagttgttgactaaggttttgagaatcggtgttaacgttggtgctactgaattggttaagaagggtaagttgatctctagagctcaagttgaaaaggctgttagagaagttatcggtggtgaaaaggctgaagaaagaagattgtgggctaagaagttgggtgaaatggctaaggctgctgttgaagaaggtggttcttcttacaacgacgttaacaagttcatggaagaattgaacggtagaaagtag

SEQ ID NO: 27 - protein, 455 aa, Arabidopsis thaliana
MEKSNGLRVILFPLPLQGCINPMIQLAKILHSRGFSITVIHTCFNAPKASSHPLFTFLEIPDGLSETEKRTNNTKLLLTLLNRNCESPFRECLSKLLQSADSETGEEKQRISCLIADSGWMFTQPIAQSLKLPILVLSVFTVSFFRCQFVLPKLRREVYLPLQDSEQEDLVQEFPPLRKKDIVRILDVETDILDPFLDKVLQMTKASSGLIFMSCEELDHDSVSQAREDFKIPIFGIGPSHSHFPATSSSLSTPDETCIPWLDKQEDKSVIYVSYGSIVTISESDLIEIAWGLRNSDQPFLLVVRVGSVRGREWIETIPEEIMEKLNEKGKIVKWAPQQDVLKHRAIGGFLTHNGWSSTVESVCEAVPMICLPFRWDQMLNARFVSDVWMVGINLEDRVERNEIEGAIRRLLVEPEGEAIRERIEHLKEKVGRSFQQNGSAYQSLQNLIDYISSF

SEQ ID NO: 28 - DNA, 1368 bp, Arabidopsis thaliana
atggaaaagtctaacggtttgagagttatcttgttcccattgccattgcaaggttgtatcaacccaatgatccaattggctaagatcttgcactctagaggtttctctatcactgttatccacacttgtttcaacgctccaaaggcttcttctcacccattgttcactttcttggaaatcccagacggtttgtctgaaactgaaaagagaactaacaacactaagttgttgttgactttgttgaacagaaactgtgaatctccattcagagaatgtttgtctaagttgttgcaatctgctgactctgaaactggtgaagaaaagcaaagaatctcttgtttgatcgctgactctggttggatgttcactcaaccaatcgctcaatctttgaagttgccaatcttggttttgtctgttttcactgtttctttcttcagatgtcaattcgttttgccaaagttgagaagagaagtttacttgccattgcaagactctgaacaagaagacttggttcaagaattcccaccattgagaaagaaggacatcgttagaatcttggacgttgaaactgacatcttggacccattcttggacaaggttttgcaaatgactaaggcttcttctggtttgatcttcatgtcttgtgaagaattggaccacgactctgtttctcaagctagagaagacttcaagatcccaatcttcggtatcggtccatctcactctcacttcccagctacttcttcttctttgtctactccagacgaaacttgtatcccatggttggacaagcaagaagacaagtctgttatctacgtttcttacggttctatcgttactatctctgaatctgacttgatcgaaatcgcttggggtttgagaaactctgaccaaccattcttgttggttgttagagttggttctgttagaggtagagaatggatcgaaactatcccagaagaaatcatggaaaagttgaacgaaaagggtaagatcgttaagtgggctccacaacaagacgttttgaagcacagagctatcggtggtttcttgactcacaacggttggtcttctactgttgaatctgtttgtgaagctgttccaatgatctgtttgccattcagatgggaccaaatgttgaacgctagattcgtttctgacgtttggatggttggtatcaacttggaagacagagttgaaagaaacgaaatcgaaggtgctatcagaagattgttggttgaaccagaaggtgaagctatcagagaaagaatcgaacacttgaaggaaaaggttggtagatctttccaacaaaacggttctgcttaccaatctttgcaaaacttgatcgactacatctcttctttctag

SEQ ID NO: 29 - protein, 481 aa, Arabidopsis thaliana
MSSDPHRKLHVVFFPFMAYGHMIPTLDMAKLFSSRGAKSTILTTPLNSKIFQKPIERFKNLNPSFEIDIQIFDFPCVDLGLPEGCENVDFFTSNNNDDRQYLTLKFFKSTRFFKDQLEKLLETTRPDCLIADMFFPWATEAAEKFNVPRLVFHGTGYFSLCSEYCIRVHNPQNIVASRYEPFVIPDLPGNIVITQEQIADRDEESEMGKFMIEVKESDVKSSGVIVNSFYELEPDYADFYKSVVLKRAWHIGPLSVYNRGFEEKAERGKKASINEVECLKWLDSKKPDSVIYISFGSVACFKNEQLFEIAAGLETSGANFIWVVRKNIGIEKEEWLPEGFEERVKGKGMIIRGWAPQVLILDHQATCGFVTHCGWNSLLEGVAAGLPMVTWPVAAEQFYNEKLVTQVLRTGVSVGAKKNVRTTGDFISREKVVKAVREVLVGEEADERRERAKKLAEMAKAAVEGGSSFNDLNSFIEEFTS

SEQ ID NO: 30 - DNA, 1446 bp, Arabidopsis thaliana
atgtcttctgacccacacagaaagttgcacgttgttttcttcccattcatggcttacggtcacatgatcccaactttggacatggctaagttgttctcttctagaggtgctaagtctactatcttgactactccattgaactctaagatcttccaaaagccaatcgaaagattcaagaacttgaacccatctttcgaaatcgacatccaaatcttcgacttcccatgtgttgacttgggtttgccagaaggttgtgaaaacgttgacttcttcacttctaacaacaacgacgacagacaatacttgactttgaagttcttcaagtctactagattcttcaaggaccaattggaaaagttgttggaaactactagaccagactgtttgatcgctgacatgttcttcccatgggctactgaagctgctgaaaagttcaacgttccaagattggttttccacggtactggttacttctctttgtgttctgaatactgtatcagagttcacaacccacaaaacatcgttgcttctagatacgaaccattcgttatcccagacttgccaggtaacatcgttatcactcaagaacaaatcgctgacagagacgaagaatctgaaatgggtaagttcatgatcgaagttaaggaatctgacgttaagtcttctggtgttatcgttaactctttctacgaattggaaccagactacgctgacttctacaagtctgttgttttgaagagagcttggcacatcggtccattgtctgtttacaacagaggtttcgaagaaaaggctgaaagaggtaagaaggcttctatcaacgaagttgaatgtttgaagtggttggactctaagaagccagactctgttatctacatctctttcggttctgttgcttgtttcaagaacgaacaattgttcgaaatcgctgctggtttggaaacttctggtgctaacttcatctgggttgttagaaagaacatcggtatcgaaaaggaagaatggttgccagaaggtttcgaagaaagagttaagggtaagggtatgatcatcagaggttgggctccacaagttttgatcttggaccaccaagctacttgtggtttcgttactcactgtggttggaactctttgttggaaggtgttgctgctggtttgccaatggttacttggccagttgctgctgaacaattctacaacgaaaagttggttactcaagttttgagaactggtgtttctgttggtgctaagaagaacgttagaactactggtgacttcatctctagagaaaaggttgttaaggctgttagagaagttttggttggtgaagaagctgacgaaagaagagaaagagctaagaagttggctgaaatggctaaggctgctgttgaaggtggttcttctttcaacgacttgaactctttcatcgaagaattcacttcttag

SEQ ID NO: 31 - protein, 474 aa, Stevia rebaudiana
MSTSELVFIPSPGAGHLPPTVELAKLLLHRDQRLSVTIIVMNLWLGPKHNTEARPCVPSLRFVDIPCDESTMALISPNTFISAFVEHHKPRVRDIVRGIIESDSVRLAGFVLDMFCMPMSDVANEFGVPSYNYFTSGAATLGLMFHLQWKRDHEGYDATELKNSDTELSVPSYVNPVPAKVLPEVVLDKEGGSKMFLDLAERIRESKGIIVNSCQAIERHALEYLSSNNNGIPPVFPVGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 32 - DNA, 1425 bp, Stevia rebaudiana
atgtctacttctgaattggttttcatcccatctccaggtgctggtcacttgccaccaactgttgaattggctaagttgttgttgcacagagaccaaagattgtctgttactatcatcgttatgaacttgtggttgggtccaaagcacaacactgaagctagaccatgtgttccatctttgagattcgttgacatcccatgtgacgaatctactatggctttgatctctccaaacactttcatctctgctttcgttgaacaccacaagccaagagttagagacatcgttagaggtatcatcgaatctgactctgttagattggctggtttcgttttggacatgttctgtatgccaatgtctgacgttgctaacgaattcggtgttccatcttacaactacttcacttctggtgctgctactttgggtttgatgttccacttgcaatggaagagagaccacgaaggttacgacgctactgaattgaagaactctgacactgaattgtctgttccatcttacgttaacccagttccagctaaggttttgccagaagttgttttggacaaggaaggtggttctaagatgttcttggacttggctgaaagaatcagagaatctaagggtatcatcgttaactcttgtcaagctatcgaaagacacgctttggaatacttgtcttctaacaacaacggtatcccaccagttttcccagttggtccaatcttgaacttggaaaacaagaaggacgacgctaagactgacgaaatcatgagatggttgaacgaacaaccagaatcttctgttgttttcttgtgtttcggttctatgggttctttcaacgaaaagcaagttaaggaaatcgctgttgctatcgaaagatctggtcacagattcttgtggtctttgagaagaccaactccaaaggaaaagatcgaattcccaaaggaatacgaaaacttggaagaagttttgccagaaggtttcttgaagagaacttcttctatcggtaaggttatcggttgggctccacaaatggctgttttgtctcacccatctgttggtggtttcgtttctcactgtggttggaactctactttggaatctatgtggtgtggtgttccaatggctgcttggccattgtacgctgaacaaactttgaacgctttcttgttggttgttgaattgggtttggctgctgaaatcagaatggactacagaactgacactaaggctggttacgacggtggtatggaagttactgttgaagaaatcgaagacggtatcagaaagttgatgtctgacggtgaaatcagaaacaaggttaaggacgttaaggaaaagtctagagctgctgttgttgaaggtggttcttcttacgcttctatcggtaagttcatcgaacacgtttctaacgttactatctag

SEQ ID NO: 33 - protein, 478 aa, Oryza sativa
MKQTVVLYPGGGVGHVVPMLELAKVFVKHGHDVTMVLLEPPFKSSDSGALAVERLVASNPSVSFHVLPPLPAPDFASFGKHPFLLVIQLLRQYNERLESFLLSIPRQRLHSLVIDMFCVDAIDVCAKLGVPVYTFFASGVSVLSVLTQLPPFLAGRETGLKELGDTPLDFLGVSPMPASHLVKELLEHPEDELCKAMVNRWERNTETMGVLVNSFESLESRAAQALRDDPLCVPGKVLPPIYCVGPLVGGGAEEAAERHECLVWLDAQPEHSVVFLCFGSKGVFSAEQLKEIAVGLENSRQRFMWVVRTPPTTTEGLKKYFEQRAAPDLDALFPDGFVERTKDRGFIVTTWAPQVDVLRHRATGAFVTHCGWNSALEGITAGVPMLCWPQYAEQKMNKVFMTAEMGVGVELDGYNSDFVKAEELEAKVRLVMESEEGKQLRARSAARKKEAEAALEEGGSSHAAFVQFLSDVENLVQN

SEQ ID NO: 34 - DNA, 1437 bp, Oryza sativa
atgaagcaaactgttgttttgtacccaggtggtggtgttggtcacgttgttccaatgttggaattggctaaggttttcgttaagcacggtcacgacgttactatggttttgttggaaccaccattcaagtcttctgactctggtgctttggctgttgaaagattggttgcttctaacccatctgtttctttccacgttttgccaccattgccagctccagacttcgcttctttcggtaagcacccattcttgttggttatccaattgttgagacaatacaacgaaagattggaatctttcttgttgtctatcccaagacaaagattgcactctttggttatcgacatgttctgtgttgacgctatcgacgtttgtgctaagttgggtgttccagtttacactttcttcgcttctggtgtttctgttttgtctgttttgactcaattgccaccattcttggctggtagagaaactggtttgaaggaattgggtgacactccattggacttcttgggtgtttctccaatgccagcttctcacttggttaaggaattgttggaacacccagaagacgaattgtgtaaggctatggttaacagatgggaaagaaacactgaaactatgggtgttttggttaactctttcgaatctttggaatctagagctgctcaagctttgagagacgacccattgtgtgttccaggtaaggttttgccaccaatctactgtgttggtccattggttggtggtggtgctgaagaagctgctgaaagacacgaatgtttggtttggttggacgctcaaccagaacactctgttgttttcttgtgtttcggttctaagggtgttttctctgctgaacaattgaaggaaatcgctgttggtttggaaaactctagacaaagattcatgtgggttgttagaactccaccaactactactgaaggtttgaagaagtacttcgaacaaagagctgctccagacttggacgctttgttcccagacggtttcgttgaaagaactaaggacagaggtttcatcgttactacttgggctccacaagttgacgttttgagacacagagctactggtgctttcgttactcactgtggttggaactctgctttggaaggtatcactgctggtgttccaatgttgtgttggccacaatacgctgaacaaaagatgaacaaggttttcatgactgctgaaatgggtgttggtgttgaattggacggttacaactctgacttcgttaaggctgaagaattggaagctaaggttagattggttatggaatctgaagaaggtaagcaattgagagctagatctgctgctagaaagaaggaagctgaagctgctttggaagaaggtggttcttctcacgctgctttcgttcaattcttgtctgacgttgaaaacttggttcaaaactag

SEQ ID NO: 35 - protein, 530 aa, Homo Sapiens
MARAGWTSPVPLCVCLLLTCGFAEAGKLLVVPMDGSHWFTMQSVVEKLILRGHEVVVVMPEVSWQLERSLNCTVKTYSTSYTLEDQNREFMVFAHAQWKAQAQSIFSLLMSSSSGFLDLFFSHCRSLFNDRKLVEYLKESSFDAVFLDPFDTCGLIVAKYFSLPSVVFTRGIFCHHLEEGAQCPAPLSYVPNDLLGFSDAMTFKERVWNHIVHLEDHLFCQYLFRNALEIASEILQTPVTAYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 36 - DNA, 1590 bp, Homo Sapiens
atggctagagctggttggacttctccagttccattgtgtgtttgtttgttgttgacttgtggtttcgctgaagctggtaagttgttggttgttccaatggacggttctcactggttcactatgcaatctgttgttgaaaagttgatcttgagaggtcacgaagttgttgttgttatgccagaagtttcttggcaattggaaagatctttgaactgtactgttaagacttactctacttcttacactttggaagaccaaaacagagaattcatggttttcgctcacgctcaatggaaggctcaagctcaatctatcttctctttgttgatgtcttcttcttctggtttcttggacttgttcttctctcactgtagatctttgttcaacgacagaaagttggttgaatacttgaaggaatcttctttcgacgctgttttcttggacccattcgacacttgtggtttgatcgttgctaagtacttctctttgccatctgttgttttcactagaggtatcttctgtcaccacttggaagaaggtgctcaatgtccagctccattgtcttacgttccaaacgacttgttgggtttctctgacgctatgactttcaaggaaagagtttggaaccacatcgttcacttggaagaccacttgttctgtcaatacttgttcagaaacgctttggaaatcgcttctgaaatcttgcaaactccagttactgcttacgacttgtactctcacacttctatctggttgttgagaactgacttcgttttggactacccaaagccagttatgccaaacatgatcttcatcggtggtatcaactgtcaccaaggtaagccattgccaatggaattcgaagcttacatcaacgcttctggtgaacacggtatcgttgttttctctttgggttctatggtttctgaaatcccagaaaagaaggctatggctatcgctgacgctttgggtaagatcccacaaactgttttgtggagatacactggtactagaccatctaacttggctaacaacactatcttggttaagtggttgccacaaaacgacttgttgggtcacccaatgactagagctttcatcactcacgctggttctcacggtgtttacgaatctatctgtaacggtgttccaatggttatgatgccattgttcggtgaccaaatggacaacgctaagagaatggaaactaagggtgctggtgttactttgaacgttttggaaatgacttctgaagacttggaaaacgctttgaaggctgttatcaacgacaagtcttacaaggaaaacatcatgagattgtcttctttgcacaaggacagaccagttgaaccattggacttggctgttttctgggttgaattcgttatgagacacaagggtgctccacacttgagaccagctgctcacgacttgacttggtaccaataccactctttggacgttatcggtttcttgttggctgttgttttgactgttgctttcatcactttcaagtgttgtgcttacggttacagaaagtgtttgggtaagaagggtagagttaagaaggctcacaagtctaagactcac

SEQ ID NO: 37 - protein, 530 aa, Homo Sapiens
MACTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 38 - DNA, 1590 bp, Homo Sapiens
atggcttgtactggttggacttctccattgccattgtgtgtttgtttgttgttgacttgtggtttcgctgaagctggtaagttgttggttgttccaatggacggttctcactggttcactatgagatctgttgttgaaaagttgatcttgagaggtcacgaagttgttgttgttatgccagaagtttcttggcaattgggtagatctttgaactgtactgttaagacttactctacttcttacactttggaagacttggacagagaattcaaggctttcgctcacgctcaatggaaggctcaagttagatctatctactctttgttgatgggttcttacaacgacatcttcgacttgttcttctctaactgtagatctttgttcaaggacaagaagttggttgaatacttgaaggaatcttctttcgacgctgttttcttggacccattcgacaactgtggtttgatcgttgctaagtacttctctttgccatctgttgttttcgctagaggtatcttgtgtcactacttggaagaaggtgctcaatgtccagctccattgtcttacgttccaagaatcttgttgggtttctctgacgctatgactttcaaggaaagagttagaaaccacatcatgcacttggaagaacacttgttgtgtcacagattcttcaagaacgctttggaaatcgcttctgaaatcttgcaaactccagttactgaatacgacttgtactctcacacttctatctggttgttgagaactgacttcgttttggactacccaaagccagttatgccaaacatgatcttcatcggtggtatcaactgtcaccaaggtaagccattgccaatggaattcgaagcttacatcaacgcttctggtgaacacggtatcgttgttttctctttgggttctatggtttctgaaatcccagaaaagaaggctatggctatcgctgacgctttgggtaagatcccacaaactgttttgtggagatacactggtactagaccatctaacttggctaacaacactatcttggttaagtggttgccacaaaacgacttgttgggtcacccaatgactagagctttcatcactcacgctggttctcacggtgtttacgaatctatctgtaacggtgttccaatggttatgatgccattgttcggtgaccaaatggacaacgctaagagaatggaaactaagggtgctggtgttactttgaacgttttggaaatgacttctgaagacttggaaaacgctttgaaggctgttatcaacgacaagtcttacaaggaaaacatcatgagattgtcttctttgcacaaggacagaccagttgaaccattggacttggctgttttctgggttgaattcgttatgagacacaagggtgctccacacttgagaccagctgctcacgacttgacttggtaccaataccactctttggacgttatcggtttcttgttggctgttgttttgactgttgctttcatcactttcaagtgttgtgcttacggttacagaaagtgtttgggtaagaagggtagagttaagaaggctcacaagtctaagactcac

SEQ ID NO: 39 - protein, 529 aa, Homo Sapiens
MSVKWTSVILLIQLSFCFSSGNCGKVLVWAAEYSHWMNIKTILDELIQRGHEVTVLASSASILFDPNNSSALKIEIYPTSLTKTELENFIMQQIKRWSDLPKDTFWLYFSQVQEIMSIFGDITRKFCKDVVSNKKFMKKVQESRFDVIFADAIFPCSELLAELFNIPFVYSLSFSPGYTFEKHSGGFIFPPSYVPVVMSELTDQMTFMERVKNMIYVLYFDFWFEIFDMKKWDQFYSEVLGRPTTLSETMGKADVWLIRNSWNFQFPYPLLPNVDFVGGLHCKPAKPLPKEMEDFVQSSGENGVVVFSLGSMVSNMTEERANVIASALAQIPQKVLWRFDGNKPDTLGLNTRLYKWIPQNDLLGHPKTRAFITHGGANGIYEAIYHGIPMVGIPLFADQPDNIAHMKARGAAVRVDFNTMSSTDLLNALKRVINDPSYKENVMKLSRIQHDQPVKPLDRAVFWIEFVMRHKGAKHLRVAAHDLTWFQYHSLDVIGFLLVCVATVIFIVTKCCLFCFWKFARKAKKGKND

SEQ ID NO: 40 - DNA, 1587 bp, Homo Sapiens
atgtctgttaagtggacttctgttatcttgttgatccaattgtctttctgtttctcttctggtaactgtggtaaggttttggtttgggctgctgaatactctcactggatgaacatcaagactatcttggacgaattgatccaaagaggtcacgaagttactgttttggcttcttctgcttctatcttgttcgacccaaacaactcttctgctttgaagatcgaaatctacccaacttctttgactaagactgaattggaaaacttcatcatgcaacaaatcaagagatggtctgacttgccaaaggacactttctggttgtacttctctcaagttcaagaaatcatgtctatcttcggtgacatcactagaaagttctgtaaggacgttgtttctaacaagaagttcatgaagaaggttcaagaatctagattcgacgttatcttcgctgacgctatcttcccatgttctgaattgttggctgaattgttcaacatcccattcgtttactctttgtctttctctccaggttacactttcgaaaagcactctggtggtttcatcttcccaccatcttacgttccagttgttatgtctgaattgactgaccaaatgactttcatggaaagagttaagaacatgatctacgttttgtacttcgacttctggttcgaaatcttcgacatgaagaagtgggaccaattctactctgaagttttgggtagaccaactactttgtctgaaactatgggtaaggctgacgtttggttgatcagaaactcttggaacttccaattcccatacccattgttgccaaacgttgacttcgttggtggtttgcactgtaagccagctaagccattgccaaaggaaatggaagacttcgttcaatcttctggtgaaaacggtgttgttgttttctctttgggttctatggtttctaacatgactgaagaaagagctaacgttatcgcttctgctttggctcaaatcccacaaaaggttttgtggagattcgacggtaacaagccagacactttgggtttgaacactagattgtacaagtggatcccacaaaacgacttgttgggtcacccaaagactagagctttcatcactcacggtggtgctaacggtatctacgaagctatctaccacggtatcccaatggttggtatcccattgttcgctgaccaaccagacaacatcgctcacatgaaggctagaggtgctgctgttagagttgacttcaacactatgtcttctactgacttgttgaacgctttgaagagagttatcaacgacccatcttacaaggaaaacgttatgaagttgtctagaatccaacacgaccaaccagttaagccattggacagagctgttttctggatcgaattcgttatgagacacaagggtgctaagcacttgagagttgctgctcacgacttgacttggttccaataccactctttggacgttatcggtttcttgttggtttgtgttgctactgttatcttcatcgttactaagtgttgtttgttctgtttctggaagttcgctagaaaggctaagaagggtaagaacgac

SEQ ID NO: 41-44 - skipped (4 entries)

SEQ ID NO: 45 - protein, 296 aa, Arabidopsis thaliana
MFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 46 - DNA, 891 bp, Arabidopsis thaliana
atgttcgacttcaacaagtacatggactctaaggctatgactgttaacgaagctttgaacaaggctatcccattgagatacccacaaaagatctacgaatctatgagatactctttgttggctggtggtaagagagttagaccagttttgtgtatcgctgcttgtgaattggttggtggtactgaagaattggctatcccaactgcttgtgctatcgaaatgatccacactatgtctttgatgcacgacgacttgccatgtatcgacaacgacgacttgagaagaggtaagccaactaaccacaagatcttcggtgaagacactgctgttactgctggtaacgctttgcactcttacgctttcgaacacatcgctgtttctacttctaagactgttggtgctgacagaatcttgagaatggtttctgaattgggtagagctactggttctgaaggtgttatgggtggtcaaatggttgacatcgcttctgaaggtgacccatctatcgacttgcaaactttggaatggatccacatccacaagactgctatgttgttggaatgttctgttgtttgtggtgctatcatcggtggtgcttctgaaatcgttatcgaaagagctagaagatacgctagatgtgttggtttgttgttccaagttgttgacgacatcttggacgttactaagtcttctgacgaattgggtaagactgctggtaaggacttgatctctgacaaggctacttacccaaagttgatgggtttggaaaaggctaaggaattctctgacgaattgttgaacagagctaagggtgaattgtcttgtttcgacccagttaaggctgctccattgttgggtttggctgactacgttgctttcagacaaaactag

SEQ ID NO: 47 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 48 - DNA, 2163 bp, Cannabis sativa
atgggtaagaactacaagtctttggactctgttgttgcttctgacttcatcgctttgggtatcacttctgaagttgctgaaactttgcacggtagattggctgaaatcgtttgtaactacggtgctgctactccacaaacttggatcaacatcgctaaccacatcttgtctccagacttgccattctctttgcaccaaatgttgttctacggttgttacaaggacttcggtccagctccaccagcttggatcccagacccagaaaaggttaagtctactaacttgggtgctttgttggaaaagagaggtaaggaattcttgggtgttaagtacaaggacccaatctcttctttctctcacttccaagaattctctgttagaaacccagaagtttactggagaactgttttgatggacgaaatgaagatctctttctctaaggacccagaatgtatcttgagaagagacgacatcaacaacccaggtggttctgaatggttgccaggtggttacttgaactctgctaagaactgtttgaacgttaactctaacaagaagttgaacgacactatgatcgtttggagagacgaaggtaacgacgacttgccattgaacaagttgactttggaccaattgagaaagagagtttggttggttggttacgctttggaagaaatgggtttggaaaagggttgtgctatcgctatcgacatgccaatgcacgttgacgctgttgttatctacttggctatcgttttggctggttacgttgttgtttctatcgctgactctttctctgctccagaaatctctactagattgagattgtctaaggctaaggctatcttcactcaagaccacatcatcagaggtaagaagagaatcccattgtactctagagttgttgaagctaagtctccaatggctatcgttatcccatgttctggttctaacatcggtgctgaattgagagacggtgacatctcttgggactacttcttggaaagagctaaggaattcaagaactgtgaattcactgctagagaacaaccagttgacgcttacactaacatcttgttctcttctggtactactggtgaaccaaaggctatcccatggactcaagctactccattgaaggctgctgctgacggttggtctcacttggacatcagaaagggtgacgttatcgtttggccaactaacttgggttggatgatgggtccatggttggtttacgcttctttgttgaacggtgcttctatcgctttgtacaacggttctccattggtttctggtttcgctaagttcgttcaagacgctaaggttactatgttgggtgttgttccatctatcgttagatcttggaagtctactaactgtgtttctggttacgactggtctactatcagatgtttctcttcttctggtgaagcttctaacgttgacgaatacttgtggttgatgggtagagctaactacaagccagttatcgaaatgtgtggtggtactgaaatcggtggtgctttctctgctggttctttcttgcaagctcaatctttgtcttctttctcttctcaatgtatgggttgtactttgtacatcttggacaagaacggttacccaatgccaaagaacaagccaggtatcggtgaattggctttgggtccagttatgttcggtgcttctaagactttgttgaacggtaaccaccacgacgtttacttcaagggtatgccaactttgaacggtgaagttttgagaagacacggtgacatcttcgaattgacttctaacggttactaccacgctcacggtagagctgacgacactatgaacatcggtggtatcaagatctcttctatcgaaatcgaaagagtttgtaacgaagttgacgacagagttttcgaaactactgctatcggtgttccaccattgggtggtggtccagaacaattggttatcttcttcgttttgaaggactctaacgacactactatcgacttgaaccaattgagattgtctttcaacttgggtttgcaaaagaagttgaacccattgttcaaggttactagagttgttccattgtcttctttgccaagaactgctactaacaagatcatgagaagagttttgagacaacaattctctcacttcgaatag

SEQ ID NO: 49 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 50 - DNA, 1158 bp, Cannabis sativa
atgaaccacttgagagctgaaggtccagcttctgttttggctatcggtactgctaacccagaaaacatcttgttgcaagacgaattcccagactactacttcagagttactaagtctgaacacatgactcaattgaaggaaaagttcagaaagatctgtgacaagtctatgatcagaaagagaaactgtttcttgaacgaagaacacttgaagcaaaacccaagattggttgaacacgaaatgcaaactttggacgctagacaagacatgttggttgttgaagttccaaagttgggtaaggacgcttgtgctaaggctatcaaggaatggggtcaaccaaagtctaagatcactcacttgatcttcacttctgcttctactactgacatgccaggtgctgactaccactgtgctaagttgttgggtttgtctccatctgttaagagagttatgatgtaccaattgggttgttacggtggtggtactgttttgagaatcgctaaggacatcgctgaaaacaacaagggtgctagagttttggctgtttgttgtgacatcatggcttgtttgttcagaggtccatctgaatctgacttggaattgttggttggtcaagctatcttcggtgacggtgctgctgctgttatcgttggtgctgaaccagacgaatctgttggtgaaagaccaatcttcgaattggtttctactggtcaaactatcttgccaaactctgaaggtactatcggtggtcacatcagagaagctggtttgatcttcgacttgcacaaggacgttccaatgttgatctctaacaacatcgaaaagtgtttgatcgaagctttcactccaatcggtatctctgactggaactctatcttctggatcactcacccaggtggtaaggctatcttggacaaggttgaagaaaagttgcacttgaagtctgacaagttcgttgactctagacacgttttgtctgaacacggtaacatgtcttcttctactgttttgttcgttatggacgaattgagaaagagatctttggaagaaggtaagtctactactggtgacggtttcgaatggggtgttttgttcggtttcggtccaggtttgactgttgaaagagttgttgttagatctgttccaatcaagtactag

SEQ ID NO: 51 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 52 - DNA, 306 bp, Cannabis sativa
atggctgttaagcacttgatcgttttgaagttcaaggacgaaatcactgaagctcaaaaggaagaattcttcaagacttacgttaacttggttaacatcatcccagctatgaaggacgtttactggggtaaggacgttactcaaaagaacaaggaagaaggttacactcacatcgttgaagttactttcgaatctgttgaaactatccaagactacatcatccacccagctcacgttggtttcggtgacgtttacagatctttctgggaaaagttgttgatcttcgactacactccaagaaagtag

SEQ ID NO: 53 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 54 - DNA, 1197 bp, Cannabis sativa
atgggtttgtctttggtttgtactttctctttccaaactaactaccacactttgttgaacccacacaacaagaacccaaagaactctttgttgtcttaccaacacccaaagactccaatcatcaagtcttcttacgacaacttcccatctaagtactgtttgactaagaacttccacttgttgggtttgaactctcacaacagaatctcttctcaatctagatctatcagagctggttctgaccaaatcgaaggttctccacaccacgaatctgacaactctatcgctactaagatcttgaacttcggtcacacttgttggaagttgcaaagaccatacgttgttaagggtatgatctctatcgcttgtggtttgttcggtagagaattgttcaacaacagacacttgttctcttggggtttgatgtggaaggctttcttcgctttggttccaatcttgtctttcaacttcttcgctgctatcatgaaccaaatctacgacgttgacatcgacagaatcaacaagccagacttgccattggtttctggtgaaatgtctatcgaaactgcttggatcttgtctatcatcgttgctttgactggtttgatcgttactatcaagttgaagtctgctccattgttcgttttcatctacatcttcggtatcttcgctggtttcgcttactctgttccaccaatcagatggaagcaatacccattcactaacttcttgatcactatctcttctcacgttggtttggctttcacttcttactctgctactacttctgctttgggtttgccattcgtttggagaccagctttctctttcatcatcgctttcatgactgttatgggtatgactatcgctttcgctaaggacatctctgacatcgaaggtgacgctaagtacggtgtttctactgttgctactaagttgggtgctagaaacatgactttcgttgtttctggtgttttgttgttgaactacttggtttctatctctatcggtatcatctggccacaagttttcaagtctaacatcatgatcttgtctcacgctatcttggctttctgtttgatcttccaaactagagaattggctttggctaactacgcttctgctccatctagacaattcttcgaattcatctggttgttgtactacgctgaatacttcgtttacgttttcatctag

SEQ ID NO: 55 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 56 - DNA, 1638 bp, Cannabis sativa
atgaactgttctgctttctctttctggttcgtttgtaagatcatcttcttcttcttgtctttccacatccaaatctctatcgctaacccaagagaaaacttcttgaagtgtttctctaagcacatcccaaacaacgttgctaacccaaagttggtttacactcaacacgaccaattgtacatgtctatcttgaactctactatccaaaacttgagattcatctctgacactactccaaagccattggttatcgttactccatctaacaactctcacatccaagctactatcttgtgttctaagaaggttggtttgcaaatcagaactagatctggtggtcacgacgctgaaggtatgtcttacatctctcaagttccattcgttgttgttgacttgagaaacatgcactctatcaagatcgacgttcactctcaaactgcttgggttgaagctggtgctactttgggtgaagtttactactggatcaacgaaaagaacgaaaacttgtctttcccaggtggttactgtccaactgttggtgttggtggtcacttctctggtggtggttacggtgctttgatgagaaactacggtttggctgctgacaacatcatcgacgctcacttggttaacgttgacggtaaggttttggacagaaagtctatgggtgaagacttgttctgggctatcagaggtggtggtggtgaaaacttcggtatcatcgctgcttggaagatcaagttggttgctgttccatctaagtctactatcttctctgttaagaagaacatggaaatccacggtttggttaagttgttcaacaagtggcaaaacatcgcttacaagtacgacaaggacttggttttgatgactcacttcatcactaagaacatcactgacaaccacggtaagaacaagactactgttcacggttacttctcttctatcttccacggtggtgttgactctttggttgacttgatgaacaagtctttcccagaattgggtatcaagaagactgactgtaaggaattctcttggatcgacactactatcttctactctggtgttgttaacttcaacactgctaacttcaagaaggaaatcttgttggacagatctgctggtaagaagactgctttctctatcaagttggactacgttaagaagccaatcccagaaactgctatggttaagatcttggaaaagttgtacgaagaagacgttggtgctggtatgtacgttttgtacccatacggtggtatcatggaagaaatctctgaatctgctatcccattcccacacagagctggtatcatgtacgaattgtggtacactgcttcttgggaaaagcaagaagacaacgaaaagcacatcaactgggttagatctgtttacaacttcactactccatacgtttctcaaaacccaagattggcttacttgaactacagagacttggacttgggtaagactaaccacgcttctccaaacaactacactcaagctagaatctggggtgaaaagtacttcggtaagaacttcaacagattggttaaggttaagactaaggttgacccaaacaacttcttcagaaacgaacaatctatcccaccattgccaccacaccaccactag

SEQ ID NO: 57 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 58 - DNA, 1635 bp, Cannabis sativa
atgaagtgttctactttctctttctggttcgtttgtaagatcatcttcttcttcttctctttcaacatccaaacttctatcgctaacccaagagaaaacttcttgaagtgtttctctcaatacatcccaaacaacgctactaacttgaagttggtttacactcaaaacaacccattgtacatgtctgttttgaactctactatccacaacttgagattcacttctgacactactccaaagccattggttatcgttactccatctcacgtttctcacatccaaggtactatcttgtgttctaagaaggttggtttgcaaatcagaactagatctggtggtcacgactctgaaggtatgtcttacatctctcaagttccattcgttatcgttgacttgagaaacatgagatctatcaagatcgacgttcactctcaaactgcttgggttgaagctggtgctactttgggtgaagtttactactgggttaacgaaaagaacgaaaacttgtctttggctgctggttactgtccaactgtttgtgctggtggtcacttcggtggtggtggttacggtccattgatgagaaactacggtttggctgctgacaacatcatcgacgctcacttggttaacgttcacggtaaggttttggacagaaagtctatgggtgaagacttgttctgggctttgagaggtggtggtgctgaatctttcggtatcatcgttgcttggaagatcagattggttgctgttccaaagtctactatgttctctgttaagaagatcatggaaatccacgaattggttaagttggttaacaagtggcaaaacatcgcttacaagtacgacaaggacttgttgttgatgactcacttcatcactagaaacatcactgacaaccaaggtaagaacaagactgctatccacacttacttctcttctgttttcttgggtggtgttgactctttggttgacttgatgaacaagtctttcccagaattgggtatcaagaagactgactgtagacaattgtcttggatcgacactatcatcttctactctggtgttgttaactacgacactgacaacttcaacaaggaaatcttgttggacagatctgctggtcaaaacggtgctttcaagatcaagttggactacgttaagaagccaatcccagaatctgttttcgttcaaatcttggaaaagttgtacgaagaagacatcggtgctggtatgtacgctttgtacccatacggtggtatcatggacgaaatctctgaatctgctatcccattcccacacagagctggtatcttgtacgaattgtggtacatctgttcttgggaaaagcaagaagacaacgaaaagcacttgaactggatcagaaacatctacaacttcatgactccatacgtttctaagaacccaagattggcttacttgaactacagagacttggacatcggtatcaacgacccaaagaacccaaacaactacactcaagctagaatctggggtgaaaagtacttcggtaagaacttcgacagattggttaaggttaagactttggttgacccaaacaacttcttcagaaacgaacaatctatcccaccattgccaagacacagacactag

SEQ ID NO: 59 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 60 - DNA, 1638 bp, Cannabis sativa
atgaactgttctactttctctttctggttcgtttgtaagatcatcttcttcttcttgtctttcaacatccaaatctctatcgctaacccacaagaaaacttcttgaagtgtttctctgaatacatcccaaacaacccagctaacccaaagttcatctacactcaacacgaccaattgtacatgtctgttttgaactctactatccaaaacttgagattcacttctgacactactccaaagccattggttatcgttactccatctaacgtttctcacatccaagcttctatcttgtgttctaagaaggttggtttgcaaatcagaactagatctggtggtcacgacgctgaaggtttgtcttacatctctcaagttccattcgctatcgttgacttgagaaacatgcacactgttaaggttgacatccactctcaaactgcttgggttgaagctggtgctactttgggtgaagtttactactggatcaacgaaatgaacgaaaacttctctttcccaggtggttactgtccaactgttggtgttggtggtcacttctctggtggtggttacggtgctttgatgagaaactacggtttggctgctgacaacatcatcgacgctcacttggttaacgttgacggtaaggttttggacagaaagtctatgggtgaagacttgttctgggctatcagaggtggtggtggtgaaaacttcggtatcatcgctgcttgtaagatcaagttggttgttgttccatctaaggctactatcttctctgttaagaagaacatggaaatccacggtttggttaagttgttcaacaagtggcaaaacatcgcttacaagtacgacaaggacttgatgttgactactcacttcagaactagaaacatcactgacaaccacggtaagaacaagactactgttcacggttacttctcttctatcttcttgggtggtgttgactctttggttgacttgatgaacaagtctttcccagaattgggtatcaagaagactgactgtaaggaattgtcttggatcgacactactatcttctactctggtgttgttaactacaacactgctaacttcaagaaggaaatcttgttggacagatctgctggtaagaagactgctttctctatcaagttggactacgttaagaagttgatcccagaaactgctatggttaagatcttggaaaagttgtacgaagaagaagttggtgttggtatgtacgttttgtacccatacggtggtatcatggacgaaatctctgaatctgctatcccattcccacacagagctggtatcatgtacgaattgtggtacactgctacttgggaaaagcaagaagacaacgaaaagcacatcaactgggttagatctgtttacaacttcactactccatacgtttctcaaaacccaagattggcttacttgaactacagagacttggacttgggtaagactaacccagaatctccaaacaactacactcaagctagaatctggggtgaaaagtacttcggtaagaacttcaacagattggttaaggttaagactaaggctgacccaaacaacttcttcagaaacgaacaatctatcccaccattgccaccaagacaccactag

SEQ ID NO: 61 - DNA, 26 bp, artificial sequence
  Synthetic
acctgcacuttgtaattaaaacttag

SEQ ID NO: 62 - DNA, 26 bp, artificial sequence
  Synthetic
atgacagauttgttttatatttgttg

SEQ ID NO: 63 - DNA, 37 bp, Artificial
  Synthetic
agtgcagguaaaacaatggctgttaagcacttgatcg

SEQ ID NO: 64 - DNA, 30 bp, Artificial
  Synthetic
cgtgcgauctttcttggagtgtagtcgaag

SEQ ID NO: 65 - DNA, 38 bp, Artificial
  Synthetic
atctgtcauaaaacaatgaaccacttgagagctgaagg

SEQ ID NO: 66 - DNA, 32 bp, Artificial
  Synthetic
cacgcgaugtacttgattggaacagatctaac

SEQ ID NO: 67 - DNA, 34 bp, Artificial
  Synthetic
acctgcacutttgtttgtttatgtgtgtttattc

SEQ ID NO: 68 - DNA, 26 bp, Artificial
  Synthetic
atgacagauttgtaattaaaacttag

SEQ ID NO: 69 - DNA, 42 bp, Artificial
  Synthetic
agtgcagguaaaacaatgggtttgtctttggtttgtactttc

SEQ ID NO: 70 - DNA, 32 bp, Artificial
  Synthetic
cgtgcgaugatgaaaacgtaaacgaagtattc

SEQ ID NO: 71 - DNA, 40 bp, Artificial
  Synthetic
atctgtcauaaaacaatgttcgacttcaacaagtacatgg

SEQ ID NO: 72 - DNA, 33 bp, Artificial
  Synthetic
cacgcgauctagttttgtctgaaagcaacgtag

SEQ ID NO: 73 - DNA, 25 bp, Artificial
  Synthetic
cgtgcgauggaagtaccttcaaaga

SEQ ID NO: 74 - DNA, 26 bp, Artificial
  Synthetic
atgacagauttgttttatatttgttg

SEQ ID NO: 75 - DNA, 40 bp, Artificial
  Synthetic
atctgtcauaaaacaatgggtaagaactacaagtctttgg

SEQ ID NO: 76 - DNA, 33 bp, Artificial
  Synthetic
cacgcgauttcgaagtgagagaattgttgtctc

SEQ ID NO: 77 - DNA, 26 bp, Artificial
  Synthetic
acctgcacuttgtaattaaaacttag

SEQ ID NO: 78 - DNA, 25 bp, Artificial
  Synthetic
cacgcgaugcacacaccatagcttc

SEQ ID NO: 79 - DNA, 42 bp, Artificial
  Synthetic
agtgcagguaaaacaatgaactgttctgctttctctttctgg

SEQ ID NO: 80 - DNA, 29 bp, Artificial
  Synthetic
cgtgcgaugtggtggtgtggtggcaatgg

SEQ ID NO: 81 - DNA, 42 bp, Artificial
  Synthetic
agtgcagguaaaacaatgaagtgttctactttctctttctgg

SEQ ID NO: 82 - DNA, 29 bp, Artificial
  Synthetic
cgtgcgaugtgtctgtgtcttggcaatgg

SEQ ID NO: 83 - DNA, 39 bp, Artificial
  Synthetic
agtgcagguaaaacaatgaactgttctactttctctttc

SEQ ID NO: 84 - DNA, 29 bp, Artificial
  Synthetic
cgtgcgaugtggtgtcttggtggcaatgg

SEQ ID NO: 85 - DNA, 28 bp, Artificial
  Synthetic
ggatccatggctgttaagcacttgatcg

SEQ ID NO: 86 - DNA, 31 bp, Artificial
  Synthetic
aagcttctactttcttggagtgtagtcgaag

SEQ ID NO: 87 - DNA, 31 bp, Artificial
  Synthetic
cgccggcgatgaaccacttgagagctgaagg

SEQ ID NO: 88 - DNA, 33 bp, Artificial
  Synthetic
cttaagctagtacttgattggaacagatctaac

SEQ ID NO: 89 - DNA, 33 bp, Artificial
  Synthetic
ggatccatgggtttgtctttggtttgtactttc

SEQ ID NO: 90 - DNA, 33 bp, Artificial
  Synthetic
aagcttctagatgaaaacgtaaacgaagtattc

SEQ ID NO: 91 - DNA, 33 bp, Artificial
  Synthetic
cgccggcgatgttcgacttcaacaagtacatgg

SEQ ID NO: 92 - DNA, 34 bp, Artificial
  Synthetic
cttaagctactagttttgtctgaaagcaacgtag

SEQ ID NO: 93 - DNA, 31 bp, Artificial
  Synthetic
ggatccatgggtaagaactacaagtctttgg

SEQ ID NO: 94 - DNA, 34 bp, Artificial
  Synthetic
aagcttctattcgaagtgagagaattgttgtctc

SEQ ID NO: 95 - DNA, 35 bp, Artificial
  Synthetic
cgccggcgatgaactgttctgctttctctttctgg

SEQ ID NO: 96 - DNA, 30 bp, Artificial
  Synthetic
cttaagctagtggtggtgtggtggcaatgg

SEQ ID NO: 97 - DNA, 35 bp, Artificial
  Synthetic
cgccggcgatgaagtgttctactttctctttctgg

SEQ ID NO: 98 - DNA, 30 bp, Artificial
  Synthetic
cttaagctagtgtctgtgtcttggcaatgg

SEQ ID NO: 99 - DNA, 32 bp, Artificial
  Synthetic
cgccggcgatgaactgttctactttctctttc

SEQ ID NO: 100 - DNA, 30 bp, Artificial
  Synthetic
cttaagctagtggtgtcttggtggcaatgg

SEQ ID NO: 101 - protein, 477 aa, P. trichocarpa
MEDTIVLYPSPGRGHLFSMVELGKQILEHHPSISITIIISAMPTESISIDDPYFSTLCNTNPSITLIHLPQVSLPPNTSFSPLDFVASFFELPELNNTNLHQTLLNLSKSSNIKAFIIDFFCSAAFEFVSSRHNIPIYFFYTTCASGLSMFLHLPILDKIITKSLKDLDIIIDLPGIPKIPSKELPPAISDRSHRVYQYLVDTAKLMIKSAGLIINTFELLERKALQAIQEGKCGAPDEPVPPLFCVGPLLTTSESKSEHECLTWLDSQPTRSVLFLCFGSMGVFNSRQLRETAIGLEKSGVRFLWVVRPPLADSQTQAGRSSTPNEPCLDLLLPEGFLERTKDRGFLVNSWAPQVEILNHGSVGGFVTHCGWNSVLEALCAGVPMVAWPLYAEQRMNRIFLVEEMKVALAFREAGDDHFVNAAELEERVIELMNSKKGEAVRERVLKLREDAVVAKSDGGSSCIAMAKLVDCFKKG

SEQ ID NO: 102 - DNA, 1434 bp, P. trichocarpa
atggaagataccattgttctgtatccgagtcctggtcgtggtcacctgtttagcatggttgaactgggtaaacaaatcctggaacatcatccgagcattagcattaccattattatcagcgcaatgccgaccgaaagcatcagcattgatgatccgtattttagcaccctgtgtaataccaatccgagtattaccctgattcatctgccgcaggttagcctgcctccgaataccagctttagtccgctggattttgttgccagcttttttgaactgccggaactgaataatacgaatctgcatcagaccctgctgaatctgagcaaaagcagcaacattaaagccttcatcatcgactttttttgcagcgcagcatttgaatttgttagcagccgtcataacatcccgatctattttttctataccacctgtgcaagcggtctgagcatgtttctgcatctgccgattctggataaaatcattaccaaaagcctgaaggatctggatattatcattgatctgcctggcattccgaaaattccgagcaaagaactgcctccggcaattagcgatcgtagccatcgtgtttatcagtatctggttgataccgccaaactgatgattaaaagcgcaggtctgattatcaacacctttgagctgctggaacgtaaagcactgcaggcaattcaagagggtaaatgtggtgcaccggatgaaccggtgcctccgctgttttgtgttggtccgctgctgaccaccagtgaaagcaaaagcgaacatgaatgtctgacctggctggatagccagccgacacgtagcgttctgtttctgtgttttggtagcatgggtgtgtttaatagccgtcagctgcgtgaaaccgcaattggtctggaaaaaagcggtgttcgttttctgtgggttgttcgtccgcctctggcagatagtcagacccaggcaggtcgtagcagcaccccgaatgaaccgtgtctggatctgctgctgccggaaggttttctggaacgcaccaaagatcgtggctttctggttaatagctgggcaccgcaggttgaaattctgaatcatggtagcgttggtggttttgttacccattgtggttggaatagcgtgctggaagcactgtgtgccggtgttccgatggttgcatggcctctgtatgcagaacagcgtatgaatcgtatttttctggtggaagaaatgaaagttgcactggcatttcgtgaagccggtgatgatcattttgttaatgcagcagaactggaagaacgtgtgattgaactgatgaatagcaaaaaaggtgaagccgttcgtgaacgtgttctgaaactgcgtgaagatgcagttgttgcaaaaagtgatggtggtagcagttgtattgcaatggcaaaactggttgactgctttaaaaagggctaa

SEQ ID NO: 103 - protein, 467 aa, H. annuus
MESSTVVMYPSPGIGHLVSMVELGKLIHTHHPSLSVIILILTAPYETGATGKYINTVSATTPAITFHHLPAIALPPDFSSEFIDLAFGLPELYNSVVHNTLVAISQKSTIKAVILDFFSNAAFQVSTNLSLPTYYFFTSGTFGLCAFLYLTTLHKTTSKSIKDLNTLLDFPGVPPIHSSHMPTAIFDRESNSYKNFMKTSNNMAKCSGIIVNSFLELEERAVATLRDGKCITDGPTPPIYFIGPLIASGSQVDPNENECLKWLKTQPSKSVVFLCFGSMGVFEKEQLKEIAVGLERSGQRFLWVVRNPPLESSSGAKEFELDDILPEGFLTRTKDKGLVVKNWAPQPAILGHESVGGFVSHCGWNSSLEAVVSGVPMVAWPLYAEQQMNRVYLVEEIKVALWLRMSADGFVGAEAVEETVRKLMEGEEGRAVREQILEMSGGAKAAVEDGGSSRLDFLKLTRPWTDQ

SEQ ID NO: 104 - DNA, 1404 bp, H. annuus
atggaaagcagcaccgttgttatgtatccgagtcctggtattggtcatctggttagcatggttgaactgggtaaactgattcatacccatcatccgagcctgagcgttattattctgattctgaccgcaccgtatgaaaccggtgcaaccggcaaatatatcaataccgttagcgcaaccacaccggcaattacctttcatcatctgcctgcaattgccctgcctccggattttagcagcgaatttattgatctggcatttggtctgccggaactgtataatagcgttgttcataataccctggttgccattagccagaaaagcaccattaaagcagttatcctggatttctttagcaacgcagcatttcaggttagcaccaatctgagcctgccgacctattatttctttaccagcggcacctttggtctgtgtgcatttctgtatctgaccacactgcataaaaccacgagcaaaagcattaaagatctgaataccctgctggattttccgggtgttccgcctattcatagcagccatatgccgaccgcaatttttgatcgtgaaagcaacagctacaaaaactttatgaaaaccagcaacaacatggccaaatgcagcggtattattgtgaatagctttctggaactggaagaacgtgcagttgcaaccctgcgtgatggtaaatgtattaccgatggtccgacacctccgatttatttcattggtccgctgattgcaagcggtagccaggttgatccgaatgaaaatgaatgtctgaaatggctgaaaacccagccgagcaaatcagttgtttttctgtgttttggtagcatgggcgtgtttgaaaaagaacagctgaaagaaattgccgttggtctggaacgtagcggtcagcgttttctgtgggttgttcgtaatccgcctctggaaagctcaagcggtgcaaaagaatttgaactggatgatatcctgccggaaggttttctgacccgtaccaaagataaaggtctggttgtgaaaaattgggcaccgcagcctgccattctgggtcatgaaagcgttggtggttttgttagccattgtggttggaatagcagcctggaagcagttgttagcggtgttccgatggttgcatggcctctgtatgcagaacagcagatgaatcgtgtttatctggtggaagaaattaaagttgcactgtggctgcgtatgagcgcagatggttttgtgggtgcagaagccgttgaagaaaccgttcgcaaactgatggaaggtgaagagggtcgtgcagttcgtgagcagattctggaaatgagcggtggtgccaaagcagcagttgaagatggtggtagcagccgtctggatttcctgaaactgacccgtccgtggaccgatcagtaa

SEQ ID NO: 105 - protein, 458 aa, S. rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 106 - DNA, 1377 bp, S. rebaudiana
atggaaaacaaaaccgaaaccaccgtgcgtcgtcgtcgccgtattattctgtttccggttccgtttcagggtcatattaatccgattctgcagctggcaaatgtgctgtatagcaaaggttttagcatcaccatctttcacaccaacttcaacaaaccgaaaaccagcaattatccgcattttacctttcgctttatcctggataatgatccgcaggatgaacgtattagcaatctgccgacacatggtccgctggcaggtatgcgtattccgattattaacgaacatggtgcagatgaactgcgtcgtgaactggaactgctgatgctggcaagcgaagaagatgaagaagttagctgtctgattaccgatgcactgtggtattttgcacagagcgttgcagatagcctgaatctgcgtcgcctggttctgatgaccagcagcctgtttaactttcatgcacatgttagcctgccgcagtttgatgaactgggttatctggatccggatgataaaacccgtctggaagaacaggcaagcggttttccgatgctgaaagtgaaagatatcaaaagcgcatatagcaactggcagatcctgaaagaaattctgggcaaaatgatcaaacagaccaaagcaagcagcggtgttatttggaatagctttaaagaactggaagagagcgaactggaaaccgttattcgtgaaattccggcaccgagctttctgattccgctgccgaaacatctgaccgcaagcagcagcagtctgctggatcacgatcgtaccgtttttcagtggctggatcagcagcctccgagcagcgttctgtatgttagctttggtagcaccagcgaagttgatgaaaaagactttctggaaattgcacgtggtctggttgatagcaaacagagttttctgtgggttgttcgtccgggttttgttaaaggtagcacctgggttgaaccgctgccggatggttttctgggtgaacgtggtcgtattgttaaatgggttccgcagcaagaggttctggcacatggtgccattggtgcattttggacccatagcggttggaatagtaccctggaaagcgtttgtgaaggtgttccgatgatttttagcgattttggtctggatcaaccgctgaatgcacgttatatgagtgatgttctgaaagtgggtgtgtatctggaaaatggttgggaacgtggtgaaattgcaaatgcaattcgtcgtgttatggttgatgaagagggtgaatatatccgtcagaatgcccgtgtgctgaaacagaaagcagatgtgagcctgatgaaaggtggtagcagctatgaaagcctggaaagtctggttagctatatcagctcactgtaa

SEQ ID NO: 107 - protein, 495 aa, A. thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 108 - DNA, 1488 bp, A. thaliana
atggttagcgaaaccaccaaaagcagtccgctgcattttgttctgtttccgtttatggcacagggtcatatgattccgatggttgatattgcacgtctgctggcacagcgtggtgtgattattaccattgttaccacaccgcataatgcagcacgctttaaaaacgttctgaatcgtgcaattgaaagcggtctgccgattaatctggttcaggttaaatttccgtatctggaagcaggtctgcaagaaggtcaagaaaatattgatagcctggataccatggaacgcatgattccgtttttcaaagccgtgaattttctggaagaaccggtgcagaaactgatcgaagaaatgaatccgcgtccgagctgtctgattagcgatttttgtctgccgtataccagcaaaatcgccaaaaaattcaacatcccgaaaatcctgtttcatggtatgggttgtttttgcctgctgtgtatgcatgttctgcgtaaaaatcgtgaaatcctggataacctgaaaagcgataaagaactgtttaccgttccggattttccggatcgtgtggaatttacccgtacacaggttccggttgaaacctatgttccggcaggcgattggaaagatatttttgatggtatggtggaagccaacgaaaccagctatggtgttattgtgaatagctttcaagaactggaaccggcatatgcgaaagattacaaagaagttcgtagcggtaaagcatggaccattggtccggttagcctgtgtaataaagttggtgcagataaagcagaacgcggtaataaaagtgatatcgatcaggatgaatgcctgaaatggctggatagcaaaaaacatggtagcgttctgtatgtttgtctgggtagcatttgcaatctgccgctgagccagctgaaagaattaggtctgggtttagaagaaagccagcgtccgtttatttgggttattcgtggttgggagaaatacaaagaactggttgaatggttttccgaaagcggttttgaagatcgtattcaggatcgtggcctgctgattaaaggttggagtccgcagatgctgattctgagccatccgagcgttggtggctttctgacccattgtggttggaatagcaccctggaaggtattacagctggcctgccgctgctgacctggcctctgtttgcagatcagttttgtaatgaaaaactggtggtggaagttctgaaagccggtgtgcgtagcggtgttgaacagccgatgaaatggggtgaagaagaaaaaattggcgtcctggttgataaagaaggtgttaaaaaagccgtggaagaactgatgggtgaaagtgatgatgcaaaagaacgtcgtcgtcgtgcaaaagagctgggcgatagcgcacataaagcagttgaagaaggtggtagcagccatagcaatattagctttctgctgcaggatattatggaactggcagaaccgaataactaa

SEQ ID NO: 109 - protein, 467 aa, A. thaliana
MRNVELIFIPTPTVGHLVPFLEFARRLIEQDDRIRITILLMKLQGQSHLDTYVKSIASSQPFVRFIDVPELEEKPTLGSTQSVEAYVYDVIERNIPLVRNIVMDILTSLALDGVKVKGLVVDFFCLPMIDVAKDISLPFYVFLTTNSGFLAMMQYLADRHSRDTSVFVRNSEEMLSIPGFVNPVPANVLPSALFVEDGYDAYVKLAILFTKANGILVNSSFDIEPYSVNHFLQEQNYPSVYAVGPIFDLKAQPHPEQDLTRRDELMKWLDDQPEASVVFLCFGSMARLRGSLVKEIAHGLELCQYRFLWSLRKEEVTKDDLPEGFLDRVDGRGMICGWSPQVEILAHKAVGGFVSHCGWNSIVESLWFGVPIVTWPMYAEQQLNAFLMVKELKLAVELKLDYRVHSDEIVNANEIETAIRYVMDTDNNVVRKRVMDISQMIQRATKNGGSSFAAIEKFIYDVIGIKP

SEQ ID NO: 110 - DNA, 1404 bp, A. thaliana
atgcgtaatgtggaactgatttttatcccgacaccgaccgttggtcatctggttccgtttctggaatttgcacgtcgtctgattgaacaggatgatcgtattcgtattaccatcctgctgatgaaactgcagggtcagagccatctggatacctatgttaaaagcattgcaagcagccagccgtttgttcgttttattgatgtgccggaactggaagaaaaaccgacactgggtagcacccagagcgttgaagcatatgtttatgatgtgattgaacgcaatattccgctggtgcgtaatattgttatggatattctgaccagcctggcactggatggtgttaaagttaaaggtctggttgtggattttttctgcctgccgatgattgatgttgccaaagatattagcctgccgttttatgtttttctgaccaccaatagcggttttctggcaatgatgcagtatctggcagatcgtcatagccgtgataccagcgtttttgttcgtaatagcgaagaaatgctgagcattccgggttttgttaatccggttccggcaaatgttctgccgagcgcactgtttgttgaagatggttatgatgcgtatgttaaactggccatcctgtttaccaaagccaatggtattctggtgaatagcagctttgatatcgaaccgtatagcgtgaatcactttctgcaagaacagaattatccgagcgtttatgcagttggtccgatctttgatctgaaagcacagccgcatccggaacaggatctgacccgtcgtgatgaactgatgaaatggctggatgatcagccggaagcaagcgttgtgtttctgtgttttggtagcatggcacgtctgcgtggtagcctggttaaagaaattgcacatggtctggaactgtgccagtatcgttttctgtggtcactgcgtaaagaagaagttaccaaagacgacctgccggaaggctttctggatcgtgttgatggtcgtggtatgatttgtggttggagtccgcaggttgaaattctggcacataaagcagttggtggttttgtgagccattgcggttggaatagcattgttgaaagcctgtggtttggtgttccgattgttacctggccgatgtatgcagaacagcagctgaatgcatttctgatggtgaaagaactgaaactggcagttgaactgaagctggattatcgtgttcattccgatgaaattgtgaacgccaatgaaattgaaaccgccattcgttatgtgatggataccgataacaatgttgtgcgtaaacgtgtcatggatatcagccagatgattcagcgtgcaaccaaaaatggtggtagcagttttgcagccatcgagaaatttatctatgacgtgattggcatcaagccgtaa

SEQ ID NO: 111 - protein, 480 aa, A. thaliana
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 112 - DNA, 1443 bp, A. thaliana
atggaagaaagcaaaacaccgcatgttgcaattattccgagtcctggtatgggtcatctgattccgctggttgaatttgcaaaacgtctggttcatctgcatggtctgaccgttacctttgttattgccggtgaaggtccgcctagcaaagcacagcgtaccgttctggatagcctgccgagcagcattagcagcgtttttctgcctccggttgatctgaccgatctgagcagcagcacccgtattgaaagccgtattagcctgacagttacccgtagcaatccggaactgcgtaaagtttttgatagctttgttgaaggtggtcgtctgccgaccgcactggttgttgacctgtttggcaccgatgcatttgatgttgcagttgaatttcatgtgcctccgtatatcttttatccgaccaccgcaaatgttctgagcttttttctgcatctgccgaaactggatgaaaccgttagctgtgaatttcgtgaactgaccgaaccgctgatgctgcctggttgtgttccggttgcaggtaaagattttctggatccggcacaggatcgtaaagatgatgcatataaatggctgctgcataacaccaaacgttataaagaagcagaaggcattctggtcaacaccttttttgaactggaaccgaatgcaattaaagccctgcaagaacctggtctggataaaccgcctgtttatccggttggtcctctggttaatattggtaaacaagaagccaaacagaccgaagaaagcgaatgtctgaaatggctggataatcagccgctgggtagcgttctgtatgttagctttggtagcggtggcaccctgacctgtgaacagctgaatgaactggcactgggtttagcagatagcgaacagcgttttctgtgggttattcgtagcccgagcggtattgcaaatagcagttattttgatagtcacagccagacagatccgctgacctttctgccaccgggttttctggaacgtaccaaaaaacgtggttttgtgattccgttttgggcaccgcaggcacaggttctggcacatccgagcaccggtggttttctgacccattgtggttggaatagcaccctggaaagcgttgttagcggtattccgctgattgcatggcctctgtatgcagaacagaaaatgaatgcagttctgctgagcgaagatattcgtgcagcactgcgtccgcgtgccggtgatgatggtctggttcgtcgtgaagaagttgcacgcgttgttaaaggtctgatggaaggtgaagaaggtaaaggcgttcgcaacaaaatgaaagaactgaaagaggcagcctgtcgcgttctgaaagatgacggcaccagcaccaaagcactgagcctggttgcactgaaatggaaagcacataaaaaagagctggaacagaacggcaaccactaa

SEQ ID NO: 113 - protein, 474 aa, S. rebaudiana; identical to SEQ ID NO: 31

SEQ ID NO: 114 - DNA, 1425 bp, S. rebaudiana
atgagcaccagcgaactggtttttattccgagtcctggtgcaggtcatctgcctccgaccgttgaactggcaaaactgctgctgcatcgtgatcagcgtctgagcgttaccattattgttatgaatctgtggctgggtccgaaacataataccgaagcacgtccgtgtgttccgagcctgcgttttgttgatattccgtgtgatgaaagcaccatggcactgattagcccgaatacctttattagcgcatttgtggaacatcataaaccgcgtgttcgtgatattgtgcgtggtattattgaaagcgatagcgttcgtctggcaggttttgttctggatatgttttgtatgccgatgagtgatgtggccaatgaatttggtgtgccgagctataactattttaccagcggtgcagcaaccctgggtctgatgtttcatctgcagtggaaacgtgatcatgaaggttatgatgcaaccgaactgaaaaatagcgataccgaactgtcagttccgagctatgttaatccggttccggcaaaagttctgcctgaagttgtgctggataaagaaggtggtagcaaaatgtttctggatctggcagaacgtattcgtgaaagcaaaggcattattgtgaatagctgtcaggcaattgaacgtcatgcactggaatatctgagcagcaataacaatggtattccgcctgtttttccggttggtccgattctgaatctggaaaacaaaaaagatgatgccaaaaccgatgaaattatgcgctggctgaatgaacagccggaaagcagcgttgtttttctgtgttttggtagcatgggcagctttaatgagaaacaggttaaagaaattgccgtggccattgaacgtagcggtcatcgttttctgtggtcactgcgtcgtccgacaccgaaagaaaaaattgaatttccgaaagaatatgagaacctggaagaagtgctgccggaaggttttctgaaacgtaccagcagcattggtaaagttattggttgggcaccgcagatggcagttctgagccatccgagcgttggtggttttgttagccattgtggttggaatagcaccctggaaagcatgtggtgtggtgttccgatggcagcatggcctctgtatgcagaacagaccctgaatgcatttctgctggttgttgaattaggtctggcagccgaaattcgtatggattatcgtaccgataccaaagcaggctatgatggtggtatggaagttaccgttgaagaaattgaagatggcattcgcaaactgatgtcagatggtgaaattcgcaacaaagtgaaggacgtgaaagagaaaagtcgcgcagcagttgttgaaggtggttcaagctatgcaagtatcggcaaattcatcgaacatgttagcaacgtgaccatttaa

SEQ ID NO: 115 - protein, 462 aa, O. sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 116 - DNA, 1389 bp, O. sativa
atggatagcggttatagcagcagctatgcagcagcagccggtatgcatgttgttatttgtccgtggctggcatttggtcatctgctgccgtgtctggatctggcacagcgtctggcaagccgtggtcatcgtgttagctttgttagcacaccgcgtaatattagccgtctgcctccggttcgtccggcactggcaccgctggttgcatttgttgcactgccgctgcctcgtgttgaaggtctgccggatggtgcagaaagcaccaatgatgttccgcatgatcgtccggatatggttgaactgcatcgtcgtgcatttgatggtctggcagcaccgtttagcgaatttctgggcaccgcatgtgcagattgggttattgttgatgtttttcatcattgggcagccgcagcagcactggaacataaagttccgtgtgcaatgatgctgctgggtagcgcacatatgattgcaagcattgcagatcgtcgtctggaacgtgcagaaaccgaaagtcctgcggcagcaggtcagggtcgtcctgcagccgcaccgacctttgaagttgcacgtatgaaactgattcgtaccaaaggtagcagcggtatgagcctggcagaacgttttagtctgaccctgagccgtagcagcctggttgttggtcgtagctgtgttgaatttgaaccggaaaccgttccgctgctgagcaccctgcgtggtaaaccgattacctttctgggtctgatgcctccgctgcatgaaggtcgtcgcgaagatggtgaagatgcaaccgttcgttggctggatgcacagcctgcaaaaagcgttgtttatgttgccctgggtagtgaagttccgctgggtgttgaaaaagtgcatgaactggcactgggtttagaactggcaggcacccgttttctgtgggcactgcgtaaaccgaccggtgttagtgatgccgatctgcttccggcaggttttgaagaacgtacccgtggtcgtggtgttgttgcaacccgttgggttccgcagatgagcattctggcacatgcagcagtgggtgcatttctgacccattgtggttggaatagcaccattgaaggcctgatgtttggccatccgctgattatgctgccgatttttggtgatcagggtccgaatgcacgtctgattgaagcaaaaaatgcaggtctgcaggttgcccgtaatgatggtgatggtagctttgatcgtgaaggtgttgcagcagccattcgtgcagttgcagttgaagaagaaagcagcaaagtttttcaggccaaagccaaaaaactgcaagaaattgttgcagatatggcctgccatgaacgttatattgatggttttattcagcagctgcgtagctacaaagattaa

SEQ ID NO: 117 - protein, 487 aa, S. pennellii
MGVLTIEPHFVLFPFMAQGHTIPMIDIARLLAQREVIITIVTTHLNANRFKKVIDRAIESGLKIQVVHLYFPSLEAGLPEGCENFDMLPSMDLGLKFFDATKRLQPQVEEMLQEMKPSPSCIISDMCFPWTTNVAQKFNIPRIVFHGMGCFSLLCLHNLKDWEGLEKIESDTEYFQVPGLFDKIELTKNQLGNAARPRNEEWRVISDQMKKAEEEAYGMVVNSFEDLEKEYIEGLMNVKNRKIWTIGPVSLCNKEKQDKAERGNKASIDEHKCLNWLDSREQNSVLFVCLGSLSRLSTSQMVELGLGLESSRRPFIWVVRHMSDEFKNWLVEEDFEERVKGQGLLIRGWAPQVLILSHPSIGAFLTHCGWNSSLEGITAGVAMITWPMFAEQFCNERLIVDVLKTGVRSGIERQVMFGEEEKLGTQVSRDDIKKVIEQVMGEEMRRKRAKELGEKAKRAMEEEGSSHFNLTQLIQDVTEQAKILKPM

SEQ ID NO: 118 - DNA, 1464 bp, S. pennellii
atgggtgttctgaccattgaaccgcattttgttctgtttccgtttatggcacagggtcataccattccgatgattgatattgcacgtctgctggcacagcgtgaagtgattattaccattgttaccacacatctgaatgccaaccgtttcaaaaaagttattgatcgtgcaatcgagagcggtctgaaaattcaggttgttcatctgtattttccgagcctggaagcaggtctgccggaaggttgtgaaaattttgatatgctgccgagcatggatctgggtctgaaatttttcgatgcaaccaaacgtctgcagccgcaggttgaagaaatgctgcaagaaatgaaaccgagtccgagctgtattattagcgatatgtgttttccgtggaccaccaatgttgcacagaaatttaacattccgcgtatcgtgtttcatggtatgggttgttttagcctgctgtgtctgcataatctgaaagattgggaaggcctggaaaaaattgaaagcgataccgaatattttcaggttccgggtctgtttgataaaatcgaactgaccaaaaatcagctgggtaatgcagcacgtccgcgtaatgaagaatggcgtgtgattagcgatcagatgaaaaaagccgaagaagaggcatatggtatggtggttaatagctttgaggatctggaaaaagaatacatcgaaggcctgatgaatgtgaaaaaccgtaaaatttggaccattggtccggttagcctgtgcaataaagaaaaacaggataaagccgaacgcggtaataaagcaagcatcgatgaacataaatgcctgaattggctggatagccgtgaacagaatagcgttctgtttgtttgtctgggtagcctgagccgtctgagcaccagccagatggttgaattaggtctgggtttagaaagcagccgtcgtccgtttatttgggttgttcgtcatatgtccgatgagtttaaaaactggctggtcgaagaggattttgaagaacgtgttaaaggtcagggtctgctgattcgtggttgggcaccgcaggttctgattctgagccatccgagcattggtgcatttctgacccattgtggttggaatagcagtctggaaggtattaccgcaggcgttgcaatgattacctggccgatgtttgcagaacagttttgtaatgaacgtctgattgtggatgttctgaaaaccggtgttcgtagcggtattgaacgtcaggttatgtttggtgaagaagaaaaactgggtacacaggttagccgtgatgatatcaaaaaggtgattgaacaggtgatgggtgaagagatgcgtcgtaaacgtgcaaaagaactgggtgaaaaagcaaaacgtgccatggaagaagaaggtagcagccattttaatctgacacagctgattcaggatgttaccgaacaggcaaaaattctgaaaccgatgtaa

SEQ ID NO: 119 - protein, 463 aa, O. sativa
MAIGSVESVAVVAVPFPAQGHLNQLMHLSLLLASRGLDVHYAAPPAHLRQARSRLHGWDPDALRSIRFHDLDVPAYESPPPDPTAPPFPSHMMPMIQSFAVAARAPFAALLERISASYSRVVVVYDRLNSFAAAQAARLPNGEAFGLQCVAMSYNIGWLDPENRLVREHGLKFHPVEACMPKEFVEFISREEQDEENATSSGMLMNTSRAIEAEFIDEIAAHPMFKEMKLFAVGPLNPLLDATARTPGQTRHECMDWLDKQPAASVLYVSFGTTSSLRGDQVAELAAALKGSKQRFIWVLRDADRADIFADSGESRHAELLSRFTAETEGVGLVITGWAPQLEILAHGATAAFMSHCGWNSTMESLSHGKPILAWPMHSDQPWDAELVCKYLKAGLLVRPLEKHSEVVPAEAIQEVIEEAMLPEKGMAIRRRAMELGEVVRASVADGGSSRKDLDDFVGYITR

SEQ ID NO: 120 - DNA, 1392 bp, O. sativa
atggcaattggtagcgttgaaagcgttgcagttgttgccgttccgtttccggcacagggtcatctgaaccagctgatgcatctgagcctgctgctggcaagccgtggtctggatgttcattatgcagcaccgcctgcacatctgcgtcaggcacgtagccgtctgcatggttgggatcctgatgcactgcgtagcattcgttttcatgatctggatgtgcctgcatatgaaagtccgcctccggatccgaccgcaccgccttttccgagccatatgatgccgatgattcagagctttgcagttgcagcacgtgcaccgtttgcagcactgctggaacgtattagcgcaagctatagccgtgttgttgttgtgtatgatcgtctgaatagctttgccgcagcacaggcagcacgtctgccgaatggtgaagcatttggtctgcagtgtgttgcaatgagctataacattggttggctggatccggaaaatcgtctggttcgtgaacatggtctgaaattccatccggttgaagcatgtatgccgaaagaatttgttgaatttatcagccgtgaagaacaggatgaagaaaatgcaaccagcagcggtatgctgatgaataccagccgtgcaattgaagccgaatttattgatgaaattgcagcgcacccgatgttcaaagaaatgaaactgtttgccgttggtccgctgaatcctctgctggatgcaaccgcacgtacaccgggtcagacccgtcatgaatgtatggattggctggacaaacagcctgcagcaagcgttctgtatgttagctttggcaccaccagtagcctgcgtggtgatcaggttgcagaactggcagcagcactgaaaggtagcaaacagcgttttatttgggttctgcgtgatgcagatcgtgcagatatttttgcagatagcggtgaaagccgtcatgccgaactgctgagccgttttaccgcagaaaccgaaggtgttggtctggttattaccggttgggcaccgcagctggaaattctggcacatggtgccaccgcagcatttatgagccattgtggttggaatagcaccatggaaagcctgagccatggtaaaccgattctggcatggccgatgcatagcgatcagccttgggatgctgaactggtttgtaaatatctgaaagcaggtctgctggttcgtccgctggaaaaacatagcgaagttgttccggcagaagcaattcaagaagttattgaagaagcaatgctgccggaaaaaggtatggcaattcgtcgtcgtgcaatggaactgggtgaagttgtgcgtgcaagcgttgccgatggtggtagcagccgtaaagatctggacgattttgttggttatatcacccgctaa

SEQ ID NO: 121 - protein, 456 aa, A. thaliana
MGSSEGQETHVLMVTLPFQGHINPMLKLAKHLSLSSKNLHINLATIESARDLLSTVEKPRYPVDLVFFSDGLPKEDPKAPETLLKSLNKVGAMNLSKIIEEKRYSCIISSPFTPWVPAVAASHNISCAILWIQACGAYSVYYRYYMKTNSFPDLEDLNQTVELPALPLLEVRDLPSFMLPSGGAHFYNLMAEFADCLRYVKWVLVNSFYELESEIIESMADLKPVIPIGPLVSPFLLGDGEEETLDGKNLDFCKSDDCCMEWLDKQARSSVVYISFGSMLETLENQVETIAKALKNRGLPFLWVIRPKEKAQNVAVLQEMVKEGQGVVLEWSPQEKILSHEAISCFVTHCGWNSTMETVVAGVPVVAYPSWTDQPIDARLLVDVFGIGVRMRNDSVDGELKVEEVERCIEAVTEGPAAVDIRRRAAELKRVARLALAPGGSSTRNLDLFISDITIA

SEQ ID NO: 122 - DNA, 1371 bp, A. thaliana
atgggtagcagcgaaggtcaagaaacccatgttctgatggttaccctgccgtttcagggtcatattaatccgatgctgaaactggcaaaacatctgagcctgagcagcaaaaatctgcatattaacctggcaaccattgaaagcgcacgtgatctgctgagcaccgttgaaaaaccgcgttatccggttgatctggtgttttttagtgatggtctgccgaaagaagatccgaaagcaccggaaacactgctgaaaagcctgaataaagttggtgcaatgaacctgagcaaaatcatcgaagaaaaacgctatagctgcattattagcagcccgtttacaccgtgggttccagcagttgcagcaagccataacattagctgtgcaattctgtggattcaggcatgtggtgcatatagcgtgtattatcgctattatatgaaaaccaacagcttcccggatctggaagatctgaatcagaccgttgaactgcctgcactgccgctgctggaagttcgcgatctgccgagctttatgctgccgagcggtggtgcacatttctataatctgatggcagaatttgcagattgcctgcgttatgttaaatgggtgttagtgaacagcttctatgaactggaaagcgaaattattgaaagcatggcagatctgaaaccggttattccgattggtccgctggttagcccgtttctgttaggtgatggtgaagaagaaaccctggacggtaaaaatctggatttttgtaaatccgatgattgctgcatggaatggctggataaacaggcacgtagcagcgttgtgtatattagctttggtagcatgctggaaacgctggaaaatcaggttgaaaccattgcaaaagccctgaaaaatcgcggtctgccttttctgtgggttattcgtccgaaagaaaaagcacagaatgttgcagttctgcaagagatggttaaagaaggtcagggcgttgttctggaatggtcaccgcaagaaaaaattctgagccatgaagcgattagctgctttgttacccattgtggttggaatagcaccatggaaaccgttgttgccggtgttccggttgttgcatatccgagctggaccgatcagccgattgatgcacgtctgctggttgatgtttttggtattggtgttcgtatgcgtaatgatagcgtggatggtgaactgaaagttgaagaagttgaacgttgtattgaagccgttaccgaaggtccggcagcagttgatattcgtcgtcgtgcagcagaactgaaacgtgttgcccgtctggcactggcacctggtggtagcagcacccgtaatctggacctgtttattagcgatattaccattgcctaa

SEQ ID NO: 123 - protein, 483 aa, S. rebaudiana
MDQMAKIDEKKPHVVFIPFPAQSHIKCMLKLARILHQKGLYITFINTDTNHERLVASGGTQWLENAPGFWFKTVPDGFGSAKDDGVKPTDALRELMDYLKTNFFDLFLDLVLKLEVPATCIICDGCMTFANTIRAAEKLNIPVILFWTMAACGFMAFYQAKVLKEKEIVPVKDETYLTNGYLDMEIDWIPGMKRIRLRDLPEFILATKQNYFAFEFLFETAQLADKVSHMIIHTFEELEASLVSEIKSIFPNVYTIGPLQLLLNKITQKETNNDSYSLWKEEPECVEWLNSKEPNSVVYVNFGSLAVMSLQDLVEFGWGLVNSNHYFLWIIRANLIDGKPAVMPQELKEAMNEKGFVGSWCSQEEVLNHPAVGGFLTHCGWGSIIESLSAGVPMLGWPSIGDQRANCRQMCKEWEVGMEIGKNVKRDEVEKLVRMLMEGLEGERMRKKALEWKKSATLATCCNGSSSLDVEKLANEIKKLSRN

SEQ ID NO: 124 - DNA, 1452 bp, S. rebaudiana
atggatcagatggccaaaatcgatgaaaaaaaaccgcatgtggtgtttattccgtttccggcacagagccatatcaaatgtatgctgaaactggcacgtatcctgcatcagaaaggtctgtatattaccttcattaacaccgataccaatcatgaacgtctggttgcaagcggtggcacccagtggctggaaaatgcacctggtttttggtttaaaaccgttccggatggttttggtagcgcaaaagatgatggtgttaaaccgaccgatgcactgcgtgaactgatggattatctgaaaaccaactttttcgacctgtttctggatctggtgctgaaattagaagttccggcaacctgtattatttgtgatggttgtatgacctttgccaataccattcgtgcagcagaaaaactgaatattccggtgattctgttttggaccatggcagcctgtggttttatggcattttatcaggcaaaagtgctgaaagaaaaagaaatcgttccggtgaaagatgaaacctatctgaccaatggttatctggatatggaaatcgattggattccgggtatgaaacgtattcgtctgcgtgatctgccggaatttattctggcaaccaaacagaactatttcgcctttgaatttctgttcgaaaccgcacagctggcagataaagttagccatatgattatccacaccttcgaagaactggaagcaagcctggttagcgaaatcaaaagcatttttccgaacgtgtatacaattggtccgctgcagctgctgctgaacaaaattacccagaaagaaaccaacaacgatagctatagcctgtggaaagaagaaccggaatgtgttgaatggctgaatagcaaagaaccgaatagcgttgtgtatgtgaattttggtagtctggcagttatgagcctgcaggatctggttgaatttggttggggtttagttaacagcaaccactattttctgtggattattcgtgccaatctgattgatggtaaaccggcagtgatgccgcaagaactgaaagaagcaatgaacgaaaaaggttttgttggtagctggtgtagccaagaagaagttctgaatcatccggcagttggtggttttctgacccattgcggttggggtagcattattgaaagcctgagtgccggtgttccgatgttaggttggccgagcattggtgatcagcgtgcaaattgtcgtcagatgtgtaaagaatgggaagttggtatggaaattggcaaaaacgtgaaacgtgatgaggttgaaaaactggttcgtatgctgatggaaggtctggaaggtgaacgtatgcgtaaaaaagcactggaatggaaaaaaagcgcaaccctggccacctgttgtaatggtagcagcagcctggatgttgagaaactggccaatgaaattaagaaactgagccgcaactaa

SEQ ID NO: 125 - protein, 498 aa, P. abies
MNGNEQHALHAVIVPFPAQGHVNALMNLAQLLAIRGVFVTFVNTDWIHKRTVEASKKSKSGVLNDNPEFEQQGRRIRFLSIPDGLPPGDGRTSNLGELFVALQKLGPVLEDLLRTADEKSPSFPPITFIVTDAFMSCTEQVASSMKVPRVIFWPVCAAISISQYYADLLISEGYIPVNLSQAKNPEKLITCLPGNIPPLKPTDLVSFYRAQDPTDILFNAFLHESRKQSKGDYVLVNTFEELEGRDAVTALSLDGCPALAIGPLFLPNFLEGRDSCSSLWEEEKSCLTWLDMHQPGSVIYVSFGSIAVKSEQQLEQLALGLEGSGQPFLWVLRLDIAEGQAAVLPDGFEARTKDRALFVRWAPQWNVLAHPSVGLFLTHCGWNSTLESMSMGVPVVGFPYFGDQFLNCRFAKDVWRIGLDFKDVDLDDRKVVMKEEVEDVVRRMMRTPEGKKLRDNVLRLKESAAKAVLPGGSSFLNLNTFVKDMTTGKGFQSKNETM

SEQ ID NO: 126 - DNA, 1497 bp, P. abies
atgaatggcaatgaacagcatgccctgcatgccgttattgttccgtttccggcacagggtcatgttaatgcactgatgaatctggcacagctgctggcaattcgtggtgtttttgttacctttgttaacaccgattggatccataaacgtaccgttgaagcaagcaaaaaaagcaaaagcggtgtgctgaatgataacccggaatttgaacagcagggtcgtcgtattcgttttctgagcattccggatggtctgcctccaggtgatggtcgtaccagcaatctgggtgaactgtttgttgcactgcagaaactgggtcctgttctggaagatctgctgcgtaccgcagatgaaaaaagcccgagctttccgcctattacctttattgttaccgatgcctttatgagctgtaccgaacaggttgcaagcagcatgaaagttccgcgtgtgattttttggcctgtttgtgcagcaattagcatcagccagtattatgccgatctgctgattagcgaaggttatattccggttaatctgagccaggcgaaaaatccggaaaaactgattacctgtctgcctggtaatattccgcctctgaaaccgaccgatctggttagcttttatcgtgcacaggatccgaccgatattctgtttaatgcatttctgcatgaaagccgcaaacagagcaaaggtgattatgttctggtgaacacctttgaagaactggaaggtcgtgatgcagttaccgcactgagcctggatggttgtccggcactggcaattggtccgctgtttctgccgaattttctggaaggacgcgatagctgtagcagcctgtgggaagaagaaaaaagctgtctgacctggctggatatgcatcagcctggtagcgttatttatgttagctttggtagcattgccgtgaaaagcgaacagcagctggaacagctggcactgggtttagaaggtagcggtcagccgtttctgtgggttctgcgtctggatattgcagaaggtcaggcagcagttctgccggatggttttgaagcacgtaccaaagatcgtgccctgtttgttcgttgggcaccgcagtggaatgttctggcacatccgagcgttggtctgtttctgacccattgtggttggaatagcaccctggaaagcatgagcatgggtgttccggttgttggttttccgtattttggtgatcagtttctgaattgccgtttcgcaaaagatgtttggcgtattggtctggatttcaaagatgttgatctggatgatcgtaaagtggtgatgaaagaagaagttgaggacgttgttcgtcgtatgatgcgtacaccggaaggtaaaaaactgcgtgataatgtgctgcgtctgaaagaaagcgcagcaaaagccgttctgccaggtggtagcagctttctgaatctgaatacctttgtgaaagatatgaccaccggtaaaggtttccagagcaaaaatgaaaccatgtaa

SEQ ID NO: 127 - protein, 487 aa, C. roseus
MVNQLHIFNFPFMAQGHMLPALDMANLFTSRGVKVTLITTHQHVPMFTKSIERSRNSGFDISIQSIKFPASEVGLPEGIESLDQVSGDDEMLPKFMRGVNLLQQPLEQLLQESRPHCLLSDMFFPWTTESAAKFGIPRLLFHGSCSFALSAAESVRRNKPFENVSTDTEEFVVPDLPHQIKLTRTQISTYERENIESDFTKMLKKVRDSESTSYGVVVNSFYELEPDYADYYINVLGRKAWHIGPFLLCNKLQAEDKAQRGKKSAIDADECLNWLDSKQPNSVIYLCFGSMANLNSAQLHEIATALESSGQNFIWVVRKCVDEENSSKWFPEGFEERTKEKGLIIKGWAPQTLILEHESVGAFVTHCGWNSTLEGICAGVPLVTWPFFAEQFFNEKLITEVLKTGYGVGARQWSRVSTEIIKGEAIANAINRVMVGDEAVEMRNRAKDLKEKARKALEEDGSSYRDLTALIEELGAYRSQVERKQQD

SEQ ID NO: 128 - DNA, 1464 bp, C. roseus
atggtgaaccagctgcacatttttaactttccgtttatggcacagggtcatatgctgcctgcactggatatggcaaacctgtttaccagccgtggtgttaaagttaccctgattaccacacatcagcatgttccgatgtttaccaaaagcattgaacgtagccgtaatagcggttttgatattagcattcagagcatcaaatttccggcaagcgaagttggtctgccggaaggtattgaaagcctggatcaggttagcggtgatgatgaaatgctgccgaaatttatgcgtggtgtgaatctgctgcaacagccgctggaacagctgctgcaagaaagccgtccgcattgtctgctgagcgatatgttttttccgtggaccaccgaaagcgcagcaaaatttggtattccgcgtctgctgtttcatggtagctgtagctttgcactgagcgcagcagaaagcgttcgtcgtaataaaccgtttgaaaatgttagcaccgataccgaagaatttgttgttccggatctgccgcatcagattaaactgacccgtacacagattagcacctatgaacgtgaaaacatcgaaagcgatttcaccaagatgctgaaaaaagttcgtgatagcgaaagcaccagctatggtgttgttgtgaatagcttttatgaactggaaccggattatgccgattactatattaacgttctgggtcgtaaagcctggcatattggtccgtttctgctgtgtaataaactgcaggccgaagataaagcacagcgtggtaaaaaaagcgcaattgatgcagatgaatgtctgaattggctggatagcaaacagccgaatagcgttatttatctgtgttttggtagcatggccaatctgaatagcgcacagctgcatgaaattgcaaccgcactggaaagcagcggtcagaactttatttgggttgttcgtaaatgcgtggatgaagaaaatagcagcaaatggtttccggaaggctttgaagaacgtaccaaagaaaaaggcctgattatcaaaggttgggcaccgcagacactgattctggaacatgaaagcgttggtgcatttgttacccattgtggttggaatagcaccctggaaggcatttgtgccggtgttccgctggttacctggccgttttttgcagaacagttttttaacgagaaactgatcacggaagttctgaaaaccggttatggtgtgggtgcacgtcagtggtcacgtgtgagcaccgaaatcattaaaggtgaagcaattgccaatgccattaatcgtgttatggttggtgatgaagcagtggaaatgcgtaatcgtgcaaaagatctgaaagagaaagcacgtaaagcactggaagaagatggtagcagctatcgtgatctgaccgcactgattgaagaactgggtgcatatcgtagccaggttgaacgtaaacagcaggattaa

SEQ ID NO: 129 - protein, 481 aa, A. thaliana; identical to SEQ ID NO: 29

SEQ ID NO: 130 - DNA, 1446 bp, A. thaliana
atgagcagcgatccgcatcgtaaactgcatgttgttttttttccgtttatggcctatggtcatatgattccgacactggatatggcaaaactgtttagcagccgtggtgcaaaaagcaccattctgaccacaccgctgaatagcaaaatctttcagaaaccgattgagcgcttcaaaaatctgaatccgagctttgaaatcgacatccagatctttgattttccgtgtgttgatctgggtctgccggaaggttgtgaaaatgttgattttttcaccagcaacaacaacgatgatcgtcagtatctgaccctgaaatttttcaaaagcacccgctttttcaaagatcagctggaaaaactgctggaaaccacacgtccggattgtctgattgcagatatgttttttccttgggcaaccgaagcagccgaaaaattcaatgttccgcgtctggtttttcatggcaccggttattttagcctgtgtagcgaatattgcattcgtgttcataatccgcagaatattgttgccagccgttatgaaccgtttgtgattccggatctgcctggtaatattgttattacccaagagcagattgccgatcgtgatgaagaaagcgaaatgggcaaatttatgatcgaagttaaagagagcgacgtcaaaagcagcggtgttattgttaacagcttttatgaactggaaccggattatgccgatttctataaaagcgttgttctgaaacgtgcctggcatattggtccgctgagcgtttataatcgtggctttgaagaaaaagccgagcgtggtaaaaaagccagcattaatgaagttgaatgcctgaaatggctggacagcaaaaaaccggatagcgttatctatattagctttggtagcgttgcctgctttaaaaacgagcagctgtttgaaattgcagcaggtctggaaacctcaggtgcaaactttatttgggttgtgcgtaaaaacatcggcatcgaaaaagaagaatggctgcctgaaggttttgaggaacgtgttaaaggtaaaggcatgattattcgtggttgggcaccgcaggttctgattctggatcatcaggcaacctgtggttttgttacccattgtggttggaatagcctgctggaaggtgtggcagccggtctgccgatggttacctggcctgttgcagcagaacagttttataacgaaaaactggttacccaggttctgcgtaccggtgttagcgttggtgccaaaaaaaacgttcgtaccaccggtgatttcatcagccgtgaaaaagttgttaaagccgttcgtgaagttctggttggtgaagaggcagatgaacgtcgtgaacgtgcaaaaaaactggcagaaatggcaaaagccgcagttgaaggtggtagcagctttaatgatctgaacagctttatcgaagagtttaccagctaa

SEQ ID NO: 131 - protein, 474 aa, Artificial
  Synthetic
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 132 - DNA, 1425 bp, Artificial
  Synthetic
atgggcaaacaagaagatgccgaactggttattattccgtttccgtttagcggtcatattctggcaaccattgaactggcaaaacgtctgattagccaggataatccgcgtattcataccattaccattctgtattggggtctgccgtttattccgcaggcagataccattgcatttctgcgtagcctggttaaaaatgaaccgcgtatccgtctggttaccctgccggaagttcaggatccgcctccgatggaactgtttgttgaatttgcagaaagctatatcctggaatatgtgaaaaaaatggtgccgattattcgtgaagcactgagcaccctgctgagcagccgtgatgaaagcggtagcgttcgtgttgcaggtctggttctggattttttctgtgttccgatgattgatgtgggcaacgaatttaatctgccgagctatatctttctgacctgtagcgcaggttttctgggtatgatgaaatatctgccggaacgtcatcgtgaaatcaaaagcgaatttaaccgcagctttaacgaagaactgaatctgattccgggttatgttaatagcgttccgaccaaagtgctgccgagcggtctgtttatgaaagaaacctatgaaccgtgggtagaactggccgaacgttttccggaagcaaaaggtattctggttaatagctataccgcactggaaccgaatggcttcaaatatttcgatcgttgtccggataactacccgaccatttatccgattggtccgattctgtgtagcaatgatcgtccgaatctggatctgagcgaacgtgatcgtattctgaaatggctggatgatcagccggaaagcagcgttgtgtttctgtgctttggtagcctgaaaagcctggcagcaagccagattaaagaaattgcacaggccctggaactggttggtattcgttttctgtggtcaattcgtaccgatccgaaagaatatgcaagcccgaacgaaatcctgccggatggttttatgaatcgtgttatgggtctgggtttagtttgtggttgggcaccgcaggttgaaattctggcacataaagcaattggtggttttgttagccattgcggttggaatagcattctggaaagcctgcgttttggtgtgccgattgcaacctggccgatgtatgcagaacagcagctgaatgcatttaccattgtgaaagaattaggtctggcactggaaatgcgtctggattatgttagcgaatatggcgaaattgtcaaagccgatgaaattgccggtgcagttcgtagcctgatggatggtgaagatgttccgcgtcgtaaactgaaagaaatcgcagaagcaggtaaagaagcagttatggatggcggtagcagctttgttgcagttaaacgttttattgatggcctgtaa

SEQ ID NO: 133 - protein, 456 aa, P. abies
MDDGGLSWPNRIYAAPGVFGCGRPGQIAYMQRLASSAVGAIDFLELPGVEIEGDHPNMNIRTRLSLLMEETKILVEDALRSFRFPVCAFIADLFATAMFDVTAKLKIPSYIFFTSSASLLCILLYLPTLAQEIEISFKDVDFPIEVPGLPPIPGRDLPSHLQDRSDNVSFNRSIQHSSQLREAHGILINTFQDIEAEQVKALLEGKVLSAAEMPSIYPIGPIVSSSRLESESDKEECVEWLDGQPASSVLFVSFGSRGTLSDDQIKELALGLEASGQRFLWALLNPPPPSIQCENSVSTTSAEPDMRLLLPEGFENRTKDRGLVVHSWVPQIPVLSHPSTGGFLSHCGWNSTLESILHGVPLIALPLIHDQRTNAFLLVNEAVAIEAKNGPDGLVSKEEVERVARELMEGDGGVKIKKRVRKLMEKAKNALVEGGSSYNSMATVAAVWKELDGHSC

SEQ ID NO: 134 - DNA, 1371 bp, P. abies
atggatgatggtggtctgagctggccgaatcgtatttatgcagcaccgggtgtttttggttgtggtcgtccgggtcagattgcctatatgcagcgtctggcaagcagcgcagttggtgcaattgattttctggaactgcctggtgttgaaattgaaggtgatcatccgaatatgaatattcgtacccgtctgagcctgctgatggaagaaaccaaaattctggttgaagatgcactgcgtagctttcgttttccggtttgtgcatttattgcagacctgtttgcaaccgcaatgtttgatgttaccgccaaactgaaaattccgagctatatcttttttaccagcagcgcaagcctgctgtgtattctgctgtatctgccgacactggcacaagaaattgaaatcagctttaaagatgtggacttcccgattgaagttccgggtctgcctccgattccgggtcgtgatctgccgagccatctgcaggatcgtagcgataatgttagctttaatcgtagcattcagcatagcagccagctgcgtgaagcacatggtattctgattaatacctttcaggatatcgaagccgaacaggttaaagcactgctggaaggtaaagttctgagcgcagcagaaatgccgagcatttatccgattggtccgattgttagcagcagccgtctggaaagcgaaagcgataaagaagaatgtgttgaatggctggatggtcagcctgccagcagcgttctgtttgtgagctttggtagccgtggcaccctgagtgatgatcagattaaagaactggcactgggtttagaagcaagcggtcagcgttttctgtgggcactgctgaatccgcctccgccaagcattcagtgtgaaaatagcgttagcaccaccagtgcagaaccggatatgcgtctgctgctgccggaaggttttgaaaatcgtaccaaagatcgtggtctggttgttcatagctgggttccgcagattccggtgctgagccatccgagcaccggtggttttctgagccattgtggttggaatagcaccctggaaagcattctgcatggtgttccgctgattgcactgccgctgattcacgatcagcgtaccaatgcctttctgctggttaatgaagcagttgcaattgaagcaaaaaatggtccggatggtctggtgagcaaagaagaagttgaacgcgttgcacgtgaattaatggaaggtgatggtggcgtgaaaatcaaaaaacgtgttcgtaaactgatggaaaaggccaaaaatgccctggtggaaggtggtagcagctataatagcatggcaaccgttgcagcagtttggaaagaattagatggtcacagctgctaa

SEQ ID NO: 135 - protein, 484 aa, A. thaliana; identical to SEQ ID NO: 25

SEQ ID NO: 136 - DNA, 1455 bp, A. thaliana
atgaatcgtgaagtgagcgaacgcattcacattctgttttttccgtttatggcacagggtcatatgattccgattctggatatggcaaaactgtttagccgtcgtggtgcaaaaagcaccctgctgaccacaccgattaatgcaaaaatctttgaaaaaccgatcgaggccttcaaaaatcagaatccggatctggaaattggcatcaagatttttaactttccgtgcgttgaactgggtctgccggaaggttgtgaaaatgcagattttatcaacagctaccagaaaagcgatagcggtgacctgtttctgaaatttctgttcagcaccaaatacatgaaacagcagctggaaagctttatcgaaaccaccaaaccgagcgcactggttgcagatatgtttttcccgtgggcaaccgaaagcgcagaaaaactgggtgttccgcgtctggtttttcatggcaccagcttttttagcctgtgttgcagctataatatgcgcattcataaaccgcataaaaaagttgcaaccagcagcaccccgtttgttattccgggtctgcctggtgatattgttattaccgaagatcaggcaaatgtggccaaagaagaaaccccgatgggcaaatttatgaaagaagttcgcgaaagcgaaaccaatagctttggtgttctggtgaacagcttttatgaactggaaagcgcatatgccgatttttatcgtagctttgttgcaaaacgtgcctggcatattggtccgctgagcctgagcaatcgcgaactgggtgaaaaagcgcgtcgcggtaaaaaagcaaatatcgatgaacaagaatgcctgaaatggctggatagcaaaacaccgggtagcgttgtttatctgagctttggtagcggcaccaattttaccaatgatcagctgctggaaatcgcatttggtctggaaggtagcggtcagagctttatttgggttgttcgcaaaaatgaaaaccagggcgataatgaagaatggctgcctgaaggttttaaagaacgtaccaccggtaaaggtctgattattcctggttgggcaccgcaggttctgatcctggatcacaaagcaattggtggctttgttacccattgtggttggaatagcgcaattgaaggtattgcagcaggtctgccgatggttacctggccgatgggtgcagaacagttttataacgaaaaactgctgacaaaagtgctgcgcattggtgttaatgttggtgcaaccgaactggtcaaaaaaggtaaactgattagtcgtgcccaggttgaaaaagcagttcgtgaagttattggtggcgaaaaagccgaagaacgtcgtctgtgggcaaaaaaacttggtgaaatggcaaaagcagcagttgaagaaggtggtagcagttataatgacgtgaacaagtttatggaagaactgaacggtcgcaaataa

SEQ ID NO: 137 - protein, 490 aa, Artificial
  Synthetic
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 138 - DNA, 1473 bp, Artificial
  Synthetic
atgggcaaacaagaagatgccgaactggttattattccgtttccgtttagcggtcatattctggcaaccattgaactggcaaaacgtctgattagccaggataatccgcgtattcataccattaccattctgtattggggtctgccgtttattccgcaggcagataccattgcatttctgcgtagcctggttaaaaatgaaccgcgtatccgtctggttaccctgccggaagttcaggatccgcctccgatggaactgtttgttgaatttgcagaaagctatatcctggaatatgtgaaaaaaatggtgccgattattcgtgaagcactgagcaccctgctgagcagccgtgatgaaagcggtagcgttcgtgttgcaggtctggttctggattttttctgtgttccgatgattgatgtgggcaacgaatttaatctgccgagctatatctttctgacctgtagcgcaggttttctgggtatgatgaaatatctgccggaacgtcatcgtgaaatcaaaagcgaatttaaccgcagctttaacgaagaactgaatctgattccgggttatgttaatagcgttccgaccaaagtgctgccgagcggtctgtttatgaaagaaacctatgaaccgtgggtagaactggccgaacgttttccggaagcaaaaggtattctggttaatagctataccgcactggaaccgaatggcttcaaatatttcgatcgttgtccggataactacccgaccatttatccgattggtccgattctgaatctggaaaacaaaaaagatgatgccaaaaccgatgaaattatgcgctggctgaatgaacagccggaaagcagcgttgtgtttctgtgctttggtagcatgggtagctttaatgaaaaacaggtgaaagaaattgccgtggcaattgaacgtagtggtcatcgttttctgtggtcactgcgtcgtccgacaccgaaagaaaaaattgaatttccgaaagaatatgagaacctggaagaagttctgcctgaaggctttctgaaacgtaccagcagcattggtaaagttattggttgggcaccgcagatggcagttctgagccatccgagcgttggtggttttgttagccattgtggttggaatagcaccctggaaagcatgtggtgtggtgtgccgatggcagcatggcctctgtatgcagaacagaccctgaatgcctttctgctggttgttgaactgggtttagcagcagaaattcgtatggattatcgtaccgataccaaagccggttatgatggtggtatggaagttaccgttgaagaaattgaagatggcattcgcaaactgatgagtgatggtgaaattcgcaacaaagtgaaggatgtcaaagaaaaatcacgtgcagcagttgttgaaggtggtagcagctatgcaagtattggcaaattcattgaacatgtgagcaacgtgaccatttaa

SEQ ID NO: 139 - protein, 479 aa, C. papaya
MGKPVNDKHVLVIPFPAQGHMIPLLDLTQQLAISGLTITILVTPKNLPILSPLLASHSSIQTLLLPFPSHPSIPAGAENTKDMPATSFFTMMPVLGQLHDPLVHWFNTHPSPPCAVISDIFLGWTHRLATELGVRRFVFSPSGAFALSIIYSLWREMPKRTNHDNQTEVISFPKLPNAPKFNWRSVSTIYQSYVEGDPDSEFVKQGFWDDMASWGLVINTFTELEKVYLDHLRAELGHDRIWGVGPLHLLADESSSEPKQRGGASSVSVPELMTWLDSCEDRKVVYICFGSQAVLTNSQMAALASALEKSRVRFVWSVKNPTRGTGNSDKDGVIPVGFENRVEDRGRVIKGWAPQVSILNHRAVGAFLTHCGWNSVFEAVVAGVPMLAWPMRADQFSNATLLVDYFKVATKVCEGPQTVPDSTELARHFVELLSENRVEREKAMELRNAAVKAIKDGGSSARDLEKLVQQIEELEIQSN

SEQ ID NO: 140 - DNA, 1440 bp, C. papaya
atgggtaaaccggtgaatgataaacatgttctggttattccgtttccggcacagggtcatatgattccgctgctggatctgacacagcagctggcaattagcggtctgaccattaccattctggttaccccgaaaaatctgccgattctgagccctctgctggcaagccatagcagcattcagaccctgctgctgccgtttccgagccatccgagcattccggcaggcgcagaaaataccaaagatatgcctgcaaccagcttttttaccatgatgccggttctgggtcagctgcatgatccgctggttcattggtttaatacccatccgagtccgccttgtgcagttattagcgatatttttcttggttggacccatcgtctggcaaccgaactgggtgttcgtcgttttgtttttagcccgagcggtgcatttgcactgagcattatctatagcctgtggcgtgaaatgccgaaacgtaccaatcatgataatcagaccgaagtgattagctttccgaaactgccgaatgcaccgaaatttaactggcgtagcgttagcaccatttatcagagctatgttgaaggtgatccggatagcgaatttgtgaaacaaggtttttgggatgatatggcaagctggggtttagtgattaatacctttacggaactggaaaaggtgtatctggatcatctgcgtgcagaactgggtcatgatcgtatttggggtgttggtccgctgcatctgctggccgatgaaagcagcagcgaaccgaaacagcgtggtggtgcaagcagcgttagcgtgccggaactgatgacctggctggatagctgtgaagatcgtaaagttgtgtatatttgctttggtagccaggcagttctgaccaatagccagatggcagcactggcaagcgcactggaaaaaagccgtgttcgctttgtttggagcgttaaaaatccgacacgtggcaccggtaatagcgataaagatggtgttattccggtgggttttgaaaatcgtgtggaagatcgtggtcgtgttattaaaggttgggcaccgcaggttagcattctgaatcatcgtgcagttggtgcatttctgacccattgtggttggaatagcgtttttgaagcagttgttgccggtgttccgatgctggcatggccgatgcgtgccgatcagtttagcaatgcaaccctgctggttgattatttcaaagttgcaaccaaagtttgtgaaggtccgcagaccgtgccggatagcacagaactggcacgtcattttgttgaactgctgagcgaaaatcgcgttgaacgtgaaaaagcaatggaactgcgtaatgcagcagtgaaagcaattaaagatggcggtagcagcgcacgtgatctggaaaaactggttcagcagattgaagaacttgaaatccagagcaactaa

SEQ ID NO: 141 - protein, 479 aa, S. pennellii
MSENHPHVLIFPYPAQGHMLPLLDFTHQLVNNGVHITILVTPKNLPFLNPLLSRNPSIKTLVLPFPSHPSIPAGVENVKDLPANGFLSMMCNLGKLRDPILDWFGNHPSPPSAIISDMFLGFTHEIATQLGIRRYVFSPSGALALSVVYSLWREMPKRKDPNDENENFHFPNIPNSPKFPFWQISPIYRSYVEGDPSTEFIRECYLADIASHGIVFNTFIELENVYLDYLMKYLGHNRVWSVGPVLPPGEDDVSVQSNRGGSSSVLASEILAWLDRCEDHSVVYVCFGSQAVLTNKQMEELAIALDKSGVHFILSAKRATKGHASNDYGVIPSWFEEKVAGRGLVVRDWAPQVLILKHRAIAAFLTHCGWNSTLESLIAGVPLLTWPMGADQFANANLLVDEHEVAIRACEGAQTVPNSDELAALLAEAVQGNKVEERRLRASKLRKIAINGIKEGGNSFKELAAFVKHLREEATIIEA

SEQ ID NO: 142 - DNA, 1440 bp, S. pennellii
atgagcgaaaatcatccgcatgttctgatttttccgtatccggcacagggtcatatgctgccgctgctggattttacccatcagctggttaataatggtgtgcatattaccattctggtgaccccgaaaaatctgccgtttctgaatccgctgctgagccgtaatccgagcattaaaaccctggttctgccttttccgagccatccgagtattccggcaggcgttgaaaatgttaaagatctgcctgcaaatggctttctgagcatgatgtgtaatctgggtaaactgcgtgatccgattctggattggtttggtaatcatccgagtccgcctagcgcaattattagcgatatgtttctgggctttacccatgaaattgcaacacagctgggtattcgtcgttatgtttttagcccgagcggtgcactggcactgagcgttgtttatagcctgtggcgtgaaatgccgaaacgtaaagatccgaatgatgaaaacgagaactttcactttccgaatattccgaacagcccgaaatttccgttttggcagattagcccgatttatcgtagctatgttgaaggtgatccgagcaccgaatttattcgtgaatgttatctggcagatattgcgagccatggcattgtgtttaacacctttattgaactggaaaacgtgtacctggactacctgatgaaatatctgggtcataatcgtgtttggagcgttggtccggttctgccaccgggtgaagatgatgttagcgttcagagcaatcgtggtggtagcagcagcgttctggcaagcgaaattctggcatggctggatcgttgtgaagatcatagcgttgtgtatgtttgttttggtagccaggcagttctgaccaataaacaaatggaagaactggcaattgcgctggataaaagcggtgttcattttattctgagcgcaaaacgtgcaaccaaaggtcatgcaagcaatgattatggtgttattccgagctggtttgaagaaaaagttgcaggtcgtggtctggttgttcgtgattgggcacctcaggttctgattctgaaacatcgtgcaattgccgcatttctgacccattgtggttggaatagcaccctggaaagcctgattgccggtgttcctctgctgacctggccgatgggtgcagatcagtttgcaaatgcaaatctgctggttgatgaacatgaagttgcaattcgtgcatgtgaaggtgcacagaccgttccgaatagtgatgaactggcagcactgctggcagaagcagttcagggtaataaagttgaagaacgtcgtctgcgtgcaagcaaactgcgtaaaattgcgattaacggtattaaagaaggtggcaacagctttaaagagctggcagcatttgtaaaacatctgcgtgaagaagcgaccattattgaagcataa

SEQ ID NO: 143 - protein, 470 aa, T. cacao
MDTISSNCSSHHAVLFPFMSKGHTIPILHLARLLLRRGLAVTVFTTPGNRPFIAKSLADTSASIIDINYPENIPEIPAGVESTDALPSISLFVPFCAATKLMQHEFERKLQSLLPVSFVVSDGFLWWTLESATKFGLPRLMFNGMSQYASTVSKAVAEDRLLFGPESDDELITVTQFPWIRVTRNDFEPILSSKPDPDSPPMRLFMDQVIAAENSKGKLVNSFYELEKYFFDSCNLEERLKAWSVGPLCLSEPPKVEHEHEPKKKPSWIKWLDQKLDEGCSVLYVAFGSQADISSEQLKQIATGLEESKVNFLWVVRKKESELGEGFEERVKETGIVVREWVDQKEILMHQSVQGFLSHCGWNSVLESICAGVPILAWPMMADQPLNARMVVEEIKVGLRVETCDGTVKGLVKWEGLMKMVRELMEGEMGKEVRIKVKELAELAKMAMEENTGSSWRTLDMLINEFCNNK

SEQ ID NO: 144 - DNA, 1413 bp, T. cacao
atggataccattagcagcaattgtagcagccatcatgcagttctgtttccgtttatgagcaaaggtcataccattccgattctgcatctggcacgtctgctgctgcgtcgtggtctggcagttaccgtttttaccacaccgggtaatcgtccgtttattgcaaaaagcctggcagataccagcgcaagcattatcgatattaactatccggaaaacatcccggaaattccggcaggcgttgaaagcaccgatgcactgccgagcattagcctgtttgttccgttttgtgcagcaaccaaactgatgcagcatgaatttgaacgtaaactgcagagcctgctgccggttagctttgttgttagtgatggttttctgtggtggaccctggaaagcgcaacaaaatttggtctgcctcgtctgatgtttaatggcatgagccagtatgcaagcaccgttagcaaagcagttgcagaagatcgtctgctgtttggtccggaaagtgatgatgaactgattaccgttacacagtttccgtggattcgtgttacccgtaatgattttgaaccgattctgagcagcaaaccggatcctgatagccctccgatgcgtctgtttatggatcaggttattgcagccgaaaacagcaaaggtaaactggtgaatagcttctacgagctggaaaagtattttttcgatagctgcaatctggaagaacgtctgaaagcatggtcagttggtccgctgtgtctgagcgaaccgcctaaagttgaacatgaacacgaaccgaaaaaaaagccgagctggattaaatggctggatcagaaactggatgaaggttgtagcgttctgtatgttgcatttggtagccaggcagatattagcagcgaacagctgaaacaaattgcaacaggcctggaagaaagcaaagtgaactttctgtgggttgtgcgtaaaaaagaaagcgaattaggtgaaggttttgaagaacgcgttaaagaaaccggtattgttgttcgtgaatgggtcgatcagaaagaaattctgatgcaccagagcgttcagggttttctgagccattgtggttggaatagcgtgctggaaagcatttgtgccggtgtgccgattctggcatggccgatgatggcagatcagccgctgaatgcacgtatggttgttgaagaaattaaagttggtctgcgtgtggaaacctgtgatggcaccgttaaaggtctggttaaatgggaaggtctgatgaaaatggttcgtgaactgatggaaggtgaaatgggtaaagaagtgcgcatcaaagttaaagaactggccgaactggcaaaaatggcaatggaagaaaataccggtagcagctggcgtaccctggatatgctgattaatgaattctgcaacaacaaataa

SEQ ID NO: 145 - protein, 478 aa, S. indicum
MDTRKRSIRILMFPWLAHGHISAFLELAKSLAKRNFVIYICSSQVNLNSISKNMSSKDSISVKLVELHIPTTILPPPYHTTNGLPPHLMSTLKRALDSARPAFSTLLQTLKPDLVLYDFLQSWASEEAESQNIPAMVFLSTGAAAISFIMYHWFETRPEEYPFPAIYFREHEYDNFCRFKSSDSGTSDQLRVSDCVKRSHDLVLIKTFRELEGQYVDFLSDLTRKRFVPVGPLVQEVGCDMENEGNDIIEWLDGKDRRSTVFSSFGSEYFLSANEIEEIAYGLELSGLNFIWVVRFPHGDEKIKIEEKLPEGFLERVEGRGLVVEGWAQQRRILSHPSVGGFLSHCGWSSVMEGVYSGVPIIAVPMHLDQPFNARLVEAVGFGEEVVRSRQGNLDRGEVARVVKKLVMGKSGEGLRRRVEELSEKMREKGEEEIDSLVEELVTVVRRRERSNLKSENSMKKLNVMMMENREGMLSENA

SEQ ID NO: 146 - DNA, 1437 bp, S. indicum
atggatacccgtaaacgtagcattcgcattctgatgtttccgtggctggcacatggtcatattagcgcatttctggaactggcaaaaagcctggcaaaacgtaatttcgtgatttatatctgtagcagccaggtgaatctgaacagcattagcaaaaatatgagcagcaaagatagcatcagcgtgaaactggttgaactgcatattccgaccaccattctgcctccgccttatcataccaccaatggtctgccaccgcatctgatgagcaccctgaaacgtgcactggatagcgcacgtccggcatttagcaccctgctgcagacactgaaaccggatctggttctgtatgattttctgcagagctgggcaagcgaagaagcagaaagccagaatattccggcaatggtttttctgagtaccggtgcagcagcaattagctttattatgtatcactggtttgaaacccgtccggaagaatatccgtttcctgcaatctattttcgcgaacacgagtatgataacttttgccgttttaaaagcagcgatagcggcaccagcgatcagctgcgtgttagcgattgtgtgaaacgtagccatgatctggtgctgattaaaacctttcgtgaactggaaggtcagtatgtggattttctgagcgatctgacccgcaaacgttttgttccggttggtccgctggttcaagaggttggttgtgatatggaaaatgaaggcaacgatatcatcgaatggctggatggtaaagatcgtcgtagcaccgtttttagcagctttggtagcgaatattttctgtccgccaacgaaattgaagaaattgcatatggcctggaactgagcggtctgaactttatttgggttgttcgttttccgcacggtgacgaaaaaatcaaaatcgaagaaaaactgccggaaggtttcctggaacgtgttgaaggtcgtggtctggttgtggaaggttgggcacagcagcgtcgtattctgagccatccgagcgttggtggttttctgtcacattgtggttggagcagcgttatggaaggtgtttatagcggtgttccgattattgcagttccgatgcatctggatcagccgtttaatgcacgtctggttgaagcagttggttttggtgaagaagttgttcgtagccgtcagggtaatctggatcgtggtgaagttgcacgtgttgttaaaaaactggttatgggtaaaagcggtgaaggtctgcgtcgtcgtgtggaagaactgagtgaaaaaatgcgtgaaaaaggcgaagaagaaatcgatagcctggtagaagaactggttaccgttgttcgtcgtcgcgaacgtagcaatctgaaaagcgaaaacagcatgaaaaagctgaacgtgatgatgatggaaaaccgtgaaggtatgctgagcgaaaatgcataa

SEQ ID NO: 147 - protein, 477 aa, P. trichocarpa; identical to SEQ ID NO: 101

SEQ ID NO: 148 - DNA, 1434 bp, P. trichocarpa; identical to SEQ ID NO: 102

SEQ ID NO: 149 - protein, 467 aa, H. annuus; identical to SEQ ID NO: 103

SEQ ID NO: 150 - DNA, 1404 bp, H. annuus; identical to SEQ ID NO: 104

SEQ ID NO: 151 - protein, 486 aa, A. chinensis
MATQAHQPHFIVFPLMAQGHMIPMIDIAKLLAQRGVKVTIVTTPLNAEQFKTIIARAKLSIQFLELGFPCKEAGLPEGCENLDKLPSFDWASKFFVATSLLKEPLEQKLGEMKPKPSCIISDMGFPWTSDLATKFHIPRLVFHGTCCFSLLCSLNVKAHNVLDQVNSDSEYFVVPGLPHKIELTKAQLPGFNPSSSSGLKSVSDQIRKAEKEVYGVVVNTFEELEAEYVMGYKKAKGERVWCIGPVSMCNKEVLDKADRGKKASIDEHHCLKWLDSHDPGSVIYACLGSLSRLTTPQMIEIGLGLEESNRPFIWVVRENSDGLEKWMLEEGFEERTRERGLLIRGWAPQVLILSHPSIGAFFTHCGWNSTLEGVCAGVPMMTWPMFAEQFCNEKLVVQVLRIGVSLGVEVPMRWGEEEKVGVLVKKDTVKEAIDELMDGGIEGEERRTRARQLGEMANRATEEAGSSHLNITMLIQDVMEYANSDQ

SEQ ID NO: 152 - DNA, 1461 bp, A. chinensis
atggcaacccaggcacatcagccgcattttattgtttttccgctgatggcacagggtcatatgattccgatgattgatattgcaaaactgctggcacagcgtggtgttaaagttaccattgttaccacaccgctgaatgccgaacagtttaaaaccattattgcacgtgccaaactgagcattcagtttctggaactgggttttccgtgtaaagaagcaggtctgccggaaggttgtgaaaatctggataaactgccgagctttgattgggcaagcaaatttttcgttgcaaccagcctgctgaaagaaccgctggaacagaaactgggtgaaatgaaaccgaaaccgagctgtattattagcgatatgggctttccgtggaccagcgatctggcaaccaaatttcatattccgcgtctggtttttcatggcacctgttgttttagcctgctgtgtagcctgaatgttaaagcacataatgttctggatcaggtgaatagcgatagcgaatattttgttgttccgggtctgccgcataaaattgaactgaccaaagcacagctgcctggttttaatccgagcagcagcagcggtctgaaaagcgttagcgatcagattcgtaaagccgaaaaagaagtttacggcgttgttgtgaatacctttgaagaactggaagccgaatatgtgatgggttacaaaaaagcaaaaggtgaacgtgtttggtgtattggtccggttagcatgtgtaataaagaggtgctggataaagcagaccgtggtaaaaaagccagcattgatgaacatcattgtctgaaatggctggatagccatgatccgggtagcgttatttatgcatgtctgggtagcctgagccgtctgacaacaccgcagatgattgaaatcggtctgggtttagaagaaagcaaccgtccgtttatttgggttgttcgtgaaaatagtgatggcctggaaaaatggatgctggaagaaggttttgaggaacgtacccgtgaacgtggtctgctgattcgtggttgggcaccgcaggttctgattctgagccatccgagcattggtgcattttttacccattgtggttggaatagcaccctggaaggtgtttgtgccggtgtgccgatgatgacctggccgatgtttgcagaacagttttgtaatgaaaaactggtggttcaggttctgcgtattggtgttagcctgggtgttgaagttccgatgcgttggggtgaagaagaaaaagttggcgttctggttaaaaaggatacagtgaaagaagccattgacgaactgatggatggtggtattgaaggtgaagaacgtcgcacccgtgcacgtcagctgggcgaaatggcaaatcgtgcaaccgaagaagccggtagcagccatctgaatatcaccatgctgattcaggatgttatggaatatgccaacagcgatcagtaa

SEQ ID NO: 153 - protein, 492 aa, S. indicum
MASQSHQLHFVLFPLMAPGHMIPMIDIAKLLAQRSVLVSVITTPQNASRFGSTVARAVRAGLQIQLVEIRFPSVEAGLPEGCENLDTLPSLDMATNFFVALNLLQKEVEQVFDEMKPRPSCLISDMGLPWTTQIAEKFHIPRIVFHGTCCFSLLCSHNTMASQILDTLNSDSDYFEVPNLPDRIKLRKSQVTGSTTRKSAAWKDVADQIRAAEKTSYGVVVNSFQELEAEYVKEYSKVKGEKVWCIGPVSLCNKESLDLAQRGNSAAVDEQNCLKWLDSYEPGSVVYASLGSLARLTVQQMTELALGLEESNRPFIWALGGDKSGALEGWISENGFEERTKNRGLLIRGWAPQLLILSHQATGGFLTHCGWNSTVEGISAGVPMVTWPLFAEQFCNEKLVVEVLRIGVSIGVEVPVKWGEEEKVGVVVKKDDVKKALDLLMDEEEEGKERRRKARELGKLANKAIEEGGSSHVSMTLLIEEIMAKANHGGST

SEQ ID NO: 154 - DNA, 1479 bp, S. indicum
atggcaagccagagccatcagctgcattttgttctgtttccgctgatggcaccgggtcatatgattccgatgattgatattgcaaaactgctggcacagcgtagcgttctggttagcgttattaccacaccgcagaatgcaagccgttttggtagcaccgttgcacgtgccgttcgtgcaggtctgcagattcagctggttgaaattcgttttccgagcgttgaagccggtctgccggaaggttgtgaaaatctggataccctgccgagcctggatatggcaaccaacttttttgttgcactgaacctgctgcagaaagaagttgaacaggttttcgatgaaatgaaaccgcgtccgagctgtctgattagcgatatgggtctgccgtggaccacacagattgcagaaaaatttcatattccgcgtatcgtgtttcatggcacctgttgttttagcctgctgtgtagccataataccatggccagccagattctggatacactgaatagcgatagcgattattttgaagttccgaatctgccggatcgtattaaactgcgtaaaagccaggttaccggtagcaccacacgtaaaagcgcagcatggaaagatgttgcagatcagattcgtgcagcagaaaaaaccagctatggtgttgttgtgaacagctttcaagaactggaagccgaatatgtgaaagaatacagcaaagtgaaaggcgaaaaagtgtggtgtattggtccggttagcctgtgtaataaagaaagtctggatctggcccagcgtggtaatagcgcagccgttgatgaacagaattgtctgaaatggctggatagctatgaaccgggtagcgttgtttatgcaagcctgggtagcctggcacgtctgaccgttcagcagatgaccgaactggcactgggtttagaagaaagcaatcgtccgtttatttgggcattaggtggtgataaaagcggtgcactggaaggttggattagcgaaaatggttttgaagaacgtaccaaaaatcgcggtctgctgattcgtggctgggcaccgcagctgctgatcctgagtcatcaggcaaccggtggttttctgacccattgtggttggaatagcaccgtggaaggtattagtgccggtgttccgatggttacctggcctctgtttgcagaacagttttgtaatgaaaaactggtggttgaagtgctgcgtattggtgttagcattggtgtggaagttccggttaaatggggtgaagaagagaaagttggcgttgtggttaaaaaagacgatgtgaaaaaagcactggatctgctgatggatgaagaagaagagggtaaagaacgtcgtcgtaaagcacgtgaactgggtaaactggcaaataaagcaattgaagagggtggtagcagccatgttagcatgaccctgctgattgaagaaattatggcaaaagcaaatcatggtggcagcacctaa

SEQ ID NO: 155 - protein, 458 aa, T. cacao
MESKVDQPHVIVLPYPAQGHINPMFQFSKRLASKGFKATLAITVFISNTMKLESSGSVQIDTISDGYDAGGLASSGGIQHYLPRLEAIGSKTLAELIIKHKRTSRPIDCIIYDAAMPWALDVAKQYGLHGAAFFTQMCAVNYIYYNVHHKLLNLPICSTPISIPGLPLLQPGDLPSFVCSSEGSYIAYLGRVLNQFKNIDKADFILINTFYKLENEAVESMSKVYPVLTIGPTVPSIYLDKPVENDKAYGLDLFDFNSSTSTDWLSTKPPGSVIYVSFGSVTSISSKQMEEIARGLNNSNFYFLWVVRASEEAKLPKGFKEESGEKGLIVNWSPQLDVLSNEAVGCFFTHCGWNSTTEALSLGVPMVAMPQWTDQPTVGKYIEDVWKVGVRVKIDDVSGIVNREEIESCIRQVMEGERGKEIKENAKKWRELALEAVGEGGTSDRNIDEFMSKLRRTA

SEQ ID NO: 156 - DNA, 1377 bp, T. cacao
atggaaagcaaagttgatcagccgcatgttattgttctgccgtatccggcacagggtcatattaatccgatgtttcagtttagcaaacgtctggcaagcaaaggttttaaagcaaccctggcaattaccgtgtttattagcaataccatgaaactggaaagcagcggtagcgttcagattgataccattagtgatggttatgatgccggtggtctggccagcagcggtggtattcagcattatctgcctcgtctggaagccattggtagcaaaaccctggccgaactgattatcaaacataaacgtaccagccgtccgattgattgcattatctatgatgcagcaatgccgtgggcattagatgttgcaaaacagtatggtctgcatggtgcagcattttttacccagatgtgtgcagtgaactacatctattataacgtgcatcacaaactgctgaatctgccgatttgtagcaccccgattagcattccgggtctgccgctgctgcagcctggtgatctgccgagctttgtttgtagcagcgaaggtagctatattgcatatctgggtcgtgttctgaaccagttcaaaaacattgataaagccgacttcatcctgatcaacaccttctataagctggaaaatgaagccgttgaaagcatgagcaaagtttatccggttctgaccattggtccgaccgttccgagcatttatctggataaaccggttgaaaacgataaagcatatggtctggacctgtttgattttaacagcagcaccagcaccgattggctgagcaccaaaccgcctggtagcgttatttatgttagctttggtagcgtgaccagcattagcagcaaacaaatggaagaaattgcacgcggtctgaataacagcaacttttatttcctgtgggttgttcgtgcaagcgaagaagcaaaactgccgaaaggctttaaagaagaatcaggcgaaaaaggcctgattgttaattggagtccgcagctggatgttctgagcaatgaagcagttggttgcttttttacacattgcggttggaatagcaccaccgaagcactgagcctgggtgttccgatggttgcaatgccgcagtggaccgatcagccgaccgttggcaaatatatcgaagatgtttggaaagttggtgtgcgcgtgaaaattgatgatgttagcggtattgtgaaccgcgaagaaatcgaaagctgtattcgtcaggttatggaaggtgaacgtggcaaagaaattaaagaaaacgccaaaaaatggcgtgaactggcactggaagcggttggtgaaggtggcaccagcgatcgtaatattgatgaatttatgagcaaactgcgtcgcaccgcataa

SEQ ID NO: 157 - protein, 480 aa, C. sativus
MGSEGRQLHIFMFPFMAHGHMIPIVDMAKLFASRGIKITIVTTPLNSISISKSLHNCSPNSLIQLLILKFPAAEAGLPDGCENADSIPSMDLLPKFFEAVSLLQPPFEEALHNNRPDCLISDMFFPWTNDVADRVGIPRLIFHGTSCFSLCSSEFMRLHKPYQHVSSDTEPFTIPYLPGDIKLTKMKLPIFVRENSENEFSKFITKVKESESFCYGVVVNSFYELEAEYVDCYKDVLGRKTWTIGPLSLTNTKTQEITLRGRESAIDEHECLKWLDSQKPNSVVYVCFGSLAKFNSAQLKEIAIGLEASGKKFIWVVRKGKGEEEEEEQNWLPEGYEERMEGTGLIIRGWAPQVLILDHPSVGGFVTHCGWNSTLEGVAAGVPMVTWPVGAEQFYNEKLVTEVLKTGVGVGVQKWAPGVGDFIESEAVEKAIRRIMEKEGEEMRNRAIELGKKAKWAVGEEGSSYSNLDALIEELKSLAF

SEQ ID NO: 158 - DNA, 1443 bp, C. sativus
atgggtagcgaaggtcgtcagctgcatatctttatgtttccgtttatggcacatggtcatatgattccgattgtggatatggcaaaactgtttgcaagccgtggtatcaaaattaccattgttaccacaccgctgaacagcattagcattagtaaaagcctgcataattgtagcccgaatagcctgattcagctgctgattctgaaatttccggcagccgaagcaggtctgccggatggttgtgaaaatgcagatagcattccgagcatggatctgctgccgaaattctttgaagcagttagcctgctgcagcctccgtttgaagaagcactgcataacaatcgtccggattgtctgattagcgatatgttttttccgtggaccaatgatgttgcagatcgtgttggtattccgcgtctgatttttcatggcaccagctgttttagcctgtgtagcagcgaatttatgcgtctgcataaaccgtatcagcatgttagcagcgataccgaaccgtttaccattccgtatctgcctggtgatattaaactgaccaaaatgaaactgccgatctttgtgcgtgaaaacagcgaaaatgaattcagcaaattcatcaccaaggtgaaagaaagcgaaagcttttgctatggtgttgtggtgaacagcttttatgaactggaagccgaatatgtggattgctataaagatgttctgggtcgtaaaacctggaccattggtccgctgagcctgaccaataccaaaacacaagaaattaccctgcgtggtcgtgaaagcgcaattgatgaacatgaatgtctgaaatggctggatagccagaaaccgaatagcgttgtttatgtttgctttggtagcctggccaaatttaacagcgcacagctgaaagaaattgccattggtctggaagcaagcggcaaaaaattcatttgggttgtgcgtaaaggtaaaggcgaagaagaagaggaagaacagaattggctgcctgaaggttatgaagaacgtatggaaggcaccggtctgattattcgtggttgggcaccgcaggttctgattctggatcatccgagcgttggtggttttgttacccattgtggttggaatagcaccctggaaggtgttgcagccggtgttccgatggttacctggcctgttggtgcagaacagttctataatgaaaaactggttaccgaggtgctgaaaaccggtgttggtgtgggtgttcagaaatgggcacctggtgttggcgattttattgaaagcgaagcagttgaaaaagccattcgtcgcattatggaaaaagaaggtgaagaaatgcgtaaccgtgcaattgaactgggtaaaaaagcaaaatgggcagttggtgaagaaggtagcagctatagtaatctggatgcactgattgaagaactgaaaagcctggccttttaa

SEQ ID NO: 159 - protein, 485 aa, P. trichocarpa
MGSLGHQLHIFFLPFFAHGHMIPSVDMAKLFASRGIKTTIITTPLNAPFFSKTIQKTKELGFDINILTIKFPAAEAGLPEGYENTDAFIFSENAREMTIKFIKATTFLQAPFEKVLQECHPDCIVADVFFPWATDAAAKFGIPRLVFHGTSNFALSASECVRLYEPHKKVSSDSEPFVVPDLPGDIKLTKKQLPDDVRENVENDFSKFLKASKEAELRSFGVVVNSFYELEPAYADYYKKVLGRRAWNVGPVSLCNRDTEDKAGRGKETSIDHHECLKWLDSKKPNSVVYICFGSTTNFSDSQLKEIAAGLEASGQQFIWVVRRNKKGQEDKEDWLPEGFEERMEGVGLIIRGWAPQVLILDHEAIGAFVTHCGWNSTLEGITAGKPMVTWPIFAEQFYNEKLVTDVLKTGVGVGVKEWFRVHGDHVKSEAVEKTITQIMVGEEAEEMRSRAKKLGETARKAVEEGGSSYSDFNALIEELRWRRP

SEQ ID NO: 160 - DNA, 1458 bp, P. trichocarpa
atgggtagcctgggtcatcagctgcatatcttttttctgccgttttttgcacatggccatatgattccgagcgttgatatggcaaaactgtttgcaagccgtggtattaaaaccaccattattaccacaccgctgaacgcaccgttttttagcaaaaccattcagaaaaccaaagagctgggcttcgatattaacatcctgaccatcaaatttccggcagcagaagcaggtctgccggaaggttatgaaaataccgatgcatttatcttcagcgaaaatgcacgtgagatgacgatcaaattcattaaagcaaccacctttctgcaggcaccgtttgaaaaagttctgcaagaatgtcatccggattgtattgttgccgatgttttttttccgtgggcaaccgatgcagcagcaaaatttggtattccgcgtctggtttttcatggcaccagcaattttgcactgagcgcaagcgaatgtgttcgtctgtatgaaccgcataaaaaagttagcagcgatagcgaaccgtttgttgttccggatctgcctggtgatattaaactgaccaaaaaacagctgccggatgatgttcgtgaaaatgtggaaaatgacttcagcaaattcctgaaagcaagcaaagaagcagaactgcgtagctttggtgttgttgtgaatagcttttatgaactggaaccggcatatgcggactactacaaaaaagtgctgggtcgtcgtgcatggaatgttggtccggttagcctgtgtaatcgtgataccgaagataaagcaggtcgtggtaaagaaaccagcattgatcatcatgaatgtctgaaatggctggacagcaaaaaaccgaatagcgttgtgtatatttgctttggtagcaccacgaattttagcgatagccagctgaaagaaattgcagccggtctggaagcaagcggtcagcagtttatttgggttgttcgtcgtaacaaaaaaggccaagaggataaagaagattggctgcctgaaggctttgaagaacgtatggaaggtgttggtctgattattcgtggttgggcaccgcaggttctgattctggatcatgaagcaattggtgcatttgttacccattgtggttggaatagcaccctggaaggtattaccgcaggtaaaccgatggttacctggccgatttttgcagaacagttctataatgaaaaactggtgaccgatgtgctgaaaaccggtgttggtgtgggtgttaaagaatggtttcgtgttcatggtgatcacgttaaaagcgaagcagtggaaaaaaccattacgcagattatggttggtgaagaggccgaagaaatgcgtagccgtgccaaaaaactgggtgaaaccgcacgtaaagcagttgaagaaggtggtagcagctatagtgattttaatgccctgattgaagaactgcgctggcgtcgtccgtaa

SEQ ID NO: 161 - protein, 484 aa, A. chinensis
MVSKPHKLHIYFFPMIASGHLIPMVDMARLFAQRGVKATIILTPFNAALFSKTIERDRELGLETSIRLINFPFAEVGMPEGCENLSSITSPEMFPKIFKATELLQQPLEKLLEEDRPDCLVADMYFPWATEVASKHGIPRLAFHGTGAYALCVHHVISQQEPYKNVESDSEVFTVPDLPDTITMTKRQLPDHIRDGTKNHMEKFIEKVTEAEMKSYGVLVNSFHELEPAYSEYYKEVVGRRTWHIGPVSLSNRDNEDKARRGNKTSIDEHECLSWLASKKPNSVLYVCFGSLSSFSTAQLLEIAMGLEASGQQFIWVVRKDKSKEKENEEWLPEAFEQRLEGRGIIIRGWAPQVLILDHESVGGFMTHCGWNSILEGVTAGVPMITWPHFAEQFYNEKLVTNILRVGVGVGAQEWCRWPDDCKIYVKKEDIEKAVAQLMDSEEAEETRSRAKALGAMAKKAVEKGGSSYSDLSAFLEELELNRN

SEQ ID NO: 162 - DNA, 1455 bp, A. chinensis
atggttagcaaaccgcataaactgcacatctattttttcccgatgattgcaagcggtcatctgattccgatggttgatatggcacgtctgtttgcacagcgtggtgttaaagcaaccattattctgaccccgtttaatgcagcactgtttagcaaaaccattgaacgtgatcgtgaactgggtttagaaaccagcattcgtctgattaactttccgtttgccgaagttggtatgccggaaggttgtgaaaatctgagcagcattaccagtccggaaatgtttccgaaaatctttaaagccaccgaactgctgcaacagccgctggaaaaactgctggaagaagatcgtccggattgtctggttgcagatatgtattttccgtgggcaaccgaagttgcaagcaaacatggtattccgcgtctggcatttcatggtacaggtgcctatgcactgtgtgttcatcatgttattagccagcaagagccgtataaaaacgttgaaagcgatagcgaagtttttaccgttccggatctgccggataccattaccatgaccaaacgtcagctgccggatcatattcgtgatggcaccaaaaatcacatggaaaagtttatcgaaaaagtgaccgaagccgagatgaaaagctatggtgttctggttaatagctttcatgaactggaaccggcatatagcgaatattacaaagaagttgttggtcgtcgtacctggcatattggtccggttagcctgagcaatcgtgataatgaagataaagcacgtcgcggtaataaaacgagcattgatgaacatgaatgtctgagctggctggcaagcaaaaaaccgaatagcgttctgtatgtttgttttggtagcctgagtagctttagcaccgcacagctgttagaaattgcaatgggcttagaagccagcggtcagcagtttatttgggttgttcgtaaagacaaatccaaagaaaaagaaaacgaagagtggctgccggaagcatttgaacagcgtctggaaggtcgtggtattatcattcgtggttgggcaccgcaggttctgattctggatcatgaaagtgttggtggttttatgacccattgtggttggaatagcattctggaaggcgttaccgcaggcgttccgatgattacctggcctcattttgcagaacagttctataatgaaaaactggtgaccaacattctgcgtgttggtgttggcgttggtgcacaagaatggtgtcgttggcctgatgattgtaaaatctacgtgaaaaaagaggacatcgagaaagcagttgcacagctgatggatagtgaagaagccgaagaaacccgtagccgtgcaaaagcactgggtgcaatggcaaaaaaagccgttgaaaaaggtggtagcagctatagcgatctgagcgcctttctggaagaactggaattaaatcgcaactaa

SEQ ID NO: 163 - protein, 478 aa, B. vulgaris
MEEQKPHFLLVTFPAQGHVNPALQFAKRLLRTGAHVTFSTAASAHRCFDKAKIPSGMSFATFSDGYDAGFRATDGDVLDYLSTFRQRGAETLATLLENSVAEGRPVTCLVYTLLLPWVAEVARKFHVPSALLWIQPATVFDIYYYYFNGYHDIIYDCEKDPLWSLELPNLPLKLKSHDIPSFLLPSNPFLYTFALPTFEEQMEELDKEEKPKILVNTFEALEVDALKAIEKFKLIPIGPLLPSAFLNGKDPFDKSFGGDLFQKTKNSDYMKWLDSQEEYSSVIYVSFGSISVLSKAQMEELAKALIQIHRPFLWVIRENEKDEKDLREEHNEGELSCMEELKALGLIVPWCSQVEVLSHPSIGCFVTHCGWNSTLESLTCGVPMVGFPQWTDQTTNSKLIEDVWKIGVRVKVSKEEGGLVKSEEIKRCLEVVMESEEMKENAKNWKELAVEAAKEGGSSDRNLKAFMEELFNVDCKKP

SEQ ID NO: 164 - DNA, 1437 bp, B. vulgaris
atggaagaacagaaaccgcattttctgctggttacctttccggcacagggtcatgttaatccggcactgcagtttgcaaaacgtctgctgcgtaccggtgcacatgttacctttagcaccgcagcaagcgcacatcgttgttttgataaagcaaaaattccgagcggtatgagctttgcaacctttagtgatggttatgatgcaggttttcgtgcaaccgatggtgatgttctggattatctgagcacctttcgtcagcgtggtgcagaaaccctggcaaccctgctggaaaattcagttgcagaaggtcgtccggttacctgtctggtttataccctgctgctgccgtgggttgccgaagttgcacgtaaatttcatgttccgagcgcactgctgtggattcagcctgcaaccgtttttgatatctattactattatttcaacggctaccacgacatcatctatgattgtgaaaaagatccgctgtggtcactggaactgccgaatctgccgctgaaactgaaaagccatgatattccgagctttctgctgccgagcaatccgtttctgtatacctttgcactgccgacctttgaagaacaaatggaagaattggacaaagaagagaagccgaaaattctggtgaatacatttgaagccctggaagttgatgcactgaaagccattgaaaaattcaaactgattccgattggtccgctgctgcctagcgcatttctgaatggtaaagatccgtttgataaaagctttggtggtgacctgtttcagaaaaccaaaaacagcgattacatgaaatggctggatagccaagaagagtatagcagcgttatttatgttagctttggtagcattagcgttctgagcaaagcacagatggaagagttagcaaaagcactgattcagattcatcgtccttttctgtgggtgattcgtgaaaatgaaaaagacgagaaagatctgcgcgaagaacataatgaaggtgaactgagctgtatggaagaactgaaggcactgggtctgattgttccgtggtgtagccaggttgaagttctgagccatccgagcattggttgttttgttacccattgtggttggaatagcaccctggaaagcctgacctgtggtgttccgatggttggttttccgcagtggaccgatcagaccaccaatagtaaactgattgaagatgtgtggaaaattggtgtgcgtgtgaaagtgagcaaagaagaaggcggtctggttaaaagcgaagaaatcaaacgttgtctggaagtggttatggaatccgaagaaatgaaagagaatgccaagaactggaaagaactggcagttgaagcagcaaaagaaggtggtagcagcgatcgtaatctgaaagcattcatggaagaacttttcaacgtggactgcaaaaaaccgtaa

SEQ ID NO: 165 - protein, 450 aa, P. trichocarpa
MSEARNDLKHIAVLAFPVATHGPPLLSLVRRLSASASYAKFSFFSTKESNSKLFSKEDGLENIKPYNVSDGLPENYNFAGNLDEVMNYFFKATPGNFKQAMEVAVKEVGKDFTCIMSDAFLWFAADFAQELHVPWVPLWTSSSRSLLLVLETDLVHQKMRSIINEPEDRTIDILPGFSELRGSDIPKELFHDVKESQFAAMLCKIGLALPQAAVVASNSFEELDPDAVILFKSRLPKFLNIGPFVLTSPDPFMSDPHGCLEWLDKQKQESVVYISFGSVISLPPQELAELVEALKECKLPFLWSFRGNPKEELPEEFLERTKEKGKVVSWTPQLKVLRHKAIGVFVTHSGWNSVLDSIAGCVPMICRPFFGDQTVNTRTIEAVWGTGLEIEGGRITKGGLMKALRLIMSTDEGNKMRKKLQHLQGLALDAVQSSGSSTKNFETLLEVVAK

SEQ ID NO: 166 - DNA, 1353 bp, P. trichocarpa
atgagcgaagcacgtaatgacctgaaacatattgcagttctggcatttccggttgcgacccatggtccgcctctgctgagcctggttcgtcgtctgagcgcaagcgcaagctatgcaaaatttagcttttttagcaccaaagaaagcaacagcaagctgtttagcaaagaagatggtctggaaaacatcaaaccgtataatgttagtgatggcctgccggaaaattacaattttgcaggtaatctggatgaagtgatgaactactttttcaaagcaacccctggcaactttaaacaggcaatggaagttgcagttaaagaggtgggtaaagattttacctgcattatgagtgatgcctttctgtggtttgcagcagattttgcacaagaactgcatgttccgtgggttccgctgtggaccagcagcagccgtagcctgctgttagttctggaaaccgatctggttcatcagaaaatgcgtagcattattaacgaaccggaagatcgcaccattgatattctgcctggttttagcgaactgcgtggtagcgatattccgaaagaactgtttcatgatgtgaaagaaagccagtttgcagccatgctgtgtaaaattggtctggcactgccgcaggcagcagttgttgcaagcaatagctttgaagaactggatccggatgccgtgattctgtttaaaagccgtctgccgaaatttctgaatattggtccgtttgttctgaccagtccggatccgtttatgagcgatccgcatggttgtctggaatggctggataaacagaaacaagaaagcgtggtgtatattagctttggtagcgttattagcctgcctccgcaagaactggcagaactggttgaagcactgaaagaatgtaaactgccgttcctgtggtcatttcgtggtaacccgaaagaagaactgcctgaagaatttctggaacgcacaaaagaaaaaggtaaagttgttagctggacaccgcagctgaaagttctgcgtcataaagcaattggtgtttttgttacccatagcggttggaatagcgttctggatagcattgcaggttgtgttccgatgatttgtcgtccgttttttggtgatcagaccgttaatacccgtaccattgaagcagtttggggcacaggcctggaaattgaaggtggtcgtattaccaaaggtggtctgatgaaagcactgcgtctgattatgagcaccgatgaaggcaataaaatgcgcaaaaaactgcagcatctgcaaggtctggccctggatgcagttcagagcagcggtagcagcaccaaaaactttgaaaccctgctggaagttgtggccaaataa

SEQ ID NO: 167 - protein, 449 aa, S. indicum
MTLMKKRTIILIPYPAQGHVTPMLRLASLLSNLGLRPVVITPEFIHRRISPQINPEDGIRCLSITDGLDAETPPDFFSIERAMEENMPPILEALLRKMIDEEEEEGGGIACLVADLLASWAVDVARRCGVAAAGFWPAMHATYRLIAAIPHLIRTGVISESGCPRNPSAPICLSSNEPILTPNDLPWLIGSSSARISRFKFWTRTLQRAKTLRWLLTNTFPDECQSRKMTRCSNAQQVLEIGSLIMQALEISTGSFWENDLTCLDWLDKQTMGSVMYVSFGSWVSPIGEAKVKTLALSLQALRRPFIWVLGPTWRRGLPDGYVKSVAGHGRIVSWAPQLEVLQHPSVGCYLTHCGWNSTMEAIQCKKPLLCYPIAGDQFLNCAYIVNTWRIGVKIEGFGIEEVEDGIIKVTEDEQVSWRIERLYENLYGKEGSSKAMANLSTFIQDLGK

SEQ ID NO: 168 - DNA, 1350 bp, S. indicum
atgaccctgatgaaaaaacgcaccattattctgattccgtatccggcacagggtcatgttaccccgatgctgcgtctggcaagcctgctgagcaatctgggtctgcgtccggttgttattacaccggaatttattcatcgtcgtattagtccgcagattaatccggaagatggtattcgttgtctgagcattaccgatggtctggatgcagaaacccctccggattttttcagcattgaacgtgcaatggaagaaaacatgcctccgattctggaagcactgctgcgtaaaatgattgatgaagaggaagaagagggcggaggtattgcatgtctggttgccgatctgctggcaagctgggcagttgatgttgcacgtcgttgtggtgttgcagcagcaggtttttggcctgcaatgcatgcaacctatcgtctgattgcagcaattccgcatctgattcgtaccggtgttattagcgaaagcggttgtccgcgtaatccgagcgcaccgatttgcctgagcagcaatgaaccgattctgaccccgaatgatctgccgtggctgattggtagcagcagcgcacgtattagccgtttcaaattttggacccgtacactgcagcgtgcaaaaaccctgcgttggctgctgaccaatacctttccggatgaatgtcagagccgcaaaatgacccgttgtagcaatgcccagcaggttctggaaattggtagcctgattatgcaggcactggaaattagcaccggtagcttttgggaaaatgatctgacctgtctggattggctggataaacagaccatgggtagcgttatgtatgttagctttggtagctgggttagcccgattggtgaagcaaaagttaaaaccctggcactgagtctgcaggccctgcgtcgtccgtttatttgggttctgggtccgacctggcgtcgtggtctgccggatggttatgttaaaagcgttgcaggtcatggtcgtattgttagctgggcaccgcagctggaagttctgcagcatccgagcgttggttgttatctgacccattgtggttggaatagcaccatggaagcaattcagtgtaaaaaaccactgctgtgttatccgattgccggtgatcagtttctgaattgtgcctatattgttaatacctggcgcattggcgttaaaattgaaggttttggtattgaagaggtcgaggatggtattatcaaagtgaccgaagatgaacaggttagctggcgtattgaacgtctgtatgaaaatctgtatggtaaagaaggttccagcaaagcaatggcaaatctgagcacctttattcaggatctgggcaaataa

SEQ ID NO: 169 - protein, 453 aa, A. duranensis
MEKENGKAVHCVVLAYPAQGHINPMIQFSKRLLHEGVKVTLVTTLFYGKSLENFPPSMSFETISDGFDNGRHGEGLKLTVYNEVFAQRGSQTLSEVLEKCAISGYPVDCIIYDSFMPWALDVAKKFGIAGASYLTQNMPVNSVYYHVHIGKLRAPLTEDEILIPMLPKLQHRDMPSFFLSYQEDPAFLEMLVEQFSNIHEADWVLCNAFYELEKEVIDWTTKIWPKFRTIGPSIPSMFLDKRLKDDEEYGVTQFKSEECMDWLDKKAKGSVLYVSFGSLVPLDEEQIREVAYGLRDSGRYFLWVVRASEEAKLPKDFAKNSEKGLVVTWCSQLKVLSHEAVGCFVTHCGWNSTLEALSLGVPVIAVPQWSDQATNAKYLVDVWKVGIRPVVDEKKIMRKEALEDCIKELMESDKGKEIRINAVKLKNLAIEAVSEGGSSNKNIIEFVNSLKGY

SEQ ID NO: 170 - DNA, 1362 bp, A. duranensis
atggaaaaagaaaatggcaaagccgttcattgtgttgttctggcatatccggcacagggtcatattaatccgatgattcagtttagcaaacgcctgctgcatgaaggtgttaaagttaccctggttaccacactgttttatggtaaaagcctggaaaactttccgcctagcatgagctttgaaaccattagtgatggttttgataatggccgtcatggtgaaggtctgaaactgaccgtttataatgaagtttttgcacagcgtggtagtcagaccctgagcgaagttctggaaaaatgtgcaattagcggttatccggttgattgcattatctatgatagctttatgccgtgggcattagatgtggccaaaaaattcggtattgccggtgcaagctatctgacccagaatatgccggttaatagcgtgtattatcatgtgcatattggcaaactgcgtgcaccgctgaccgaagatgaaattctgattccgatgctgccgaaactgcagcatcgtgatatgccgagcttttttctgagctatcaagaagatcctgcctttctggaaatgctggttgaacagttttccaacattcatgaagcagattgggttctgtgcaacgcattctatgaacttgaaaaagaagtgatcgactggaccaccaaaatctggcctaaatttcgtaccattggtccgagcattccgagtatgtttctggataaacgtctgaaagatgatgaagaatatggcgtgacccagtttaaaagcgaagaatgtatggattggctggacaaaaaagcaaaaggtagcgttctgtatgttagctttggtagcctggttccgctggatgaagaacaaattcgtgaagttgcatatggtctgcgtgatagcggtcgttattttctgtgggttgttcgtgccagcgaagaagcaaaactgccgaaagattttgccaaaaacagcgaaaaaggtctggttgttacctggtgtagccagctgaaagttctgagccatgaagccgttggttgttttgttacccattgtggttggaatagcaccctggaagcactgagcctgggtgttccggttattgccgttccgcagtggtcagatcaggcaaccaatgcaaaatatctggttgatgtttggaaagtgggtattcgtccggttgttgatgagaaaaaaatcatgcgtaaagaggccctggaagattgtattaaagaactgatggaaagcgacaaaggcaaagaaattcgtattaatgccgtgaagctgaaaaacctggcaattgaagcagttagcgaaggtggtagcagcaacaaaaacattatcgaatttgtgaacagcctgaaaggctattaa

SEQ ID NO: 171 - protein, 468 aa, C. sinensis
MENIEKKAASCRLVHCLVLSYPAQGHINPLLQFAKRLDHKGLKVTLVTTCFISKSLHRDSSSSSTSIALEAISDGYDEGGSAQAESIEAYLEKFWQIGPRSLCELVEEMNGSGVPVDCIVYDSFLPWALDVAKKFGLVGAAFLTQSCAVDCIYYHVNKGLLMLPLPDSQLLLPGMPPLEPHDMPSFVYDLGSYPAVSDMVVKYQFDNIDKADWVLCNTFYELEEEVAEWLGKLWSLKTIGPTVPSLYLDKQLEDDKDYGFSMFKPNNESCIKWLNDRAKGSVVYVSFGSYAQLKVEEMEELAWGLKATNQYFLWVVRESEQAKLPENFSDETSQKGLVVNWCPQLEVLAHEATGCFLTHCGWNSTMEALSLGVPMVAMPQWSDQSTNAKYIMDVWKTGLKVPADEKGIVRREAIAHCIREILEGERGKEIRQNAGEWSNFAKEAVAKGGSSDKNIDDFVANLISSKSF

SEQ ID NO: 172 - DNA, 1407 bp, C. sinensis
atggaaaacatcgagaaaaaagcagcaagctgtcgtctggttcattgtctggttctgagctatccggcacagggtcatattaatccgctgctgcagtttgcaaaacgtctggatcataaaggtctgaaagttaccctggttaccacctgttttattagcaaaagcctgcatcgtgatagcagcagcagctcaaccagcattgcactggaagcaattagtgatggttatgatgaaggtggtagcgcacaggcagaaagcattgaagcatatctggaaaaattctggcagattggtccgcgtagcctgtgtgaactggttgaagaaatgaatggtagcggtgttccggttgattgcattgtttatgatagttttctgccgtgggcattagatgtggccaaaaaattcggtctggttggtgcagcatttctgacccagagctgtgcagttgattgtatctattatcatgtgaacaaaggcctgctgatgctgccgctgccggattcacagctgctgttaccgggtatgcctccgctggaaccgcatgatatgccgagctttgtgtatgatctgggtagttatccggcagttagcgatatggttgtgaaatatcagttcgacaacatcgataaagcagattgggttctgtgcaacaccttttatgaactggaagaagaggttgcagaatggctgggtaaactgtggtcactgaaaaccattggtccgaccgttccgagcctgtatctggataaacagctggaagatgataaagattatggctttagcatgtttaaaccgaacaacgagagctgcattaaatggctgaatgatcgtgcaaaaggtagcgttgtttatgttagctttggtagctatgcacagctgaaagtggaagaaatggaagaactggcatggggactgaaagcaaccaatcagtattttctgtgggttgttcgtgaaagcgaacaggcaaaactgcctgaaaactttagtgatgaaaccagccagaaaggtctggtggttaattggtgtccgcaactggaagttctggcacatgaagccaccggttgttttctgacacattgtggttggaatagcaccatggaagcactgagcctgggtgttccgatggttgcaatgccgcagtggtcagatcagagcaccaatgccaaatatatcatggatgtttggaaaacaggcctgaaagttccggcagatgaaaaaggtattgttcgtcgtgaagcaattgcccattgtattcgtgaaattctggaaggtgaacgcggtaaagaaattcgtcagaatgccggtgaatggtccaattttgccaaagaagcagttgcaaaaggcggtagcagcgataaaaacattgatgattttgtggccaacctgatcagcagcaaatccttttaa

SEQ ID NO: 173 - protein, 473 aa, A. duranensis
MESKTIRIALVSAPVYSHLRSILEFAKRLIRFYQDLHVTCLVPINGSPCNKTKALLQSLPPTIDYIFVSPKNLEDEVQDTHPAFLVRTLITRSLPLIHDEVKKLISKSRLIAIISDGIITQVLELVKDLNVLSYTYFPSSAMLLALCLYSENLDETTTSEYKDLLEPIKIPGCIPVQGSDLPDPFNDRTSETYKEFLEGSRRFFLADGILVNTFFDLEASTIKELQEQERRGIVPSIHAIGPFVQHESSMIEGNDNNTLECLNWLDKQQENSVLYVSFGSGGTISHKQIIELALGLELSGQKFLWLLKPPSKFDIIFDFGHFSEDPLKYLPSGFLERTKEQGIIVPYWAPQIKILGHAAIGGYLCHCGWNSILESVAHGIPMIAWPLFAEQRMNAALFCNGLKVAIRAKVNEMGIVERGEVAKVIKNLMIGDEGKEIRQRMRELKGSAEDAINEGGSSTRTLTQLVQKWKNLE

SEQ ID NO: 174 - DNA, 1422 bp, A. duranensis
atggaaagcaaaaccattcgtattgcactggttagcgcaccggtttatagccatctgcgtagcattctggaatttgcaaaacgtctgattcgcttctatcaggatctgcatgttacctgtctggttccgattaatggtagcccgtgtaataaaaccaaagcactgctgcagagcctgcctccgaccattgattatatctttgttagcccgaaaaaccttgaagatgaagttcaggatacccatccggcatttctggttcgtaccctgattacccgtagcctgccgctgattcatgatgaagttaaaaaactgatcagcaaaagccgtctgattgccattatttccgatggtattattacccaggttctggaactggtgaaagatctgaatgttctgagctatacctattttccgagcagcgcaatgctgctggcactgtgtctgtatagcgaaaatctggatgaaaccaccacgagcgaatataaagatctgctggaaccgatcaaaattccgggttgtattccggttcagggtagcgatctgccggatccgtttaatgatcgtaccagcgaaacctataaagaatttctggaaggtagccgtcgtttttttctggcagatggtattctggtgaacaccttttttgatctggaagccagcaccattaaagaactgcaagaacaagaacgtcgtggtattgtgccgagcattcatgcaattggtccgtttgttcagcatgaaagcagcatgattgaaggcaatgataataacaccctggaatgtctgaattggctggataaacagcaagaaaatagcgttctgtatgtgagctttggtagcggtggcaccattagccataaacaaattattgaactggccctgggtttagaactgagcggtcagaaattcctgtggctgctgaaaccgcctagcaaatttgatatcatctttgattttggccacttcagcgaagatccgctgaaatatctgccgagcggttttctggaacgtaccaaagaacagggtattattgttccgtattgggcaccgcagattaaaatcctgggtcatgcagcaattggtggttatctgtgtcattgtggttggaatagtattctggaaagcgttgcacatggtattccgatgattgcatggcctctgtttgcagaacagcgtatgaatgcagcactgttttgtaatggtctgaaagttgcaattcgtgccaaagtgaatgaaatgggtattgttgaacgtggtgaagttgcgaaagtgatcaaaaatctgatgattggtgatgaaggcaaagaaattcgtcagcgtatgcgtgaactgaaaggtagtgccgaagatgcaattaatgaaggtggtagcagcacccgtacactgacccagctggtgcagaaatggaaaaacctggaataa

SEQ ID NO: 175 - protein, 476 aa, S. indicum
MSADQKLTSLVFVPFPIMSHLATAVKTAKLLADRDERLSITVLVMKLPIDTLISSYTKNSPDARVKVVQLPEDEPTFTKLMKSSKNFFFRYIESQKGTVRDAVAEIMKSSRACRIAGFVIDMFCTPMIDVANELGVPTYMFFSSGSATLGLMFHLQSLRDDNNVDVMEYKNSDAAISIPTYVNPVPVAVWPSPVFEEDSGFLDFAKRFRETKGIIVNTFLEFETHQIRSLSDDKKIPPVYPVGPILQADENKIEQEKEKHAEIMRWLDKQPDSSVVFLCFGTHGCLEGDQVKEIAVALENSGHRFLWSLRKPPPKEKVEFPGEYENSEEVLPEGFLGRTTDMGKVIGWAPQMAVLSHPAVGGFVSHCGWNSVLESVWCGVPMAVWPLSAEQQANAFLLVKEFEMAVEIKMDYKKNANVIVGTETIEEAIRQLMDPENEIRVKVRALKEKSRMALMEGGSSYNYLKRFVENVVNNIS

SEQ ID NO: 176 - DNA, 1431 bp, S. indicum
atgagcgcagatcagaaactgaccagcctggtttttgttccgtttccgattatgagccatctggcaaccgcagttaaaaccgcaaaactgctggcagatcgtgatgaacgtctgagcattaccgttctggttatgaaactgccgattgataccctgattagcagctataccaaaaattcaccggatgcgcgtgttaaagttgttcagctgccggaagatgaaccgacctttaccaaactgatgaaaagcagcaaaaacttcttcttccgctatatcgaaagccagaaaggcaccgttcgtgatgcagttgcagaaattatgaaaagctcacgtgcatgtcgtattgccggttttgttattgatatgttttgcaccccgatgattgatgttgcaaatgaactgggtgttccgacctatatgttttttagcagcggtagcgcaaccctgggtctgatgtttcatctgcagagcctgcgtgatgataataatgttgatgtgatggaatacaaaaacagcgacgcagcaattagcattccgacatatgttaatccggttccggttgcagtttggccgagtccggtttttgaagaagatagcggttttctggattttgccaaacgttttcgtgaaaccaaaggcattattgtgaacacgtttctggaatttgaaacccatcagattcgtagcctgtccgatgataaaaagattccgcctgtttatccggttggtccgattctgcaggccgatgaaaacaaaattgaacaagagaaagaaaaacacgccgaaattatgcgttggctggataaacaaccggattcaagcgttgtttttctgtgttttggcacccatggttgtctggaaggtgatcaggttaaagaaattgcagttgccctggaaaatagcggtcatcgttttctttggagtctgcgtaaaccgcctcctaaagaaaaagttgaatttccgggtgaatatgagaacagcgaagaagttctgcctgaaggctttctgggtcgtaccaccgatatgggtaaagttattggttgggcaccgcagatggcagttctgagtcatccggcagttggtggttttgtgagccattgtggttggaatagcgttctggaaagcgtttggtgtggtgtgccgatggccgtttggcctctgagtgcagaacagcaggccaatgcatttctgctggtgaaagaattcgaaatggccgtggaaatcaaaatggactataaaaagaacgccaacgttatcgttggtacggaaaccattgaagaagcaattcgtcagctgatggatccggaaaatgaaattcgtgtgaaagttcgtgccctgaaagaaaagtcacgtatggcactgatggaaggtggtagctcatataactatctgaaacgctttgtggaaaacgtggtgaacaacatcagctaa

SEQ ID NO: 177 - protein, 473 aa, V. vinifera
MEQTELVFIPFPVIGHLASALEIAKLITKRDPRFSITIFIMKFPFGSTDGMDTDSDSIRFVTLPPVEVSSETTPSGHFFSEFLKVHIPLVRDAVHELTRSNSVRLSGFVIDMFCTHMIDVADEFGVPSYLFFSSGAAVLGFLLHVQFLHDYEGLDINEFKDSDAELDVPTFVNSIPGKVFPAGMFDKESGGAEMLLYHTRRFREVKGILVNTFIELESHAIQSLSGSTVPEVYPVGPILNTRMGSGGGQQDASAIMNWLDDQPPSSVVFLCFGSMGSFGADQIKEIAHALEHSGHRFLWSLRQPPPKGKMIPSDHENIEQVLPEGFLHRTARIGKVIGWAPQIAVLAHSAVGGFVSHCGWNSLLESVWYGVPVATWPIYAEQQINAFQMVKDLGLAVEIKIDYNKDRDHIVSAHEIENGLRNLMNINSEVRKKRKEMEKISHKVMIDGGSSHFSLGHFIEDMDSKVMKGKDAL

SEQ ID NO: 178 - DNA, 1422 bp, V. vinifera
atggaacagaccgaactggtgtttattccgtttccggttattggtcatctggcaagcgcactggaaattgcaaaactgattaccaaacgtgatccgcgttttagcattaccatcttcattatgaaatttccgtttggtagcaccgatggtatggataccgatagcgatagcattcgttttgttaccctgcctccggttgaagttagcagcgaaaccacaccgagcggtcacttttttagcgaatttctgaaagttcatattccgctggttcgtgatgcagtgcatgaactgacccgtagcaatagcgttcgtctgagcggttttgttattgatatgttttgcacccacatgattgatgtggcagatgaatttggtgttccgagctacctgttttttagcagcggtgcagcagttctgggttttctgctgcatgttcagtttctgcatgattatgaaggcctggatatcaacgagtttaaagatagtgatgcggaactggatgttccgacctttgttaatagcattccgggtaaagtttttccggcaggcatgtttgataaagaaagcggtggtgcagaaatgctgctgtatcacacccgtcgttttcgtgaagttaaaggtattctggtgaacacctttatcgaactggaaagccatgcaattcagagcctgagcggtagtaccgttccggaagtttatccggttggtccgattctgaatacccgtatgggtagtggtggtggtcagcaggatgcaagcgcaattatgaattggctggatgatcagcctccgagcagcgttgtttttctgtgttttggttcaatgggtagctttggtgcagatcagattaaagaaattgcacatgcactggaacatagcggtcatcgttttctttggagcctgcgtcagcctcctccgaaaggtaaaatgattccgagcgatcatgaaaacattgaacaggttctgccggaaggctttctgcatcgtaccgcacgtattggtaaagttattggttgggcaccgcagattgccgttctggcacatagcgcagttggtggttttgtgagccattgtggttggaatagcctgctggaaagcgtttggtatggtgtgccggttgccacctggccgatttatgcagaacagcagattaatgcattccagatggtgaaagatctgggtttagcagtggaaatcaaaatcgactataacaaagatcgcgaccatattgttagcgcacatgaaatcgaaaatggtctgcgtaatctgatgaacattaatagcgaagtgcgcaaaaaacgcaaagaaatggaaaaaatcagccacaaggttatgatcgatggtggtagcagccattttagcctgggtcattttattgaagatatggacagcaaagtgatgaaaggcaaagatgcactgtaa

SEQ ID NO: 179 - protein, 470 aa, H. annuus
MERTPHIAIVPSPGMGHLIPLVEFAKRLKNNHNISSTFIIPNEGPLTKSQQAFLDSLPNGLNHVILPPVSFDDLPNDIRMETRISLMVTRSLDSLREAVKSLVVETNMVALFVDLFGTDAFDVAIEFGVSPYVFFPSTAMALSLFLYLPKLDQMVSCEYRDLPEPVQIPGCIPVRGEDLLDPVQERKNDAYKWVLHNAKRYRMAEGIAVNSFKELEGGALKALLEDQPGKPRVYPVGPLVQAGSSSDVDGSGCLRWLDGQPCGSVLYISFGSGGTLSSNQLNELALGLELSEQRFIWVVRSPNDKPNATYFNSHGHEDPLGFLPKGFLERTKGIGFVVPSWAPQAQILSHSSTGGFLTHCGWNSILETVVHGVPVIAWPLYAEQRMNAVSLTEGIKVALRPKVDENGIVSRVEIARVVKGLIEGEEGKPIRSRIRELKDAASNVLSKDGCSTKTLEQLASKLKAKNNISI

SEQ ID NO: 180 - DNA, 1413 bp, H. annuus
atggaacgtacaccgcatattgcaattgttccgagtcctggtatgggtcatctgattccgctggttgaatttgcaaaacgcctgaaaaacaaccacaatattagcagcacctttatcattccgaatgaaggtccgctgaccaaaagccagcaggcatttctggatagcctgccgaatggtctgaatcatgttattctgcctccggttagctttgatgatctgccgaacgatattcgtatggaaacccgtattagcctgatggttacccgtagcctggatagtctgcgtgaagcagttaaaagcctggttgttgaaaccaatatggttgcactgtttgttgacctgtttggcaccgatgcatttgatgttgcaattgaatttggtgttagcccgtatgttttttttccgagcaccgcaatggcactgagcctgtttctgtatctgcctaaactggatcagatggttagctgtgaatatcgcgatctgccggaaccggtgcagattccgggttgtattccggttcgtggtgaagatctgctggatccggttcaagaacgtaaaaatgatgcctataaatgggtgctgcataacgcaaaacgttatcgtatggcagaaggtattgccgtcaatagctttaaagaactggaaggtggtgcactgaaagcactgctggaagatcagcctggtaaaccgcgtgtttatccggttggtccgctggtgcaggcaggtagcagcagtgatgttgatggtagcggttgtctgcgttggctggatggtcagccgtgtggtagcgttctgtatattagctttggtagtggtggcaccctgagcagcaatcagctgaatgaactggcactgggtttagaactgagcgaacagcgttttatttgggttgttcgtagccctaatgataaaccgaatgccacctattttaacagccatggtcatgaagatcctctgggttttctgccgaaaggttttctggaacgcaccaaaggtattggttttgttgtgccgagctgggcaccgcaggcacagattctgagccatagcagtaccggtggttttctgacccattgtggctggaatagcattctggaaaccgttgttcatggtgttccggttattgcatggcctctgtatgcagaacagcgtatgaatgcagttagcctgaccgaaggtattaaagttgcactgcgtccgaaagttgatgaaaatggtattgttagtcgtgtggaaattgcccgtgttgttaaaggtctgattgaaggtgaagaaggtaaaccgattcgtagccgtattcgtgaactgaaagatgcagcaagcaatgttctgagcaaagatggttgtagcaccaaaacactggaacagctggcaagcaaactgaaagccaaaaacaacatcagcatttaa

SEQ ID NO: 181 - protein, 476 aa, S. pennellii
MSPLHFFFFPMVAQGHMIPTLDMAKLVASRGVKATIITTPLNESVFSDSIERNKHLGIEIDIRLITFQAVENDLPIGCERLDLVPSPVLFNNFFKATAMMQEPFENLVKECRPDCIVSDMLYPWSTDSAAKFNIPRIVFHGTGFFALCVAESIKRNKPFKNVSTDSETFVVPNLPHQIRLTRTQLSPFDLEEKEAIIFKIFHEVREADSKSYGVIFNSFYELETDYFEYYTKFQDNKSWAIGPLSLCNRYIEDKAERGMKSCIDTHECLKWLDSKKSGSIVYICFGSGVTFTGSQIEELAMGIEDSGQEFIWVIREQENENSCLPEGFEERTKEKGLIIRGWAPQVLILDHEGVGAFVTHCGWNSTLEGISAGVPLVAWPVFAEQFLNEKLVTDVLRIGVGVGSVKWEAAASEGVKREEISKAIKRVMVGEEAEGFKNRAKEYKEKAREAIEEGGSSYNGLTNLLQDVSMFGTKID

SEQ ID NO: 182 - DNA, 1431 bp, S. pennellii
atgagtccgctgcacttttttttctttccgatggttgcacagggtcatatgattccgacactggatatggcaaaactggttgcaagccgtggtgttaaagcaaccattattaccacaccgctgaatgaaagcgtttttagcgatagcattgaacgcaataaacatctgggcatcgaaattgatattcgcctgattacctttcaggccgttgaaaatgatctgccgattggttgtgaacgtctggatctggttccgagtccggttctgtttaataactttttcaaagcaaccgccatgatgcaagaaccgtttgaaaatctggttaaagaatgtcgtccggattgcattgttagcgatatgctgtatccgtggtcaaccgatagcgcagccaaatttaacattccgcgtattgtttttcatggcaccggtttttttgcactgtgtgttgcagaaagcatcaaacgtaataaaccgttcaaaaacgttagcacggatagcgaaacctttgttgttccgaatctgccgcatcagattcgtctgacccgtacacagctgagcccgtttgatctggaagaaaaagaagccatcatcttcaaaatctttcacgaagtgcgtgaagcagatagcaaaagctatggtgttatcttcaacagcttctatgaactggaaaccgactatttcgagtactacaccaaattccaggataacaaaagctgggcaattggtccgctgagcctgtgtaatcgttatatcgaagataaagcagagcgtggtatgaaaagctgtattgatacccatgaatgtctgaaatggctggacagcaaaaaatcaggtagcattgtgtatatttgctttggtagcggtgttacctttaccggtagccagattgaagaactggcaatgggtattgaagatagcggtcaagaatttatctgggtgattcgcgaacaagaaaatgaaaatagctgtctgccggaaggttttgaagaacgtaccaaagaaaaaggcctgattattcgtggttgggcaccgcaggttctgattctggatcatgaaggtgttggtgcatttgttacccattgtggttggaatagcaccctggaaggtattagtgccggtgttccgctggttgcctggcctgtttttgcagaacagtttctgaacgaaaaactggtgaccgatgttctgcgtattggtgttggcgttggtagcgttaaatgggaagcagcagcaagcgaaggtgttaaacgtgaagaaatttccaaagccattaaacgtgttatggttggtgaagaagccgaaggctttaaaaaccgtgcgaaagagtataaagagaaagcacgcgaagcaattgaagaaggtggtagcagctataatggtctgaccaatctgctgcaggatgttagcatgtttggcaccaaaatcgattaa

SEQ ID NO: 183 - protein, 494 aa, B. vulgaris
MGAEPQRLHVVFFPLMAAGHLIPTLDIAKLFAAHHVKTTIITTPLNAPCFTKPLESYKNLGHRIDIEIIPFPSKEAGLPEGLENFDQFTSDQMAVKFLKATELLQESFEKFLEKHKPNCIVTDMLMPFTNNVAAKFNIPRIVFHGCSYFALCMMHTLLKYQPHKSLLSDDEEFLVPNLPHEINLTRSRLPDMMRGQGDKELNDAWMKIFIHAMEAEENSFGVIMNSFYELEPEYVEYYRNVMGRKAWHIGPVSLCNRENEAKFQRGKDSSINEHECLKWLDSKKPKSVVYICFGSLAEVPTLQLREIAMGLEASEQDFIWVVRRGKENVEEEKIEEWLPYDFEDRMEGKGLIIRGWAPQVLILDHEAIGAFVTHCGWNSTLEGISCGVPMVTWPVFAEQFYNEKLVTEVLKTGVAVGAKKWSRILEVNLKSEDIKNAIRRVMVGEEALVLRSKAKKLKELARKAVEIGGSSYSDMHSLIQDLSSYNANGYKQYL

SEQ ID NO: 184 - DNA, 1485 bp, B. vulgaris
atgggtgcagaaccgcagcgtctgcatgttgttttttttccgctgatggcagcaggtcatctgattccgacactggatattgcaaaactgtttgcagcacatcatgtgaaaaccaccattattaccacaccgctgaatgcaccgtgttttacaaaaccgctggaaagctataaaaacctgggtcatcgtattgacattgaaattattccgtttccgagcaaagaagcaggtctgccggaaggtctggaaaattttgatcagtttaccagcgatcagatggccgtgaaatttctgaaagcaaccgaactgctgcaagaaagctttgaaaaattcctggaaaaacacaagccgaactgcattgttaccgatatgctgatgccgtttaccaataatgttgcagccaaatttaacatccctcgcattgtttttcatggctgtagctattttgcactgtgtatgatgcataccctgctgaaatatcagccgcataaaagcctgctgagtgatgatgaagaatttctggttccgaatctgccgcatgaaattaatctgacccgtagtcgcctgccggacatgatgcgtggtcagggtgataaagaactgaatgatgcatggatgaaaatctttatccacgcaatggaagccgaagaaaatagctttggtgtgatcatgaacagcttctatgaactggaaccggaatatgtggaatactatcgtaatgtgatgggtcgtaaagcatggcatattggtccggttagcctgtgtaatcgtgaaaatgaagcaaaatttcagcgtggcaaagatagcagcattaacgaacatgaatgtctgaaatggctggacagcaaaaaaccgaaaagcgttgtgtatatttgctttggtagcctggcagaagtgccgacactgcagctgcgtgaaattgcaatgggtttagaagcaagcgaacaggatttcatttgggttgttcgtcgtggtaaagaaaacgtggaagaagaaaaaatcgaagagtggctgccgtatgattttgaagatcgtatggaaggtaaaggcctgattattcgtggttgggcaccgcaggttctgattctggatcatgaagcaattggtgcatttgttacccattgtggttggaatagcaccctggaaggtattagctgtggtgttccgatggttacctggcctgtttttgcagaacagttctataatgaaaaactggtgaccgaagttctgaaaaccggtgttgcagttggtgcaaaaaaatggtcacgtattctggaagtgaacctgaaaagcgaggatatcaaaaatgcaattcgtcgtgttatggttggtgaagaagcactggttctgcgtagcaaagcaaaaaaactgaaagaactggcacgtaaagccgttgaaattggtggtagcagctatagcgatatgcatagcctgattcaggatctgagcagttataatgccaatggctataaacagtatctgtaa

SEQ ID NO: 185 - protein, 478 aa, P. trichocarpa
MAETDSPPHVAILPSPGMGHLIPLVELAKRLVHQHNLSVTFIIPTDGSPSKAQRSVLGSLPSTIHSVFLPPVNLSDLPEDVKIETLISLTVARSLPSLRDVLSSLVASGTRVVALVVDLFGTDAFDVAREFKASPYIFYPAPAMALSLFFYLPKLDEMVSCEYSEMQEPVEIPGCLPIHGGELLDPTRDRKNDAYKWLLHHSKRYRLAEGVMVNSFIDLERGALKALQEVEPGKPPVYPVGPLVNMDSNTSGVEGSECLKWLDDQPLGSVLFVSFGSGGTLSFDQITELALGLEMSEQRFLWVARVPNDKVANATYFSVDNHKDPFDFLPKGFLDRTKGRGLVVPSWAPQAQVLSHGSTGGFLTHCGWNSTLESVVNAVPLIVWPLYAEQKMNAWMLTKDVEVALRPKASENGLIGREEIANIVRGLMEGEEGKRVRNRMKDLKDAAAEVLSEAGSSTKALSEVARKWKNHKCTQDCN

SEQ ID NO: 186 - DNA, 1437 bp, P. trichocarpa
atggcagaaaccgatagtccgcctcatgttgcaattctgccgagtcctggtatgggtcatctgattccgctggttgaactggcaaaacgtctggttcatcagcataatctgagcgtgacctttattatcccgaccgatggtagcccgagcaaagcacagcgtagcgttctgggtagcctgccgagcaccattcatagcgtttttctgcctccggttaatctgagtgatctgccggaagatgttaaaattgaaaccctgattagcctgaccgttgcacgttcactgccgagcctgcgtgatgttctgagcagcctggttgcaagcggcacccgtgttgttgcactggttgttgacctgtttggcaccgatgcatttgatgttgcacgtgaatttaaagcaagcccgtatatcttttatccggcaccggcaatggcactgagcctgtttttctatctgccgaaactggatgaaatggtgagctgtgaatatagcgaaatgcaagaaccggttgaaattccgggttgtctgccgattcatggtggtgaactgctggatccgacacgtgatcgtaaaaatgatgcatataaatggctgctgcatcacagcaaacgttatcgtctggccgaaggtgttatggtgaatagctttattgatctggaacgtggtgcactgaaagcactgcaagaagttgaaccgggtaaaccgcctgtttatccggttggtccgctggtgaatatggatagcaataccagcggtgttgaaggtagcgaatgtctgaaatggctggatgatcagccgctgggtagcgtgctgtttgttagctttggtagcggtggcaccctgagctttgatcagattaccgaactggcactgggtttagaaatgagcgaacagcgttttctgtgggttgcccgtgttccgaatgataaagttgcaaatgcaacctatttcagcgtggataatcacaaagatccgtttgattttctgccgaagggttttctggatcgtaccaaaggtcgtggtctggttgttccgagctgggcaccgcaggcacaggttctgagccatggtagcaccggtggttttctgacccattgtggttggaatagcaccctggaaagcgttgttaatgcagttccgctgattgtttggcctctgtatgcagaacagaaaatgaatgcatggatgctgaccaaagatgttgaagttgcactgcgtccgaaagcaagcgaaaatggtctgattggtcgtgaagaaattgccaatattgtgcgtggtctgatggaaggtgaagaaggtaaacgcgttcgtaatcgtatgaaagatctgaaagatgcagccgcagaagttctgagcgaagcaggtagcagcaccaaagcactgagtgaagttgcccgtaaatggaaaaaccataaatgtacccaggactgcaactaa

SEQ ID NO: 187 - protein, 469 aa, Q. suber
MEQKPHIALLPSPGMGHLIPLVEFAKQFVLHHDFHITCIIPVLGSPSKAMKAVLQALPTTIDHVFLPPVILEEEEIKGLKFEVQTILTLTRSLPPLREVLKTTRFSAFVVDPFGIDALDIAKELNISPYIFFPSNAFALSLIFHLPKLDETVSCEYRDLPEPLKLPGCIPIHGRDLIEPVQDRTSELYKMFLRNAKRFRLAEGIIVNTFMELEGSAIKALLDEEAKNLPLYPIGPIQSGSSNLQVDKSVSDCLRWLDNQPHGSVLFVCFGSGGTLSYDQTNELALGLELSGQKFLWVVRTPNNESADAAYLSDQILDNNPLDFLPKGFVERTEGQGLAVPSWAPQAQVLSHGSTGGFLTHCGWNSTLESIMQGIPLIAWPLYAEQKMNAPLLAEDLKVALRPKTNKSGLIDQEEIAKVVKGLMIGEEGKKVYNRMKDIKMAAEKALSADGSSTKALSELASQWKNHPGF

SEQ ID NO: 188 - DNA, 1410 bp, Q. suber
atggaacagaaaccgcatattgcactgctgccgagtcctggtatgggtcatctgattccgctggttgaatttgcaaaacagtttgtgctgcatcatgatttccatatcacctgtattattccggttctgggtagcccgagcaaagcaatgaaagcagttctgcaggcactgccgaccaccattgatcatgtttttctgcctccggttattctggaagaagaagaaattaaaggcctgaaatttgaagtgcagaccattctgaccctgacacgtagcctgcctccgctgcgtgaagttctgaaaaccacacgttttagcgcatttgttgttgatccgtttggtattgatgcactggatattgccaaagaactgaacattagcccgtatatcttttttccgagcaatgcatttgcactgagcctgatttttcatctgccgaaactggatgaaaccgttagctgtgaatatcgtgatctgccggaaccgctgaaactgcctggttgtattccgattcatggtcgcgatctgattgaaccggtgcaggatcgtaccagcgaactgtataaaatgtttctgcgtaatgccaaacgttttcgtctggcagaaggcattattgtcaatacctttatggaactggaaggcagcgcaattaaagcactgctggatgaagaagcaaaaaatctgccgctgtatccgattggtccgattcagagcggtagcagcaatctgcaggttgataaaagcgttagcgattgtctgcgttggctggataatcagccgcatggtagcgttctgtttgtttgttttggtagcggtggcaccctgagctatgatcagaccaatgaactggcactgggtttagaactgagcggtcagaaattcctgtgggttgttcgtaccccgaataatgaaagcgcagatgcagcatatctgagcgatcagattctggataataatccgctggattttctgccaaaaggttttgttgaacgtaccgaaggtcaaggtctggcagttccgagctgggcaccgcaggcacaggttctgagccatggtagcaccggtggttttctgacccattgtggttggaatagcaccctggaaagcattatgcagggtattccgctgattgcatggcctctgtatgcagaacagaaaatgaatgcaccgctgctggccgaagatctgaaagttgcactgcgtccgaaaaccaataaaagcggtctgattgatcaagaagagatcgccaaagttgttaagggtctgatgattggtgaagagggcaaaaaagtgtacaatcgcatgaaagacattaagatggcagcagaaaaagcactgagtgcagatggtagcagtaccaaagcgctgagcgaactggcaagccagtggaaaaatcatccgggtttttaa

SEQ ID NO: 189 - protein, 475 aa, A. duranensis
MAKTMRIAVITSPGLTHLVPILEFSKRFLELHPNFHVTCMIPSLGPHPDSTKSYLQTLPSNIHSILLPPINKQDLPQGAYPGVLIQKTVTLSLPSIRDTLKSLTLREPLAALIADAYAFEALSFAKEFNFLSYIYFPSSVMALSLCLHLPKLDEQVTGEYKDLKDPIYLPGCVPVFGRDLPFPMQNRSSDAYKLYLERSKGFSNVDGFIINSFLELESAAMKALAREKSCFSFYDVGPITQKRSSSNDGDEELECLRWLDKQPHSSVLYVSFGSGGTLSQSAINELAFGLELSGQRFLWVLRAPSDSSSAAYLDNQKNEDPLKFLPSGFLERTKEKGLVLPSWAPQVQILSHDSVGGFLSHCGWNSVLESVQVGVPIITWPLFAEQRMNAVLLVDGLKVAVRPNVGEDGVVGKEEVSNVIKCLMEQEEGKAMRKRMEDLKAYAADAVNKDAGSSTHALSHLATKWENFSGIEDNN

SEQ ID NO: 190 - DNA, 1428 bp, A. duranensis
atggcaaaaaccatgcgtattgccgttattaccagtccgggtctgacccatctggttccgattctggaatttagcaaacgttttctggaactgcatccgaattttcatgttacctgtatgattccgagcctgggtccgcatccggatagcaccaaaagctatctgcagaccctgccgagcaatattcatagcattctgctgcctccgattaacaaacaggatctgccgcagggtgcatatccgggtgttctgattcagaaaaccgttacactgagcctgccgagtattcgtgataccctgaaaagtctgaccctgcgtgaaccgctggcagcactgattgcagatgcatatgcctttgaagcactgagctttgccaaagaattcaactttctgagctatatctatttcccgagcagcgttatggccctgagcctgtgtctgcatctgccgaaactggatgaacaggttaccggtgaatataaagatctgaaagatccgatttatctgcctggttgtgttccggtttttggtcgtgatctgccgtttccgatgcagaatcgtagcagtgatgcatataaactgtatctggaacgcagcaaaggttttagcaatgtggatggctttatcatcaacagctttcttgaactggaaagcgcagcaatgaaagcactggcacgtgaaaaaagctgctttagcttttatgatgtgggtccgattacacagaaacgtagctcaagcaatgatggtgatgaagaactggaatgtctgcgttggctggataaacagccgcatagcagcgttctgtatgttagctttggtagcggtggcaccctgagccagagcgcaattaatgaactggcatttggcctggaactgagcggtcagcgttttctgtgggttctgcgtgcaccgagcgatagcagcagcgcagcatatctggataatcagaaaaatgaagatccgctgaaatttctgccgagcggtttcctggaacgtaccaaagaaaaaggtctggtgctgccgagctgggcaccgcaggttcagattctgagccatgatagcgttggtggttttctgtcacattgtggttggaatagcgttctggaaagtgttcaggttggtgttccgattattacctggcctctgtttgcagaacagcgtatgaatgcagttctgctggttgatggtctgaaagttgcagttcgtccgaatgttggtgaagatggtgttgttggtaaagaagaagttagcaacgttatcaagtgcctgatggaacaagaagagggtaaagcaatgcgtaaacgtatggaagatttaaaagcatatgcagccgatgccgttaataaagatgcaggtagcagcacccatgcactgagccatctggcaaccaaatgggaaaactttagcggtattgaggacaacaactaa

SEQ ID NO: 191 - protein, 495 aa, C. papaya
MGSEVLHHDYSQLNIFFFPFMAHGHMIPTLDMAKLFATHGAKTSIITTPLNLPFFSKSIERFSKQTGLEIGVKLLNFPSVEVGLPSGCENADSLPAGEPLIVNKFFAAAGMLKDPLERLLQEFKPDCLIADMFFPWTTDAAAKFDIPRLVFHGTSFFALSASECIRLYTPFNNVSSDSEPFLVPTLPDEIRLTRNQLADFAMKEGDENGIHRLIKEAKESELKSYGVVVNSFYELEPAYADHYRNFLKRKAWHIGPVSLCNKTVEDKAERGKRASIDEDECLKWLNSKAPNSVIYICFGSMANFNSAQLMEIATALDASGQEFIWVVRREKNENNQEDWLPEGFEQRTEGKGLIIRGWAPQVLILEHEAVGGFVTHCGWNSTLEGVTAGMPMVTWPVSAEQFYNEKLVTEVLKIGLSVGVKKWVRSEGDFVSREKVEQAVREIMVGSEAVERRMRAKAMADMARAAVEKGGSSYNDLNALLREVSLMRRQQSQNQ

SEQ ID NO: 192 - DNA, 1488 bp, C. papaya
atgggtagcgaagttctgcatcatgattatagccagctgaacatctttttctttccgtttatggcacatggtcatatgattccgacactggatatggcaaaactgtttgcaacccatggtgcaaaaaccagcattattaccacaccgctgaatctgccgttttttagcaaaagcattgaacgctttagcaaacagacaggtctggaaattggtgtgaaactgctgaattttccgagcgttgaagttggtctgccgagcggttgtgaaaatgcagatagcctgcctgccggtgaaccgctgattgtgaataaattctttgcagcagcaggcatgctgaaagatccgctggaacgtctgctgcaagagtttaaaccggattgtctgattgccgatatgttttttccgtggaccaccgatgcagcagccaaatttgatattccgcgtctggtttttcatggcaccagcttttttgcactgagcgcaagcgaatgtattcgtctgtataccccgtttaataacgttagcagcgatagcgaaccgtttctggtgccgacactgccggatgaaattcgtctgacccgtaatcagctggcagattttgcaatgaaagaaggtgacgaaaacggtattcatcgtctgattaaagaagccaaagaaagcgagctgaaaagctatggtgttgtggtgaatagcttttatgaactggaaccggcatatgcggatcattatcgtaattttctgaaacgcaaagcctggcatattggtccggttagcctgtgtaataaaaccgttgaagataaagccgaacgtggtaaacgtgcaagcattgatgaagatgaatgtctgaaatggctgaatagcaaagcaccgaatagcgtgatttatatctgctttggtagcatggccaattttaacagcgcacagctgatggaaattgcaaccgcactggatgcaagcggtcaagaattcatttgggttgttcgtcgcgaaaaaaacgaaaacaatcaagaagattggctgccggaaggttttgaacagcgtaccgaaggtaaaggtctgattattcgtggttgggcaccgcaggttctgattctggaacatgaagcagttggtggttttgttacccattgtggttggaatagcaccctggaaggtgttaccgcaggtatgccgatggttacctggcctgttagcgcagaacagttttataacgaaaaactggttaccgaggtgctgaaaattggtctgagcgtgggtgtgaaaaaatgggttcgtagcgaaggtgattttgtgagccgtgaaaaagttgaacaggcagttcgtgaaattatggttggtagtgaagccgttgaacgtcgtatgcgtgcaaaagcaatggcagatatggcacgtgcagcagttgaaaaaggtggtagcagctataatgatctgaatgcactgctgcgtgaagttagcctgatgcgtcgtcagcagagtcagaatcagtaa

SEQ ID NO: 193 - protein, 491 aa, Z. jujube
MKKAELVFIPIPGRGHLLSMVEFAKLLVARDPHLYVTILIMKLPFDTKVGAYTASLVSSSSNRINCIDLPINEKVYTESNPPVFMTSFIEDQKPHVKNAVTQLIQSRDVDDEDSPRLAGFVIDMFCTTMIDVANEFGIPTYVFFASGAGFLGLLFHLQHLSDNHNVNITEFENDPEAELVIPSFVNPFPSKVLPVLVLDKDGGPVMMNHARRIRETKGIIVNTFIELESHAVYSLSNGDHEFPPVYPVGPILYLKSDESHVGSVNQIQNSDIIRWLDNQPPSSVVFVCFGSMGSFSEDQVKEIAYGLEQSGQRFIWSLRPPPPKDKMGFPSDYLDPTVVLPEGFLDRTAEVGKVIGWAPQVEILSHCATGGFVSHCGWNSTLESLWFGVPIATWPIFAEQQLNAFQMVKEFGCAVEIKLDYRREFNSDGDDQAVVSAQEIERGIRRVMDDDSDIRKRTKEISEQSRRTLVDGGTSFSCLGHLINDILENVS

SEQ ID NO: 194 - DNA, 1476 bp, Z. jujube
atgaaaaaagccgaactggtgtttattccgattcctggtcgtggtcatctgctgagcatggttgaatttgcaaaactgctggttgcacgtgatccgcatctgtatgttaccattctgattatgaaactgccgttcgataccaaagttggtgcatataccgcaagcctggttagcagcagcagtaatcgtattaattgtattgatctgccgatcaacgagaaagtgtataccgaaagcaatccgcctgtttttatgaccagctttatcgaagatcagaaaccgcatgttaaaaatgcagttacccagctgattcagagccgtgatgttgatgatgaagatagtccgcgtctggcaggttttgttattgatatgttttgcaccaccatgatcgatgtggcaaatgaatttggtattccgacctatgttttttttgcaagcggtgcaggttttctgggtctgctgtttcatctgcagcatctgagcgataatcataacgtgaacatcaccgaatttgagaatgatccggaagcagaactggttattccgagctttgttaatccgtttccgagcaaagttctgccggttctggttctggataaagatggtggtccggttatgatgaatcatgcacgtcgtattcgtgaaaccaaaggcattattgtgaacacctttattgaactggaaagccatgcagtttatagcctgagcaatggtgatcatgaatttccgccagtttatccggttggtccgattctgtatctgaaaagtgatgaaagtcatgtgggtagcgttaatcagattcagaacagcgatattattcgctggctggataatcagcctccgagcagcgttgtttttgtttgttttggtagcatgggtagctttagtgaggatcaggttaaagaaattgcctatggtctggaacagagcggtcagcgttttatttggagcctgcgtccgcctccgcctaaagataaaatgggttttccgagcgattatctggatccgaccgttgtgctgccggaaggctttctggatcgtaccgcagaagttggtaaagttattggttgggcaccgcaggttgaaattctgagccattgtgcaaccggtggttttgtttcacattgtggttggaatagcaccctggaaagtctgtggtttggtgttccgattgcaacctggccgatttttgcagaacagcagctgaatgcatttcagatggtgaaagaatttggttgtgccgtggaaatcaaactggattatcgtcgtgaatttaacagcgacggtgatgatcaggcagttgttagcgcacaagaaattgaacgtggtattcgtcgtgttatggatgatgatagcgatattcgtaaacgcaccaaagaaattagcgaacagagccgtcgtaccctggttgatggtggtacaagctttagctgtctgggtcatctgatcaatgatattctggaaaacgtgagctaa

SEQ ID NO: 195 - protein, 483 aa, H. annuus
MANAVAELIFIPTPGLGHIMSTIELAKLLVNRDQRLAITVLVIKPPGMTSGSAITTYIESLTETTMDRISFIQLPQVESSPTHGGPTEFIRSHSKYVRNAVVDLRSQSGSCQVVGFVVDMFCTSMIDVANEFNVPTFVFFTSSAAFLGFTLFIKLLCDDLNRDVVELSNSDTEISVPSFVKPVPTKVFWSLVKTREGLDSVQRLAKKLGEAKGIIVNTFLDLETHAIESLSADISIPPVYPVGPILNLEGGSGGGKPFDDDVIRWLDSQPPSSVVFLCFGSMGSFDEAQVKEIARGLEQSGHRFLWSLRRPPSEQTTTRIPSDYEDPSVVLPEGFLDRTRGIGKVIGWAPQVAVLAHDAVGGFVSHCGWNSLLESLWFGVPSATWPMYAEQQMNAFEMVVDLGLAVEIKLDYEKDVFNPFNPKANKIINVTAGEIESGMRRVMEDNEVRVRVKEMSAKSRAAVVEGGSSYAFVGRLIQDFIRD

SEQ ID NO: 196 - DNA, 1452 bp, H. annuus
atggcaaatgcagttgcagaactgatttttatcccgacacctggtctgggtcatattatgagcaccattgaactggcaaaactgctggttaatcgtgatcagcgtctggcaattaccgttctggttattaaaccgcctggtatgaccagcggtagcgcaattaccacctatattgaaagcctgaccgaaaccaccatggatcgtattagctttattcagctgccgcaggttgaaagcagcccgacacatggtggtccgaccgaatttattcgtagccatagcaaatatgttcgtaatgccgttgttgatctgcgtagccagagcggtagctgtcaggttgttggttttgttgttgatatgttttgcaccagcatgattgatgtggccaatgaatttaatgttccgacctttgtgtttttcaccagtagcgcagcatttctgggttttaccctgtttatcaaactgctgtgtgatgatctgaatcgtgatgttgttgaactgagcaatagcgataccgaaatttcagtgccgagctttgttaaaccggttccgaccaaagttttttggagcctggttaaaacccgtgaaggtctggatagcgttcagcgcctggcgaaaaaactgggtgaagcaaaaggtattatcgtgaacacctttctggatctggaaacccatgcaattgaaagtctgagcgcagatattagcattcctccggtttatccggttggtccgattctgaacctggaaggtggtagcggtggtggtaaaccgtttgatgatgatgttattcgttggctggatagccagcctccgagcagcgttgtttttctgtgttttggtagcatgggtagctttgatgaagcacaggttaaagaaattgcacgtggtctggaacagagcggtcatcgttttctgtggtcactgcgtcgtccgcctagcgaacagaccaccacacgtattccgagcgattatgaagatccgagcgttgttctgccggaaggtttcctggatcgtacccgtggtattggtaaagttattggttgggcacctcaggttgcagttctggcacatgatgcagttggtggctttgttagccattgtggttggaatagcctgctggaaagcctgtggtttggtgttccgagcgcaacctggccgatgtatgcagaacagcagatgaatgcatttgaaatggttgtggatctgggtttagccgtggaaattaaactggattatgagaaggatgtgtttaacccgtttaatccgaaagccaacaaaatcattaatgtgaccgcaggcgaaattgaaagcggtatgcgtcgtgttatggaagataatgaagttcgtgttcgcgtgaaagaaatgagcgcaaaaagccgtgcagcagttgttgaaggtggttcaagctatgcatttgttggtcgtctgattcaggattttatccgcgattaa

SEQ ID NO: 197 - protein, 507 aa, A. commosus
MKDVTPHFVLVPLAAQGHMIPMVDMARLLAERGVRVTLITTPVNAARIRTIIDRVRRSNLPVEFVELRFPCAEFGLPEGSENIDLLSTLEHYKAFFDAMKLLKEPIEALLRSQHRRPDCMIADMCNGWTKDVARRLGIPRLLFHGPSCFYILCAYNMAQHRVYDRVTHEFEPVVVPDVPVEVVTNKAESPGFFNWSGWEDLRAEVLEAESTADGVVINTFYDLEPSFVDCYEKIMQKKVWTVGPLCLYSKDVDSKAARGNKAAVDHRDITTWLDRKGASSVFYVSFGSLVLMRPTQLIEIGKGLLECSDHRSFIWVVKEAELVPEVEKWLSEEHFAERTKERGLLIKGWAPQTVILLHPAIGGFLTHCGWNSTLEAISAGVPMLTWPHFADQFLNEKLVVDVLKIGRSLDVKVPRTHVTDDSTLLVTKEKLRKAVSELMEGEEGEEMRRRAKALAEKAKKAMEEGGSSYRNMDDMIECMAGRYGEEEKVEDAVKELSNGFSAHVVVT

SEQ ID NO: 198 - DNA, 1524 bp, A. commosus
atgaaagatgtgacaccgcattttgttctggttccgctggcagcacagggtcatatgattccgatggttgatatggcacgtctgctggcagaacgtggtgttcgtgttaccctgattaccacaccggttaatgcagcacgtattcgtaccattattgatcgtgttcgtcgtagcaatctgccggttgaatttgttgaactgcgttttccgtgtgcagaatttggtctgccggaaggtagcgaaaatattgatctgctgagcaccctggaacactataaagcattttttgatgccatgaaactgctgaaagaaccgattgaagcactgctgcgtagccagcatcgtcgtccggattgtatgattgcagatatgtgtaatggttggaccaaagatgttgcacgtcgtctgggtattccgcgtctgctgtttcatggtccgagctgcttttatatcctgtgtgcctataatatggcacagcatcgtgtttatgatcgtgtgacccatgaatttgaaccggttgttgttccggatgttccggttgaagtggttaccaataaagcagaaagtccgggttttttcaattggagcggttgggaagatctgcgtgcagaagttctggaagccgaaagcaccgcagatggtgttgtgattaataccttttatgatctggaaccgagcttcgttgattgctatgaaaaaatcatgcagaaaaaggtttggaccgttggtccgctgtgtctgtatagcaaagatgtggatagcaaagcagcacgtggtaataaagccgcagttgatcatcgtgacattaccacctggctggatcgtaaaggtgcaagcagcgttttttatgttagctttggtagcctggttctgatgcgtccgacacagctgattgaaattggtaaaggtctgctggaatgcagcgatcatcgtagctttatttgggttgttaaagaagcagaactggttccggaagttgaaaaatggctgagcgaagaacattttgcagaacgtaccaaagaacgcggtctgctgattaaaggttgggctccgcagaccgttattctgctgcatccggcaattggtggttttctgacccattgtggttggaatagtaccctggaagcaattagtgccggtgttccgatgctgacctggcctcattttgccgatcagtttctgaatgaaaaactggttgttgacgtgctgaaaattggtcgtagcctggatgttaaagttccgcgtacacatgttaccgatgatagcaccctgctggtgaccaaagaaaaactgcgtaaagcagttagcgaactgatggaaggtgaagagggtgaagaaatgcgtcgtcgtgcaaaagcactggccgaaaaagcaaaaaaagccatggaagaaggtggtagcagctatcgtaatatggatgatatgattgaatgcatggcaggtcgttatggcgaagaagaaaaagttgaggacgcagttaaagaactgagcaatggttttagcgcacatgttgttgttacctaa

SEQ ID NO: 199 - protein, 484 aa, C. papaya
MTGELIFIPMPSLSHIASTMEIAKLLVHRDDRLSITVLLISSQYTTSITTYINSLIASSDYDRIRFIHLPELDSEEEPKRPFMSVIDDNKPIVKEAVTNLALSFDPSHRLAGFVIDMFCVGMIEVADELGLPSYPFFTSSTSFLALQFHVQTLADEEEVDITEFKNSDVMLPIPGLVNPLPAKTILPSAMLNKDWLPYVLNGARGFRKTKGIMVNSFAEIESNAVTSLSNSTVPPVYTVGPIINFKGDGQDSDTCTAHKYSNIMTWLDDQPPSSVLFLCFGSLGSFDEEQVKEIARALEGSGHRFLWSLRRPPPKDKTMSFPTEYENFEEVLPEGFVDRTVGMGKVMGWAPQVAVLAHPSIGGFVTHCGWNSILESVWFGVPMAAWPLYAEQQFNAFHMVVELGLAVEIKMDYRKDYAILGLQEERVSAEVIEKGIRCLMEEDNDARKKVKEMSEISRKALMDGGSSHAVLGQFIEDVMNNISA

SEQ ID NO: 200 - DNA, 1455 bp, C. papaya
atgaccggtgaactgatttttatcccgatgccgagcctgagccatattgcaagcaccatggaaattgcaaaactgctggttcatcgtgatgatcgtctgagcattaccgttctgctgattagcagccagtataccacctcaattaccacctatattaacagcctgattgccagcagcgattatgatcgtattcgttttattcatctgccggaactggatagcgaagaagaaccgaaacgtccgtttatgagcgtgattgatgataacaaaccgatcgttaaagaagccgttaccaatctggcactgagctttgatccgagccatcgtctggcaggttttgttattgatatgttttgcgtgggcatgattgaagttgcagatgaactgggtctgccgagctatccgttttttaccagcagcaccagctttctggccctgcagtttcatgttcagaccctggccgatgaagaagaagttgatattaccgagtttaagaactccgatgttatgctgccgattcctggtctggttaatccgctgcctgcaaaaaccattctgccgagtgcaatgctgaataaagattggctgccgtatgttctgaatggtgcacgtggttttcgtaaaacgaaaggcattatggttaacagctttgccgaaattgaaagcaatgcagttaccagcctgagcaatagcaccgttccgcctgtttataccgttggtccgattattaactttaaaggtgatggtcaggatagcgatacctgtaccgcacacaaatatagcaatattatgacctggctggatgatcagcctccgagcagcgttctgtttctgtgttttggtagcctgggtagctttgatgaagaacaggttaaagaaattgcacgtgccctggaaggtagcggtcatcgttttctgtggtcactgcgtcgtccgcctccgaaagataaaaccatgagctttccgaccgaatatgaaaactttgaagaagtgctgccggaaggttttgtggatcgcaccgttggtatgggtaaagttatgggttgggcaccgcaggttgcagttctggcacatccgagcattggtggttttgtgacccattgtggttggaatagcattctggaaagcgtttggtttggtgttccgatggcagcatggcctctgtatgcagaacagcagtttaatgcatttcatatggtggtggaactgggtttagcagtggaaatcaaaatggattatcgcaaagattatgccattctgggcctgcaagaagaacgcgttagcgcagaagttattgaaaaaggtattcgttgtctgatggaagaggataatgatgcccgtaaaaaagtgaaagaaatgagcgaaattagccgcaaagcactgatggatggtggtagcagccatgccgttctgggtcagtttattgaagatgtgatgaataacatcagcgcctaa

SEQ ID NO: 201 - protein, 470 aa, H. annuus
MERTPHIAIVPSPGMGHLIPLVEFAKRLKNNHNISSTFIIPNDGPLSISQKAFLDSLPMGLNHIILPPVNFDDLPQDTQMETRISLMVTRSLDSLREVFKSLVAEHNMVALFIDLFGTDAFDVAIEFGVSPYVFFPSTAMALSLFLYLPKLDQMTSCEYRDLPEPVQIPGCLPVRGQDLLDPVQDRKNDAYKWVLHNAKRYMMAEGIAVNSFKELEGGALKALLEAEPGKPKIYPVGPLIQTGSSSDVDGSGCLKWLDGQPCGSVLYISFGSGGTLSSNQLNELAMGLELSEQRFIWVVRSPSDQANATYFNSHGHKDPLGFLPKGFLERTKGNGFVVSSWAPQAQILSHSSTGGFLTHCGWNSILETVVHGVPVIAWPLYAEQKMNAVSLTEGIKVALRPTVGENGIIGRVEIARVVKSLLEGEEGKAIRSRIRDLKDAAANVISKDGCSTKTLDKLASMLKNKNKLSL

SEQ ID NO: 202 - DNA, 1413 bp, H. annuus
atggaacgtacaccgcatattgcaattgttccgagtcctggtatgggtcatctgattccgctggttgaatttgcaaaacgcctgaaaaacaaccacaatattagcagcacctttatcattccgaacgatggtccgctgagcattagccagaaagcatttttagatagcctgccgatgggtctgaaccatattattctgcctccggtgaattttgatgatctgccgcaggatacccagatggaaacccgtattagcctgatggttacccgtagcctggatagtctgcgtgaagtgtttaaaagcctggttgcagaacataacatggtggcactgtttattgacctgtttggcaccgatgcatttgatgttgcaattgaatttggtgttagcccgtatgttttttttccgagcaccgcaatggcactgagcctgtttctgtatctgccgaaactggatcaaatgaccagctgtgaatatcgcgatctgccggaaccggtgcagattccgggttgtctgccggttcgtggtcaggatctgctggatccggttcaggatcgtaaaaatgatgcatataaatgggtgctgcataacgccaaacgttatatgatggcagaaggtattgccgtcaacagctttaaagaactggaaggtggtgcactgaaagcactgctggaagcagaaccgggtaaaccgaaaatctatccggttggtcctctgattcagaccggtagcagcagtgatgttgatggtagcggttgtctgaaatggctggatggtcagccgtgtggtagcgttctgtatattagctttggtagtggtggcaccctgagcagcaatcagctgaatgaactggcaatgggtttagaactgagcgaacagcgttttatttgggttgttcgtagcccgagcgatcaggcaaatgcaacctattttaacagccatggtcataaagatccgctgggttttctgcctaaaggttttctggaacgcaccaaaggtaatggttttgttgttagcagctgggcaccgcaggcacagattctgagccatagcagtaccggtggttttctgacccattgtggctggaatagcattctggaaaccgttgttcatggtgttccggttattgcatggcctctgtatgcagaacagaaaatgaatgcagttagcctgaccgaaggtattaaagttgcactgcgtccgaccgttggtgaaaatggtattattggtcgtgttgaaattgcccgtgttgtgaaaagcctgttagaaggtgaagaaggtaaagcaattcgtagccgtattcgtgatctgaaagatgcagcagcaaatgtgattagcaaagatggttgtagcaccaaaacactggataaactggcaagcatgctgaagaacaaaaacaaactgtccctgtaa

SEQ ID NO: 203 - protein, 485 aa, S. pennellii
MDKRADQLHVYFLPMMAPGHMIPLVDMARQFSRHGVKVTIVTTPLNATKFSKTIQKDREFGSDICIRTTEFPCKEAGLPEGCENLASTTTSEMTMKFIKALYLFEQPVEKFMEEDHPDCLVAGTFFAWAVDVAAKLGIPRLAFNGTGLLPMCAYNCLMEHKPHLKVESETEEFVIPGLPDTIKMSRSKLSQHWVDEKETPMTPIIKDFMRAEATSYGAIVNSFYELEPNYVQHFREVVGRKVWHVGPVSLCNKDNEDKSQRGQDSSLSEQKCLDWLNTKEPKSVIYICFGSMSIFSSDQLLEIATALEASDQQFIWVVRQNTTNEEQEKWMPEGFEEKVNGRGLIIKGWAPQVLILDHEATGGFVTHCGWNSLLEGVSAGVPMVTWPLSAEQFFNEKLLVEILKIGVPVGVQAWSQRTDSRVPINRENILRAVTKLMVGQEAEEMQGRAAALGKSAKMAVEKGGSSDNSLVSLLEELRNGKSSSN

SEQ ID NO: 204 - DNA, 1458 bp, S. pennellii
atggataaacgtgcagatcagctgcatgtttattttctgccgatgatggcaccgggtcatatgattccgctggttgatatggcacgtcagtttagccgtcatggtgttaaagttaccattgttaccacaccgctgaatgcaaccaaatttagcaaaaccattcagaaagatcgcgaatttggtagcgatatttgtattcgtaccaccgaatttccgtgtaaagaagcaggtctgccggaaggttgtgaaaatctggcaagcaccaccaccagtgaaatgaccatgaaatttatcaaagccctgtacctgtttgaacagccggttgaaaaattcatggaagaagatcatccggattgtctggttgcaggcaccttttttgcatgggcagttgatgttgcagcaaaactgggtattccgcgtctggcatttaatggtacaggtctgctgccgatgtgtgcatataattgtctgatggaacataaaccgcacctgaaagttgaaagcgaaaccgaagaatttgttattccgggtctgcctgatacgattaaaatgagccgtagcaaactgagccagcattgggttgatgaaaaagaaaccccgatgacaccgatcatcaaagattttatgcgtgccgaagcaaccagctatggtgcaattgttaatagcttttatgagctggaaccgaactatgtgcagcattttcgtgaagttgttggtcgtaaagtttggcatgttggtccggttagcctgtgcaataaagataatgaagataaaagccagcgtggtcaggatagcagcctgagcgaacagaaatgtctggattggctgaataccaaagaaccgaaaagcgtgatctatatttgctttggtagcatgagcatctttagcagcgatcaactgctggaaattgcaaccgcactggaagcaagcgatcagcagtttatttgggttgttcgtcagaataccaccaacgaagaacaagaaaaatggatgcctgaaggctttgaagaaaaagttaatggtcgtggcctgattatcaaaggttgggcaccgcaggttctgattctggatcatgaagcaaccggtggttttgttacccattgtggttggaatagcctgctggaaggtgttagtgccggtgttccgatggttacctggcctctgagcgcagaacagttttttaacgaaaaactgctggtcgagattctgaaaattggtgttccggttggtgttcaggcatggtcacagcgtaccgatagccgtgttcctattaatcgtgaaaatattctgcgtgccgttaccaaactgatggttggtcaagaggccgaagaaatgcagggtcgtgcagcagcactgggtaaaagcgcaaaaatggcagttgaaaaaggtggcagcagcgataatagcctggttagcttactggaagaactgcgtaatggtaaaagcagcagcaactaa

SEQ ID NO: 205 - protein, 471 aa, S. pennellii
MAQIPHIAILPSPGMGHLIPLVEFAKRIFLHHQFSVSLILPTDGPISNAQKIFLNSLPSSMDYHLLPPVNFDDLPEDVKIETRISLTVSRSLTSLRQVLDSIIESKRTVALVVDLFGTDAFDVAIDLKISPYIFFPSTAMALSLFLYLPNLDETVSCEYRDLPDPIQIPGCTPIHGKDLLDPVQDRNDESYKWLLHHVKRYGMAEGIIVNSFKELEGGAIGALQKDEPGKPTVYPVGPLIQMDSGSKVDGSECMTWLDEQPRGSVLYISYGSGGTLSHEQLIEVAAGLEMSEQRFLWVVRCPNDKIANATFFNVQDSTNPLEFLPKGFLERTKGFGLVLPNWAPQARILSHESTGGFLTHCGWNSTLESVVHGVPLIAWPLYAEQKMNAVMLSEDIKVALRPKVNEENGIVGRLEIAKVVKGLMEGEEGKGVRSRMRDLKDAAAKVLSEDGSSTKALAELATKLKKKVSNN

SEQ ID NO: 206 - DNA, 1416 bp, S. pennellii
atggcacagattccgcatattgcaattctgccgagtcctggtatgggtcatctgattccgctggttgaatttgccaaacgtatttttctgcatcaccagtttagcgttagcctgatcctgccgaccgatggtccgattagcaatgcacagaaaatctttctgaatagcctgccgagcagcatggattatcatctgctgcctccggttaattttgatgatctgccggaagatgtgaaaattgaaacccgtattagcctgaccgttagccgtagtctgaccagcctgcgtcaggttctggatagcattattgaaagcaaacgtaccgttgcactggttgttgacctgtttggcaccgatgcatttgatgttgcaattgatctgaaaatcagcccgtatatcttttttccgagcaccgcaatggcactgagcctgtttctgtatctgccgaatctggatgaaaccgttagctgtgaatatcgtgatctgcctgatccgattcagattccgggttgtaccccgattcatggtaaagatctgctggatccggtgcaggatcgtaatgatgaaagctataaatggctgctgcatcacgttaaacgttatggtatggcagaaggcattatcgtcaacagctttaaagaactggaaggtggtgcaattggtgcactgcagaaagatgaaccgggtaaaccgaccgtttatccggttggtccgctgattcagatggatagcggtagcaaagttgatggtagcgaatgtatgacctggctggatgaacagcctcgtggtagcgttctgtatattagctatggtagcggtggcaccctgagccatgaacagctgattgaagttgcagcaggtctggaaatgagcgaacagcgttttctgtgggttgttcgttgtccgaatgataaaattgcaaacgccaccttttttaacgttcaggatagcaccaatccgctggaatttctgccgaaaggttttctggaacgtaccaaaggttttggtctggtgctgccgaattgggcaccgcaggcacgtattctgagtcatgaaagcaccggtggttttctgacccattgtggttggaatagcaccctggaaagcgttgttcatggtgtgccgctgattgcatggcctctgtatgcagaacagaaaatgaatgcagttatgctgagcgaggatattaaagttgcactgcgtccgaaagtgaatgaagaaaatggtattgttggtcgcctggaaattgccaaagttgttaaaggtctgatggaaggtgaagaaggtaaaggcgttcgtagccgtatgcgcgatctgaaagatgccgcagcaaaagttctgagcgaagatggtagcagcaccaaagcactggcagaactggcaaccaaactgaaaaaaaaggtcagcaacaattaa

SEQ ID NO: 207 - protein, 480 aa, C. Sativus; identical to SEQ ID NO: 157

SEQ ID NO: 208 - DNA, 1443 bp, C. Sativus
atgggttctgaaggtagacaattgcacattttcatgttcccattcatggctcatggtcatatgattccaatagttgatatggctaagttgttcgcctcaagaggtattaagattaccatcgttactacgcccttgaactccatttctatctctaagtcattgcacaactgctccccaaattctttgattcagttgctgattttgaagttcccagctgctgaagctggtttgccagatggttgtgaaaatgctgattctatcccatctatggacttgttgccaaagtttttcgaagccgtttctttgttgcaaccaccatttgaagaagccttgcataacaatagaccagactgcttgatttccgatatgttttttccatggaccaacgatgttgctgatagagttggtattccaagattgatcttccatggcacctcttgcttttctttgtgttcttctgaattcatgaggctgcataagccataccaacatgtttcttcagatactgagccattcaccattccatatttgccaggtgatattaagctgaccaaaatgaagttgccaatcttcgtcagagaaaactccgaaaacgaattctccaagttcatcaccaaggtcaaagaatctgaatctttctgctacggtgttgtcgttaactctttctatgaattggaagccgaatacgttgattgctacaaagatgttttgggtagaaagacttggactatcggtccattgtctttgactaacactaagacccaagaaatcaccttgagaggtagagaatctgccattgatgaacatgaatgtttgaagtggttggactctcaaaagccaaactctgttgtttacgtttgctttggttctttggccaagtttaactccgctcagttgaaagaaattgctattggtttggaagcctccggtaagaagtttatttgggttgttagaaaaggtaagggcgaagaagaagaggaagaacaaaattggttgccagaaggttacgaagaaagaatggaaggtactggtttgattattagaggttgggctccacaagttttgattttggatcatccatctgttggtggtttcgttactcattgtggttggaattctactttggaaggtgttgctgctggtgttccaatggttacttggccagttggtgctgaacaattttacaacgaaaagttggttaccgaggtcttgaaaactggtgttggtgtaggtgttcaaaaatgggctccaggtgtcggtgattttattgaatctgaagctgttgagaaggccatcagacgtattatggaaaaagaaggtgaagagatgagaaacagagccattgaattgggtaaaaaagctaaatgggctgtcggtgaagaaggttcttcttactctaatttggatgccttgatcgaagagttgaagtctttggctttctaa

SEQ ID NO: 209 - protein, 805 aa, Glycine Max
MATDRLTRVHSLRERLDETLTANRNEILALLSRIEAKGKGILQHHQVIAEFEEIPEENRQKLTDGAFGEVLRSTQEAIVLPPWVALAVRPRPGVWEYLRVNVHALVVEELQPAEYLHFKEELVDGSSNGNFVLELDFEPFNAAFPRPTLNKSIGNGVQFLNRHLSAKLFHDKESLHPLLEFLRLHSVKGKTLMLNDRIQNPDALQHVLRKAEEYLGTVPPETPYSEFEHKFQEIGLERGWGDNAERVLESIQLLLDLLEAPDPCTLETFLGRIPMVFNVVILSPHGYFAQDNVLGYPDTGGQVVYILDQVRALENEMLHRIKQQGLDIVPRILIITRLLPDAVGTTCGQRLEKVFGTEHSHILRVPFRTEKGIVRKWISRFEVWPYLETYTEDVAHELAKELQGKPDLIVGNYSDGNIVASLLAHKLGVTQCTIAHALEKTKYPESDIYWKKLEERYHFSCQFTADLFAMNHTDFIITSTFQEIAGSKDTVGQYESHTAFTLPGLYRVVHGIDVFDPKFNIVSPGADQTIYFPHTETSRRLTSFHPEIEELLYSSVENEEHICVLKDRSKPIIFTMARLDRVKNITGLVEWYGKNAKLRELVNLVVVAGDRRKESKDLEEKAEMKKMYGLIETYKLNGQFRWISSQMNRVRNGELYRVICDTRGAFVQPAVYEAFGLTVVEAMTCGLPTFATCNGGPAEIIVHGKSGFHIDPYHGDRAADLLVDFFEKCKLDPTHWDKISKAGLQRIEEKYTWQIYSQRLLTLTGVYGFWKHVSNLDRRESRRYLEMFYALKYRKLAESVPLAAE

SEQ ID NO: 210 - DNA, 2418 bp, Glycine Max
atggcaaccgatcgtctgacccgtgttcatagcctgcgtgaacgtctggatgaaaccctgaccgcaaatcgtaatgaaattctggcactgctgagccgtattgaagcaaaaggtaaaggtattctgcagcatcatcaggtgattgccgaatttgaagaaattccggaagaaaatcgtcagaaactgaccgatggtgcatttggtgaagttctgcgtagcacccaagaagcaattgttctgcctccgtgggttgcactggcagttcgtccgcgtcctggtgtttgggaatatctgcgtgttaatgttcatgcactggttgttgaagaactgcagcctgcagagtatctgcattttaaagaagaactggtagacggtagcagcaatggtaattttgttctggaactggattttgagccgtttaatgcagcatttccgcgtccgacactgaataaaagcattggtaatggtgttcagttcctgaatcgtcatctgagcgcaaaactgtttcatgataaagaaagcctgcatccgctgctggaatttctgcgtctgcatagcgttaaaggtaaaaccctgatgctgaatgatcgtattcagaatccggatgcactgcagcatgtgctgcgtaaagcagaagaatatctgggcaccgttccgcctgaaacaccgtatagtgaatttgaacacaagtttcaagaaatcggtctggaacgtggttggggtgataatgcagaacgtgtgctggaaagcattcagctgctgctggatctgctggaagcaccggatccgtgtacactggaaacctttctgggtcgtattccgatggtttttaatgtggttattctgagtccgcatggttattttgcacaggataatgttctgggttatcctgataccggtggtcaggttgtttatattctggatcaggttcgtgcactggaaaatgagatgctgcatcgtattaaacagcaaggcctggatattgttccgcgtattctgattattacccgtctgctgccggatgcagttggcaccacctgtggtcagcgtctggaaaaagtttttggcaccgaacatagccatattctgcgtgtgccgtttcgtaccgaaaaaggtattgttcgtaaatggattagccgctttgaagtttggccgtatctggaaacatataccgaagatgttgcacatgaactggcaaaagagctgcagggtaaaccggatctgattgttggtaattatagcgacggtaatattgttgcaagcctgctggcacataaactgggtgttacccagtgtaccattgcacatgccctggaaaaaaccaaatatccggaaagcgatatctactggaagaagctggaagaacgttatcattttagctgtcagtttaccgcagacctgtttgcaatgaatcataccgattttatcatcaccagcacctttcaagagattgcaggtagcaaagataccgtgggtcagtatgaaagccataccgcatttacactgcctggtctgtatcgtgttgttcatggtattgatgtgttcgacccgaaatttaacattgttagtccgggtgcagatcagaccatctattttccgcataccgaaaccagccgtcgcctgaccagctttcatccggaaattgaggaactgctgtatagcagcgttgaaaacgaagaacatatttgcgttctgaaagatcgtagcaaaccgatcatttttaccatggcacgcctggatcgtgttaaaaacattaccggtctggttgaatggtatggcaaaaatgcaaaactgcgcgaactggttaatctggttgtggttgccggtgatcgtcgtaaagaaagtaaagatctggaagaaaaagccgaaatgaagaaaatgtatggcctgatcgaaacctataaactgaatggccagtttcgttggattagcagccagatgaatcgtgttcgtaatggtgaactgtatcgcgttatttgtgatacccgtggtgcctttgttcagcctgccgtttatgaagcctttggtctgaccgttgtggaagcaatgacctgcggtctgccgacctttgcaacctgtaatggtggtccggcagaaattattgtgcatggtaaatccggttttcacatcgatccgtatcatggtgatcgtgcagcagacctgctggttgatttttttgaaaaatgtaaactggatccgacgcactgggataaaatcagcaaagccggtctgcagcgcattgaagagaaatatacctggcagatttatagccagcgtctgctgaccctgacaggtgtttatggtttttggaaacatgtgagcaatctggatcgtcgtgaatcacgtcgttacctggaaatgttttatgccctgaaatatcgcaaactggcagaaagcgttccgctggcagcagaataa

SEQ ID NO: 211 - protein, 339 aa, B. subtillis
MAILVTGGAGYIGSHTCVELLNSGYEIVVLDNLSNSSAEALNRVKEITGKDLTFYEADLLDREAVDSVFAENEIEAVIHFAGLKAVGESVAIPLKYYHNNLTGTFILCEAMEKYGVKKIVFSSSATVYGVPETSPITEDFPLGATNPYGQTKLMLEQILRDLHTADNEWSVALLRYFNPFGAHPSGRIGEDPNGIPNNLMPYVAQVAVGKLEQLSVFGNDYPTKDGTGVRDYIHVVDLAEGHVKALEKVLNSTGADAYNLGTGTGYSVLEMVKAFEKVSGKEVPYRFADRRPGDIATCFADPAKAKRELGWEAKRGLEEMCADSWRWQSSNVNGYKSAE

SEQ ID NO: 212 - DNA, 1020 bp, B. subtillis
atggcaatacttgttactggcggtgccggttacattggcagccacacatgtgttgaactattgaacagcggctacgagattgttgttcttgataatctgtccaacagttcagctgaagcgctgaaccgtgtcaaggagattacaggaaaagatttaacgttctacgaagcggatttattggaccgggaagcggtagattccgtttttgctgaaaatgaaatcgaagctgtgattcattttgcagggttaaaagcagtcggcgaatctgtggcgattcccctcaaatattatcataacaatttgacaggaacgtttattttatgcgaggccatggagaaatacggcgtcaagaaaatcgtattcagttcatctgcgacagtatacggcgttccggaaacatcgccgattacggaagactttccattaggcgcgacaaatccttatgggcagacgaagctcatgcttgaacaaatattgcgtgatttgcatacagccgacaatgagtggagcgttgcgctgcttcgttactttaacccgttcggcgcgcatccaagcggacggatcggtgaagacccgaacggaatcccaaataaccttatgccgtatgtggcacaggtagcagtcgggaagctcgagcaattaagcgtattcggaaatgactatccgacaaaagacgggacaggcgtacgcgattatattcacgtcgttgatctcgcagaaggccacgtcaaggcgctggaaaaagtattgaactctacaggagccgatgcatacaaccttggaacaggcacaggctacagcgtgctggaaatggtcaaagcctttgaaaaagtgtcagggaaagaggttccataccgttttgcggaccgccgtccgggagacatcgccacatgctttgcagatcctgcgaaagccaagcgagaactaggctgggaagcgaaacgcggccttgaggaaatgtgtgctgattcctggagatggcagtcttctaatgtgaatgggtataagagtgcggaataa

SEQ ID NO: 213 - protein, 342 aa, A. thaliana
MAATSEKQNTTKPPPSPSPLRNSKFCQPNMRILISGGAGFIGSHLVDKLMENEKNEVVVADNYFTGSKENLKKWIGHPRFELIRHDVTEPLLIEVDRIYHLACPASPIFYKYNPVKTIKTNVIGTLNMLGLAKRVGARILLTSTSEVYGDPLIHPQPESYWGNVNPIGVRSCYDEGKRVAETLMFDYHRQHGIEIRIARIFNTYGPRMNIDDGRVVSNFIAQALRGEALTVQKPGTQTRSFCYVSDMVDGLIRLMEGNDTGPINIGNPGEFTMVELAETVKELINPSIEIKMVENTPDDPRQRKPDISKAKEVLGWEPKVKLREGLPLMEEDFRLRLNVPRN

SEQ ID NO: 214 - DNA, 1029 bp, A. thaliana
atggcagctacaagtgagaaacagaacaccacaaagcctcctccttctccttctcctctccgcaattccaagttttgtcagcccaatatgaggatcttgatctctggaggagctggcttcattggttctcacttggttgataagcttatggaaaatgagaagaatgaggtggttgttgctgataactatttcactggctcaaaagaaaacctcaagaagtggatcggtcaccccaggtttgaacttattcgtcacgatgttaccgagcctttgttgatcgaggttgatcggatttaccatcttgcttgtcctgcctctcctatcttctacaaatacaaccctgttaagacaatcaagaccaatgtgattggtacactcaacatgctcggtcttgccaagcgtgttggagcaagaattttactaacctcaacctctgaagtgtatggagatcctctcatccaccctcaaccagagagctactggggaaatgtcaaccctattggggttcggagttgctatgacgaaggcaagcgggtagccgaaaccttgatgtttgactaccacagacaacatggcattgaaatccgcattgctagaatcttcaacacatatggtcctcgaatgaacatcgatgatgggcgtgttgtgagcaacttcattgctcaagcactccggggtgaggcattgacagttcagaaaccggggacacagacccgcagtttctgttatgtctccgacatggtggatggacttatccgtcttatggaaggcaatgatactggccctatcaacatcggtaacccaggtgagttcacaatggtggaactggctgagacggttaaggagcttattaacccaagcatagagataaagatggtggagaacacaccagatgatccaagacagaggaaaccagacattagtaaagccaaagaagtgttgggttgggagccaaaggtgaagctcagagaaggacttcctctcatggaagaagatttccgactaaggcttaacgtcccaagaaactaa

SEQ ID NO: 215 - protein, 297 aa, A. thaliana
TPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 216 - DNA, 894 bp, A. thaliana
acacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 217 - protein, 370 aa, A. thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVV

SEQ ID NO: 218 - DNA, 1113 bp, A. thaliana
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtataa

SEQ ID NO: 219 - protein, 667 aa, A. thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 220 - DNA, 2004 bp, A. thaliana
atggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 221 - protein, 481 aa, A. thaliana
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKAADLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 222 - DNA, 1446 bp, A. thaliana
atggtgaaaatttgttgtattggcgcaggttatgttggtggtccgaccatggcagttatggcactgaaatgtccggaaattgaagttgttgttgtggatattagcgaaccgcgtattaatgcatggaatagcgatcgtctgccgatttatgaacctggtctggaagatgttgttaaacagtgtcgtggtaaaaacctgttttttagcaccgatgtggaaaagcatgtgtttgaaagcgatattgttttcgtgagcgttaataccccgaccaaaacacaaggtttaggtgcaggtaaagcagccgatctgacctattgggaaagcgcagcacgtatgattgcagatgttagcaaaagcagcaaaatcgtggttgaaaaaagcaccgttccggttcgtaccgcagaagcaattgaaaaaattctgacccataacagcaaaggcatcgaatttcagattctgagcaatccggaatttctggcagaaggcaccgcaattaaagatctgtataatccggatcgtgttctgattggtggtcgtgataccgcagcaggtcagaaagccattaaagcactgcgtgatgtttatgcacattgggttccagttgagcagattatttgtaccaatctgtggtcagcagaactgagcaaactggcagcaaatgcctttctggcacagcgtattagcagcgttaatgcaatgagcgcactgtgtgaagcaaccggtgccgatgttacccaggttgcacatgcagttggtacagatacccgtattggtccgaaatttctgaatgcaagcgttggttttggtggtagctgttttcagaaagatattctgaacctgatctacatctgcgaatgtaatggtctgccggaagcagccaattattggaaacaggttgttaaagtgaacgattaccagaaaattcgctttgccaatcgtgttgttagcagcatgtttaataccgtgagcggcaaaaaaatcgccattctgggttttgccttcaaaaaagataccggtgatacccgtgaaacaccggcaattgatgtttgtaatcgtctggttgcagataaagccaaactgagcatttatgatccgcaggttctggaagaacaaattcgtcgtgatctgagcatggcacgttttgattgggatcatccggttccgctgcagcagattaaagcagaaggtatttcagaacaggtgaacgttgttagtgatgcatatgaagccaccaaagatgcacatggtctgtgtgttctgaccgaatgggatgaattcaaaagcctggatttcaaaaagatcttcgataacatgcagaaaccggcatttgtttttgatggtcgtaatgttgttgatgccgttaaactgcgtgaaatcggctttattgtttacagcattggtaaaccgctggatagctggctgaaagatatgcctgcagttgcataa

SEQ ID NO: 223 - protein, 419 aa, A. thaliana
MFSFGRARSQGRQNRSMSLGGLDYADPKKKNNYLGKILLTASLTALCIFMLKQSPTFNTPSVFSRHEPGVTHVLVTGGAGYIGSHAALRLLKESYRVTIVDNLSRGNLAAVRILQELFPEPGRLQFIYADLGDAKAVNKIFTENAFDAVMHFAAVAYVGESTQFPLKYYHNITSNTLVVLETMAAHGVKTLIYSSTCATYGEPDIMPITEETPQVPINPYGKAKKMAEDIILDFSKNSDMAVMILRYFNVIGSDPEGRLGEAPRPELREHGRISGACFDAARGIMPGLQIKGTDYKTADGTCVRDYIDVTDLVDAHVKALQKAKPRKVGIYNVGTGKGSSVKEFVEACKKATGVEIKIDYLPRRAGDYAEVYSDPSKIRKELNWTAKHTNLKESLETAWRWQKLHRNGYGLTTSSVSVY

SEQ ID NO: 224 - DNA, 1260 bp, A. thaliana
atgtttagctttggtcgtgcacgtagccagggtcgtcagaatcgtagcatgagcttaggtggtctggattatgcagatccgaaaaagaaaaataactatctgggcaaaattctgctgaccgcaagcctgaccgcactgtgcatttttatgctgaaacagagcccgacctttaataccccgagcgtttttagccgtcatgaaccgggtgttacccatgttctggttaccggtggtgcaggttatattggtagccatgcagcactgcgtctgctgaaagaaagctatcgtgttaccattgttgataatctgagccgtggtaatctggcagcagttcgtattctgcaagaactgtttccggaaccgggtcgtctgcagtttatctatgccgatctgggtgatgcaaaagccgtgaataaaatctttaccgaaaatgcctttgatgccgtgatgcattttgcagcagttgcatatgttggtgaaagcacccagtttccgctgaaatattaccataacattaccagcaataccctggttgttctggaaaccatggcagcacatggtgttaaaaccctgatttatagcagcacctgtgcaacctatggtgaaccggatattatgccgattaccgaagaaacaccgcaggttccgattaatccgtatggtaaagccaaaaaaatggccgaagatatcatcctggatttcagcaaaaatagcgatatggccgttatgattctgcgctattttaacgtgattggtagcgatccggaaggtcgtctgggtgaagcaccgcgtccggaactgcgtgaacatggtcgtattagcggtgcatgttttgatgcagcacgtggtattatgcctggtctgcagattaaaggcaccgattacaaaaccgcagatggcacctgtgttcgtgattatattgatgttaccgatctggtggatgcccatgttaaagcactgcagaaagcaaaaccgcgtaaagtgggtatctataatgttggcaccggtaaaggtagcagcgttaaagaatttgttgaggcctgtaaaaaagccaccggtgtggaaatcaaaatcgattatctgcctcgtcgtgccggtgattatgcggaagtttatagtgatccgagcaaaattcgcaaagaactgaattggaccgccaaacataccaacctgaaagaatcactggaaaccgcatggcgttggcagaaactgcatcgtaatggttatggcctgaccaccagtagcgttagcgtttattaa

SEQ ID NO: 225 - protein, 345 aa, P. shigelloides
MDIYMSRYEEITQQLIFSPKTWLITGVAGFIGSNLLEKLLKLNQVVIGLDNFSTGHQYNLDEVKTLVSTEQWSRFCFIEGDIRDLTTCEQVMKGVDHVLHQAALGSVPRSIVDPITTNATNITGFLNILHAAKNAQVQSFTYAASSSTYGDHPALPKVEENIGNPLSPYAVTKYVNEIYAQVYARTYGFKTIGLRYFNVFGRRQDPNGAYAAVIPKWTAAMLKGDDVYINGDGETSRDFCYIDNVIQMNILSALAKDSAKDNIYNVAVGDRTTLNELSGYIYDELNLIHHIDKLSIKYREFRSGDVRHSQADVTKAIDLLKYRPNIKIREGLRLSMPWYVRFLKG

SEQ ID NO: 226 - DNA, 1038 bp, P. shigelloides
atggacatttatatgagccgctatgaagaaattacccagcagctgatttttagcccgaaaacctggctgattaccggtgttgcaggttttattggtagcaatctgctggaaaaactgctgaaactgaatcaggttgtgattggcctggataatttcagcaccggtcatcagtataatctggatgaagttaaaaccctggttagcaccgaacagtggtcacgtttttgttttattgaaggcgatattcgtgatctgaccacctgtgaacaggttatgaaaggtgttgatcatgttctgcatcaggcagcactgggtagcgttccgcgtagcattgttgatccgattaccaccaatgcaaccaatattaccggctttctgaatattctgcatgccgcaaaaaatgcacaggttcagagctttacctatgcagcaagcagcagcacctatggtgatcatccggcactgccgaaagttgaagaaaatattggtaatccgctgagcccgtatgcagttaccaaatatgtgaatgaaatttatgcccaggtttacgcacgtacctatggctttaaaaccattggtctgcgctatttcaatgtgtttggtcgtcgtcaggatccgaatggtgcatatgccgcagttattccgaaatggaccgcagcaatgctgaaaggtgatgacgtttatatcaatggtgatggtgaaaccagccgtgatttttgctatattgataacgtgatccagatgaacattctgagcgcactggcaaaagatagcgccaaagataacatttataacgttgcagttggtgatcgtaccacactgaatgaactgagcggttatatctatgatgaactgaacctgatccaccacattgataaactgagcatcaaatatcgcgaatttcgtagcggtgatgttcgtcatagccaggcagatgttaccaaagcaattgatctgctgaaatatcgtccgaacattaaaatccgtgaaggtctgcgtctgagcatgccgtggtatgttcgttttctgaaaggttaa

SEQ ID NO: 227 - protein, 520 aa, artificial fusion construct
  Synthetic
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYAATSGSTGSTGSTGSGRSTGSTGSTGSGRSHMVAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKGS

SEQ ID NO: 228 - DNA, 1563 bp, artificial fusion construct
  Synthetic
atgaatcatttaagagctgaaggtccagcctccgttttggccatcggtaccgctaaccctgaaaacattttgttgcaagacgaattcccagactactacttcagagtcactaagtccgaacacatgacccaattgaaggagaagttcagaaagatttgtgacaagtccatgattagaaagagaaactgtttcttgaacgaagaacacttgaagcaaaacccaagattggttgaacatgaaatgcaaactttggacgctagacaagacatgttggttgttgaagtccctaagttgggtaaggatgcctgtgctaaggccattaaagaatggggtcaacctaagtccaagattacccacttgattttcacctctgcctccaccactgacatgcctggtgctgattaccactgcgctaagttattgggtttgtctccatccgttaagagagttatgatgtaccaattgggttgctacggtggtggtactgttttaagaattgctaaggatattgctgaaaacaacaagggtgccagagtcttagctgtctgctgtgacattatggcttgtttattcagaggtccatctgaatccgacttggaattgttggttggtcaagctatcttcggtgacggtgctgctgccgttattgttggtgctgaaccagacgaatccgttggtgaaagaccaatttttgaattggtttccaccggtcaaactattttgccaaattccgaaggtaccatcggtggtcatatcagagaagccggtttgatcttcgacttacataaggatgtcccaatgttgatctctaacaacattgaaaagtgtttgatcgaagcttttaccccaattggtatttctgactggaactctatcttctggattacccatcctggtggtaaggctattttggataaggtcgaggaaaaattgcacttgaagtctgacaagttcgttgactctagacacgtcttgtccgaacatggtaatatgtcctcttccaccgttttattcgttatggatgagttgagaaagagatccttagaagaaggtaagtccaccaccggtgatggttttgagtggggtgttttgttcggtttcggtccaggtttgaccgtcgaaagagttgttgttagatctgtcccaattaagtacgcagccacaagcggttctacgggctccacgggctctaccggcagtgggaggagcactgggtcaacgggatcaacaggtagtggaagatcacacatggttgccgtcaagcacttgatcgttttgaagttcaaggatgaaatcactgaagctcaaaaggaagaattcttcaaaacctacgtcaacttagtcaatattattccagccatgaaggacgtctattggggtaaggacgttactcaaaagaataaggaggaaggttatactcatatcgttgaggtcactttcgaatctgttgagactattcaagactacatcatccacccagcccacgttggtttcggtgatgtttatcgttccttctgggaaaaattgttgatcttcgactacacccctagaaagggatcctaa

SEQ ID NO: 229 - protein, 381 aa, A. Grandis
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 230 - DNA, 1146 bp, A. Grandis
atggcttactctgctatggctactatgggttataatggtatggctgcttcttgtcataccttgcatccaacttctccattgaaaccatttcatggtgcttccacatctttggaagcttttaatggtgaacacatgggtttgttgagaggttactctaagagaaagctgtcctcttacaaaaacccagcttctagatcttctaacgctaccgttgctcaattattgaatccaccacaaaaaggtaagaaggccgttgaatttgacttcaacaagtacatggattccaaggctatgactgttaacgaagctttgaacaaggctatcccattgagatacccacaaaagatctacgaatctatgaggtactctttgttggctggtggtaaaagggttagaccagttttgtgtattgctgcttgtgaattggttggtggtactgaagaattggctattccaactgcttgtgccattgaaatgattcacactatgtccttgatgcacgatgatttgccatgcattgataacgatgacttgagaagaggtaagccaactaaccataagatcttcggtgaagatactgctgttactgctggtaatgctttacattcttacgccttcgaacatattgctgtctctacttctaaaaccgttggtgccgatagaatcttgagaatggtttctgaattgggtagagctactggttctgaaggtgttatgggtggtcaaatggttgatattgcttcagaaggtgatccatccattgacttgcaaactttggaatggattcatatccataagaccgccatgttgttggaatgttctgttgtttgtggtgctattattggtggtgcttctgaaatcgttattgaaagagctagaagatacgctagatgcgttggtttgttgttccaagttgttgatgatatcctggatgtcaccaagtcatctgatgaattaggtaaaaccgctggtaaggatttgatttctgataaggctacttacccaaagttgatgggtttagaaaaggccaaagaattctccgatgagttgttgaatagagccaaaggtgaattgtcttgtttcgatccagttaaggctgctccattattgggtttagctgattacgttgctttcaggcaaaactaa

SEQ ID NO: 231 - protein, 541 aa, Artificial
  Synthetic
MIFDGTTMSIAIGLLSTLGIGAEANPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 232 - DNA, 1626 bp, Artificial
  Synthetic
atgattttcgatgggaccacgatgtccattgcgatagggctactttcaacgctgggcataggcgcagaagcgaacccgcaagaaaactttctaaaatgcttttctgaatacattcctaacaaccctgccaacccgaagtttatctacacacaacacgatcaattgtatatgagcgtgttgaatagtacaatacagaacctgaggtttacatccgacacaacgccgaaaccgctagtgatcgtcacaccctccaacgtaagccacattcaggcaagcattttatgcagcaagaaagtcggactgcagataaggacgaggtccggaggacacgacgccgaagggatgagctatatctcccaggtaccttttgtggtggtagacttgagaaatatgcactctatcaagatagacgttcactcccaaaccgcttgggttgaggcgggagccacccttggtgaggtctactactggatcaacgaaaagaatgaaaattttagctttcctgggggatattgcccaactgtaggtgttggcggccacttctcaggaggcggttatggggccttgatgcgtaactacggacttgcggccgacaacattatagacgcacatctagtgaatgtagacggcaaagttttagacaggaagagcatgggtgaggatcttttttgggcaattagaggcggagggggagaaaattttggaattatcgctgcttggaaaattaagctagttgcggtaccgagcaaaagcactatattctctgtaaaaaagaacatggagatacatggtttggtgaagctttttaataagtggcaaaacatcgcgtacaagtacgacaaagatctggttctgatgacgcattttataacgaaaaatatcaccgacaaccacggaaaaaacaaaaccacagtacatggctacttctctagtatatttcatgggggagtcgattctctggttgatttaatgaacaaatcattcccagagttgggtataaagaagacagactgtaaggagttctcttggattgacacaactatattctattcaggcgtagtcaactttaacacggcgaatttcaaaaaagagatccttctggacagatccgcaggtaagaaaactgcgttctctatcaaattggactatgtgaagaagcctattcccgaaaccgcgatggtcaagatacttgagaaattatacgaggaagatgtgggagttggaatgtacgtactttatccctatggtgggataatggaagaaatcagcgagagcgccattccatttccccatcgtgccggcatcatgtacgagctgtggtatactgcgagttgggagaagcaagaagacaacgaaaagcacattaactgggtcagatcagtttacaatttcaccaccccatacgtgtcccagaatccgcgtctggcttacttgaactaccgtgatcttgacctgggtaaaacgaacccggagtcacccaacaattacactcaagctagaatctggggagagaaatactttgggaagaacttcaacaggttagtaaaggttaaaaccaaggcagatccaaacaacttttttagaaatgaacaatccattcccccgctacccccgcaccatcactaa

SEQ ID NO: 233 - protein, 540 aa, Artificial
  Synthetic
MIFDGTTMSIAIGLLSTLGIGAEANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 234 - DNA, 1623 bp, Artificial
  Synthetic
atgatcttcgacggcacaaccatgagtatcgccattggtttgcttagcaccctgggaataggggcagaagcgaatccaagagaaaatttcttgaagtgtttttctcagtatatcccgaataatgcgacgaaccttaagttagtatacactcagaacaaccctctatatatgagcgttctaaattctacaatccacaacctaagatttacgtccgacacgactccgaaacccctagttatagtgacaccgtcacatgttagccatatacagggcaccatactatgttccaaaaaagttgggttacaaatacgtacccgtagcgggggacacgacagtgaggggatgagttatattagtcaggtgcctttcgtcatagtggatttaagaaatatgaggtcaattaaaatcgacgttcactcacaaactgcctgggttgaggcgggggccacattgggtgaagtatattactgggtcaatgagaagaacgagaatctttcactagcagccggttattgtcccacagtctgcgccggcggtcactttggcggcggcggatacggtcccttaatgagaaattacgggcttgccgcagacaatatcatagatgctcacttagttaatgttcatggaaaagtgttagaccgtaaaagcatgggggaggatctgttttgggcgcttagagggggaggggcagaatcatttggaataatagtggcatggaaaatcaggcttgtggctgttccaaagagtaccatgttctcagtaaagaaaataatggagatccatgagctagttaaacttgtgaataaatggcaaaacatagcctataaatatgataaggacttgctgcttatgactcatttcataaccagaaacattacggataaccaagggaagaacaaaacagccatccatacctactttagctccgttttcttgggtggtgtagacagcttagttgacctgatgaacaagagttttccggaactaggtatcaagaagacagattgtagacaactttcctggattgataccataatcttttacagcggagtcgtcaattatgacactgacaacttcaacaaggaaattttattagataggagtgcgggtcaaaatggggccttcaagatcaaactagactacgttaaaaaacccattcctgaaagtgtttttgttcagattctggagaagctgtatgaagaagatattggcgcggggatgtacgctctttatccgtacggcggcataatggatgagattagtgaaagcgccatccctttcccccacagagctggtatcctgtacgagttgtggtatatctgctcctgggagaaacaggaggataacgaaaagcacttaaattggattaggaatatctacaatttcatgacgccctacgtttccaagaaccccaggttggcctatttgaactacagggatcttgatattggaatcaacgaccccaaaaacccaaacaactacacccaggcaaggatttggggagagaagtacttcgggaagaacttcgacaggctagttaaggtgaaaacgctagttgatccaaataattttttcagaaacgaacagagtatccctcccttaccgcgtcataggcactaa

SEQ ID NO: 235 - protein, 323 aa, Artificial
  Synthetic
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 236 - DNA, 972 bp, Artificial
  Synthetic
atgtctgctggctctgaccaaattgaaggttccccgcatcacgaatcagataatagtattgccacaaagatcttaaactttgggcatacatgttggaaattacaaaggccctacgtcgtcaaaggaatgataagcatcgcttgcggtctgttcggaagggaattatttaacaataggcatctattcagctgggggttaatgtggaaagctttcttcgcgttagtgccaatcctaagctttaactttttcgccgccatcatgaaccagatttatgatgttgatatcgacaggataaataagccagatcttccattggtatccggtgaaatgtcaatagaaactgcatggatattatctattatcgttgcgctgaccggactgatagtaacaatcaaattgaaatctgcacccctgtttgtttttatatatatatttggtattttcgctggattcgcttactcagtgccacctatcaggtggaagcagtacccattcacgaattttctgatcacgatctctagccacgtcgggttagcgttcacatcttactctgcaaccacgagtgccttggggcttcctttcgtctggcgtccagcttttagttttatcattgcctttatgaccgtaatgggaatgacgatcgcattcgcaaaggacatttctgacatagagggggatgcaaaatacggtgtctccactgtggcgacaaaattaggagctaggaatatgactttcgtggtgtccggtgtattattactaaattatctggtatctataagtatcggcatcatatggccgcaagtgtttaaatccaacattatgatactgagtcatgctattttggctttttgtctgatttttcagacgcgtgagttggcgcttgcaaactatgcctctgcgcccagcaggcagttttttgaattcatatggttattgtactatgccgagtatttcgtctacgtatttatttaa

SEQ ID NO: 237 - protein, 305 aa, Artificial
  Synthetic
MSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRFLLKAFDALED

SEQ ID NO: 238 - DNA, 918 bp, Artificial
  Synthetic
atgtctggtgctgctgatgttgaaagggtttatgctgctatggaagaagctgctggtttgttggatgtttcttgtgctagagaaaagatctaccctttgttgaccgttttccaagatactttgactgatggtgttgtcgttttctctatggcttctggtagaagatctactgaattggacttctccatttccgttccagtttctcaaggtgatccatatgctactgttgtcaaagaaggtttgtttcaagctactggttctccagttgatgaattattggctgatactgttgctcacttgccagtttctatgtttgctattgatggtgaagttaccggtggtttcaaaaagacttacgcttttttcccaaccgatgatatgccaggtgttgctcaattggctgctattccatctatgccagcttcagttgctgaaaacgctgaattatttgccagatacggtttggataaggtccaaatgacttccatggattacaagaagagacaggtcaacttgtacttctccgatttgaagcaagaatacttgcaaccagaatccgttgttgctttggctagagaattgggtttgagagttccaggtgaattaggtttggaattctgcaagagatctttcgctgtttacccaactttgaattgggataccggtaagattgatagattgtgctttgctgctatttccaccgatccaactttggttccatctgaagatgaacgtgatatcgagatgtttagaaactacgctactaaggctccatacgcttatgttggtgagaaaagaacattggtttacggcttgactttgtcctctaccgaagaatattacaagttgggtgcctactaccatatcaccgatattcaaagattcttgctgaaggctttcgatgccttggaagattaa

SEQ ID NO: 239 - protein, 722 aa, C. Sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFEGS

SEQ ID NO: 240 - DNA, 2169 bp, C. Sativa
atgggtaagaattacaaatccttggattctgttgttgcttctgacttcatcgctttgggtatcacttccgaggtcgctgaaaccttacacggtcgtttggctgaaattgtttgtaactacggtgctgctaccccacaaacctggattaacatcgctaatcatattttgtctccagatttgccattttctttgcatcaaatgttgttctacggttgttataaggatttcggtccagctcctccagcttggattccagatccagaaaaggttaagtccactaacttgggtgccttattggaaaaaagaggtaaggaattcttaggtgttaaatacaaagacccaatctcttctttctctcacttccaagaattctctgttagaaacccagaagtttactggagaaccgttttaatggacgagatgaagatctccttttccaaggatccagaatgtatcttaagacgtgatgatattaataacccaggtggttccgaatggttgccaggtggttacttgaactccgctaagaactgcttgaacgttaattccaacaagaagttaaacgacactatgatcgtttggagggacgaaggtaacgatgacttgcctttgaacaaattaactttggaccaattaagaaagagagtctggttggttggttacgctttggaagaaatgggtttggaaaaaggttgtgccattgctatcgacatgccaatgcacgtcgacgctgtcgttatttacttggctattgtcttggctggttacgttgttgtttctatcgccgactccttctccgccccagaaatttccactagattgagattgtctaaggctaaggccatttttacccaagatcatatcattcgtggtaagaagcgtattccattatactctagagtcgttgaagctaagtctccaatggccattgttattccatgctctggttccaatatcggtgccgaattgagggacggtgatatctcttgggactattttttggaaagagctaaagaatttaagaactgcgaattcaccgccagagaacaaccagttgacgcttacactaacatcttattctcttctggtaccaccggtgaaccaaaagctattccatggacccaagctactcctttgaaagccgctgctgatggttggtcccacttagatattagaaagggtgacgttattgtttggccaaccaacttgggttggatgatgggtccatggttggtttatgcttccttgttgaatggtgcctccatcgctttgtacaacggttctccattggtttccggttttgctaagtttgttcaagatgctaaggtcactatgttaggtgttgttccttctatcgtcagatcctggaaatctactaactgtgtttctggttacgattggtctactatccgttgcttctcctcttccggtgaagcttctaacgttgacgaatatttatggttgatgggtagagccaattataagcctgtcattgaaatgtgtggtggtactgagattggtggtgctttctccgctggttccttcttgcaagctcaatctttgtcctctttttcttctcaatgtatgggttgcactttgtacatcttggataagaatggttacccaatgccaaagaataaaccaggtattggtgaattggccttgggtccagttatgttcggtgcttccaagactttattgaacggtaaccaccatgatgtttactttaagggtatgcctactttgaacggtgaagttttgagaagacacggtgacattttcgaattaacttccaacggttactaccatgctcacggtagagctgatgataccatgaacatcggtggtatcaagatctcttccattgaaatcgagcgtgtttgtaacgaagttgacgacagagttttcgaaactactgccatcggtgtcccacctttgggtggtggtcctgaacaattggtcattttcttcgtcttgaaggattctaacgataccaccatcgacttgaaccaattgagattgtctttcaacttgggtttgcaaaagaagttgaacccattgttcaaagtcaccagagttgttccattgtcctccttgccacgtaccgccactaacaagattatgagaagagtcttgagacaacaattttctcatttcgagggatcctaa

SEQ ID NO: 241 - DNA, 39 bp, Artificial
  Synthetic primer sequence
atctgtcauaaaacaatgtctgactctggtggtttcgac

SEQ ID NO: 242 - DNA, 34 bp, Artificial
  Synthetic primer sequence
cacgcgauctagtgagtgttgttgttacacttcc

SEQ ID NO: 243 - DNA, 39 bp, Artificial; identical to SEQ ID NO: 241
  Synthetic primer sequence

SEQ ID NO: 244 - DNA, 34 bp, Artificial; identical to SEQ ID NO: 242
  Synthetic primer sequence

SEQ ID NO: 245 - DNA, 39 bp, Artificial; identical to SEQ ID NO: 241
  Synthetic primer sequence

SEQ ID NO: 246 - DNA, 34 bp, Artificial; identical to SEQ ID NO: 242
  Synthetic primer sequence

SEQ ID NO: 247 - DNA, 41 bp, Artificial
  Synthetic primer sequence
atctgtcauaaaacaatgccatcttctggtgacgctgctgg

SEQ ID NO: 248 - DNA, 32 bp, Artificial
  Synthetic primer sequence
cacgcgauctagttagttctacaagtaccacc

SEQ ID NO: 249 - DNA, 38 bp, Artificial
  Synthetic primer sequence
atctgtcauaaaacaatgatgggtgacttgactacttc

SEQ ID NO: 250 - DNA, 30 bp, Artificial
  Synthetic primer sequence
cacgcgauctatctcttcaaagaaccgatg

SEQ ID NO: 251 - DNA, 37 bp, Artificial
  Synthetic primer sequence
atctgtcauaaaacaatgtcttcttctgaaggtgttg

SEQ ID NO: 252 - DNA, 30 bp, Artificial
  Synthetic primer sequence
cacgcgauctagttagcttgagcgtttctc

SEQ ID NO: 253 - DNA, 37 bp, Artificial
  Synthetic primer sequence
atctgtcauaaaacaatggctgctaacggtggtgacc

SEQ ID NO: 254 - DNA, 31 bp, Artificial
  Synthetic primer sequence
cacgcgauctactttctttcagcgtctctac

SEQ ID NO: 255 - DNA, 36 bp, Artificial
  Synthetic primer sequence
atctgtcauaaaacaatgtctgcttctgacgctttg

SEQ ID NO: 256 - DNA, 34 bp, Artificial
  Synthetic primer sequence
cacgcgauctaagtctttctagaagtcttcttcc

SEQ ID NO: 257 - DNA, 37 bp, Artificial
  Synthetic primer sequence
atctgtcauaaaacaatgggttctttgactaacaacg

SEQ ID NO: 258 - DNA, 32 bp, Artificial
  Synthetic primer sequence
cacgcgauctacttagtaccagtctttctagc

SEQ ID NO: 259 - DNA, 40 bp, Artificial
  Synthetic primer sequence
atctgtcauaaaacaatggaattcagattgttgatcttgg

SEQ ID NO: 260 - DNA, 31 bp, Artificial
  Synthetic primer sequence
cacgcgauctagttcttcttcaacttttcag

SEQ ID NO: 261 - DNA, 39 bp, Artificial
  Synthetic primer sequence
atctgtcauaaaacaatgactttgttgagagacttgttg

SEQ ID NO: 262 - DNA, 30 bp, Artificial
  Synthetic primer sequence
cacgcgauctacttagtcaacattctgaag

SEQ ID NO: 263 - DNA, 38 bp, Artificial
  Synthetic primer sequence
atctgtcauaaaacaatgatcttcttctacttcttgac

SEQ ID NO: 264 - DNA, 31 bp, Artificial
  Synthetic primer sequence
cacgcgauctagttgtccttaaccttcttag

SEQ ID NO: 265 - DNA, 38 bp, Artificial
  Synthetic primer sequence
atctgtcauaaaacaatgaacagagaagtttctgaaag

SEQ ID NO: 266 - DNA, 33 bp, Artificial
  Synthetic primer sequence
cacgcgauctactttctaccgttcaattcttcc

SEQ ID NO: 267 - DNA, 38 bp, Artificial
  Synthetic primer sequence
atctgtcauaaaacaatggaaaagtctaacggtttgag

SEQ ID NO: 268 - DNA, 30 bp, Artificial
  Synthetic primer sequence
cacgcgauctagaaagaagagatgtagtcg

SEQ ID NO: 269 - DNA, 39 bp, Artificial
  Synthetic primer sequence
atctgtcauaaaacaatgtcttctgacccacacagaaag

SEQ ID NO: 270 - DNA, 30 bp, Artificial
  Synthetic primer sequence
cacgcgauctaagaagtgaattcttcgatg

SEQ ID NO: 271 - DNA, 39 bp, Artificial
  Synthetic primer sequence
atctgtcauaaaacaatgtctacttctgaattggttttc

SEQ ID NO: 272 - DNA, 30 bp, Artificial
  Synthetic primer sequence
cacgcgauctagatagtaacgttagaaacg

SEQ ID NO: 273 - DNA, 39 bp, Artificial
  Synthetic primer sequence
atctgtcauaaaacaatgaagcaaactgttgttttgtac

SEQ ID NO: 274 - DNA, 32 bp, Artificial
  Synthetic primer sequence
cacgcgauctagttttgaaccaagttttcaac

SEQ ID NO: 275 - DNA, 35 bp, Artificial
  Synthetic primer sequence
atctgtcauaaaacaatggctagagctggttggac

SEQ ID NO: 276 - DNA, 32 bp, Artificial
  Synthetic primer sequence
cacgcgauctagtgagtcttagacttgtgagc

SEQ ID NO: 277 - DNA, 38 bp, Artificial
  Synthetic primer sequence
atctgtcauaaaacaatggcttgtactggttggacttc

SEQ ID NO: 278 - DNA, 32 bp, Artificial; identical to SEQ ID NO: 276
  Synthetic primer sequence

SEQ ID NO: 279 - DNA, 35 bp, Artificial
  Synthetic primer sequence
atctgtcauaaaacaatgtctgttaagtggacttc

SEQ ID NO: 280 - DNA, 31 bp, Artificial
  Synthetic primer sequence
cacgcgauctagtcgttcttacccttcttag

SEQ ID NO: 281 - DNA, 30 bp, Artificial
  Synthetic primer sequence
ggatccatgtctgactctggtggtttcgac

SEQ ID NO: 282 - DNA, 32 bp, Artificial
  Synthetic primer sequence
aagcttctagtgagtgttgttgttacacttcc

SEQ ID NO: 283 - DNA, 30 bp, Artificial; identical to SEQ ID NO: 281
  Synthetic primer sequence

SEQ ID NO: 284 - DNA, 32 bp, Artificial; identical to SEQ ID NO: 282
  Synthetic primer sequence

SEQ ID NO: 285 - DNA, 30 bp, Artificial; identical to SEQ ID NO: 281
  Synthetic primer sequence

SEQ ID NO: 286 - DNA, 32 bp, Artificial; identical to SEQ ID NO: 282
  Synthetic primer sequence

SEQ ID NO: 287 - DNA, 32 bp, Artificial
  Synthetic primer sequence
ggatccatgccatcttctggtgacgctgctgg

SEQ ID NO: 288 - DNA, 30 bp, Artificial
  Synthetic primer sequence
aagcttctagttagttctacaagtaccacc

SEQ ID NO: 289 - DNA, 29 bp, Artificial
  Synthetic primer sequence
ggatccatgatgggtgacttgactacttc

SEQ ID NO: 290 - DNA, 28 bp, Artificial
  Synthetic primer sequence
aagcttctatctcttcaaagaaccgatg

SEQ ID NO: 291 - DNA, 28 bp, Artificial
  Synthetic primer sequence
ggatccatgtcttcttctgaaggtgttg

SEQ ID NO: 292 - DNA, 28 bp, Artificial
  Synthetic primer sequence
aagcttctagttagcttgagcgtttctc

SEQ ID NO: 293 - DNA, 28 bp, Artificial
  Synthetic primer sequence
ggatccatggctgctaacggtggtgacc

SEQ ID NO: 294 - DNA, 29 bp, Artificial
  Synthetic primer sequence
aagcttctactttctttcagcgtctctac

SEQ ID NO: 295 - DNA, 27 bp, Artificial
  Synthetic primer sequence
ggatccatgtctgcttctgacgctttg

SEQ ID NO: 296 - DNA, 32 bp, Artificial
  Synthetic primer sequence
aagcttctaagtctttctagaagtcttcttcc

SEQ ID NO: 297 - DNA, 28 bp, Artificial
  Synthetic primer sequence
ggatccatgggttctttgactaacaacg

SEQ ID NO: 298 - DNA, 30 bp, Artificial
  Synthetic primer sequence
aagcttctacttagtaccagtctttctagc

SEQ ID NO: 299 - DNA, 31 bp, Artificial
  Synthetic primer sequence
ggatccatggaattcagattgttgatcttgg

SEQ ID NO: 300 - DNA, 29 bp, Artificial
  Synthetic primer sequence
aagcttctagttcttcttcaacttttcag

SEQ ID NO: 301 - DNA, 30 bp, Artificial
  Synthetic primer sequence
ggatccatgactttgttgagagacttgttg

SEQ ID NO: 302 - DNA, 28 bp, Artificial
  Synthetic primer sequence
aagcttctacttagtcaacattctgaag

SEQ ID NO: 303 - DNA, 29 bp, Artificial
  Synthetic primer sequence
ggatccatgatcttcttctacttcttgac

SEQ ID NO: 304 - DNA, 29 bp, Artificial
  Synthetic primer sequence
aagcttctagttgtccttaaccttcttag

SEQ ID NO: 305 - DNA, 29 bp, Artificial
  Synthetic primer sequence
ggatccatgaacagagaagtttctgaaag

SEQ ID NO: 306 - DNA, 31 bp, Artificial
  Synthetic primer sequence
aagcttctactttctaccgttcaattcttcc

SEQ ID NO: 307 - DNA, 29 bp, Artificial
  Synthetic primer sequence
ggatccatggaaaagtctaacggtttgag

SEQ ID NO: 308 - DNA, 28 bp, Artificial
  Synthetic primer sequence
aagcttctagaaagaagagatgtagtcg

SEQ ID NO: 309 - DNA, 30 bp, Artificial
  Synthetic primer sequence
ggatccatgtcttctgacccacacagaaag

SEQ ID NO: 310 - DNA, 28 bp, Artificial
  Synthetic primer sequence
aagcttctaagaagtgaattcttcgatg

SEQ ID NO: 311 - DNA, 30 bp, Artificial
  Synthetic primer sequence
ggatccatgtctacttctgaattggttttc

SEQ ID NO: 312 - DNA, 28 bp, Artificial
  Synthetic primer sequence
aagcttctagatagtaacgttagaaacg

SEQ ID NO: 313 - DNA, 30 bp, Artificial
  Synthetic primer sequence
ggatccatgaagcaaactgttgttttgtac

SEQ ID NO: 314 - DNA, 30 bp, Artificial
  Synthetic primer sequence
aagcttctagttttgaaccaagttttcaac

SEQ ID NO: 315 - DNA, 26 bp, Artificial
  Synthetic primer sequence
ggatccatggctagagctggttggac

SEQ ID NO: 316 - DNA, 30 bp, Artificial
  Synthetic primer sequence
aagcttctagtgagtcttagacttgtgagc

SEQ ID NO: 317 - DNA, 29 bp, Artificial
  Synthetic primer sequence
ggatccatggcttgtactggttggacttc

SEQ ID NO: 318 - DNA, 30 bp, Artificial; identical to SEQ ID NO: 316
  Synthetic primer sequence

SEQ ID NO: 319 - DNA, 26 bp, Artificial
  Synthetic primer sequence
ggatccatgtctgttaagtggacttc

SEQ ID NO: 320 - DNA, 29 bp, Artificial
  Synthetic primer sequence
aagcttctagtcgttcttacccttcttag